(19) United States
(12) Patent Application Publication
(10) Pub. No.: US 2013/0072470 A1
(43) Pub. Date: Mar. 21, 2013

(54) NOVEL TRICYCLIC COMPOUNDS

(71) Applicants: Neil Wishart, Jefferson, MA (US); Dominique F. Bonafoux, Winthrop, MA (US); Kristine E. Frank, Grayslake, IL (US); Adrian D. Hobson, Shrewsbury, MA (US); Donald B. Konopacki, Charlton, MA (US); Gloria Y. Martinez, Shrewsbury, MA (US); Lu Wang, Westborough, MA (US)

(72) Inventors: Neil Wishart, Jefferson, MA (US); Dominique F. Bonafoux, Winthrop, MA (US); Kristine E. Frank, Grayslake, IL (US); Adrian D. Hobson, Shrewsbury, MA (US); Donald B. Konopacki, Charlton, MA (US); Gloria Y. Martinez, Shrewsbury, MA (US); Lu Wang, Westborough, MA (US)

(73) Assignee: ABBVIE INC., North Chicago, IL (US)

(21) Appl. No.: 13/623,393

(22) Filed: Sep. 20, 2012

Related U.S. Application Data
(60) Provisional application No. 61/537,298, filed on Sep. 21, 2011.

(51) Int. Cl. A61K 31/4985; A61K 31/501; C07D 471/14; A61P 17/06; A61K 31/5377; A61P 29/00; A61P 19/02; A61P 27/04; C07D 487/14; A61K 31/437

(52) U.S. Cl. 514/210.21; 544/346; 514/250; 544/238; 544/295; 546/82; 514/293; 544/115; 514/233.2

(57) ABSTRACT

The invention provides compounds of Formula (I)

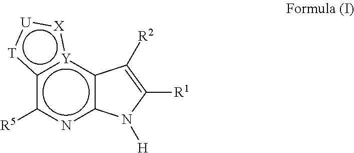

pharmaceutically acceptable salts, pro-drugs, biologically active metabolites, stereoisomers and isomers thereof wherein the variable are defined herein. The compounds of the invention are useful for treating immunological and oncological conditions.

### CROSS REFERENCE TO RELATED APPLICATION

This application claims priority to U.S. Provisional Application Ser. No. 61/537,298 filed on Sep. 21, 2011, the contents of which are incorporated herein.

### BACKGROUND OF THE INVENTION

The invention provides a novel class of compounds, pharmaceutical compositions comprising such compounds and methods of using such compounds to treat or prevent diseases or disorders associated with abnormal or deregulated kinase activity, particularly diseases or disorders that involve abnormal activation of the Jak1, Jak2, Jak3, Tyk2, KDR, Flt-3, CDK2, CDK4, TANK, Trk, FAK, Abl, Bcr-Abl, cMet, b-RAF, FGFR3, c-kit, PDGF-R, Syk, BTK, CSF1R, PKC kinases or Aurora kinases.

The protein kinases represent a large family of proteins that play a central role in the regulation of a wide variety of cellular processes and maintenance of cellular function. A partial, non-limiting, list of these kinases include: non-receptor tyrosine kinases such as the Janus kinase family (Jak1, Jak2, Jak3 and Tyk2); the fusion kinases, such as BCR-Abl, focal adhesion kinase (FAK). Fes, Lck and Syk; receptor tyrosine kinases such as platelet-derived growth factor receptor kinase (PDGF-R), the receptor kinase for stein cell factor, c-kit, the hepatocyte growth factor receptor, c-Met, and the fibroblast growth factor receptor, FGFR3; and serine/threonine kinases such as b-RAF, mitogen-activated protein kinases (e.g., MKK6) and SAPK2β. Aberrant kinase activity has been observed in many disease states including benign and malignant proliferative disorders as well as diseases resulting from inappropriate activation of the immune and nervous systems. The novel compounds of this invention inhibit the activity of one or more protein kinases and are, therefore, expected to be useful in the treatment of kinase-mediated diseases.

### SUMMARY OF THE INVENTION

In a first embodiment the invention provides a compound of Formula (I)

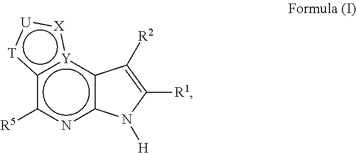

pharmaceutically acceptable salts, pro-drugs, biologically active metabolites, stereoisomers and isomers thereof wherein

T is N, U is N, X is CR³ and is N; or

T is N, U is CR⁴, X is CR³ and Y is N; or

T is N, U is N, X is NR³ and Y is C;

R¹, R² and R⁵ are each independently hydrogen or deuterium;

R³ is -A-D-F-G, wherein:
A is a bond or optionally substituted (C₃-C₁₂)cycloalkylene;
D is an optionally substituted (C₁-C₈)alkylene, optionally substituted C₃-C₁₀)cycloalkylene or an optionally substituted (C₂-C₁₀)heterocyclylene;
E is —R^{e}—, —R^{e}—C(O)—R^{e}—, —R^{e}—C(O)O—R^{e}, —R^{e}—N(R^{a})—R^{e}—, —R^{e}—N(R^{a})C(O)—R^{e}—, —R^{e}—C(O)N(R^{a})—R^{e}, —R^{e}—N(R^{a})S(O)₂—R^{e}—, or —R^{e}—S(O)₂N(R^{a})—R^{e}—, —R^{e}—O—CH₂—C(O)N(R^{a})—R^{e}—, —R^{e}—N(R^{a})C(O)—CH₂—O—R^{e}—, —R^{e}—C(O)N(R^{a})CH₂C(O)N(R^{a})—R^{e}—, —R^{e}—N(R^{a})C(O)CH₂N(R^{a})C(O)—R^{e}—, —R^{e}—C(O)N(R^{a})CH₂C(O)N(R^{a})—R^{e}—, —R^{e}—N(R^{a})C(O)CH₂N(R^{a})C(O)—R^{e}, —R^{e}—S—CH₂C(O)N(R^{a})—R^{e}—, or —R^{e}—N(R^{a})C(O)CH₂—S—R^{e}—; or
G is halogen, —CN, —CF₃, an optionally substituted —(C₁-C₆)alkyl, an -optionally substituted —(C₁-C₆)alkyl-heteroaryl, an optionally substituted —(C₃-C₁₀)cycloalkyl, optionally substituted heterocyclyl or an optionally substituted heteroaryl;

R⁴ is hydrogen or deuterium;

R^{a} and R^{b} are each independently hydrogen, deuterium, an optionally substituted (C₁-C₁₀)alkyl or an optionally substituted (C₁-C₁₀)heterocyclylene; and

R^{e} for each occurrence is independently a bond, an optionally substituted (C₁-C₁₀)alkylene, an optionally substituted (C₂-C₆)alkenylene, or an optionally substituted (C₁-C₁₀)heterocyclylene.

In a second embodiment the invention provides a compound according to the first embodiment wherein A is a bond or an optionally substituted cyclopentyl.

In a third embodiment the invention provides a compound compound according to any of the foregoing embodiments wherein D is an optionally substituted (C₁-C₆) alkylene, optionally substituted cyclopentylene or optionally substituted pyrrolidine.

In a fourth embodiment the invention provides a compound compound according to any of the foregoing embodiments wherein E is a bond, an optionally substituted (C₁-C₆)alkylene, —C(O)—, —C(O)—(C₁-C₃)alkylene-, —(C₁-C₃)alkylene-C(O)—, —N(H)—(C₁-C₃)alkylene-, —(C₁-C₃)alkylene-N(H)—, —N(H)—C(O)—, —C(O)N(H)—, —N(CH₃)—C(O)—, —C(O)N(CH₃)—, —(C₁-C₃)alkylene-N(H)—C(O)—, —C(O)N(H)—(C₁-C₃)alkylene-, —(C₁-C₃)alkylene-N(oxetanyl)-C(O)—, —C(O)N(oxetanyl)-(C₁-C₃)alkylene-, —N(R^{a})S(O)₂—(C₁-C₃)alkylene-, —(C₁-C₃)alkylene-S(O)₂N(R^{a})—, optionally substituted azetidine or optionally substituted piperidine.

In a fifth embodiment the invention provides a compound compound according to any of the foregoing embodiments wherein G is F, —CN, —CF₃, an optionally substituted —(C₁-C₆)alkyl, an optionally substituted —(C₃-C₆)cycloalkyl; optionally substituted morpholine, optionally substituted oxazole, optionally substituted 1,2,4-oxadiazole, optionally substituted pyridazine, or optionally substituted pyridine.

In a sixth embodiment the invention provides a compound compound according to any of the foregoing embodiments wherein
T is N, is N; X is CR³ and Y is N;
   wherein R³ is -A-D-E-G and
      A is a bond;
      D is optionally substituted cyclopentylene;
      E is an optionally substituted (C₁-C₆) alkylene, —C(O)—, —N(H)—C(O)—, —C(O)N(H)—, —C(O)N(H)—CH₂, —CH₂—N(H)C(O)—, —O—CH₂C(O)N(H)—, —N(H)C(O)CH₂—O—, —C(O)N(CH₃)CH₂C(O)N(H)—, —N(H)C(O)CH₂N(CH₃)C(O)—, —N(H)C(O)C(H)═C(H)—, —C(H)═C(H)C(O)N(H)—, -optionally substituted (C₁-C₃)alkylene-C(O)N(H)—, —N(H)C(O)-optionally substituted (C₁-C₃)alkylene, —C(O)N(H)CH₂C(O)N(H)—, —N(H)C(O)CH₂N(H)C(O)—, —S—CH₂C(O)N(H)—, or —N(H)C(O)CH₂—S—; and
      G is CN, CF₃, an optionally substituted benzofuranyl, an optionally substituted cyclobutyl, an optionally substituted cyclopropyl, an optionally substituted cyclohexyl, an optionally substituted benzo[c]isoxazolyl, an optionally substituted benzo[d]isoxazolyl, an optionally substituted furanyl, an optionally substituted furo[3,2-b]pyridinyl, an optionally substituted furo[3,2-b]pyrrolyl, an optionally substituted isoxazolyl, optionally substituted 1,2,4-oxadiazolyl, optionally substituted oxazolyl; an optionally substituted pyrrolidinyl, an optionally substituted tetrahydrobenzo[d]isoxazolyl, an optionally substituted tetrahydrobenzofuranyl, or a 2,5-dioxoimidazolidinyl.

In a seventh embodiment the invention provides a compound according to any of the foregoing embodiments wherein the compound is
(1R,3R,4S)-3-ethyl-4-(6H-pyrrolo[2,3-e][1,2,4]triazolo[4,3-a]pyrazin-1-yl)-N-(2,2,2-trifluoroethyl)cyclopentanecarboxamide;
3-(1S,3R,4S)-3-methyl-4-(6H-pyrrolo[2,3-e][1,2,4]triazolo[4,3-a]pyrazin-1-yl)cyclopentyl)propanenitrile;
3-((1R,3R,4S)-3-methyl-4-(6H-pyrrolo[2,3-e][1,2,4]triazolo[4,3-a]pyrazin-1-yl)cyclopentyl)propanenitrile;
3-((1S,3R,4S)-3-ethyl-4-(6H-pyrrolo[2,3-e][1,2,4]triazolo[4,3-a]pyrazin-1-yl)cyclopentyl)propanenitrile;
3-((1R,3R,4S)-3-ethyl-4-(6H-pyrrolo[2,3-e][1,2,4]triazolo[4,3-a]pyrazin-1-yl)cyclopentyl)propanenitrile;
N-((1S,3R,4S)-3-ethyl-4-(6H-pyrrolo[2,3-e][1,2,4]triazolo[4,3-a]pyrazin-1-yl)cyclopentyl)-2-methyloxazole-4-carboxamide;
N-((1S,3R,4S)-3-ethyl-4-(6H-pyrrolo[2,3-e][1,2,4]triazolo[4,3-a]pyrazin-1-yl)cyclopentyl)benzofuran-3-carboxamide;
N-(1S,3R,4S)-3-ethyl-4-(6H-pyrrolo[2,3-e][1,2,4]triazolo[4,3-a]pyrazin-1-yl)cyclopentyl)-4-methyl-4H-furo[3,2-b]pyrrole-5-carboxamide;
N-((1S,3R,4S)-3-ethyl-4-(6H-pyrrolo[2,3-e][1,2,4]triazolo[4,3-a]pyrazin-1-yl)cyclopentyl)-3-(1-methylcyclopropyl)propanamide;
3-cyclopropyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)-5-methylfuran-2-carboxamide; or
1-cyano-N-((1S,3R,4S)-3-ethyl-4-(6H-pyrrolo[2,3-e][1,2,4]triazolo[4,3-a]pyrazin-1-yl)cyclopentyl)cyclopropanecarboxamide;
N-(1S,3R,4S)-3-ethyl-4-(6H-pyrrolo[2,3-e][1,2,4]triazolo[4,3-a]pyrazin-1-yl)cyclopentyl)-5-methylisoxazole-4-carboxamide
(E)-N-((1S,3R,4S)-3-ethyl-4-(6H-pyrrolo[2,3-e][1,2,4]triazolo[4,3-a]pyrazin-1-yl)cyclopentyl)-3-(5-methylfuran-2-yl)acrylamide;
N-((1S,3R,4S)-3-ethyl-4-(6H-pyrrolo[2,3-e][1,2,4]triazolo[4,3-a]pyrazin-1-yl)cyclopentyl)-2-(5-methylisoxazol-3-yloxy)acetamide;
N-(2-((1S,3R,4S)-3-ethyl-4-(6H-pyrrolo[2,3-e][1,2,4]triazolo[4,3-a]pyrazin-1-yl)cyclopentylamino)-2-oxoethyl)-N-methylfuran-2-carboxamide;
3-(2,5-dioxoimidazolidin-4-yl)-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)-5-methyl-4,5,6,7-tetrahydrobenzo[d]isoxazole-3-carboxamide;
N-(2-((1S,3R,4S)-3-(6H-pyrrolo[2,3-e][1,2,4]triazolo[4,3-a]pyrazin-1-yl)cyclopentylamino)-2-oxoethyl)-5-methylfuran-2-carboxamide;
2-acetamido-3-cyclopropyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)benzofuran-3-carboxamide;
N-((1S,3R,4S)-3-ethyl-4-(6H-pyrrolo[2,3-e][1,2,4]triazolo[4,3-a]pyrazin-1-yl)cyclopentyl)-6-methylbenzofuran-2-carboxamide;
N-((1S,3R,4S)-3-ethyl-4-(6H-pyrrolo[2,3-e][1,2,4]triazolo[4,3-a]pyrazin-1-yl)cyclopentyl)-3-methylisoxazole-4-carboxamide;
N-((1S,3R,4S)-3-ethyl-4-(6H-pyrrolo[2,3-e][1,2,4]triazole[4,3-a]pyrazin-1-yl)cyclopentyl)-2-methyl-4,5,6,7-tetrahydrobenzofuran-3-carboxamide;
N-((1S,3R,4S)-3-ethyl-4-(6H-pyrrolo[2,3-e][1,2,4]triazolo[4,3-a]pyrazin-1-yl)cyclopentyl)-3-(3-methoxyisoxazol-5-yl)propanamide;
N-((1S,3R,4S)-3-ethyl-4-(6H-pyrrolo[2,3-e][1,2,4]triazolo[4,3-a]pyrazin-1-yl)cyclopentyl)-3-(3-methyl-1,2,4-oxadiazol-5-yl)propanamide;
N-(2-((1S,3R,4S)-3-ethyl-4-(6H-pyrrolo[2,3-e][1,2,4]triazolo[4,3-a]pyrazin-1-yl)cyclopentylamino)-2-oxoethyl)-5-methylisoxazole-4-carboxamide;
N-((1S,3R,4S)-3-ethyl-4-(6H-pyrrolo[2,3-e][1,2,4]triazolo[4,3-a]pyrazin-1-yl)cyclopentyl)-5-(furan-2-yl)-4H-pyrazole-3-carboxamide;
N-((1S,3R,4S)-3-ethyl-4-(6H-pyrrolo[2,3-e][1,2,4]triazolo[4,3-a]pyrazin-1-yl)cyclopentyl)-4-methyl-4H-furo[3,2-b]pyrrole-5-carboxamide;
2-(benzofuran-3-yl)-N-((1S,3R,4S)-3-ethyl-4-(6H-pyrrolo[2,3-e][1,2,4]triazolo[4,3-a]pyrazin-1-yl)cyclopentyl)acetamide;
N-((1S,3R,4S)-3-ethyl-4-(6H-pyrrolo[2,3-e][1,2,4]triazolo[4,3-a]pyrazin-1-yl)cyclopentyl)-5-methyloxazole-4-carboxamide;
N-((1S,3R,4S)-3-ethyl-4-(6H-pyrrolo[2,3-e][1,2,4]triazolo[4,3-a]pyrazin-1-yl)cyclopentyl)-3-methylbenzofuran-2-carbexamide;
N-((1S,3R,4S)-3-ethyl-4-(6H-pyrrolo[2,3-e][1,2,4]triazolo[4,3-a]pyrazin-1-yl)cyclopentyl)-3-(1-methylcyclopropyl)propanamide;
3-cyclopropyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)-5-methylfuran-2-carboxamide;
5-cyclopropyl-N-((1S,3R,4S)-3-ethyl-4-(6H-pyrrolo[2,3-e][1,2,4]triazolo[4,3-a]pyrazin-1-yl)cyclopentyl)oxazole-4-carboxamide;
N-((1S,3R,4S)-3-ethyl-4-(6H-pyrrolo[2,3-e][1,2,4]triazolo[4,3-a]pyrazin-1-yl)cyclopentyl)-5-methylisoxazole-3-carboxamide;
N-((1S,3R,4S)-3-ethyl-4-(6H-pyrrolo[2,3-e][1,2,4]triazolo[4,3-a]pyrazin-1-yl)cyclopentyl)-5-methylisoxazole-3-carboxamide;
N-((1S,3R,4S)-3-ethyl-4-(6H-pyrrolo[2,3-e][1,2,4]triazolo[4,3-a]pyrazin-1-yl)cyclopentyl)isoxazole-5-carboxamide;
N-((1S,3R,4S)-3-ethyl-4-(6H-pyrrolo[2,3-e][1,2,4]triazolo[4,3-a]pyrazin-1-yl)cyclopentyl)-4-methyloxazole-5-carboxamide;
N-((1S,3R,4S)-3-ethyl-4-(6H-pyrrolo[2,3-e][1,2,4]triazolo[4,3-a]pyrazin-1-yl)cyclopentyl)-2-methylfuran-3-carboxamide;
N-((1S,3R,4S)-3-ethyl-4-(6H-pyrrolo[2,3-e][1,2,4]triazolo[4,3-a]pyrazin-1-yl)cyclopentyl)-3-(furan-2-yl)propanamide;
1-cyano-N-((1S,3R,4S)-3-ethyl-4-(6H-pyrrolo[2,3-e][1,2,4]triazolo[4,3-a]pyrazin-1-yl)cyclopentyl)cyclopropanecarboxamide;
N-((1S,3R,4S)-3-ethyl-4-(6H-pyrrolo[2,3-e][1,2,4]triazolo[4,3-a]pyrazin-1-yl)cyclopentyl)-2-(3-methylisoxazol-5-yl)acetamide;
N-((1S,3R,4S)-3-ethyl-4-(6H-pyrrolo[2,3-e][1,2,4]triazolo[4,3-a]pyrazin-1-yl)cyclopentyl)furan-3-carboxamide;
N-((1S,3R,4S)-3-ethyl-4-(6H-pyrrolo[2,3-e][1,2,4]triazolo[4,3-a]pyrazin-1-yl)cyclopentyl)benzofuran-2-carboxamide;
N-((1S,3R,4S)-3-ethyl-4-(6H-pyrrolo[2,3-e][1,2,4]triazolo[4,3-a]pyrazin-1-yl)cyclopentyl)-4-oxo-4,5,6,7-tetrahydrobenzofuran-3-carboxamide;
5-cyclopropyl-N-((1S,3R,4S)-3-ethyl-4-(6H-pyrrolo[2,3-e][1,2,4]triazolo[4,3-a]pyrazin-1-yl)cyclopentyl)isoxazole-3-carboxamide;
(E)-N-((1S,3R,4S)-3-ethyl-4-(6H-pyrrolo[2,3-e][1,2,4]triazolo[4,3-a]pyrazin-1-yl)cyclopentyl)-3-(furan-2-yl)acrylamide;
N-((1S,3R,4S)-3-ethyl-4-(6H-pyrrolo[2,3-e][1,2,4]triazolo[4,3-a]pyrazin-1-yl)cyclopentyl)benzo[c]isoxazole-3-carboxamide
N-((1S,3R,4S)-3-ethyl-4-(6H-pyrrolo[2,3-e][1,2,4]triazolo[4,3-a]pyrazin-1-yl)cyclopentyl)-3-methylisoxazole-5-carboxamide;
N-((1S,3R,4S)-3-ethyl-4-(6H-pyrrolo[2,3-e][1,2,4]triazolo[4,3-a]pyrazin-1-yl)cyclopentyl)oxazole-4-carboxamide;
N-((1S,3R,4S)-3-ethyl-4-(6H-pyrrolo[2,3-e][1,2,4]triazolo[4,3-a]pyrazin-1-yl)cyclopentyl)-5-(methylthiomethyl)furan-2-carboxamide;
N-((1S,3R,4S)-3-ethyl-4-(6H-pyrrolo[2,3-e][1,2,4]triazolo[4,3-a]pyrazin-1-yl)cyclopentyl)-3-methoxyisoxazole-5-carboxamide;
5-cyclopropyl-N-((1S,3R,4S)-3-ethyl-4-(6H-pyrrolo[2,3-e][1,2,4]triazolo[4,3-a]pyrazin-1-yl)cyclopentyl)isoxazole-4-carboxamide;
3-cyclobutyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)benzo[d]isoxazole-3-carboxamide;
N-((1S,3R,4S)-3-ethyl-4-(6H-pyrrolo[2,3-e][1,2,4]triazolo[4,3-a]pyrazin-1-yl)cyclopentyl)-3-(5-methylfuran-2-yl)propanamide;
N-((1S,3R,4S)-3-ethyl-4-(6H-pyrrolo[2,3-e][1,2,4]triazolo[4,3-a]pyrazin-1-yl)cyclopentyl)-3-(2-oxocyclohexyl)propanamide;
2-(benzo[d]isoxazol-3-yl)-N-((1S,3R,4S)-3-ethyl-4-(6H-pyrrolo[2,3-e][1,2,4]triazolo[4,3-a]pyrazin-1-yl)cyclopentyl)acetamide;
N-((1S,3R,4S)-3-ethyl-4-(6H-pyrrolo[2,3-e][1,2,4]triazolo[4,3-a]pyrazin-1-yl)cyclopentyl)-4,5,6,7-tetrahydrobenzo[d]isoxazole-3-carboxamide;
N-(1S,3R,4S)-3-ethyl-4-(6H-pyrrolo[2,3-e][1,2,4]triazolo[4,3-a]pyrazin-1-yl)cyclopentyl)-5-(furan-2-yl)isoxazole-3-carboxamide;
5-ethyl-N-((1S,3R,4S)-3-ethyl-4-(6H-pyrrolo[2,3-e][1,2,4]triazolo[4,3-a]pyrazin-1-yl)cyclopentyl)oxazole-4-carboxamide;
N-((1S,3R,4S)-3-ethyl-4-(6H-pyrrolo[2,3-e][1,2,4]triazolo[4,3-a]pyrazin-1-yl)cyclopentyl)-2-(5-methyl-1,3,4-oxadiazol-2-ylthio)acetamide;
5-ethyl-N-((1S,3R,4S)-3-ethyl-4-(6H-pyrrolo[2,3-e][1,2,4]triazolo[4,3-a]pyrazin-1-yl)cyclopentyl)isoxazole-3-carboxamide;
3-cyclohexyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)furan-2-carboxamide;
N-((1S,3R,4S)-3-ethyl-4-(6H-pyrrolo[2,3-e][1,2,4]triazolo[4,3-a]pyrazin-1-yl)cyclopentyl)-3-methylfuran-2-carboxamide;
N-((1S,3R,4S)-3-ethyl-4-(6H-pyrrolo[2,3-e][1,2,4]triazolo[4,3-a]pyrazin-1-yl)cyclopentyl)-3-(5-oxopyrrolidin-2-yl)propanamide;
3-ethyl-N-((1S,3R,4S)-3-ethyl-4-(6H-pyrrolo[2,3-e][1,2,4]triazolo[4,3-a]pyrazin-1-yl)cyclopentyl)isoxazole-5-carboxamide;
N-((1S,3R,4S)-3-ethyl-4-(6H-pyrrolo[2,3-e][1,2,4]triazolo[4,3-a]pyrazin-1-yl)cyclopentyl)oxazole-5-carboxamide; or
N-((1S,3R,4S)-3-ethyl-4-(6H-pyrrolo[2,3-e][1,2,4]triazolo[4,3-a]pyrazin-1-yl)cyclopentyl)-5-methylisoxazole-4-carboxamide.

In an eighth embodiment the invention provides a compound according to the first embodiment wherein
T is N, U is CR⁴, X is CR³ and Y is N;
   R³ is -A-D-E-G, wherein:
   A is a bond or optionally substituted (C₃-C₆)cycloalkylene;
   D is an optionally substituted (C₁-C₄)alkylene, optionally substituted (C₃-C₁₀)cycloalkylene or an optionally substituted (C₂-C₁₀)heterocyclylene;
   E is —R^{e}—, —R^{e}—C(O)—R^{e}—, —R^{e}—C(O)O—R^{e}, —N(R^{a})—R^{e}—, —N(R^{a})C(O)—R^{e}—, —R^{e}—C(O)N(R^{a})—R^{e}—, —R^{e}—N(R^{a})S(O)₂—R^{e}—, or —R^{e}—S(O)₂N(R^{a})—R^{e}—;
   G is halogen, —CN, —CF₃, an optionally substituted —(C₁-C₆)alkyl, —CH₂-heteroaryl, an optionally substituted (C₃-C₁₀)cycloalkyl, an optionally substituted heterocyclyl or an optionally substituted heteroaryl;
R⁴ is hydrogen;
R^{a} and R^{b} are each independently hydrogen, an optionally substituted (C₁-C₁₀)alkyl or an optionally substituted (C₁-C₁₀)heterocyclylene, and
R^{e} for each occurrence is independently a bond, an optionally substituted (C₁-C₁₀)alkylene or an optionally substituted (C₁-C₆)heterocyclylene.

In a ninth embodiment the invention provides a compound according to the eighth embodiment wherein D —CH₂—, optionally substituted cyclopentyl, optionally substituted piperidinyl or optionally substituted pyrrolidinyl.

In a tenth embodiment the invention provides a compound according to the ninth embodiment wherein E is a bond, —C(O)—, —C(O)O(C₁-C₃)akylene-, —N(R^{a})—C(O)—, —C(O)N(R^{a})—, —C(O)—N(H)-optionally substituted (C₁-C₃)alkylene, -optionally substituted (C₁-C₃)alkylene-N(R^{a})C(O)—, (C₁-C₃)alkylene, —C(O)—(C₁-C₃)alkylene, —(C₁-C₃)alkylene-C(O)—, —(C₁-C₃)alkylene-OC(O)—, —CH₂—N(H)S(O)₂—, —N(H)—CH₂—, —CH₂—N(H)—, —S(O)₂N(H)—CH₂—, or optionally substituted azetidine.

In an eleventh embodiment the invention provides a compound according to the tenth embodiment wherein G is F, —CN, —CF₃, optionally substituted (C₁-C₄)alkyl, —CH₂-oxazolyl, optionally substituted cyclopropyl, optionally substituted cyclobutyl, optionally substituted azetidinyl, optionally substituted morpholinyl, optionally substituted oxazolyl, optionally substituted piperidinyl, optionally substituted pyridazinyl, optionally substituted pyrazinyl, optionally substituted pyridinyl or optionally substituted pyrimidinyl.

In a twelfth embodiment the invention provides a compound according to the eleventh embodiment wherein R^{a} and R^{b} are each independently hydrogen, CH₃ or oxetanyl.

In a thirteenth embodiment the invention provides a compound according to the twelfth embodiment wherein the compound is
N-((1S,3S,4R)-3-(3H-imidazo[1,2-a]pyrrolo[2,3-e]pyrazin-8-yl)-4-methylcyclopentyl)-5-methylpyrazine-2-carboxamide;
N-((1S,3S,4R)-3-(3H-imidazo[1,2-a]pyrrolo[2,3-e]pyrazin-8-yl)-4-methylcyclopentyl)pyridazine-3-carboxamide;
N-((1S,3S,4R)-3-(3H-imidazo[1,2-a]pyrrolo[2,3-e]pyrazin-8-yl)-4-methylcyclopentyl)-N-methyloxazole-4-carboxamide;
(1S,3S,4R)-3-(3H-imidazo[1,2-a]pyrrolo[2,3-e]pyrazin-8-yl)-4-methyl-N-((2-(trifluoromethyl)pyrimidin-5-yl)methyl)cyclopentanamine;
1-((1S,3S,4R)-3-(3H-imidazo[1,2-a]pyrrolo[2,3-e]pyrazin-8-yl)-4-methylcyclopentyl)piperidine-4-carbonitrile;
1-(((1R,3S,4R)-3-(3H-imidazo[1,2-a]pyrrolo[2,3-e]pyrazin-8-yl)-4-methylcyclopentyl)methyl)azetidine-3-carbonitrile;
1-(((1S,3S,4R)-3-(3H-imidazo[1,2-a]pyrrolo[2,3-e]pyrazin-8-yl)-4-methylcyclopentyl)methyl)azetidine-3-carbonitrile;
1-((1R,3S,4R)-3-(3H-imidazo[1,2-a]pyrrolo[2,3-e]pyrazin-5-yl)-4-methylcyclopentyl)piperidine-4-carbonitrile;
5-((3S,4R)-3-ethyl-4-(3H-imidazo[1,2-a]pyrrolo[2,3-e]pyrazin-8-yl)pyrrolidin-1-yl)-5-oxopentanenitrile;
(3S,4R)-3-ethyl-N-(2-fluoroethyl)-4-(3H-imidazo[1,2-a]pyrrolo[2,3-e]pyrazin-8-yl)pyrrolidine-1-carboxamide;
(3S,4R)—N-(2,2-difluoroethyl)-3-ethyl-4-(3H-imidazo[1,2-a]pyrrolo[2,3-e]pyrazin-8-yl)pyrrolidine-1-carboxamide;
1-((3S,4R)-3-ethyl-4-(3H-imidazol[1,2-a]pyrrolo[2,3-e]pyrazin-8-yl)pyrrolidin-1-yl)-4,4,4-trifluorobutan-1-one;
(3S,4R)—N-(2,2-difluoropropyl)-3-ethyl-4-(3H-imidazo[1,2-a]pyrrolo[2,3-e]pyrazin-8-yl)pyrrolidine-1-carboxamide;
(3S,4R)—N-cyclobutyl-3-ethyl-4-(3H-imidazo[1,2-a]pyrrolo[2,3-e]pyrazin-8-yl)pyrrolidine-1-carboxamide;
(3S,4R)-3-methyl-4-(3H-imidazo[1,2-a]pyrrolo[2,3-e]pyrazin-8-yl)-N-(2,2,2-trifluoroethyl)pyrrolidine-1-carboxamide;
(3R,4S)—N-(2,2-difluoroethyl)-3-(3H-imidazo[1,2-a]pyrrolo[2,3-e]pyrazin-8-yl)-4-methylpyrrolidine-1-carboxamide;
((3R,4S)-3-(3H-imidazo[1,2-a]pyrrolo[2,3-e]pyrazin-8-yl)-4-methylpyrrolidin-1-yl)(morpholino)methanone; or
1-((3R,4S)-3-(3H-imidazo[1,2-a]pyrrolo[2,3-e]pyrazin-8-yl)-4-methylpyrrolidin-1-yl)-2-cyclopropylethanone.

In a fourteenth embodiment the invention provides a compound according to the first embodiment wherein
T is N, U is N, X is NR³ and Y is C;
   R³ is -A-D-E-G, wherein:
      A is a bond;
      D is an optionally substituted (C₃-C₁₀)cycloalkyl;
      E is —R^{e}—;
      G is —CN; and
R^{e} is an optionally substituted (C₁-C₁₀)alkylene.

In a fifteenth embodiment the invention provides a compound according to the fourteenth embodiment wherein the compound is
3-((1R,3R,4S)-3-methyl-4-(pyrrolo[2,3-b][1,2,3]triazolo[4,5-d]pyridin-1(6H)-yl)cyclopentyl)propanenitrile.

In a sixteenth embodiment the invention provides a method of treating a disease comprising administering to a patient in need thereof an effective amount of a compound of Formula (I)

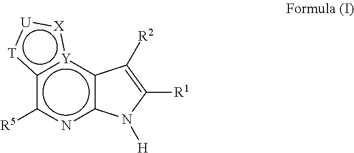

pharmaceutically acceptable salts, pro-drugs, biologically active metabolites, stereoisomers and isomers thereof wherein

T is N, U is N, X is CR³ and Y is N; or

T is N, U is CR⁴, X is CR³ and Y is N; or

T is N, U is N, X is NR³ and Y is C;

R¹, R² and R⁵ are each independently hydrogen or deuterium;

R³ is -A-D-E-G, wherein:
A is a bond or optionally substituted (C₃-C₁₂)cycloalkylene;
D is an optionally substituted (C₁-C₈)alkylene, optionally substituted (C₃-C₁₀)cycloalkylene or an optionally substituted (C₂-C₁₀)heterocyclylene;
E is —R^{e}—, —R^{e}—C(O)—R^{e}—, —R^{e}—C(O)O—R^{e}, —R^{e}—N(R^{a})—R^{e}—, —R^{e}—N(R^{a})C(O)—R^{e}—, —R^{e}—C(O)N(R^{a})—R^{e}, —R^{e}—N(R^{a})S(O)₂—R^{e}—, or —R^{e}—S(O)₂N(R^{a})—R^{e}, —R^{e}—O—CH₂—C(O)N(R^{a})—R^{e}—, —R^{e}—N(R^{a})C(O)—CH₂—O—R^{e}—, —R^{e}—C(O)N(R^{a})CH₂C(O)N(R^{a})—R^{e}—, —R^{e}—, N(R^{a})C(O)CH₂N(R^{a})C(O)—R^{e}—, —R^{e}—C(O)N(R^{a})CH₂C(O)N(R^{a})—R^{e}—, R^{e}—N(R^{a})C(O)CH₂N(R^{a})(O)—R^{e}, —R^{e}—S—CH₂C(O)N(R^{a})—R^{e}—, or —R^{e}—N(R^{a})C(O)CH₂—S—R^{e}—;
G is halogen, —CN, —CF₃, an optionally substituted —(C₁-C₆)alkyl, an optionally substituted —(C₁-C₆)alkyl-heteroaryl, an optionally substituted —(C₃-C₁₀)cycloalkyl, optionally substituted heterocyclyl or an optionally substituted heteroaryl;

R⁴ is hydrogen or deuterium;

R^{a} and R^{b} are each independently hydrogen, deuterium, or an optionally substituted (C₁-C₁₀)alkyl or an optionally substituted (C₁-C₁₀)heterocyclylene; and

R^{e} for each occurrence is independently a bond, an optionally substituted (C₁-C₁₀)alkylene, an optionally substituted (C₂-C₆)alkenylene, or an optionally substituted (C₁-C₁₀)heterocyclylene.

In a seventeenth embodiment the invention provides a method according to the sixteenth embodiment wherein the disease is rheumatoid arthritis, Crohn's Disease, juvenile rheumatoid arthritis, juvenile idiopathic arthritis, psoriasis, psoriatic arthritis, ankylosing spondylitis or dry eye.

In an eighteenth embodiment the invention provides a method of according to the seventeenth embodiment wherein the compound is a compound according to any of the foregoing embodiments.

### DETAILED DESCRIPTION OF THE INVENTION

Protein kinases are a broad and diverse class, of over 500 enzymes, that include oncogenes, growth factors receptors, signal transduction intermediates, apoptosis related kinases and cyclin dependent kinases. They are responsible for the transfer of a phosphate group to specific tyrosine, serine or threonine amino acid residues, and are broadly classified as tyrosine and serine/threonine kinases as a result of their substrate specificity.

The Jak family kinases (Jak1, Jak2, Jak3 and Tyk2) are cytoplasmic tyrosine kinases that associate with membrane bound cytokine receptors. Cytokine binding to their receptor initiates Jak kinase activation via trans and autophosphorylation processes. The activated Jak kinases phosphorylate residues on the cytokine receptors creating phosphotyrosine binding sites for SH2 domain containing proteins such as Signal Transduction Activators of Transcript (STAT) factors and other signal regulators transduction such as suppressor of cytokine signaling (SOCS) proteins and SH2 domain-containing inositol 5′-phosphatases (SHIP). Activation of STAT factors via this process leads to their dimerization, nuclear translocation and new mRNA transcription resulting in expression of immunocyte proliferation and survival factors as well as additional cytokines, chemokines and molecules that facilitate cellular trafficking (see Journal of Immunology, 2007, 178, p. 2623). Jak kinases transduce signals for many different cytokine families and hence potentially play roles in diseases with widely different pathologies including but not limited, to the following examples. Both Jak1 and Jak3 control signaling of the so-called common gamma chain cytokines (IL2, IL4, IL7, IL9, IL15 and IL21), hence simultaneous inhibition of either Jak1 or Jak3 could be predicted to impact Th1 mediated diseases such as rheumatoid arthritis via blockade of IL2, IL7 and IL15 signaling. On the other hand, IL2 signaling has recently been shown to be essential for development and homeostasis of T-regulatory cells (Malek T R et al., Immunity, 2002, 17(2), p. 167-78). Thus, based on genetic data, blockade of IL2 signaling alone is predicted to result in autoimmunity (Yamanouchi J et al., Nat Genet., 2007, 39(3), p. 329-37, and Willerford D M et al., Immunity, 1995, 3(4), p. 521-30). Th2 mediated diseases such as asthma or atopic dermatitis via IL4 and IL9 signaling blockade. Jak1 and Tyk2 mediate signaling of IL13 (see Int. Immunity, 2000, 12, p. 1499). Hence, blockade of these may also be predicted to have a therapeutic effect in asthma. These two kinases are also thought to mediate Type I interferon signaling; their blockade could therefore be predicted to reduce the severity of systemic lupus erythematosus (SLE). Tyk2 and Jak2 mediate signaling of IL12 and IL23. In fact, blockade of these cytokines using monoclonal antibodies has been effective in treating psoriasis. Therefore blockade of this pathway using inhibitors of these kinases could be predicted to be effective in psoriasis as well. In summary, this invention describes small-molecule compounds that inhibit, regulate and/or modulate Jak family kinase activity that is pivotal to several mechanisms thought critical to the progression of autoimmune diseases including, but not limited to, rheumatoid arthritis (RA), systemic lupus erythematosus (SLE), multiple sclerosis (MS), Crohn's disease, psoriasis, psoriatic arthritis, juvenile idiopathic arthritis, placque psoriasis, polyarticular juvenile idiopathic arthritis, ankylosing spondylitis and asthma.

Several pathologically significant cytokines signal via Jak1 alone (Guschin D, et al., EMBO J. 1995 Apr. 3; 14(7):1421-9; Parganas E, et al., Cell. 1998 May 1; 93(3):385-95; Rodig S. J., et al., Cell. 1998 May 1; 93(3):373-83). Blockade of one of these, IL6, using an IL6R neutralizing antibody, has been shown to significantly improve disease scores in human rheumatoid arthritis patients (Nishimoto N. et al., Ann Rheum Dis., 2007, 66(9), p. 1162-7). Similarly, blockade of GCSF signaling, which is also mediated by Jak1 alone, using neutralizing monoclonal antibodies or target gene deletion protects mice from experimental arthritis (Lawlor K. E. et al., Proc Natl Acad Sci U.S.A., 2004, 101(31), p. 11398-403). Accordingly, the identification of small-molecule compounds that inhibit, regulate and/or modulate the signal transduction of kinases, such as Jak1, is a desirable means to prevent or treat autoimmune diseases or other diseases related to abberant Jak1 function.

Jak2 is also activated in a wide variety of human cancers such as prostate, colon, ovarian and breast cancers, melanoma, leukemia and other haematopoietic malignancies. In addition, somatic point mutation of the Jak2 gene has been identified to be highly associated with classic myeloproliferative disorders (MPD) and infrequently in other myeloid disorders. Constitutive activation of Jak2 activity is also caused by chromosomal translocation in hematopoeitic malignancies. It has also been shown that inhibition of the Jak/STAT pathway, and in particular inhibition of Jak2 activity, results in anti-proliferative and pro-apoptotic effects largely due to inhibition of phosphorylation of STAT. Furthermore, pharmacological modulation or inhibition of Jak2 activity could effectively block tumor growth and induce apoptosis by reducing the STAT phosphorylation in cell culture and human tumor xenografts in vivo. Accordingly, the identification of small-molecule compounds that inhibit, regulate and/or modulate the signal transduction of kinases, particularly Jak2, is desirable as a means to treat or prevent diseases and conditions associated with cancers.

Jak kinases also transmit signals regulating essential physiological processes whose inhibition could be undesirable. For example Jak2 mediates the signaling of Erythropoetin (Epo) and Granulocyte/Monocyte-Colony Stimulating Factor (GM-CSF). Individuals with genetic, congenital or acquired defects in these signaling pathways can develop potentially life-threatening complications such as anemia and neutrophil dysfunction. Accordingly, one non-limiting aspect of this invention also relates to a method to identify compounds that may have a favorable safety profile as a result of them selectively avoiding inhibition of Jak2.

Spleen tyrosine kinase (Syk) (J. Bio. Chem, 1991, 266, 15790) is a non-receptor tyrosine kinase that plays a key role in immunoreceptor signaling in a host of inflammatory cells including B cells, mast cells, macrophages and neutrophils. Syk is related to zeta associated protein 70 (ZAP-70) but also demonstrates similarity with JAK. Src and Tec family kinases.

Syk plays a critical and specific role in B-cell receptor (BCR) signaling on auto-reactive cells and in FcR signaling on mast cells, macrophages, osteoclasts and neutrophils. (see Immunology Today, 2002, 21(3), 148 and Current Opinion in Immunology 2002, 14(3), 340. Syk plays a key role in the activation mediated by Fc receptors of sentinel cells (mast cells and macrophages) and effector cells (neutrophils, basophils and eosinophils). The importance of Syk in rheumatoid arthritis is substantiated by data demonstrating the importance of Fc receptors (FcR) function and immune complexes in disease pathogenesis. Syk also mediates the activation of B cells through the BCR, which results in their expansion and the production of antispecific immunoglobulins. Therefore any disease that revolves around antibody-Fc receptor interactions may be modulated by Syk suppression. Thus a Syk inhibitor is likely to dampen bath the initiation of the disease by blocking BCR signaling and the effector phase of the disease by blocking RR signaling on macrophages, neutrophils and mast cells. Furthermore, blocking Syk would provide the added benefit of inhibiting osteoclast maturation and therefore attenuate bony erosions, joint destruction and generalized osteopenia associated with rheumatoid arthritis. Moreover Syk acts upstream close to the receptors at the initiation of complex signaling events and thus its inhibition influences all responses elicited by the activating agent. In mast cells for example, inhibition of Syk blocks the early release of a number of granule contents, as well as the subsequent production and secretion of lipid mediators and cytokines. Syk inhibitors can thus impart multiple beneficial effects as each of these mediators play distinct roles in the integrated inflammatory response.

The protein kinase C family is a group of serine/threonine kinases that comprises twelve related isoenzymes, its members are encoded by different genes and are sub-classified according to their requirements for activation. The classical enzymes (cPKC) require diacylglycerol (DAG), phosphatidylserine (PS) and calcium for activation. The novel PKC's (nPKC) require DAG and PS but are calcium independent. The atypical PKC's (aPKC) do not require calcium or DAG.

PKCtheta is a member of the nPKC sub-family (Baier. G., et al., J. Biol. Chem., 1993, 268, 4997). It has a restricted expression pattern, found predominantly in T cells and skeletal muscle (Mischak, H. et al., FEBS Lett., 1993, 326, p. 51), with some expression reported in mast cells (Liu, Y. et al., J. Leukoc. Biol., 2001, 69, p. 831) and endothelial cells (Mattila, P. et al., Life Sci., 1994, 55, p. 1253).

Upon T cell activation, a supramolecular activation complex (SMAC) forms at the site of contact between the T cell and the antigen presenting cell (APC). PKCtheta is the only PKC isoform found to localize at the SMAC (Monks, C. et al., Nature, 1997, 385, 83), placing it in proximity with other signaling enzymes that mediate T cell activation processes.

In another study (Baier-Bitterlich, G. et al., Mol. Cell. Biol., 1996, 16, 842) the role of PKCtheta in the activation of AP-1, a transcription factor important in the activation of the IL-2 gene, was confirmed. In unstimulated T cells, constitutively active PKCtheta stimulated AP-1 activity while in cells with dominant negative PKCtheta, AP-1 activity was not induced upon activation by PMA.

Other studies showed that PKCtheta, via activation of IκB kinase beta, mediates activation of NF-κB induced by T cell receptor/CD28 co-stimulation (N. Coudronniere et al., Proc. Nat. Acad. Sci. U.S.A., 2000, 97, p. 3394; and Lin, X. et al., Mol. Cell. Biol., 2000, 20, p. 2933).

Proliferation of peripheral T cells from PKCtheta knockout mice, in response to T cell receptor (TCR)/CD28 stimulation was greatly diminished compared to T cells from wild type mice. In addition, the amount of IL-2 released from the T cells was also greatly reduced (Sun, Z. et al., Nature, 2000, 404, p. 402). It has also been shown that PKCtheta-deficient mice show impaired pulmonary inflammation and airway hyperresponsiveness (AHR) in a Th2-dependent murine asthma model, with no defects in viral clearance and Th1-dependent cytotoxic T cell function (Berg-Brown, N. N. et al., J. Exp. Med., 2004, 199, p. 743; Marsland, B. J. et al., J. Exp. Med., 2004, 200, p. 181). The impaired Th2 cell response results in reduced levels of IL-4 and immunoglobulin E (IgE), contributing to the AHR and inflammatory pathophysiology. Otherwise, the PKCtheta knockout mice seemed normal and fertile.

Evidence also exists that PKCtheta participates in the IgE receptor (FcεRI)-mediated response of mast cells (Liu, Y. et al., J. Leukoc. Biol., 2001, 69, p. 831). In human-cultured mast cells (HCMC), it has been demonstrated that PKC kinase activity rapidly localizes to the membrane following FcεRI cross-linking (Kimata, M. et al., Biochem. Biophys. Res. Commun., 1999, 257(3), p. 895). A recent study examining in vitro activity of bone marrow mast cells (BMMC) derived from wild-type and PKCtheta-deficient mice shows that upon FceRI cross linking, BMMCs from PKCtheta-deficient mice reduced levels of IL-6, tumor necrosis factor-alpha (TNFα) and IL-13 in comparison with BMMCs from wild-type mice, suggesting a potential role for PKCtheta in mast cell cytokine production in addition to T cell activation (Ciarletta, A. B. et al., poster presentation at the 2005 American Thoracic Society International Conference).

The studies cited above and others studies confirm the critical role of PKCtheta in T cells activation and in mast cell (MC) signaling. Thus an inhibitor of PKCtheta would be of therapeutic benefit in treating immunological disorders and other diseases mediated by the inappropriate activation of T cells and MC signaling.

Many of the kinases, whether a receptor or non-receptor tyrosine kinase or a S/T kinase have been found to be involved in cellular signaling pathways involved in numerous pathogenic conditions, including immunomodulation, inflammation, or proliferative disorders such as cancer.

Many autoimmune diseases and disease associated with chronic inflammation, as well as acute responses, have been linked to excessive or unregulated production or activity of one or more cytokines.

The compounds of the invention are also useful in the treatment of cardiovascular disorders, such as acute myocardial infarction, acute coronary syndrome, chronic heart failure, myocardial infarction, atherosclerosis, viral myocarditis, cardiac allograft rejection, and sepsis-associated cardiac dysfunction. Furthermore, the compounds of the present invention are also useful for the treatment of central nervous system disorders such as meningococcal meningitis, Alzheimer's disease and Parkinson's disease.

The compounds of the invention are also useful in the treatment of rheumatoid arthritis, an ocular condition, a cancer, a solid tumor, a sarcoma, fibrosarcoma, osteoma, melanoma, retinoblastoma, a rhabdomyosarcoma, glioblastoma, neuroblastoma, teratocarcinoma, hypersensitivity reactions, hyperkinetic movement disorders, hypersensitivity pneumonitis, hypertension, hypokinetic movement disorders, aordic and peripheral aneuryisms, hypothalamic-pituitary-adrenal axis evaluation, aortic dissection, arterial hypertension, arteriosclerosis, arteriovenous fistula, ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, heart transplant rejection, hemachromatosis, hemodialysis, hemolytic uremic syndrome/thrombolytic thrombocytopenic purpura, hemorrhage, idiopathic pulmonary fibrosis, antibody mediated cytotoxicity, Asthenia, infantile spinal muscular atrophy, inflammation of the aorta, influenza A, ionizing radiation exposure, iridocyclitis/uveitis/optic neuritis, juvenile spinal muscular atrophy, lymphoma, myeloma, leukaemia, malignant ascites, hematopoietic cancers, a diabetic condition such as insulin-dependent, diabetes mellitus glaucoma, diabetic retinopathy or microangiopathy, sickle cell anaemia, chronic inflammation, glomerulonephritis, graft rejection, Lyme disease, von Hippel Lindau disease, pemphigoid, Paget's disease, fibrosis, sarcoidosis, cirrhosis, thyroiditis, hyperviscosity syndrome, Osler-Weber-Rendu disease, chronic occlusive pulmonary disease, asthma or edema following burns, trauma, radiation, stroke, hypoxia, ischemia, ovarian hyperstimulation syndrome, post perfusion syndrome, post pump syndrome, post-MI cardiotomy syndrome, preeclampsia, menometrorrhagia, endometriosis, pulmonary hypertension, infantile hemangioma, or infection by Herpes simplex, Herpes Zoster, human immunodeficiency virus, parapoxvirus, protozoa or toxoplasmosis, progressive supranucleo palsy, primary pulmonary hypertension, radiation therapy, Raynaud's phenomenon, Raynaud's disease, Refsum's disease, regular narrow QRS tachycardia, renovascular hypertension, restrictive cardiomyopathy, sarcoma, senile chorea, senile dementia of Lewy body type, shock, skin allograft, skin changes syndrome, dry eye, ocular or macular edema, ocular neovascular disease, scleritis, radial keratotomy, uveitis, vitritis, myopia, optic pits, chronic retinal detachment, post-laser treatment complications, conjunctivitis, Stargardt's disease, Bales disease, retinopathy, macular degeneration, restenosis, ischemia/reperfusion injury, ischemic stroke, vascular occlusion, carotid obstructive disease, ulcerative colitis, inflammatory bowel disease, diabetes, diabetes mellitus, insulin dependent diabetes mellitus, allergic diseases, dermatitis scleroderma, graft versus host disease, organ transplant rejection (including but not limited to bone marrow and solid organ rejection), acute or chronic immune disease associated with organ transplantation, sarcoidosis, disseminated intravascular coagulation, Kawasaki's disease, nephrotic syndrome, chronic fatigue syndrome, Wegener's granulomatosis, Henoch-Schoenlein purpurea, microscopic vasculitis of the kidneys, chronic active hepatitis, septic shock, toxic shock syndrome, sepsis syndrome, cachexia, infectious diseases, parasitic diseases, acquired immunodeficiency syndrome, acute transverse myelitis. Huntington's chorea, stroke, primary biliary cirrhosis, hemolytic anemia, malignancies, Addison's disease, idiopathic Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, peripheral vascular disorders, peritonitis, pernicious anemia, myalgic encephalitis/Royal Free Disease, chronic mucocutaneous candidiasis, giant cell arteritis, primary sclerosing hepatitis, cryptogenic autoimmune hepatitis, Acquired immunodeficiency Disease Syndrome, Acquired immunodeficiency Related Diseases, Hepatitis A, Hepatitis B, Hepatitis C, His bundle arrythmias, HIV infection/HIV neuropathy, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, pneumocystis carinii pneumonia, pneumonia, connective tissue disease associated interstitial lung disease, mixed connective tissue disease, associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthritis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, acute and chronic pain (different forms of pain), Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, toxicity, transplants, and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Compounds of Formula (I) of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the compounds of this invention. Non-limiting examples of therapeutic agents for rheumatoid arthritis with which a compound of Formula (I) of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. Compounds of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7 (U.S. Pat. No. 6,090,382, HUMIRA™), CA2 (REMICADE™), SIMPONI™ (golimumab), CIMZIA™, ACTEMRA™, CDP 571, and soluble p55 or p75 TNF receptors, derivatives, thereof, (p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other preferred combinations include Interleukin I1. Yet other preferred combinations are the other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination is non-depleting anti-CD4 inhibitors. Yet other preferred combinations include antagonists of the co-stimulatory pathway CD80 (B7.1) or CD86 (B7.2) including antibodies, soluble receptors or antagonistic ligands.

A compound of Formula (I) of the invention may also be combined with agents, such as methotrexate, 6-mercaptopurine, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., NIK, IKK, p38 or MAP kinase inhibitors), IL-1β converting enzyme inhibitors, T-cell signalling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™) and p55TNTRIgG (Lenercept), siL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, S1P1 agonists (such as FTY720), PKC family inhibitors (such as Ruboxistaurin or AEB-071) and Mesopram. Preferred combinations include methotrexate or leftunomide and in moderate or severe rheumatoid arthritis cases, cyclosporine and anti-TNF antibodies as noted above.

Non-limiting examples of therapeutic agents for inflammatory bowel disease with which a compound of Formula (I) of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, IL-23, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFα or IL-1 (e.g. NIK, IKK, or MAP kinase inhibitors); IL-1β converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytkines (e.g. IL-4, IL-10, IL-11, IL-13 and TGFβ). Preferred examples of therapeutic agents for Crohn's disease with which a compound of Formula (I) can be combined include the following: TNF antagonists, for example, anti-TNF antibodies, D2E7 (U.S. Pat. No. 6,090,382, HUMIRA™), CA2 (REMICADE™), CDP 571, TNFR-Ig constructs, (p75TNFRIgG (ENBREL™) and p55TNFRIgG (LENERCEPT™) inhibitors and PDE4 inhibitors. A compound of Formula (I) can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors; 6-mercaptopurine; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam cyanocobalamin; folic acid; levofloxacin; methylprednisolone; natalizumab and interferon-gamma.

Non-limiting examples of therapeutic agents for multiple sclerosis with which a compound of Formula (I) can be combined include the following: corticosteroids; prednisolone; methylprednisolone; azathioprine; cyclophosphamide; cyclosporine; methotrexate; 4-aminopyridine; tizanidine; interferon-β1a (AVONEX®; Biogen); interferon-β1b (BETASERON®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE®; Teva Pharmaceutical Industries, Inc.); hyperbaric oxygen; intravenous immunoglobulin; cladribine, antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of Formula (I) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of Formula (I) may also be combined with agents such as methotrexate, cyclosporine, FK506, rapamycin, mycophenolate mofetil, leflunomide, an S1P1 agonist, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of Formula (I) can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of Formula (I) may also be combined with agents, such as alemtuzumab, dronabinol, daclizumab, mitoxantrone, xaliproden hydrochloride, fampridine, glatiramer acetate, natalizumab, sinnabidol, α-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for ankylosing spondylitis with which a compound of Formula (I) can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, and anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p5STNFRIgG (LENERCEPT™)

Non-limiting examples of therapeutic agents for asthma with which a compound of Formula (I) can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, anti-IL-13 antibody, and metaproterenol sulfate.

Non-limiting examples of therapeutic agents for COPD with which a compound of Formula (I) can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast and roflumilast.

Non-limiting examples of therapeutic agents for Idiopathic Pulmonary Fibrosis with which a compound of Formula (I) can be combined include the following: prednisone, azathioprine, albuterol, colchicine, albuterol sulfate, digoxin, gamma interferon, methylprednisolone sodium succinate, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and interferon-gamma-1β.

Non-limiting examples of therapeutic agents for psoriasis with which a compound of Formula (I) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine, ABT-874 and ustekinamab.

Non-limiting examples of therapeutic agents for psoriatic arthritis with which a compound of Formula (I) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, D2E7 (U.S. Pat. No. 6,090,382, HUMIRA™), and efalizumab.

Non-limiting examples of therapeutic agents for restenosis with which a compound of Formula (I) can be combined include the following: sirolimus, paclitaxel, everolimus, tacrolimus, ABT-578, and acetaminophen.

Non-limiting examples of therapeutic agents for sciatica with which a compound of Formula (I) can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol hcl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene n-pap, asa/oxycod/oxycodone ter, ibuprofen/hydrocodone bit, tramadol etodolac, propoxyphene HCl, amitriptyline HCl, carisoprodol/codeine phos/asa, morphine sulfate, multivitamins, naproxen sodium, orphenadrine citrate, and temazepam.

Preferred examples of therapeutic agents for SLE (Lupus) with which a compound of Formula (I) can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of Formula (I) may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of Formula (I) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of Formula (I) can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of Formula (I) may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™).

In this invention, the following definitions are applicable:

A “therapeutically effective amount” is an amount of a compound of Formula (I) or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g. (+) or (−)-tartaric acid or mixtures thereof), amino acids (e.g. (+) or (−)-amino acids or mixtures thereof), and the like. These salts can be prepared by methods known to those skilled, in the art.

Certain compounds of Formula (I) which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of Formula (I) and their salts may exist in more than one crystal form and the present invention includes each crystal form and mixtures thereof.

Certain compounds of Formula (I) and their salts may also exist in the form of solvates, for example hydrates, and the present invention includes each solvate and mixtures thereof.

Certain compounds of Formula (I) may contain one or more chiral centers, and exist in different optically active forms. When compounds of Formula (I) contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of Formula (I) contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of Formula (I) or Formula (II), and mixtures thereof.

Certain compounds of Formula (I) may exist in different tautomeric forms or as different geometric isomers, and the present invention includes each tautomer and/or geometric isomer of compounds of Formula (I) and mixtures thereof.

Certain compounds of Formula (I) may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Formula (I) and mixtures thereof.

Certain compounds of Formula (I) may exist in zwitterionic form and the present invention includes each zwitterionic form of compounds of Formula (I) and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may for instance, be bioavailable by oral administration whereas the parent drug is not. The pro-drug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial.

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents wherein the free hydrogen is replaced by (C₁-C₄)alkyl, (C₁-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula (I) wherein the free hydrogen of the hydroxyl substituent (e.g., R group contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₁₂)alkoxycarbonyloxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ glycosyl (the radical resulting from detachment of the hydroxyl of the hemiacetal of a carbohydrate).

The term “heterocyclic”, “heterocyclyl” or “heterocyclylene”, as used herein, include non-aromatic, ring systems, including, but not limited to, monocyclic, bicyclic, tricyclic and spirocyclic rings, which can be completely saturated or which can contain one or more units of unsaturation, for the avoidance of doubt, the degree of unsaturation does not result in an aromatic ring system) and have 5 to 12 atoms including at least one heteroatom, such as nitrogen, oxygen, or sulfur. For purposes of exemplification, which should not be construed as limiting the scope of this invention, the following are examples of heterocyclic rings: azepinyl, azetidinyl, isoindolinyl, morpholinyl, piperazinyl, piperidinyl, pyrrolidinyl, quinucludinyl, thiomorpholinyl, tetrahydropyranyl, tetrahydrofuranyl, tetrahydroindolyl, thiomorpholinyl and tropanyl.

The term “heteroaryl” or “heteroarylene” as used herein, include aromatic ring systems, including, but not limited to, monocyclic, bicyclic and tricyclic rings, and have 5 to 12 atoms including at least one heteroatom, such as nitrogen, oxygen, or sulfur. For purposes of exemplification, which should not be construed as limiting the scope of this invention: azaindolyl, benzo[b]thienyl, benzimidazolyl, benzofuranyl, benzoxazolyl, benzothiazolyl, benzothiadiazolyl, benzoxadiazolyl, furanyl, imidazolyl, imidazopyridinyl, indolyl, indazolyl, isoxazolyl, isothiazolyl, oxadiazolyl, oxazolyl, purinyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrazolyl, thiadiazolyl, or thienyl.

As used herein, “alkyl”, “alkylene” or notations such as “(C₁-C₈)” include straight chained or branched hydrocarbons which are completely saturated. Examples of alkyls are methyl, ethyl, propyl, isopropyl, butyl, pentyl, hexyl and isomers thereof. As used herein, “alkenyl”, “alkenylene”, “alkynylene” and “alkynyl” means C₂-C₈ and includes straight chained or branched hydrocarbons which contain one or more units of unsaturation, one or more double bonds for alkenyl and one or more triple bonds for alkynyl.

As used herein, “aromatic” groups (or “aryl” or “arylene” groups) include aromatic carbocyclic ring systems (e.g. phenyl) and fused polycyclic aromatic ring systems (e.g. naphthyl, biphenyl and 1,2,3,4-tetrahydronaphthyl).

As used herein, “cycloalkyl” or “cycloalkylene” means C₃-C₁₂ monocyclic or multicyclic (e.g., bicyclic, tricyclic, spirocyclic, etc.) hydrocarbons that are completely saturated or have one or more unsaturated bonds but does not amount to an aromatic group. Examples of a cycloalkyl group are cyclopropyl, cyclobutyl, cyclopentyl, cyclopentenyl, cyclohexyl and cyclohexenyl.

As used herein, many moieties or substituents are termed as being either “substituted” or “optionally substituted”. When a moiety is modified by one of these terms, unless otherwise noted, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —C(O)—(C₁-C₃)alkylene-O-optionally substituted aryl, —C(O)-optionally substituted (C₃-C₆)cycloalkyl, —NHC(O)H, —NHC(O) (C₁-C₈)alkyl groups, —NHC(O) (C₃-C₈)cycloalkyl groups, —N(C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

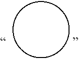

in Formula (I) represents an aromatic ring.

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few hours up to over several days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Pharmaceutical compositions suitable for use in the present invention include compositions wherein the active ingredients are contained, in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see e.g. Fingl et al., 1975, in “The Pharmacological Basis of Therapeutics”, Ch. 1 p. 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the following Examples.

### a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

### b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

### c) Enteric Coated Tablets

Tablets can be prepared by the method described in b) above. The tablets can be enteric coated in a conventional manner using a solution of 20% cellulose acetate phthalate and 3% diethyl phthalate in ethanol/dichloromethane (1:1).

### d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PKC inhibitors, PI3 kinase inhibitors, calcineurin inhibitors and immunosuppressants. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

The present invention also comprises the use of a compound of Formula (I) as a medicament.

A further aspect of the present invention provides the use of a compound of Formula (I) or a salt thereof in the manufacture of a medicament for treating vascular hyperpermeability, angiogenesis-dependent disorders, proliferative diseases and/or disorders of the immune system in mammals, particularly human beings.

The present invention also provides a method of treating vascular hyperpermeability, inappropriate neovascularization, proliferative diseases and/or disorders of the immune system which comprises the administration of a therapeutically effective amount of a compound of Formula (I) to a mammal, particularly a human being, in need thereof.

### Abbreviations

AcOH Glacial acetic acid
BSA Bovine serum albumin
BuOH Butanol
CDI 1,1′-Carbonyldiimidazole
d Doublet
dd Doublet of doublets
ddt Double doublet of triplets
dba Dibenzylideneacetone
DCE Dichloroethane
DCM Dichloromethane (methylene chloride)
DEA Diethylamine
DIBAL-H Diisobutylaluminum hydride
DIEA N,N-Diisopropylethylamine
DMEM Dulbecco's Modified Eagle Medium
DMF N,N-Dimethylformamide
DMSO Dimethyl sulfoxide
DNP-HSA Dinitrophenyl-human serum albumin
DPPA Diphenyl phosphorazidate
dppf 1,1′-Bis(diphenylphosphino)ferrocene
dq Doublet of quartets
EDC N-(3-Dimethylaminopropyl)-N′-ethylcarbodiimide
EDTA Ethylene diamine tetraacetic acid
equiv Equivalent(s)
EtOAc Ethyl acetate
Et₂O Diethyl ether
EtOH Ethanol
FBS Fetal bovine serum
FLAG DYKDDDDK peptide sequence
g Gram(s)
GST Glutathione S-transferase
h Hour(s)
HATU O-(7-Azabenzotriazol-1-yl)-N,N,N′,N′-tetramethyluronium hexafluorophosphate
HBTU O-(Benzotriazol-1-yl)-N,N,N′,N′-tetramethyluronium hexafluorophosphate
HPLC High-pressure liquid chromatography
Hz Hertz
i.d. Intradermal
IFA Incomplete Freunds Adjuvant
i-Pr Isopropyl
KOAc Potassium acetate
LC Liquid chromatography
m Multiplet
M Molar
MTBE Methyl tert-butyl ether
MeCN Acetonitrile
MeOH Methyl alcohol
2-MeTHF 2-Methyltetrahydrofuran
min Minute(s)
mL Milliliter(s)
mmol Millimole
MOPS 3-(N-morpholino)-propanesulfonic acid
MOPSO 3-(N-morpholino)-2-hydroxypropanesulfonic acid
MS Mass spectrometry
N Normal
NaOt-Bu Sodium tert-butoxide
NBS N-Bromosuccinimide
NH₄OAc Ammonium acetate
NMP N-Methylpyrrolidone
NMR Nuclear magnetic resonance
or Optical rotation
OVA Ovalbumin
PBS Phosphate buffered saline
PFPAA 2,2,3,3,3-Pentafluoropropanoic Anhydride
pH −log [H⁺]
pNAG Nitrophenyl-N-acetyl-β-D-glucosaminide
PPh₃ Triphenylphosphine
ppm Parts per million
psi Pounds per square inch
q Quartet
ref Relative centrifugal three
Rₜ Retention time
rt Room temperature
q Quartet
s Singlet
SEM 2-(Trimethylsilyl)ethoxymethyl
SFC Supercritical Fluid Chromatography
t Triplet
t- Tertiary
TBAF Tetra-n-butylammonium fluoride
TEA Triethylamine
TFA Trifluoroacetic acid
TFAA Trifluoracetic anhydride
THF Tetrahydrofuran
TLC Thin layer chromatography
USP United States Pharmacopeia
UV Ultraviolet
wt % Weight percent

### Assays

In Vitro Jak1 Kinase Activity Measured by Time-Resolved Fluorescence Resonance Energy Transfer (trFRET)

Varying concentrations of inhibitor were added to an assay well containing: laid enzyme (aa 845-1142; expressed in SF9 cells as a GST fusion and purified by glutathione affinity chromatography; 4 nM), peptide substrate (biotin-TYR2, Sequence: Biotin-(Ahx)-AEEEYFFLFA-amide; 2 μM), MOPSO pH 6.5 (50 mM), MgCl₂ (10 mM), MnCl₂ (2 mM), DTT (2.5 mM), BSA (0.01% w/v), Na₃VO₄ (0.1 mM) and ATP (0.001 mM). After about 60 min incubation at rt, the reaction was quenched by addition of EDTA (final concentration: 100 mM) and developed by addition of revelation reagents (final approximate concentrations: 30 mM HEPES pH 7.0, 0.06% BSA, 0.006% Tween-20, 0.24 M KF, 80 ng/mL PT66K (europium labeled anti-phosphotyrosine antibody cat #61T66KLB Cisbio, Bedford, Mass.) and 3.12 μg/mL SAXL (Phycolink streptavidin-allophycocyanin acceptor, cat #PJ52S, Prozyme, San Leandro, Calif.). The developed, reaction was incubated in the dark either at about 4° C. for about 14 h or for about 60 min at rt, then read via a time-resolved fluorescence detector (Rubystar, BMG) using a 337 nm laser for excitation and emission wavelength of 665 nm. Within the linear range of the assay, the observed signal at 665 nm is directly related to phosphorylated product and used to calculate the IC₅₀ values.

In Vitro Jak3 Kinase Activity Measured by Time-Resolved Fluorescence Resonance Energy Transfer (trFRET)

Varying concentrations of inhibitor were added to an assay well containing: Jak3 enzyme (aa 811-1103; expressed in SF9 cells as a GST fusion and purified by glutathione affinity chromatography; 3 nM), peptide substrate (biotin-TYR2, Sequence: Biotin-(Ahx)-AEEEYFFLFA-amide; 2 μM), MOPSO pH 6.5 (50 mM), MgCl₂ (10 mM), MnCl₂ (2 mM), DTT (2.5 mM), BSA (0.01% w/v), Na₃VO₄ (0.1 mM) and ATP (0.001 mM). After about 60 min incubation at rt, the reaction was quenched by addition of EDTA (final concentration: 100 mM) and developed by addition of revelation reagents (final approximate concentrations: 30 mM HEPES pH 7.0, 0.06% BSA, 0.006% Tween-20, 0.24 M KF, 80 ng/mL PT66K (europium labeled anti-phosphotyrosine antibody cat #61T66KLB Cisbio, Bedford, Mass.) and 0.8 μg/mL SAXL (Phycolink streptavidin-allophycocyanin acceptor, cat #PJ52S, Prozyme, San Leandro, Calif.). The developed reaction was incubated in the dark either at about 4° C., for about 14 h or for about 60 min at rt, then read via a time-resolved fluorescence detector (Rubystar, BMG) using a 337 nm laser for excitation and emission wavelength of 665 nm. Within the linear range of the assay, the observed signal at 665 nm is directly related to phosphorylated product and used to calculate the IC₅₀ values. For the purpose of the Tables and Examples below, the Jak3 IC₅₀ of each compound, which can be determined using the assay method described herein using the in vitro Jak3 kinase activity measured by time-resolved fluorescence resonance energy transfer (trFRET) is expressed as follows: A=a compound with a Jak3 IC₅₀ less than 1 μM, B=a compound with a Jak3 IC₅₀ within the range of 1 to 10.0 μM, C=a compound with a Jak3 IC₅₀ greater than 10 μM.

In Vitro Syk Kinase Activity Measured by Time-Resolved Fluorescence Resonance Energy Transfer (trFRET)

0.3 nM Syk catalytic domain (aa356-635, purified in-house at the Abbott Bioreseach Center) was mixed with 0.1 μM peptide substrate (biotin-TYR1, Sequence: Biotin-(Ahx)-GABEEIYAAFFA-COOH) at varying concentrations of inhibitor in reaction buffer: 50 mM MOPSO pH 6.5, 10 mM MgCl₂, 2 mM MnCl₂, 2.5 mM DTT, 0.01% BSA, 0.1 mM Na₃VO₄ and 0.001 mM ATP. After about 60 min incubation at rt, the reaction was quenched by addition of EDTA (final concentration: 100 mM) and developed by addition of revelation reagents (final approximate concentrations: 30 mM HEPES pH 7.0, 0.06% BSA, 0.006% Teen-20, 0.24 M KF, 90 ng/mL PT66K (europium labeled anti-phosphotyrosine antibody cat #61T66KLB Cisbio, Bedford, Mass.) and 0.6 μg/mL SAXL (Phycolink streptavidin-allophycocyanin acceptor, cat #PJ52S, Prozyme, San Leandro, Calif.). The developed reaction was incubated in the dark either at about 4° C. for about 14 h or for about 60 min at rt, her read via a time-solved fluorescence detector (Rubystar, BMG) using a 337 nm laser for excitation and emission wavelength of 665 nm. Within the linear range of the assay, the observed signal at 665 nm is directly related to phosphorylated product and used to calculate the IC₅₀ values.

Other In Vitro Kinase Assays Measured by Time-Resolved Fluorescence Resonance Energy Transfer (trFRET)

Other kinase assays were performed using a similar protocol. Additional purified enzymes Tyk2 (aa 880-1185 with an N-terminal histidine-tag and C-terminal FLAG tag; purified in-house by immobilized metal ion affinity chromatography), RET (aa 711-1072 with an N-terminal histidine-tag; purified by immobilized metal ion affinity chromatography), Syk (aa356-635 with a C-terminal histidine tag; purified by immobilized metal ion affinity chromatography), and KDR (aa 792-1354 with an N-terminal histidine-tag; purified in-house by immobilized metal ion affinity and ion-exchange chromatography) were expressed in SF9 cells and Aurora 1/B (aa1-344 with a N-terminal histidine-tag and purified by immobilized metal ion affinity chromatography) was expressed in E. coli. Other enzymes used are available from commercial sources. Enzymes were mixed with biotinylated substrates at varying concentrations of inhibitor in different reaction buffers (see Table A). After about 60 min incubation at rt, the reaction was quenched by addition of EDTA and developed by addition of revelation reagents (final approximate concentrations: 30 mM HEPES pH 7.0, 0.06% BSA, 0.006% Teen-20, 0.24 M KF, varying amounts of donor europium labeled antibodies and acceptor streptavidin labeled allophycocyanin (SAXL)). The developed reactions were incubated in the dark either at about 4° C. for about 14 h or for about 60 min at rt, then read in a time-resolved fluorescence detector (Rubystar, BMG Labtech) as described above.

TABLE A
Specific conditions (per 40 μL enzyme reaction) for the various enzymes are
detailed below:

| | | | | Enzyme | | ATP | DMSO | Reaction | |
|---|---|---|---|---|---|---|---|---|---|
| | | | Assay | Conc. | Substrate | Conc. | Conc. | Time | Detection |
| Enzyme | Construct | Substrate | Buffer | (ng/well) | Conc. | (mM) | (%) | (min) | condition |
| Jak1 | aa 845-1142 | Biotin-TYR2 | MOPSO | 5 | 2 μM | 0.001 | 5 | 60 | 8 ng/well |
| | | | | | | | | | PT66K, |
| | | | | | | | | | 0.39 |
| | | | | | | | | | μg/well |
| | | | | | | | | | SAXL |
| Jak2 | Millipore | Biotin-TYR1 | MOPSO | 2.5 | 2 μM | 0.001 | 5 | 60 | 8 ng/well |
| | cat# 14-640 | | | | | | | | PT66K, |
| | | | | | | | | | 0.078 |
| | | | | | | | | | μg/well |
| | | | | | | | | | SAXL |
| Jak3 | aa 811-1103 | Biotin-TYR2 | MOPSO | 4.5 | 2 μM | 0.001 | 5 | 60 | 8 ng/well |
| | | | | | | | | | PT66K, |
| | | | | | | | | | 0.078 |
| | | | | | | | | | μg/well |
| | | | | | | | | | SAXL |
| Tyk2 | aa880-1185 | Biotin-TYR1 | MOPSO | 9 | 2 μM | 0.001 | 5 | 60 | 8 ng/well |
| | | | | | | | | | PT66K, |
| | | | | | | | | | 0.078 |
| | | | | | | | | | μg/well |
| | | | | | | | | | SAXL |
| Aurora | aa1-344 | KinEASE | MOPS | 20 | 0.5 μM | 0.1 | 5 | 60 | 15 |
| 1/B | | S2 | | | | | | | ng/well |
| | | | | | | | | | Eu-STK- |
| | | | | | | | | | Ab, |
| | | | | | | | | | 0.34 |
| | | | | | | | | | μg/wel |
| | | | | | | | | | SAXL |
| KDR | aa789-1354 | Biotin-TYR2 | HEPES | 10 | 2 μM | 0.1 | 5 | 60 | 8 ng/well |
| | | | | | | | | | PT66K, |
| | | | | | | | | | 0.078 |
| | | | | | | | | | μg/well |
| | | | | | | | | | SAXL |
| JNK1 | Millipore | Biotin- | MOPS | 10 | 1 μM | 0.01 | 5 | 60 | 2.58 |
| | cat# 14-327 | ATF2-pep | | | | | | | ng/well |
| | | | | | | | | | Anti- |
| | | | | | | | | | pATF2- |
| | | | | | | | | | Eu, |
| | | | | | | | | | 0.6 |
| | | | | | | | | | μg/well |
| | | | | | | | | | SAXL |
| JNK2 | Millipore | Biotin- | MOPS | 5 | 0.5 μM | 0.01 | 5 | 60 | 2.58 |
| | cat# 14-329 | ATF2-pep | | | | | | | ng/well |
| | | | | | | | | | Anti- |
| | | | | | | | | | pATF2- |
| | | | | | | | | | Eu, |
| | | | | | | | | | 0.6 |
| | | | | | | | | | μg/well |
| | | | | | | | | | SAXL |
| RET | aa711-1072 | Biotin-poly | HEPES | 4 | 10 | 0.01 | 5 | 60 | 8 ng/well |
| | | GluTyr | | | ng/well | | | | PT66K, |
| | | | | | | | | | 0.078 |
| | | | | | | | | | μg/well |
| | | | | | | | | | SAXL |
| P70 S6 | Millipore | KinEASE | MOPS | 0.5 | 0.25 μM | 0.01 | 5 | 60 | 15 |
| Kinase | cat# 14-486 | S3 | | | | | | | ng/well |
| | | | | | | | | | Eu-STK- |
| | | | | | | | | | Ab, |
| | | | | | | | | | 0.34 |
| | | | | | | | | | μg/well |
| | | | | | | | | | SAXL |
| PKN2 | Invitrogen | KinEASE | MOPS | 0.7 | 0.5 μM | 0.001 | 5 | 60 | 15 |
| | cat# | S3 | | | | | | | ng/well |
| | PV3879 | | | | | | | | Eu-STK- |
| | | | | | | | | | Ab, |
| | | | | | | | | | 0.34 |
| | | | | | | | | | μg/well |
| | | | | | | | | | SAXL |
| Syk | aa356-635 | Biotin-TYR1 | MOPSO | 0.4 | 0.1 μM | 0.001 | 5 | 60 | 6.8 |
| | | | | | | | | | ng/well |
| | | | | | | | | | PT66K, |
| | | | | | | | | | 0.045 |
| | | | | | | | | | μg/well |
| | | | | | | | | | SAXL |
| CDK2/ | Millipore | Biotin-MBP | MOPS | 50 | 2 μM | 0.1 | 5 | 60 | 15 |
| Cyclin A | cat# 14-448 | | | | | | | | ng/well |
| | | | | | | | | | Anti- |
| | | | | | | | | | pMBP- |
| | | | | | | | | | Eu; 0.34 |
| | | | | | | | | | μg/well |
| | | | | | | | | | SAXL |

### Reaction Buffers:

MOPSO buffer contains: 50 mM MOPSO pH 6.5, 10 mM MgCl₂, 2 mM MnCl₇, 2.5 mM DTT, 0.01% BSA, and 0.1 mM Na₃VO₄

HEPES buffer contains: 50 mM HEPES pH 7.1, 2.5 mM DTT, 10 mM MgCl₂, 2 mM MnCl₂, 0.01% BSA, and 0.1 mM Na₃VO₄

MOPS buffer contains: 20 mM MOPS pH 7.2, 10 mM MgCl₂, 5 mM EGTA, 5 mM Beta-phosphoglycerol, 1 mM Na₃VO₄, 0.01% Triton-X-100 and 1 mM DTT

### Substrates:

Biotin-ATF2-peptide sequence: Biotin-(Ahx)-AGAGDQTPTPTRFLKRPR-amide
Biotin-TYR1-peptide sequence: Biotin-(Ahx)-GAEEEIYAAFFA-COOH
Biotin-TYR2-peptide sequence: Biotin-(Ahx)-AEEEYFFLFA-amide
Biotin-MBP-peptide sequence: Biotin-(Ahx)-VHFFKNIVTPRTPPPSQGKGAEGQR-amide
Biotin-polyGluTyr peptide was purchased from Cisbio (cat #61GT0BLA, Bedford, Mass.)
KinEASE S2 and S3 peptides were purchased from Cisbio (cat #62ST0PEB, Bedford, Mass.)

### Detection Reagents:

Anti-pATF2-Eu was custom-labeled by Cisbio (Bedford, Mass.)
Anti-pMBP-Eu was custom-labeled by Cisbio (Bedford, Mass.)
PT66K was purchased from Cisbio (cat #61T66KLB, Bedford, Mass.)
SAXL was purchased from Prozyme (cat #PJ25S, San Leandro, (A)
Human T-Blasts IL-2 pSTAT5 Cellular Assay

### Materials:

Phytohemaglutinin T-blasts were prepared from Leukopacks purchased from Biological Specialty Corporation, Colmar, Pa. 18915, and cryopreserved in 5% DMSO/media prior to assay.
For this assay the cells were thawed in assay medium with the following composition: RPMI 1640 medium (Gibco 11875093) with 2 mM L-glutamine (Gibco 25030-081), 10 mM HEPES (Gibco 15630-080), 100 μg/mL Pen/Strep (Gibco 15140-122), and 10% heat inactivated FBS (Gibco 10438026). Other materials used in the assay: DMSO (Sigma D2650), 96-well dilution plates (polypropylene) (Corning 3365), 96-well assay plates (white, ½ area, 96 well) (Corning 3642), D-PBS (Gibco 14040133), IL-2 (R&D 202-IL-10 (10 μg)), Alphascreen pSTAT5 kit (Perkin Elmer TGRS5S10K) and Alphascreen protein A kit (Perkin Elmer 6760617M)

### Methods:

T-Blasts were thawed and cultured for about 24 h without IL-2 prior to assay. Test compounds or controls we are dissolved and serially diluted in 100% DMSO. DMSO stocks are subsequently diluted 1:50 in cell culture media to create the 4× compound stocks (containing 2% DMSO). Using a Corning white 96 well, ½ area plate, cells were plated at 2×10⁵/10 μL/well in 10 μL media followed by addition of 5 μL of 4× test compound in duplicate. Cells were incubated with compound for about 0.5 h at about 37° C. Next, 5 μL of IL-2 stock was added at 20 ng/mL final concentration. IL-2 is stored as a 4 μg/mL stock solution, as specified by the manufacturer, at about −20° C. in aliquots and diluted 1:50 with assay media (to 80 ng/mL) just prior to use. The contents of the wells were mixed by carefully tapping sides of plate(s) several times followed by incubation at about 37° C. for about 15 min. The assay was terminated by adding 5 μL of 5× AlphaScreen lysis buffer and shaking on an orbital shaker for about 10 min at rt. Alphascreen acceptor bead mix was reconstituted following Perkin Elmer's protocol. 30 μL/well of reconstituted Alphascreen acceptor bead mix was added, covered with foil then shaken on orbital shaker for about 2 min on high then about 2 h on low. Donor bead mix was reconstituted following Perkin Elmer's AlphaScreen protocol; 12 μL/well were added, covered with foil then shaken for about 2 min on high, and about 2 h on low. Plates were read on an EnVision reader following Perkin Elmer's AlphaScreen protocol instructions.
TF-1 IL-6 pSTAT3 Cellular Assay

### Materials:

TF-1 cells (ATCC #CRL-2003). Culture medium: DMEM medium (Gibco 11960-044) with 2 mM L-glutamine (Gibco 25030-081), 10 mM HEPES (Gibco 15630-080), 100 μg/mL Pen/Strep (Gibco 15140-122), 1.5 g/L sodium bicarbonate (Gibco 25080-094), 1 mM sodium pyruvate (Gibco 11360-070), 10% heat inactivated FBS (Gibco 10437-028), and 2 ng/mL GM-CSF (R&D 215-GM-010). Other materials used in this assay: DMSO (Sigma D2650), 96-well dilution plates (polypropylene) (Corning 3365), 96-well assay plates (white, ½ area, 96 well) (Corning 3642), D-PBS (Gibco 14040133), IL-6 (R&D 206-IL/CF-050 (50 μg)), Alphascreen pSTAT3 kit (Perkin Elmer TGRS3S10K) and Alphascreen protein A kit (Perkin Elmer 6760617M).

### Methods:

Prior to the assay, cells were cultured for about 18 h in the culture medium without GM-CSF. Test compounds or controls were dissolved, and serially diluted in 100% DMSO. DMSO stocks were subsequently diluted 1:50 in cell culture media to create the 4× compound stocks (containing 2% DMSO). Using a Corning white 96 well, ½ area plate, cells were plated at 2×10⁷/10 μL/well in 10 μL media followed by addition of 5 μL of the 4× test compound stock in duplicate. Cells were incubated with compound for about 0.5 h at about 37° C. followed by addition of 5 μL of 400 ng/mL IL-6. IL-6 is stored in 10 μg/mL aliquots using endotoxin free D-PBS (0.1% BSA) at about −20° C. Prior to assay IL-6 was diluted to 400 ng/mL in culture media and applied (5 μL/well) to all wells, except to negative control wells where 5 μL/well of media was added. The contents of the wells were mixed carefully by tapping the side of the plate several times. Plates were incubated at about 37° C. for about 30 min. Cells are lysed by adding 5 μL of 5× AlphaScreen cell lysis buffer to all wells, shaken for about 10 min at rt then assayed. Alternatively, assay plates may be frozen at about −80° C. and thawed later at rt. Using the pSTAT3 SureFire Assay kit (Perkin Elmer #TGRS3S10K) acceptor bead mix was reconstituted following Perkin Elmer's AlphaScreen protocol instructions, 30 μL were added per well then the plate was covered with foil and shaken on an orbital shaker for about 2 min on high, then about 2 h on low at rt. Donor bead mix was reconstituted following Perkin Elmer's AlphaScreen protocol instructions, 12 μL were added per well, then covered with foil and shaken on orbital shaker for about 2 min on high, then about 2 h on low at about 37° C. Plates were read on an EnVision reader following Perkin Elmer's AlphaScreen protocol instructions at rt.
UT7/EPO pSTAT5 Cellular Assay

### Materials:

UT7/EPO cells were passaged with erythropoietin (EPO), split twice per week and fresh culture medium is thawed and added at time of split. Culture Medium: DMEM medium (Gibco 11960-044) with 2 mM L-glutamine (Gibco 25030-081), 10 mM HEPES (Gibco 15630-080), 100 U/mL Pen/Strep (Gibco 15140-122), 10% heat inactivated FBS (Gibco 10437-028), EPO (5 μL/mL=7.1 μL of a 7 μg/mL stock per mL of medium). Assay media: DMEM, 2 mM L-glutamine, 5% FBS, 10 mM HEPES. Other materials used in the assay: DMSO (Sigma D2650), 96-well dilution plates (polypropylene) (Coming 3365), 96-well assay plates (white, ½ area, 96 well) (Coming 3642), D-PBS (Gibco 14040133), IL-2 (R&D 202-IL-10 (10 μg)), Alphascreen pSTAT5 kit (Perkin Elmer TGRS5S10K) and Alphascreen protein A kit (Perkin Elmer 6760617M).

### Methods:

Cultured cells for about 16 h without EPO prior to running assay. Test compounds or controls were dissolved and serially diluted in 100% DMSO. DMSO stocks were subsequently diluted 1:50 in cell culture media to create the 4× compound stocks (containing 2% DMSO). Using a Coming white 96 well, ½ area plate, cells were plated at 2×10⁵/10 μL/well in 10 μL media followed by addition of 5 μL of 4× test compound stock in duplicate. Cells were incubated with compound for about 0.5 h at about 37° C. After incubation, 5 μL of EPO was added to afford a final concentration of 1 nM EPO. The contents of the wells were mixed by carefully tapping sides of the plate several times followed by incubation at about 37° C. for about 20 min. 5 μL of 5× AlphaScreen lysis buffer were added followed by shaking on an orbital shaker for about 10 min at rt. 30 μL/well of acceptor beads were added after reconstitution following Perkin Elmer's AlphaScreen protocol, covered with foil and shaken on orbital shaker for about 2 min on high, then about 2 h on low. Donor beads were reconstituted following Perkin Elmer's AlphaScreen protocol instructions followed by addition of 12 μL/well, covered with foil and shaken on an orbital shaker for about 2 min on high, about 2 h on low. Plates were read on an EnVision reader following Perkin Elmer's AlphaScreen protocol instructions.

### Antigen-Induced Degranulation of RBL-2H3 Cells:

RBL-2H3 cells were maintained in T75 flasks at about 37° C. and 5% CO₂, and passaged every 3-4 days. To harvest cells, 20 mL of PBS was used to rinse the Bask once, and then 3 mL of Trypsin-EDTA was added and incubated at about 37° C. for about 2 min. Cells were transferred to a tube with 20 mL, medium, spun down at 1000 RPM at rt for about 5 min and resuspended at 1×10⁶ cells/mt. Cells were sensitized by adding DNP-specific mouse IgE to a final concentration of 0.1 μg/mL. 50 μL of cells were added to each well of a 96 well flat bottom plate (50×10³ cells/well) and incubated overnight at about 37° C. in 5% CO₂. The next day, compounds were prepared in 100% DMSO at 10 mM. Each compound was then serially diluted 1:4 six times in 100% DMSO. Each compound dilution was then diluted 1:20 and then 1:25, both dilutions in Tyrode's buffer. Media was aspirated from the cell plates and the cells were rinsed twice with 100 μL of Tyrode's buffer (prewarmed to about 37° C.), 50 μL of compounds diluted in Tyrode's buffer were added to each well and the plates were incubated for about 15 min at about 37° C. in 5% CO₂. 50 μL of 0.2 μg/mL DNP-HSA in Tyrode's buffer was then added to each well and the plates were incubated for about 30 min at about 37° C. in 5% CO₂. The final concentration of the various components in the incubation mix were 0.002-10 μM compounds, 0.1% DMSO, and 0.1 μg/mL DNP-HSA. As one control, 0.2% DMSO (no compound) in Tyrode's buffer was added to a set of wells to determine maximum stimulated release. As a second control, Tyrode's buffer without DNP-HSA was added to a set of wells with containing 0.2% DMSO without compounds to determine unstimulated release. Each condition (compounds and controls) was set up in triplicate wells. At the end of the 30 min incubation, 50 μL of supernate was transferred to a new 96 well plate. The remaining supernate in the cell plates was aspirated and replaced with 50 μL of 0.1% Triton X-100 in Tyrode's buffer to lyse the cells. 50 μL of freshly prepared 1.8 mM 4-Nitrophenyl N-acetyl-β-D-glucosaminide (pNAG) was then added to each well of supernate and cell lysate and the plates were incubated for about 60 min at about 37° C. in 5% CO₂. 100 μL of 7.5 mg/mL sodium bicarbonate was added to each well to stop the reaction. The plates were then read at 405 nm on a Molecular Devices SpectraMax 250 plate reader.

### Calculation of Results

1) The plate background OD₄₀₅ obtained from wells containing Tyrode's buffer and pNAG (no supernate or lysate) is subtracted from the OD₄₀₅ reading for each well containing supernate or lysate.
2) The release for each well is expressed as the percentage of the total release for that well, where the total release is twice the release in the supernate plus the release in the cell lysate. This calculation corrects for variable cell number in each well.
3) The maximum response is the mean response of wells containing DNP-HSA but no compound.
4) The minimum response is the mean response of wells containing no DNP-HSA and no compound.
5) The response in each compound well is calculated as a percentage of the maximum response (expressed as % control) where the maximum response is 100% and the minimum response is 0%.
6) A dose response curve is generated for each compound and the IC₅₀ of the curve is calculated using Prism GraphPad software and nonlinear least squares regression analysis.
Acute in vivo measurement of JAK inhibition by compounds is measured using the:

### Concanavalin A (Con A)-Induced Cytokine Production in Lewis Rats

The test compound was formulated in an inert vehicle (for example but not limited to 0.5% hydroxypropylmethyl cellulose (Sigma, cat #H3785)/0.02% Tween 80 (Sigma, cat 4780) in water) at the desired concentration to achieve doses in the range of 0.01-100 mg/kg. Six-week-old male Lewis rats (125 g-150 g) (Charles River Laboratories) were dosed with the compound orally, at time zero (0 min). After about 30 min the rats were injected intravenously (i.v.) with 10 mg/kg Concanavalin A (Con A, AmershamBioscience, cat #17-0450-01) dissolved in PBS (Invitrogen, cat #14190). About 4 h later, the rats were cardiac bled and their plasma was analyzed for levels of IL-2 (ELISA kit: R&D Systems cat #R2000) and IFN-γ (ELISA kit: R&D Systems cat #RIF00).
Chronic in vivo effects of the compounds on an arthritis disease model were measured using the:

### Adjuvant Induced Arthritis (MA) Model in a Lewis Rat

Female Lewis rats, (6 weeks of age, 125 g-150 g in weight from Charles River Laboratories) were immunized intradermally (i.d.) in the right hind-footpad with 100 μL of a suspension of mineral oil (Sigma, cat # M5905) and containing 200 μg M. tuberculosis, H37RA (Difco, cat #231141). The inflammation appeared in the contra-lateral (left) hind paw seven days after the initial immunization. Seven days post immunization, the compound was formulated in an inert vehicle (for example but not limited to 0.5% hydroxypropylmethyl cellulose (Sigma, cat #H3785)/0.02% Tween 80 (Sigma, cat #4780) in water) and dosed orally once or twice a day for at least 10 days. Baseline paw volume was taken on day 0 using a water displacement pleythsmograph (Vgo Basile North America Inc. PA 19473, Model #7140). Rats were lightly anesthetized with an inhalant anesthetic (isoflurane) and the contra-lateral (left) hind paw was dipped into the plethysmograph and the paw volume was recorded. The rats were scored every other day up to day 17 after immunization. On day 17 after immunization, all rats were exsanguinated by cardiac puncture under isoflurane anesthesia, and the left hind paw was collected to assess the impact on bone erosion using micro-CT scans (SCANCO Medical, Southeastern, PA, Model # μCT 40) at a voxel size of 18 μm, a threshold of 400, sigma-gauss 0.8, support-gauss 1.0, Bone volume and density was determined for a 360 μm (200 slice) vertical section encompassing the tarsal section of the paw. The 360 μm section was analyzed from the base of the metatarsals to, the top of the tibia, with the lower reference point fixed at the tibiotalar junction. Drug exposure was determined in the plasma using LC/MS.
or the:

### Collagen Induced Arthritis (CIA) Model in a Lewis Rat

On day −1 Collagen Type II (CII), soluble from bovine nasal septum (Elastin Products, Cat #CN276) was weighed out for a dose of 600 μg/rat, 0.01M acetic acid (150 μL HOAc grade. J. T. Baker, order#9522-03, and 250 mL, Milli Q Water) was added for a concentration of 4 mg/mL. The vial was covered with aluminum foil and placed on a rocker at about 4° C.: overnight. On day 0 collagen stock solution was diluted 1:1 with Incomplete Freunds adjuvant (IFA) (Difco labs, cat #263910) using a glass Hamilton luer lock syringe (SUE Syringe Perfection VWR cat #007230), final concentration 2 mg/mL. Female Lewis rats (Charles River Laboratories) acclimated for 7 days at the time of immunization weighing approximately 150 g were anesthetized in an anesthesia chamber using isoflurane (5%) and oxygen. Once the rats were completely anesthetized, they were transferred to a nose cone to maintain anesthesia during the injections. Rats were shaved at the base of the tail, 300 μL of collagen was injected i.d. on the rump of the rat, n=9 per group. 100 μL at three sites with a 500 μL lent lock syringe and a 27 g needle. IFA control rats are injected in the same manner (n=6). The IFA was a 1:1 emulsion with the 0.01M acetic acid. Boost was done on day 6 of the study. Shaving was not done on this day and injections were done in the same manner as the immunization. The inflammation appeared in both hind paws 10 days after the initial immunization. 10 days post immunization, the compound was formulated in an inert vehicle (for example but not limited to 0.5% hydroxypropylmethyl cellulose (Sigma, cat #H3785)/0.02% Tween 80 (Sigma, cat #4780) in water) and dosed orally once or twice a day for at least 9 days. Baseline paw volume was taken on day 7 using a water displacement pleythsmograph (Vgo Basile North America Inc. PA 19473, Model #7140). Rats were lightly anesthetized with an inhalant anesthetic (isoflurane) and both hind paws were dipped into the plethysmograph and the paw volume was recorded. The rats were scored 2 to 3 times a week up to day 18 after immunization. On day 18 after immunization, all rats were exsanguinated by cardiac puncture under isoflurane anesthesia, and the hind paws were collected to assess the impact on bone erosion using micro-CT scans (SCANCO Medical, Southeastern, PA, Model # μCT 40) at a voxel size of 18 μm, a threshold of 400, sigma-gauss 0.8, support-gauss 1.0. Bone volume and density was determined for a 360 μm (200 slice) vertical section encompassing the tarsal section of the paw. The 360 μm section was analyzed from the base of the metatarsals to the top of the tibia, with the lower reference point fixed at the tibiotalar junction. Drug exposure was determined from plasma using LC/MS.
Chronic in vivo effects of the compounds on an asthma disease model were measured using the:

### OVA Induced Rat Asthma Model

Female Brown Norway rats (7-9 weeks of age) were sensitized on day 0 and 7 with 40 μg ovalbumin (OVA) (Sigma-Aldrich, St. Louis, Mo.) in a 20 mg/mL solution of Alum Inject (Pierce, Rockford, Ill.). The rats were subsequently challenged intratracheally on day 19 and 20 with 1.5 μg OVA in 50 μL PBS. Dosing of inhibitor began on day 18 and continued through day 22. On day 22, 48 h after the second challenge, rats were subjected to an anesthetized and restrained pulmonary function test. Airway hyperresponsiveness (AHR) was assessed using whole body plethysmography. Briefly, a surgical plane of anesthesia was induced with an intraperitoneal injection of 60 mg/kg ketamine and 5 mg/kg xylazine (Henry Schein, Inc., Melville, INA). A tracheal cannula was surgically inserted between the 3rd and 4th tracheal rings. Spontaneous breathing was prevented by jugular vein injection of 0.12 mg/kg pancuronium bromide (Sigma-Aldrich, St Louis, Mo.). Animals were placed in a whole body plethysmograph (Buxco Electronics, Inc., Wilmington, N.C.) and mechanically ventilated with 0.2 mL room air at 150 breaths per minute with a volume controlled ventilator (Harvard Apparatus, Framingham, Mass.). Pressure in the lung and flow within the plethysmograph were measured using transducers and lung resistance was calculated as pressure/flow using Biosystem Xa software (Buxco Electronics). Airway resistance was measured at baseline and following challenge with 3, 10, and 30 mg/mL methacholine (Sigma Aldrich, St, Louis, Mo.) delivered with an inline ultrasonic nebulizer. Upon completion of pulmonary function testing, the lungs were lavaged 3 times with 1 mL sterile PBS. The volume from the first wash was centrifuged at 2000 rpm for 5 min, and the supernatant is stored for subsequent analysis. The volume of washes 2 through 3 were added to the pellet derived from the first wash and subsequently processed for evaluation of cellular infiltrate by flow cytometry. Plasma was collected from blood drawn from the vena cava and was used for evaluation of drug concentrations.

The teachings of all references, including journal articles, patents and published, patent applications, are incorporated herein by reference in their entirety.

The following examples are for illustrative purposes and are not to be construed as limiting the scope of the present invention.

### Analytical Methods

Analytical data was included within the procedures below, in the illustrations of the general procedures, or in the tables of examples. Unless otherwise stated, all ¹H NMR data were collected on a Varian Mercury Plus 400 MHz or a Varian Inova 600 MHz instrument and chemical shifts are quoted in parts per million (ppm). LC/MS and HPLC data are referenced to the table of LC/MS and HPLC conditions using the lower case method letter provided in Table 1.

TABLE 1
LC/MS and HPLC methods

| Method | Conditions |
|---|---|
| a | LC/MS: The gradient was 5-60% B in 1.5 min then 60-95% B |
| | to 2.5 min with a hold at 95% B for 1.2 min (1.3 mL/min flow |
| | rate). Mobile phase A was 10 mM NH₄OAc, mobile phase B |
| | was HPLC grade MeCN. The column used for the |
| | chromatography is a 4.6 × 50 mm MAC-MOD Halo C18 |
| | column (2.7 μm particles). Detection methods are diode array |
| | (DAD) and evaporative light scattering (ELSD) detection as |
| | well as positive/negative electrospray ionization. |
| b | LC/MS: The gradient was 5-60% B in 1.5 min then 60-95% B |
| | to 2.5 min with a hold at 95% B for 1.2 min (1.3 mL/min flow |
| | rate). Mobile phase A was 10 mM NH₄OAc, mobile phase B |
| | was HPLC grade MeCN. The column used for the |
| | chromatography is a 4.6 × 50 mm MAC-MOD Halo C8 |
| | column (2.7 μm particles). Detection methods are diode array |
| | (DAD) and evaporative light scattering (ELSD) detection as |
| | well as positive/negative electrospray ionization. |
| c | LC/MS: The gradient was 5-60% B in 0.75 min then 60-95% B |
| | to 1.15 min with a hold at 95% B for 0.75 min (1.3 mL/min |
| | flow rate). Mobile phase A was 10 mM NH₄OAc, mobile phase |
| | B was HPLC grade MeCN. The column used for the |
| | chromatography was a 4.6 × 50 mm MAC-MOD Halo C8 |
| | column (2.7 μm particles). Detection methods are diode array |
| | (DAD) and evaporative light scattering (ELSD) detection as |
| | well as positive/negative electrospray ionization. |
| d | The gradient was a hold at 10% B over 2.50 min followed by |
| | 10-14.5% B over 0.50 min, 14.5-46% B for 6 min, a hold at |
| | 46% B for 0.1 min, and 46-95.5% B over 1.5 min (25 mL/min |
| | flow rate). Mobile phase A was 50 mM NH₄OAc (pH 4.5) and |
| | mobile phase B was HPLC grade MeCN. The column used for |
| | the chromatography was a 19 × 50 mm Waters Atlantis T3 |
| | OBD C18 column (5 μm particles), detection methods are |
| | Photodiode array DAD and Waters ZQ 2000 mass spectrometer. |
| e | LC/MS: The gradient was 5-60% B in 0.60 min then 60-95% |
| | B to 1.0 min with a hold at 95% B for 0.30 min (1.25 mL/min |
| | flow rate). Mobile phase A was 10 mM NH₄OAc, mobile phase |
| | B was HPLC grade MeCN. The column used for the |
| | chromatography is 2.1 × 30 mm Acquity UPLC HSS T3 |
| | column (1.8 μm particles). Detection methods are diode array |
| | (DAD) and evaporative light scattering (ELSD) detection as |
| | well as positive/negative electrospray ionization. |
| f | The gradient was a hold at 19% B over 2.50 mM followed by |
| | 19-23.5% B over 1 min, 23.5-95.5% B for 9.3 min and a hold at |
| | 95.5% for 0.7 min (25 mL/min flow rate). Mobile phase A was |
| | 50 mM NH₄OAc (pH 4.5) and mobile phase B was HPLC |
| | grade MeCN. The column used for the chromatography was a |
| | 19 × 100 mm Waters Atlantis T3 ODD C18 column (5 μm |
| | particles) detection methods are Photodiode array DAD and |
| | Waters ZQ 2000 mass spectrometer. |
| g | SFC/MS: The gradient was 10-55% B co-solvent 7 min then |
| | hold at 55% for 1 min (4 mL/min flow rate, 100 bar, 37° C.). |
| | Solvent A was SFC grade CO₂. Co-solvent B was HPLC grade |
| | methanol with 0.1% diethylamine added. The column used for |
| | the chromatography was a 4.6 × 250 mm Daicel IA column |
| | (5 μm particles). Detection methods were diode array (DAD) |
| | and evaporative light scattering (USD) detection as well as |
| | positive/negative electrospray ionization. |

TABLE 2
Chiral HPLC methods

| Method | Conditions |
|---|---|
| 1 | Isocratic 100% EtOH (200 proof) with 0.12% diethylamine |
| | for 17 min (20 mL/min flow rate). The column used for the |
| | chromatography was a Daicel IA, 20 × 250 mm column (5 μm |
| | particles). The detection methods used were evaporative light |
| | scattering (ELSD) detection and optical rotation |
| 2 | Isocratic 30% EtOH (200 proof)/heptane with 0.1% |
| | diethylamine for 18.5 min (20 mL/min flow rate). The column |
| | used for the chromatography was a Daicel IA, 20 × 250 mm |
| | column (5 μm particles). The detection methods used were |
| | evaporative light scattering (ELSD) detection and optical |
| | rotation. |
| 3 | The gradient was 15-50% EtOH (200 proof)/heptane with |
| | 0.12% diethylamine for 18 min (20 mL/min flow rate). The |
| | column used for the chromatography was a Daicel IA, |
| | 20 × 250 mm column (5 μm particles). |
| 4 | Eluted with 10% EtOH and 90% heptanes with 0.1% |
| | diethylamine added. The column used for the chromatography |
| | was a Viridis 2-ethylpyridine 30 × 100 mm column (5 μm |
| | particles). The detection methods used were UV ( λ = 340 nm) |
| | as well as optical rotation. |
| 5 | Isocratic 15% A for 35 min (20 mL/min flow rate). Mobile |
| | phase A was EtOH (200 proof), mobile phase B was HPLC |
| | grade heptane with 0.12% diethylamine added. The column |
| | used for the chromatography was a Daicel IA, 20 × 250 mm |
| | column (5 μm particles). Detection methods were evaporative |
| | light scattering (ELSD) detection as well as optical rotation. |
| 6 | Isocratic 25% A for 31 min (20 mL/min flow rate). Mobile |
| | phase A was ethanol (200 proof), mobile phase B was HPLC |
| | grade heptane with 0.1% diethylamine added. The column |
| | used for the chromatography was a Daicel IA, 20 × 250 mm |
| | column (5 μm particles). Detection methods were evaporative |
| | light scattering (ELSD) detection, and optical rotation. |
| 7 | Isocratic 10% A for 30 min (20 mL/min flow rate). Mobile |
| | phase A was EtOH (200 proof), mobile phase B was HPLC |
| | grade heptane with 0.12% diethylamine added. The column |
| | used for the chromatography was a Daicel IA, 20 × 250 mm |
| | column (5 μm particles). Detection methods were evaporative |
| | light scattering (ELSD) detection and optical rotation. |
| 8 | Isocratic 35% A for 24 min (20 mL/min flow rate). Mobile |
| | phase A was ethanol (200 proof), mobile phase B was HPLC |
| | grade heptane with 0.12% diethylamine added. The column |
| | used for the chromatography was a Daicel IC, 20 × 250 mm |
| | column (5 μm particles). Detection methods were evaporative |
| | light scattering (ELSD) detection, and/or UV (variable |
| | wavelength) as well as optical rotation. |
| 9 | The gradient was 20-50% A in 19 min then 50-60% A in 1.5 |
| | min (20 mL/min flow rate, room temperature). Mobile phase |
| | A was EtOH (200 proof), mobile phase B was HPLC grade |
| | heptane with 0.12% diethylamine added. The column used |
| | for the chromatography was a Daicel IA, 20 × 250 mm |
| | column (5 μm particles). Detection methods were evaporative |
| | light scattering (ELSD) detection, and/or UV (variable |
| | wavelength) as well as optical rotation. |
| 10 | Isocratic gradient of 100% EtOH (200 proof) with 0.12% |
| | diethylamine for 17 min (20 mL/min flow rate). The column |
| | used for the chromatography was a Daicel IA, 20 × 250 mm |
| | column (5 μm particles). The detection methods used were |
| | evaporative light scattering (ELSD) detection and optical |
| | rotation |

### General Synthetic Schemes

Compounds of the invention may be prepared using synthetic transformations such as those illustrated in Schemes I-III. Starting materials are commercially available, may be prepared by the procedures described herein, by literature procedures, or by procedures that would be well known to one skilled in the art of organic chemistry (see, for example, Larock, R. C. “Comprehensive Organic Transformations: A Guide to Functional Group Preparations, 2^{nd} edition”, 1999, Wiley-VCH or Greene, T. W. and Wuts, P. G. M. “Protective Groups in Organic Synthesis, 3^{rd} Edition”, 1999, Wiley-Interscience). Methods for preparing (1S,3S,4R)-3-(3H-imidazo[1,2-a]pyrrolo[2,3-e]pyrazin-8-yl)-4-methylcyclopentanamine-derived amides of the invention are illustrated in Scheme I. Reaction of Corey ylide with bis-benzylamine protected cyclopentyl carboxylic acid 1 (WO2011/068881) shown below gives sulfur ylide 2 as described in WO2010/099039; its reaction with 5-tosyl-5H-pyrrolo[2,3-b]pyrazin-2-amine 3 (WO2011/068881) in the presence of 1r catalyst (WO2010/099039) yields aminomethyl ketone 4. Its cyclization in the presence of TFAA/TFA mixture or alternatively by using pentafluoropropanoic anhydride followed, by the removal of the tosyl protecting group furnishes tricyclic N,N-dibenzylamine 5. The removal of N,N-dibenzyl protecting group in the presence of Pd catalyst affords primary amine 6 which could be further derivatized to yield compounds of general structure 7.

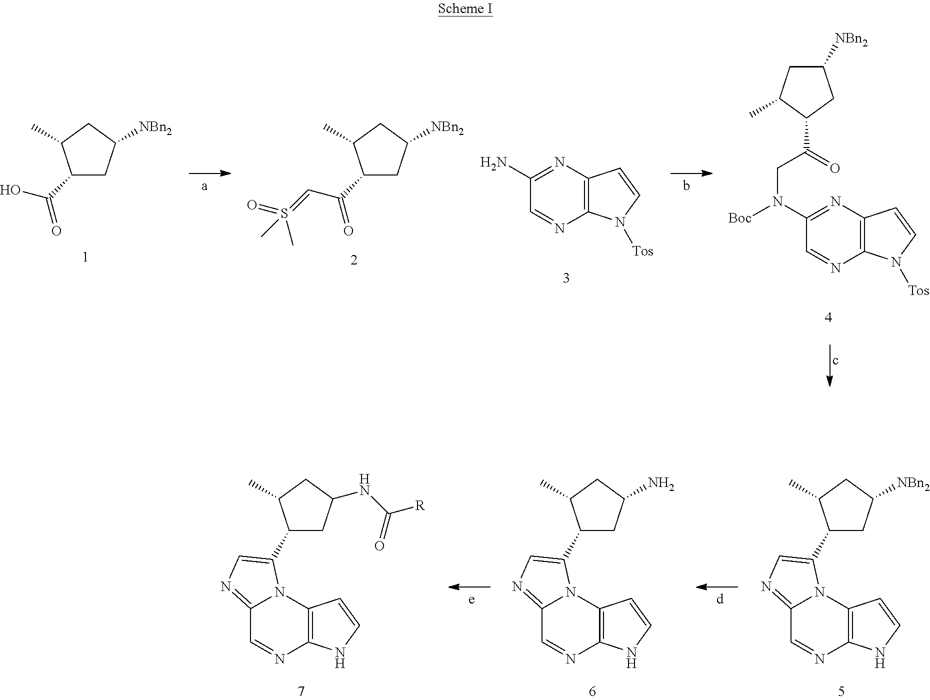

An alternate method for preparing (1S,3S,4R)-3-(3H-imidazo[1,2-a]pyrrolo[2,3-e]pyrazin-8-yl)-4-methylcyclopentanamine derivatives is shown in Scheme II. An amine 1 described above could be converted to the ketone 2 by using 3,5-di-tert-butyl-[1,2]benzoquinone. The ketone undergoes reductive amination to give mono- (R″=H) or di-substituted amine products 3. This method is most useful for the reaction of secondary amines including cyclic amines.

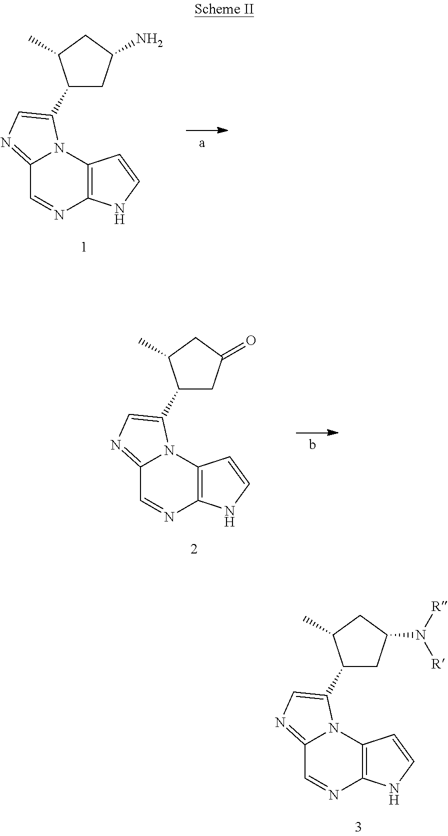

Methods for preparing pyrrolidine-containing compounds of the invention are described Scheme III. Reduction of substituted propargyl ester 1 in the presence of Lindlar catalyst affords corresponding Z-ene ester 2. Its reaction with N-benzyl-1-methoxy-N-((trimethylsilyl)methyl)methanamine 3 affords N-benzyl protected pyrrolidine ester 4 enriched in cis isomer. Ester hydrolysis followed by debenzylation and pyrrolidine nitrogen protection yields N-benzyl carbamate 5. It could be resolved, using sequential use of S and R enantiomers of 1-(naphthalen-1-yl)ethanamine or 1-phenylethanamine to yield corresponding salts of enantiomerically pure substituted pyrrolidine carboxylate 6. Generation of free acid followed the conversion to acid chloride and derivatization with TMS-diazomethane and aqueous fair yielded bromomethyl ketone 7. Its reaction with tert-butyl 5-tosyl-5,1-pyrrolo[2,3-b]pyrazin-2-ylcarbamate 8 (WO2011/068881) affords Boc-protected aminomethyl ketone 9. The removal of the Hoc group followed by cyclization in the presence of Lawesson's reagent and removal of Cbz group furnishes tricycle with free pyrrolidine 10 that could be isolated as free base or as a salt. Further derivatization followed by the removal of the tosyl protecting group yields compounds of general structure 12 or 14.

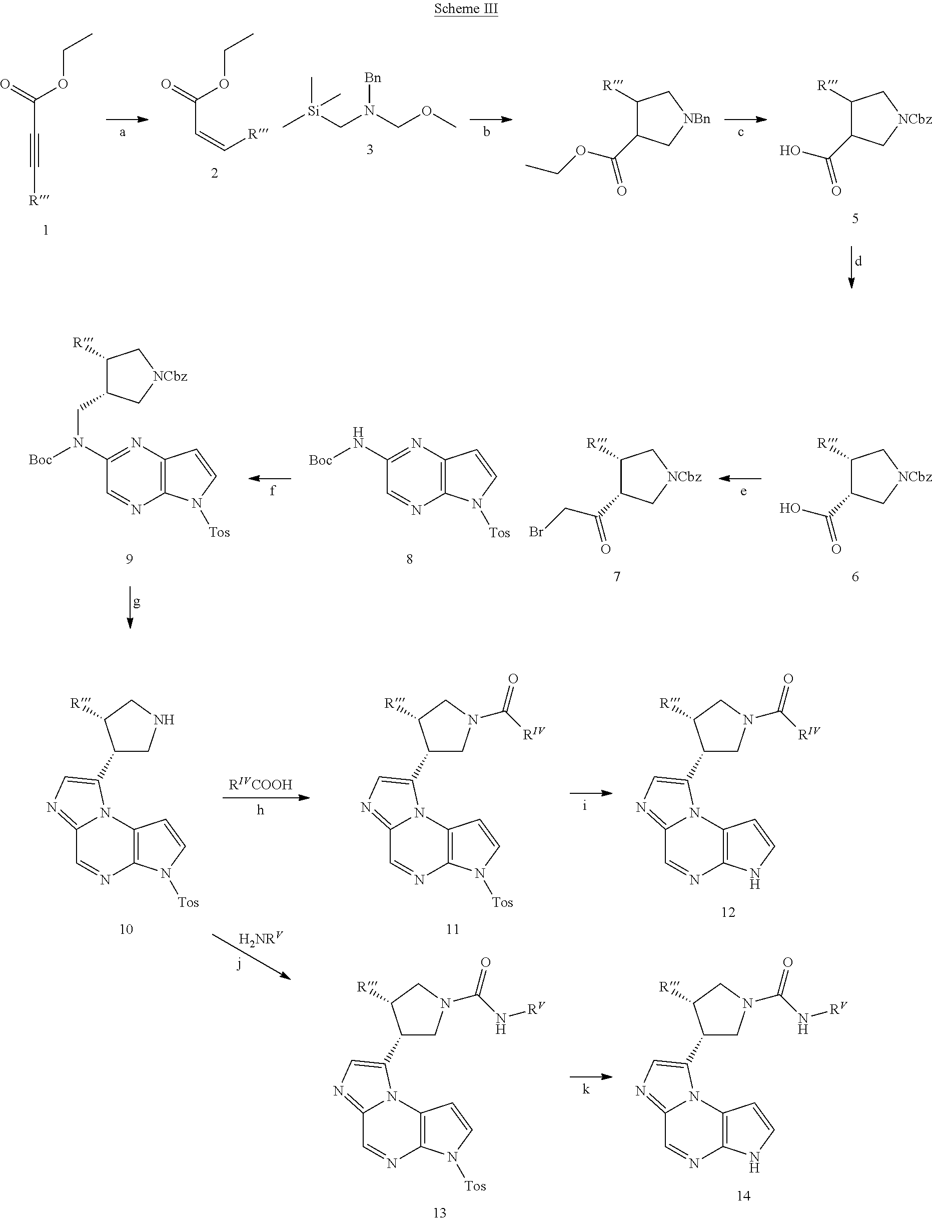

### Example #1

### N-((1S,3S,4R)-3-(3H-imidazo[1,2-a]pyrrolo[2,3-e]pyrazin-8-yl)-4-methylcyclopentyl)-5-methylpyrazine-2-carboxamide

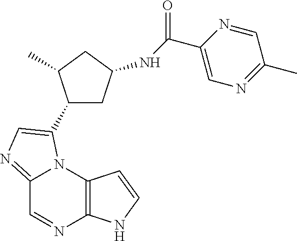 [SMILES: Cc1cnc(C(=O)N[C@H]2C[C@@H](C)[C@@H](c3cnc4cnc5[nH]ccc5n34)C2)cn1]

### Step A: (1S,2R,4S)-2-(2-methyl-4-(dibenzylamino)cyclopentyl)-dimethylsulfoxonium-2-oxo-ethylide

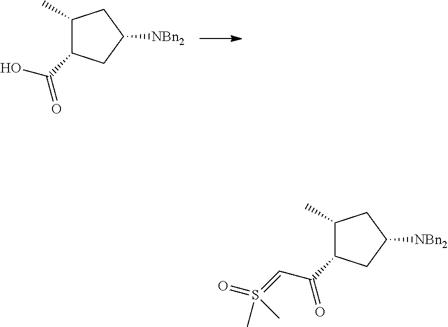

Trimethylsulfoxonium chloride (26.1 g, 198 mmol), THE (202 mL), and potassium tert-butoxide (23.4 g, 202 mmol) were added to a 500 mL jacketed flask under a nitrogen blanket. The suspension was stirred for about 2 h at about 65° C. before being cooled to about 0° C. In a separate flask, (1S,2R,4S)-4-(dibenzylamino)-2-methylcyclopentanecarboxylic acid (21.4 g, 66.2 mmol, WO2011/068881) was dissolved in THF (134 mL), HATU (31.4 g, 83 mmol) and TEA (11.5 mL, 83 mmol) were added and the solution was mixed for about 4 h. With the sulfur ylide suspension maintained between about 0 and −5° C., the activated ester solution was filtered and then added dropwise over about 3 h to the ylide suspension. The resulting bright yellow suspension was stirred for about 8 h at about 5° C. Water (340 mL) and THF (30 mL) were added, and the mixture stirred for about 30 min at about 25° C. Aqueous sodium chloride (15%, 60 mL) was added to the solution and the layers separated. The aqueous layer was extracted with EtOAc (60 mL). The combined organic layers were washed with aqueous NaCl (15%, 3×100 mL). The solution was concentrated and the crude oil was dissolved in methanol (150 mL) and water (150 mL) was added to the slurry which was stirred for about 1 h at ambient temperature before being cooled to about 10° C. and stirred overnight. The white solid was filtered and washed with chilled 1:1 MeOH/water (20 mL) and water (60 mL). The solid was dried in the vacuum oven to afford (1S,2R,4S)-2-(2-methyl-4-(dibenzylamino)cyclopentyl)-dimethylsulfoxonium-2-oxo-ethylide (23.8 g, 90%). ¹H NMR (400 MHz, DMSO) δ 7.30 (ddd, 8H), 7.21-7.14 (m, 2H), 4.67 (s, 1H), 3.71-3.52 (m, 4H), 3.39 (d, 6H), 3.13-2.99 (m, 1H), 2.48-2.39 (m, 1H), 2.05-1.84 (m, 2H), 1.82-1.66 (m, 2H), 1.43-1.30 (m, 1H), 0.90 (d, 3H).

### Step B: 1-((1S,2R,4S)-4-(dibenzylamino)-2-methylcyclopentyl)-2-(5-tosyl-5H-pyrrolo[2,3-b]pyrazin-2-ylamino)ethanone

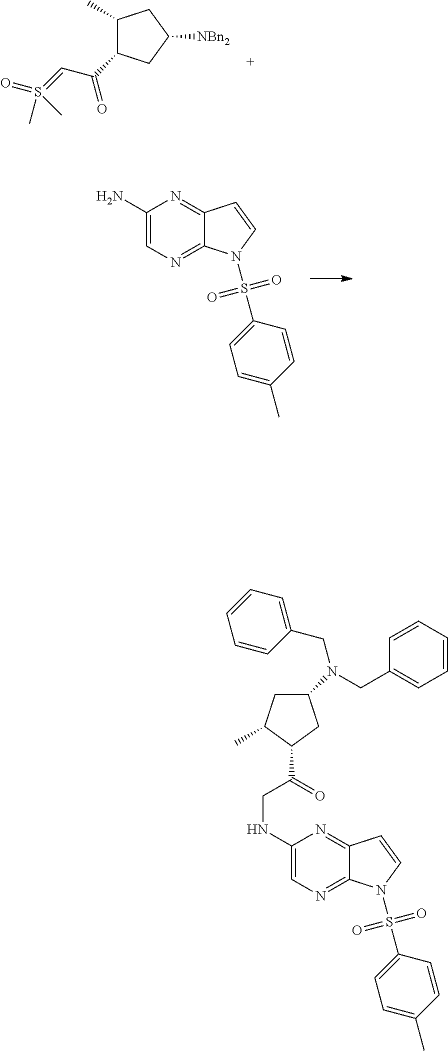

To a 40 mL vial, (1S,2R,4S)-2-(2-methyl-4-(dibenzylamino)cyclopentyl)-dimethylsulfoxonium-2-oxo-ethylide (4.02 g, 10.1 mmol), 5-tosyl-5H-pyrrolo[2,3-b]pyrazin-2-amine (2.92 g, 10.1 mmol, WO2011/068881), and chloro(1,5-cyclooctadiene) iridium(I) dimer (0.17 g, 0.3 mmol, Alfa Aesar) were added. The reaction vessel was purged with nitrogen for about 10 min. To the reaction vessel, degassed CHCl₃ (13 mL) was added via syringe. The reaction mixture was purged with nitrogen for about 10 mL and stirred under an atmosphere of nitrogen at about 70° C. for about 68 h. The reaction mixture was allowed to cool to ambient temperature. The reaction mixture was purified by silica gel flash chromatography eluting with a gradient of 0-25% EtOAc in heptane to yield 1-((1S,2R,4S)-4-(dibenzylamino)-2-methylcyclopentyl)-2-(5-tosyl-SH-pyrrolo[2,3-b]pyrazin-2-ylamino)ethanone (8.61 g, 56%) as tan foam. ¹H NMR (400 MHz, DMSO). δ 7.91-7.80 (m, 4H), 7.42-7.34 (m, 2H), 7.33-7.23 (m, 9H), 7.21-7.13 (m, 2H), 6.52 (d, 1H), 4.23-4.04 (m, 2H), 3.63-3.48 (m, 4H), 3.19-3.09 (m, 1H), 3.08-2.99 (m, 1H), 2.32 (s, 3H), 2.29-2.18 (m, 1H), 1.94-1.71 (m, 3H), 1.37-1.23 (m, 1H), 0.86 (d, 3H).

### Step C: (1S,3S,4R)—N,N-dibenzyl-3-(3H-imidazo[1,2-a]pyrrolo[2,3-e]pyrazin-8-yl)-4-methylcyclopentanamine

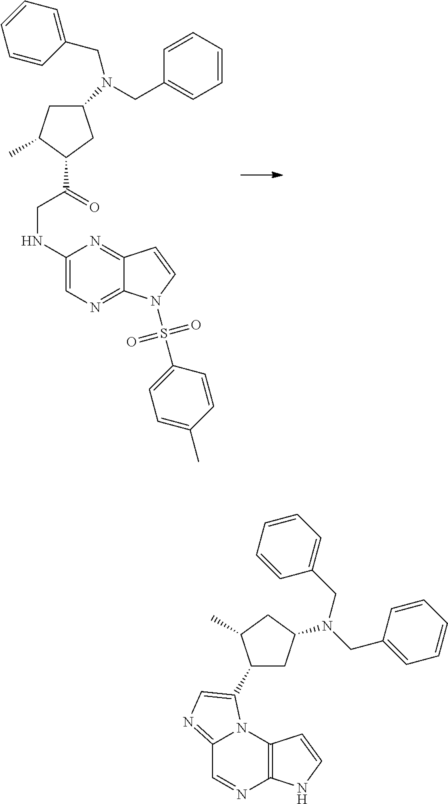 [SMILES: Cc1ccc(S(=O)(=O)n2ccc3nc(NCC(=O)[C@H]4C[C@@H](N(Cc5ccccc5)Cc5ccccc5)C[C@H]4C)cnc32)cc1 ; C[C@@H]1C[C@H](N(Cc2ccccc2)Cc2ccccc2)C[C@@H]1c1cnc2cnc3[nH]ccc3n12]

To a 250 mL, round-bottomed flask 1-((1S,2R,4S)-4-(dibenzylamino)-2-methylcyclopentyl)-2-(5-tosyl-5H-pyrrolo[2,3-b]pyrazin-2-ylamino)ethanone (11.2 e, 17.5 mmol) in acetonitrile (60 mL) was added. The mixture was cooled with an ice bath and TFA (2.70 mL, 35.0 mmol) and TFAA (24.5 mL, 175 mmol) was added. The resulting mixture was warmed and stirred at about 40° C. for about 42 h. The reaction was then cooled in an ice bath and quenched with methanol (7 mL). After warming to ambient temperature and stirring for about 1 h, it was poured into EtOAc (100 mL) and aqueous sodium carbonate (10%, 200 mL). The layers were separated and the organic layer concentrated. The residue was dissolved in THF (120 mL) and 2N aqueous NAOH (35.0 mL, 70.0 mmol) was added. The reaction mixture was warmed to about 60° C. and stirred for about 16 h. After cooling to ambient temperature, 2-methyl-tetrahydrofuran (100 mL) and brine (100 mL) were added and the layers separated. The aqueous layer was extracted with 2-methyltetrahydrofuran (50 mL) and the combined organic layers washed with brine (50 mL). The organic layer was concentrated, dissolved in EtOH (100 mL) and treated with charcoal (0.50 g) for about 1 h. The charcoal was filtered, off and the ethanol was removed under reduced pressure. The residue was taken up in CHCl₃ (50 mL), warmed to about 50° C. and heptane (50 mL) was added. After cooling to ambient temperature, the product was collected, washed with 1:2 CHCl₃:heptane (30 mL) and dried in a vacuum oven to afford (1S,3S,4R)—N,N-dibenzyl-3-(3H-imidazo[1,2-a]pyrrolo[2,3-e]pyrazin-8-yl)-4-methylcyclopentylamine as a tan solid (5.1 g, 67%). ¹H NMR (400 MHz, DMSO) δ 12.18 (s, 1H), 8.52 (s, 1H), 7.60 (s, 1H), 7.44-7.29 (m, 8H), 7.22 (t, 2H), 6.84 (d, 1H), 3.86 (dd, 1H), 3.77-3.59 (m, 4H), 3.41-3.17 (m, 2H), 2.64-2.53 (m, 1H), 2.32-2.06 (m, 3H), 1.49-1.30 (m, 1H), 0.40 (d, 3H).

### Step D: (1S,3S,4R)-3-(3H-imidazo[1,2-a]pyrrolo[2,3-e]pyrazin-8-yl)-4-methylcyclopentylamine

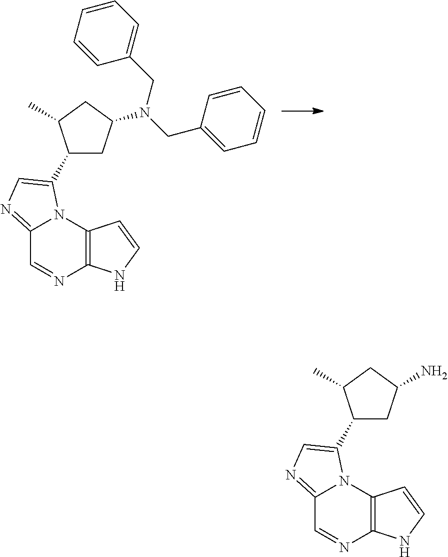 [SMILES: C[C@@H]1C[C@H](N(Cc2ccccc2)Cc2ccccc2)C[C@@H]1c1cnc2cnc3[nH]ccc3n12 ; C[C@@H]1C[C@H](N)C[C@@H]1c1cnc2cnc3[nH]ccc3n12]

To a mixture of (1S,3S,4R)—N,N-dibenzyl-3-(3H-imidazo[1,2-a]pyrrolo[2,3-e]pyrazin-8-yl)-4-methylcyclopentylamine (200 g, 459 mmol) and 10% Pd(OH)₂ on C (40.7 g, Johnson Mathey) under nitrogen was added ethanol (3 L). The reactor was purged with nitrogen then hydrogen. The vessel was pressurized with hydrogen to about 30 psi. The mixture was agitated for about 16 h at about 50° C. Since the reaction was not complete, another 5 wt % of 10% Pd(OH)₂ on C (20.3 g) was added to the reactor as a slurry in EtOH (150 mL). This was done by adding the slurry into the reactor after it was cooled to about 20° C., and purged with nitrogen. The reactor was pressurized with hydrogen to about 30 psi, heated to about 50° C., and agitated for about additional 6 h. The reactor was cooled to about 20° C., and the reactor was purged with nitrogen. The reaction mixture was filtered through a 2 micron polypropylene filter cartridge. The reactor was rinsed with EtOH (600 mL) and that too was filtered. There were catalyst fines present so the combined filtrates were filtered through a 0.45 micron nylon filter membrane. The filtrate was concentrated under reduced pressure to give a thick oil. DCM (400 mL) was added and the mixture was sonicated until the oil was broken up and a fine suspension was present. Cooled to rt and filtered to remove solid (2.6 g). The filtrate was concentrated under reduced pressure to give a tan foam which was dried in a vacuum oven at about 50° C. overnight. DCM (400 mL) was added and the mixture was sonicated to form a fine suspension prior to concentrating under reduced pressure. Repeated twice and dried under vacuum at about 20-60° C. to give crude (1S,3S,4R)-3-(3H-imidazo[1,2-a]pyrrolo[2,3-e]pyrazin-8-yl)-4-methylcyclo-pentylamine containing about 6 mol % ethanol and about) mol % DCM as excipients (112 g, ˜95% crude); LC/MS (Table 1, Method a) Rₜ=1.24 min; MS m/z: 256 (M+H)⁺.

### Step F: N-((1S,3S,4R)-3-(3H-imidazo[1,2-a]pyrrolo[2,3-e]pyrazin-8-yl)-4-methylcyclopentyl)-5-methylpyrazine-2-carboxamide

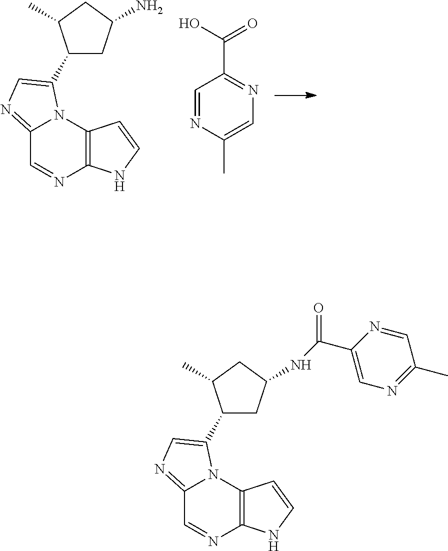

5-Methylpyrazine-2-carboxylic acid (0.135 g, 0.98 mmol) was dissolved in DMF (3 mL). HATU (0.447 g, 1.17 mmol) was added and the mixture stirred at ambient temperature for about 1 min. TEA (0.6 mL, 4.30 mmol) and (1S,3S,4R)-3-(3H-imidazo[1,2-a]pyrrolo[2,3-e]pyrazin-8-yl)-4-methylcyclopentylamine (0.250 g, 0.98 mmol) were added and the resulting mixture was stirred at ambient temperature for about 1 h. The mixture was diluted with DCM (10 mL) and washed with a saturated aqueous solution of NaHCO₃ (5 mL). The organic layer was collected, dried over MgSO₄, filtered and evaporated. The resulting solid was sequentially triturated with Et₂O and MeOH. The resulting solid was filtered. The filtrate was evaporated to dryness. The solid was triturated with DCM/DMF (20/1 by volume) to give a white solid that was combined with solid recovered from the Et₂O/MeOH filtrate. The combined solids were triturated with EtOAc, filtered and dried to give N-((1S,3S,4R)-3-(3H-imidazo[1,2-a]pyrrolo[2,3-e]pyrazin-8-yl)-4-methylcyclopentyl)-5-methylpyrazine-2-carboxamide (0.164 g, 44%); LC/MS (Table 1, Method a) Rₜ=1.7 min; MS m/z: 376 (M+H)⁺. Jak3 IC₅₀=C

### Example #2

### N-((1S,3S,4R)-3-(3H-imidazo[1,2-a]pyrrolo[2,3-e]pyrazin-8-yl)-4-methylcyclopentyl)pyridazine-3-carboxamide

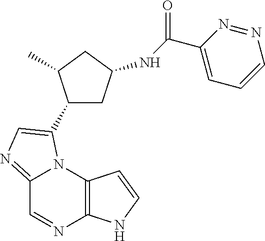 [SMILES: C[C@@H]1C[C@H](NC(=O)c2cccnn2)C[C@@H]1c1cnc2cnc3[nH]ccc3n12]

### Step A: N-((1S,3S,4R)-3-(3H-imidazo[1,2-a]pyrrolo[2,3-e]pyrazin-8-yl)-4-methylcyclopentyl)pyridazine-3-carboxamide

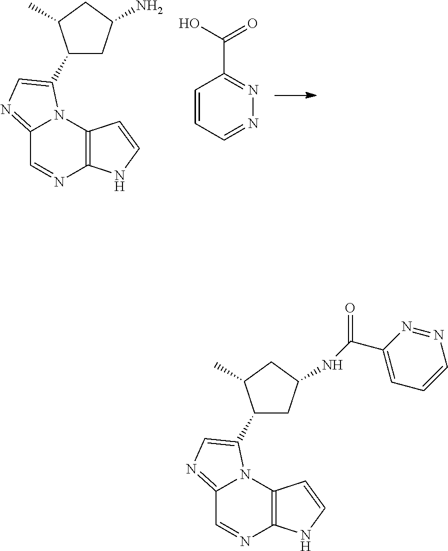 [SMILES: C[C@@H]1C[C@H](N)C[C@@H]1c1cnc2cnc3[nH]ccc3n12 ; O=C(O)c1cccnn1 ; C[C@@H]1C[C@H](NC(=O)c2cccnn2)C[C@@H]1c1cnc2cnc3[nH]ccc3n12]

Pyridazine-3-carboxylic acid (0.122 g, 0.979 mmol, Matrix) was dissolved in DMF (3 mL). HATU (0.447 g, 1.17 mmol) was added and the mixture stirred at ambient temperature for about 1 min. TEA (0.6 mL, 4.30 mmol) and (1S,3S,4R)-3-(3H-imidazo[1,2-a]pyrrolo[2,3-e]pyrazin-8-yl)-4-methylcyclopentylamine (0.250 g, 0.98 mmol, Example #1, Step D) were added and the resulting mixture was stirred at ambient temperature for about 1 h. The mixture was diluted with DCM (10 mL) and washed with saturated aqueous NaHCO₃ (5 mL). The organic layer was collected, dried over MeSO₄, filtered and evaporated. The resulting solid was sequentially triturated with Et₂O and MeOH, filtered and the resulting solid dried to give N-((1S,3S,4S)-3-(3H-imidazo[1,2-a]pyrrolo[2,3-e]pyrazin-8-yl)-4-methylcyclopentyl)pyridazine-3-carboxamide (0.092 g, 26%); LC/MS (Table 1, Method a) Rₜ=1.58 min; MS m/z: 362 (M+H)⁺. Jak3 IC₅₀=C

### Example #3

### N-((1S,3S,4R)-3-(3H-imidazo[1,2-a]pyrrolo[2,3-e]pyrazin-8-yl)-4-methylcyclopentyl)-N-methyloxazole-4-carboxamide

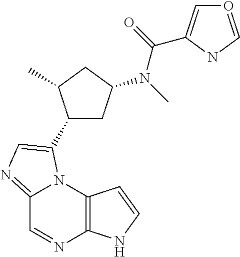

### Step A: N-((1S,3S,4R)-3-(3H-imidazo[1,2-a]pyrrolo[2,3-e]pyrazin-8-yl)-4-methylcyclopentyl)formamide

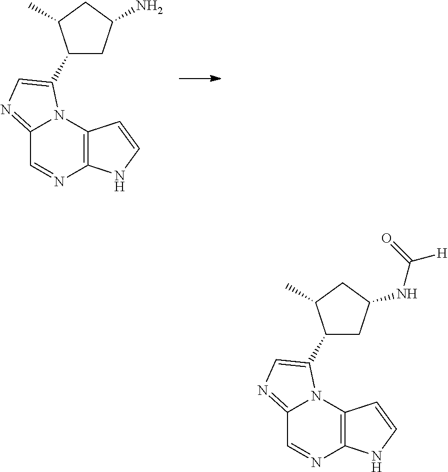 [SMILES: C[C@@H]1C[C@H](N)C[C@@H]1c1cnc2cnc3[nH]ccc3n12 ; C[C@@H]1C[C@H](NC=O)C[C@@H]1c1cnc2cnc3[nH]ccc3n12]

To an oven dried flask containing acetic anhydride (1.39 mL, 14.69 mmol) at about 0° C. was added formic acid (077 mL, 17.63 mmol) dropwise. The mixture was stirred at about 60° C. for about 2 h, then cooled to ambient temperature and added to a suspension of (1S,3S,4R)-3-(3H-imidazo[1,2-a]pyrrolo[2,3-e]pyrazin-8-yl)-4-methylcyclopentylamine (1.5 g, 5.88 mmol, Example #1, Step D) in THF (24 mL). The reaction mixture was cooled to about 0° C. and stirred at this temperature for about 1 h. The mixture was quenched with saturated aqueous NaHCO₃ (30 mL) and EtOAc (40 mL) was added. After stirring for about 20 min, the layers were separated. The aqueous layer was extracted again with EtOAc (40 mL). The combined organic layers were dried over anhydrous MgSO₄, filtered, and concentrated under reduced pressure and dried further under vacuum overnight to give N-((1S,3S,4R)-3-(3H-imidazo[1,2-a]pyrrolo[2,3-e]pyrazin-8-yl)-4-methylcyclopentyl)formamide (0.86 g, 52% crude) that was used without further purification; LC/MS (Table 1, Method e) Rₜ=0.36 min; MS m/z: 284 (M+H)⁺.

### Step B: (1S,3S,4R)-3-(3H-imidazo[1,2-a]pyrrolo[2,3-e]pyrazin-8-yl)-N,4-dimethylcyclopentylamine

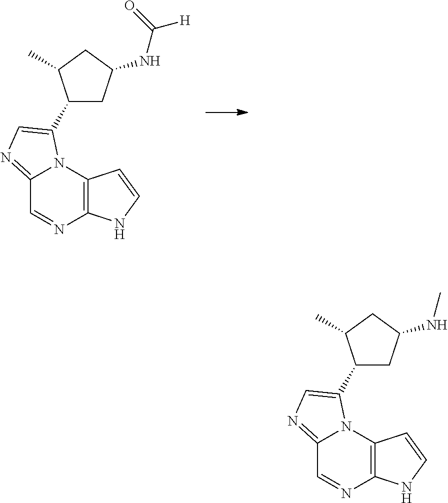 [SMILES: C[C@@H]1C[C@H](NC=O)C[C@@H]1c1cnc2cnc3[nH]ccc3n12 ; CN[C@H]1C[C@@H](C)[C@@H](c2cnc3cnc4[nH]ccc4n23)C1]

LiAlH₄ (4.57 mL, 9.15 mmol) was added portion wise to a suspension of N-((1S,3S,4R)-3-(3H-imidazo[1,2-a]pyrrolo[2,3-e]pyrazin-8-yl)-4-methylcyclopentyl)formamide (0.864 g, 3.05 mmol) in THF (30.5 mL) at about 0° C. The reaction mixture was stirred at about 0° C. for about 20 min, warmed to ambient temperature for about 20 min and heated to reflux for about 5 h. The mixture was cooled to rt and stirred overnight. The mixture was heated to reflux for about 4 h, cooled to about 0° C. and sequentially quenched with the dropwise addition of water (3 mL), 1N NaOH (3 mL) then water (2 mL). The resulting mixture was stirred at about 0° C. for about 15 min then warmed to ambient temperature for about 15 min. The mixture was filtered through Celite®. The filter cake was rinsed with EtOAc. Water (50 mL) was added to the combined filtrates and the mixture extracted with EtOAc (2×80 mL). The combined organic layers were dried over anhydrous MgSO₄, filtered, and concentrated under reduced pressure to give (1S,3S,4R)-3-(3H-imidazo[1,2-a]pyrrolo[2,3-e]pyrazin-8-yl)-N,4-dimethylcyclopentylamine (0.25 g, 31%) as a brown solid; LC/MS (Table 1, Method a) Rₜ=1.32 min; MS m/z: 270 (M+H)⁺.

### Step C: N-((1S,3S,4R)-3-(3H-imidazo[1,2-a]pyrrolo[2,3-e]pyrrazin-8-yl)-4-methylcyclopentyl)-N-methyloxazole-4-carboxamide

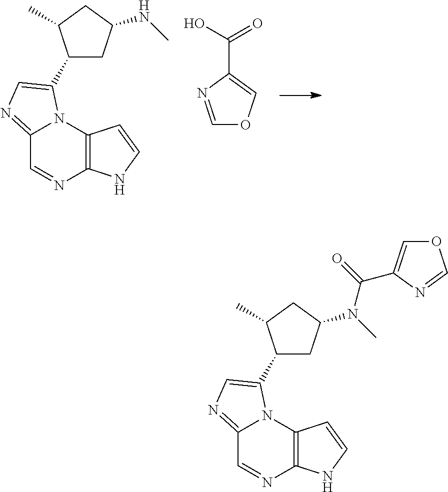

To a solution of oxazole-4-carboxylic acid (0.12 g, 1.05 mmol) and TEA (0.56 mL, 4.0 mmol) in DMF (3.00 mL) was added HBTU (0.46 g, 1.20 mmol). The mixture was stirred for about 10 min at ambient temperature. To the resulting very dark colored mixture was added (1S,3S,4R)-3-(3H-imidazo[1,2-a]pyrrolo[2,3-e]pyrazin-8-yl)-N,4-dimethylcyclopentanamine (0.27 g, 1.00 mmol) and stirred at ambient temperature for about 1.5 h. The reaction mixture was quenched with a saturated aqueous NaHCO₃ (30 mL) and extracted into DCM (2×50 mL). The combined organic layers were dried over anhydrous MgSO₄, filtered and concentrated under reduced pressure. The residue was dissolved in DCM (30 mL) and washed with brine (20 mL). The organic layer was dried over anhydrous MgSO₄, filtered, and concentrated, under reduced pressure. The material was purified by chromatography on silica gel (0-10% MeOH in DCM). The fractions containing the desired product were combined and concentrated under reduced pressure. The resulting material was purified by mass triggered purification (Table 1, method d). The recovered fractions were evaporated and taken up in acetonitrile/water. The volatiles were removed under reduced pressure, frozen and lyophilized repeatedly to remove all traces of ammonium acetate to give N-((1S,3S,4R)-3-(3H-imidazo[1,2-a]pyrrolo[2,3-e]pyrazin-8-yl)-4-methylcyclopentyl)-N-methyloxazole-4-carboxamide (0.074 g, 20%); LC/MS (Table 1, Method a) Rₜ=1.34 min; MS m/z: 365 (M+H)⁺. Jak3 IC₅₀=B

### Example #4

### (1S,3S,4R)-3-(3H-imidazo[1,2-a]pyrrolo[2,3-e]pyrazin-8-yl)-4-methyl-N-((2-(trifluoromethyl)pyrimidin-5-yl)methyl)cyclopentylamine

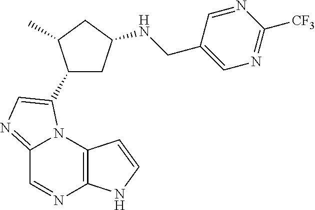 [SMILES: C[C@@H]1C[C@H](NCc2cnc(C(F)(F)F)nc2)C[C@@H]1c1cnc2cnc3[nH]ccc3n12]

### Step A: Ethyl 2-(trifluoromethyl)pyrimidine-5-carboxylate

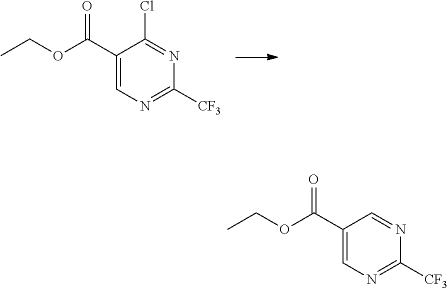 [SMILES: CCOC(=O)c1cnc(C(F)(F)F)nc1Cl ; CCOC(=O)c1cnc(C(F)(F)F)nc1]

A mixture of ethyl 4-chloro-2-(trifluoromethyl)pyrimidine-5-carboxylate (2.51 g, 9.86 mmol, Matrix) and 10% Pd/C (0.105 g, 0.099 mmol) was evacuated and purged with N₂ (3×) and EtOH (49 mL) was added followed by DIEA (4.2 mL, 24.0 mmol). The reaction was placed under a hydrogen atmosphere (via balloon) and left stirring at ambient temperature for about 4 h. The reaction was filtered through a pad of Celite®, washing with EtOH and the filtrate was concentrated under reduced pressure. The crude material was taken up in DCM (25 mL) and washed with saturated aqueous NH₄Cl (2×20 mL). The organics were dried over anhydrous MgSO₄, filtered, and concentrated under reduced pressure to give ethyl 2-(trifluoromethyl)pyrimidine-3-carboxylate (2.00 g, 92%) as a yellow solid; ¹H NMR (400 MHz, CDCl₃) δ 9.43 (s, 2H), 4.51 (q, 2H), 1.46 (t, 3H).

### Step B: (1S,3S,4R)-3-(3H-imidazo[1,2-a]pyrrolo[2,3-e]pyrazin-8-yl)-4-methyl-N-((2-(trifluoromethyl)pyrimidin-5-yl)methyl)cyclopentylamine

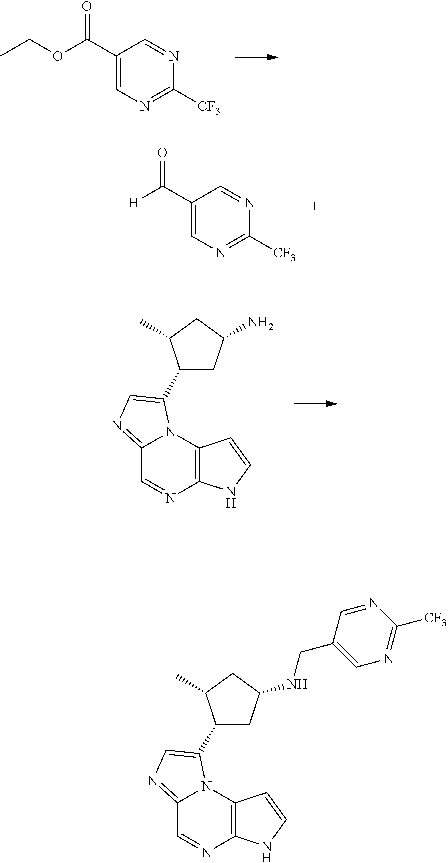 [SMILES: CCOC(=O)c1cnc(C(F)(F)F)nc1 ; O=Cc1cnc(C(F)(F)F)nc1 ; C[C@@H]1C[C@H](N)C[C@@H]1c1cnc2cnc3[nH]ccc3n12 ; C[C@@H]1C[C@H](NCc2cnc(C(F)(F)F)nc2)C[C@@H]1c1cnc2cnc3[nH]ccc3n12]

To a solution of ethyl 2-(trifluoromethyl)pyrimidine-5-carboxylate (0.750 g, 2.73 mmol) in toluene (13.6 mL) at about −78° C. was added DIBAL-H (1M in cyclohexane, 3.30 mL, 3.30 mmol) over about 15 min and the reaction was left stirring at about −78° C. for about 1 h. The reaction was quenched with the slow addition of 2N aqueous HCl (13.6 mL) and the reaction was warmed to ambient temperature. The reaction mixture was extracted with ether (3×15 mL) and the combined organics were dried over anhydrous MgSO₄, filtered, and concentrated under reduced pressure to give crude 2-(trifluoromethyl)pyrimidine-5-carbaldehyde. To a mixture of (1S,3S,4R)-3-(3H-imidazo[1,2-a]pyrrolo[2,3-e]pyrazin-8-yl)-4-methylcyclopentylamine (0.112 g, 0.437 mmol, Example #1, Step D) and 2-(trifluoromethyl)pyrimidine-5-carbaldehyde (0.100 g, 0.568 mmol) in DCE (1.00 mL) and MeOH (1.00 mL) was added acetic acid (0.038 mL, 0.655 mmol) and sodium triacetoxyborohydride (0.139 g, 0.655 mmol). The reaction was left stirring at ambient temperature for about 3 h. The reaction was concentrated under reduced pressure and the residue was taken up in DCM and saturated aqueous NaHCO₃ (10 mL each). The layers were separated and the aqueous phase was extracted with DCM (2×10 mL) and EtOAc (10 mL). The combined organics were dried over anhydrous MgSO₄, filtered and concentrated under reduced pressure. The crude material was purified by silica gel chromatography eluting with 0-10% MeOH in DCM to give (1S,3S,4R)-3-(3H-imidazo[1,2-a]pyrrolo[2,3-e]pyrazin-8-yl)-4-methyl-N-((2-(trifluoromethyl)pyrimidin-5-yl)methyl)cyclopentylamine (0.035 g, 19%) as an off-white solid; LC/MS (Table 1, Method a) Rₜ=1.28 min; MS m/z: 416 (M+H)⁺. Jak3 IC₅₀=C

### Example #5

### 1-((1S,3S,4R)-3-(3H-imidazo[1,2-a]pyrrolo[2,3-e]pyrazin-8-yl)-4-methylcyclopentyl)piperidine-4-carbonitrile

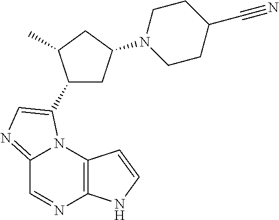 [SMILES: C[C@@H]1C[C@H](N2CCC(C#N)CC2)C[C@@H]1c1cnc2cnc3[nH]ccc3n12]

### Step A: (3S,4R)-3-(3H-imidazo[1,2-a]pyrrolo[2,3-e]pyrazin-8-yl)-4-methylcyclopentanone

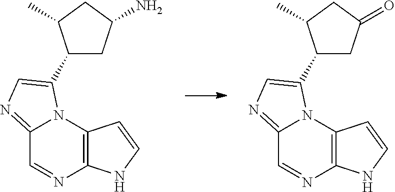 [SMILES: C[C@@H]1C[C@H](N)C[C@@H]1c1cnc2cnc3[nH]ccc3n12 ; C[C@@H]1CC(=O)C[C@@H]1c1cnc2cnc3[nH]ccc3n12]

To a solution of (1S,3S,4R)-3-(3H-imidazo[1,2-a]pyrrolo[2,3-e]pyrazin-8-yl)-4-methylcyclopentylamine (1.50 g, 5.88 mmol, Example #1, Step D) in MeOH (30 mL) was added 3,5-di-tert-butylcyclohexa-3,5-diene-1,2-dione (1.55 g, 7.05 mmol). The reaction mixture was left stirring at ambient temperature for about 2 h. THF (18 mL) and water (6 mL) were added and the pH of the reaction was adjusted to about 4 with oxalic acid; the reaction was left stirring for about 30 min. The reaction was concentrated under reduced pressure and the residue was taken up in 10% MeOH in DCM (20 mL) water (10 mL). The layers were separated and the aqueous phase was extracted with 10% MeOH in DCM (2×15 mL) and the combined organics were dried over anhydrous MgSO₄, filtered, and concentrated under reduced pressure. The crude material was purified by silica gel chromatography eluting with a gradient of 0-100% (900/95/5 DCM/MeOH/NH₄OH) in DCM. The fractions containing product were pooled together and concentrated under reduced pressure. The material was further purified by silica gel chromatography eluting with a gradient of 25-75% acetone in heptane to give (3S,4R)-3-(3H-imidazo[1,2-a]pyrrolo[2,3-e]pyrazin-8-yl)-4-methylcyclopentanone as a tan solid (0.860 g, 58%); LC/MS (Table 1, Method a) Rₜ=1.26 min; MS m/z: 255 (M+H)⁺.

### Step B: 1-((1S,3S,4R)-3-(3H-imidazo[1,2-a]pyrrolo[2,3-e]pyrazin-8-yl)-4-methylcyclopentyl)piperidine-4-carbonitrile

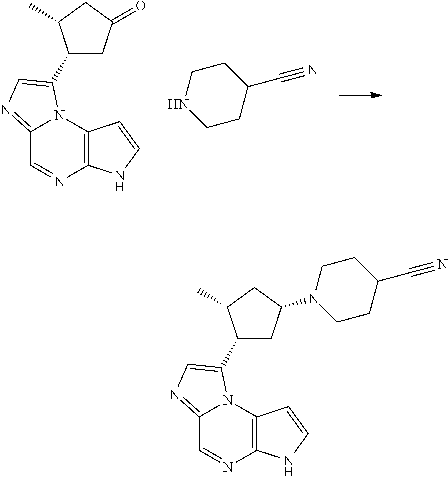 [SMILES: C[C@@H]1CC(=O)C[C@@H]1c1cnc2cnc3[nH]ccc3n12 ; N#CC1CCNCC1 ; C[C@@H]1C[C@H](N2CCC(C#N)CC2)C[C@@H]1c1cnc2cnc3[nH]ccc3n12]

To a solution of (3S,4R)-3-(3H-imidazo[1,2-a]pyrrolo[2,3-e]pyrazin-8-yl)-4-methylcyclopentanone (0.250 g, 0.885 mmol) and piperidine-4-carbonitrile (0.195 g, 1.770 mmol, Oakwood) in DCM (2 mL) and MeOH (2 mL) were added acetic acid (0.051 mL, 0.885 mmol) and sodium triacetoxyborohydride (0.281 g, 1.33 mmol) and the reaction mixture was left stirring at ambient temperature for about 16 h. The reaction was concentrated under reduced pressure and the residue was taken up in DCM and saturated aqueous NaHCO₃ (10 mL, each). The layers were separated and the aqueous phase was extracted with DCM (2×10 mL) and EtOAc (10 mL). The combined organics were dried over anhydrous MgSO₄, filtered and concentrated under reduced pressure. The crude material was purified silica gel chromatography eluting with a gradient of 0-10% MeOH in DCM. The product containing fractions were combined and concentrated under reduced pressure. The material was further purified by RP-HPLC (Table 1, Method d) to give 1-((1S,3S,4R)-3-(3H-imidazo[1,2-a]pyrrolo[2,3-e]pyrazin-8-yl)-4-methylcyclopentyl)piperidine-4-carbonitrile (0.025 g, 17%) as an off-white solid; LC/MS (Table 1, Method a) Rₜ=0.99 min; MS m/z: 349 (M+H)⁺. Jak3 IC₅₀=C

### Example #6

### (1R,3R,4S)-3-ethyl-4-(6H-pyrrolo[2,3-e][1,2,4]triazolo[4,3-a]pyrazin-1-yl)-N-(2,2,2-trifluoroethyl)cyclopentanecarboxamide

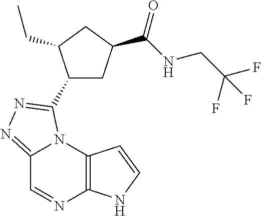 [SMILES: CC[C@@H]1C[C@@H](C(=O)NCC(F)(F)F)C[C@@H]1c1nnc2cnc3[nH]ccc3n12]

### Step A: (1R,3R,4S)-3-ethyl-4-(6H-pyrrolo[2,3-e][1,2,4]triazolo[4,3-a]pyrazin-1-yl)cyclopentanecarboxylic acid

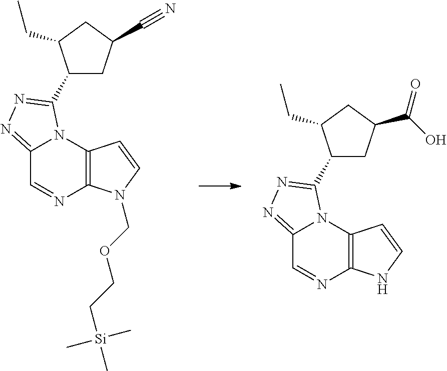 [SMILES: CC[C@@H]1C[C@@H](C#N)C[C@@H]1c1nnc2cnc3c(ccn3COCC[Si](C)(C)C)n12 ; CC[C@@H]1C[C@@H](C(=O)O)C[C@@H]1c1nnc2cnc3[nH]ccc3n12]

(1R,3R,4S)-3-ethyl-4-(6-((2-(trimethylsilyl)ethoxy)methyl)-6H-pyrrolo[2,3-e][1,2,4]triazolo[4,3-a]pyrazin-1-yl)cyclopentanecarbonitrile (0.886 g, 2.158 mmol, WO2011/068881) was dissolved in concentrated aqueous HCl (16.39 mL, 539 mmol) by using sonication. The solution was heated to about 50° C. for about 16 h. The reaction mixture was poured onto 20 mL ice slurry and pH was adjusted to 6 using 2N aqueous NaOH. The product was extracted into DCM (3×30 mL). To the aqueous phase was added concentrated NH₄OH (10 mL) and the mixture was stirred for about 2 h. The pH of the mixture was adjusted to 6 with 6N aqueous HCl. The product was extracted into DCM (2×30 mL) and EtOAc (2×30 mL). The combined organic extracts were dried over anhydrous MgSO₄, filtered and concentrated to yield (1R,3R,4S)-3-ethyl-4-(6H-pyrrolo[2,3-e][1,2,4]triazolo[4,3-a]pyrazin-1-yl)cyclopentanecarboxylic acid (0.445 g, 65%) as an off-white solid: LC/MS (Table 1, Method b) Rₜ=1.17 min; MS m/z: 300 (M+H)⁺.

### Step B: (1R,3R,4S)-3-ethyl-4-(6H-pyrrolo[2,3-e][1,2,4]triazolo[4,3-a]pyrazin-1-yl)-N-(2,2,2-trifluoroethyl)cyclopentanecarboxamide

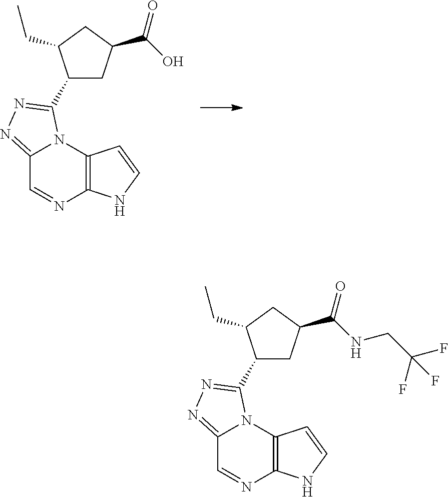 [SMILES: CC[C@@H]1C[C@@H](C(=O)O)C[C@@H]1c1nnc2cnc3[nH]ccc3n12 ; CC[C@@H]1C[C@@H](C(=O)NCC(F)(F)F)C[C@@H]1c1nnc2cnc3[nH]ccc3n12]

To a solution of (1R,3R,4S)-3-ethyl-4-(6H-pyrrolo[2,3-e][1,2,4]triazolo[4,3-a]pyrazin-1-yl)cyclopentanecarboxylic acid (0.125 g, 0.418 mmol) in a mixture of DCM (2.5 mL) and DMF (0.5 mL) were added DIEA (0.153 mL, 0.877 mmol), 2,2,2-trifluoroethanamine (0.050 g, 0.501 mmol) and HATU (0.206 g, 0.543 mmol). The reaction mixture was stirred at ambient temperature for about 2 h. Water (5 mL) was added and the aqueous phase was extracted with DCM (3×5 mL). The combined organic extracts were dried over MgSO₄, filtered and concentrated. The crude material was purified using silica gel column chromatography (0-10% MeOH in DCM over 45 min) to yield (1R,3R,4S)-3-ethyl-4-(6H-pyrrolo[2,3-e][1,2,4]triazolo[4,3-a]pyrazin-1-yl)-N-(2,2,2-trifluoroethyl)cyclopentanecarboxamide (0.036 g, 23%) as an off-white solid: LC/MS (Table 1, Method b) Rₜ=1.58 min; MS m/z: 381 (M+H)⁺. Jak3 IC₅₀=C

### Example #7

### 1-((1R,3S,4R)-3-(3H-imidazo[1,2-a]pyrrolo[2,3-e]pyrazin-8-yl)-4-methylcyclopentyl)methyl)azetidine-3-carbonitrile and 1-(((1S,3S,4R)-3-(3H-imidazo[1,2-a]pyrrolo[2,3-e]pyrazin-8-yl)-4-methylcyclopentyl)methyl)azetidine-3-carbonitrile

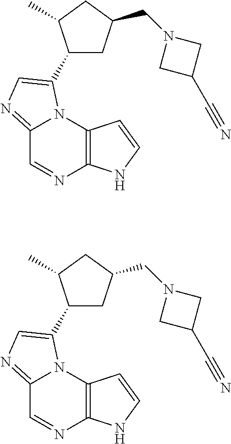 [SMILES: C[C@@H]1C[C@@H](CN2CC(C#N)C2)C[C@@H]1c1cnc2cnc3[nH]ccc3n12 ; C[C@@H]1C[C@H](CN2CC(C#N)C2)C[C@@H]1c1cnc2cnc3[nH]ccc3n12]

### Step A: (7S,8R)ethyl 8-methyl-1,4-dioxaspiro[4.4]nonane-7-carboxylate

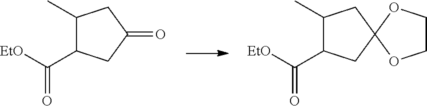

A round bottom flask was charged with a scalemic mixture enriched in (1S,2R)-ethyl 2-methyl-4-oxocyclopentanecarboxylate (150 g, 881 mmol, WO2011/068881) in DCM (2203 mL) followed by 4 Å molecular sieves (175 g), after which ethylene glycol (98 mL, 1763 mmol), trimethylorthoformate (145 mL, 1322 mmol) and toluene-4-sulfonic acid hydrate (33.5 g, 176 mmol) were added. The reaction mixture was stirred at rt for about 96 h. The reaction was filtered and the solvent was removed in vacuo to yield a yellow oil. It was subjected to silica gel column chromatography eluting with heptane/EtOAc to yield a scalemic mixture enriched in (7S,8R)-ethyl 8-methyl-1,4-dioxaspiro[4.4]nonane-7-carboxylate (179 g, 85%) as a yellow oil. ¹H NMR (400 MHz, CDCl₃) δ 4.18-4.12 (m, 2H), 3.93-3.87 (m, 4H), 3.00-2.93 (m, 1H), 2.54-2.45 (m, 1H), 2.37-2.29 (m, 1H), 2.04-1.95 (m, 2H), 1.78-1.71 (m, 1H), 1.29-1.24 (m, 3H), 0.97 (dd, 3H).

### Step B: (7S,8R)-8-methyl-1,4-dioxaspiro[4.4]nonane-7-carboxylic acid

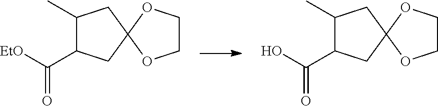

A 2 L flask was charged with a scalemic mixture enriched in (7S,8R)-ethyl 8-methyl-1,4-dioxaspiro[4.4]nonane-7-carboxylate (179 g, 835 mmol) and added to a 1N aqueous NaOH (8354 mL, 8354 mmol). The reaction was stirred at ambient temperature for about 16 h. The aqueous solution was then acidified to pH 4.5 with 20% aqueous citric acid. The product was immediately partitioned between DCM (5×350 mL) and aqueous layer. The combined extracts were dried over anhydrous MgSO₄, filtered, and concentrated under reduced pressure to afford scalemic mixture enriched in (7S,8R)-8-methyl-1,4-dioxaspiro[4.4]nonane-7-carboxylic acid (85.7 g, 55%) as a pale oil. ¹H NMR (400 MHz, CDCl₃) δ 3.96-3.88 (m, 4H), 3.05-2.98 (m, 1H), 2.59-2.47 (m, 1H), 2.36-2.29 (m, 1H), 2.07-1.98 (m, 2H), 1.80-1.73 (m, 1H), 1.08-1.02 (m, 3H).

### Step C: (7S,8R)-8-methyl-1,4-(dioxaspiro[4.4]nonane)cyclopentyl-dimethylsulfoxonium-7-oxo-ethylide

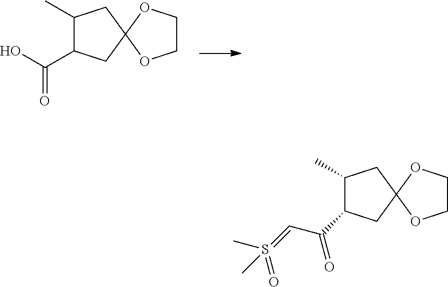 [SMILES: CC1CC2(CC1C(=O)O)OCCO2 ; C[C@@H]1CC2(C[C@@H]1C(=O)C=S(C)(C)=O)OCCO2]

Trimethylsulfoxonium chloride (27.5 g, 213 mmol) was suspended in THF (300 mL), potassium tert-butoxide (25.2 g, 224 mmol) was added and the resulting mixture was heated at about 60° C. for about 2 h and then cooled to about 0° C. in a separate flask, to the solution of a scalemic mixture enriched in (7S,8R)-8-methyl-1,4-dioxaspiro[4.4]nonane-7-carboxylic acid (13.25 g, 71.2 mmol) in THF (300 mL), TEA (34.7 mL, 249 mmol) was added followed by the addition of HATU (27.1 g, 71.2 mmol). The resulting suspension was stirred at ambient temperature for about 2 h to give a yellow solution. This solution was added dropwise to a suspension described above at about 0° C. over about 10 min. The resulting mixture was stirred at about 0° C. for about 1 h. Water (150 mL) was added and the mixture stirred at about 0° C. for about another 10 min. The organic solvent was removed under reduced pressure and the resulting suspension was diluted with water (100 mL). The precipitate was removed by filtration; the aqueous phase was saturated with sodium chloride and extracted with DCM (4×300 mL). The combined organic extracts were washed with brine (250 mL), dried over anhydrous magnesium sulfate, filtered and concentrated. The residue was suspended in EtOAc (200 mL) and the precipitate was collected by filtration and dried to give (7S,8R)-8-methyl-1,4-(dioxaspiro[4.4]nonan-cyclopentyl)-dimethylsulfoxonium-7-oxo-ethylide (9.8 g, 54%) as a white solid. LC/MS (Table 1, Method b) Rₜ=1.08 min; MS m/z: 261 (M+H)⁺.

### Step D: 1-((7S,8R)-8-methyl-1,4-dioxaspiro[4.4]nonan-7-yl)-2-(5-tosyl-5H-pyrrolo[2,3-b]pyrazin-2-ylamino)ethanone

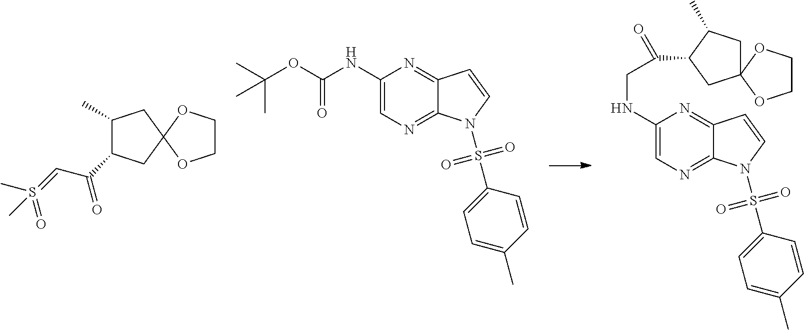

Argon was bubbled through a solution of chloro(1,5-cyclooctadiene)iridium (I) dimer (1.02 g, 1.52 mmol, Strem), (7S,8R)-8-methyl-1,4-(dioxaspiro[4.4]nonane)cyclopentyl)-dimethylsulfoxonium-7-oxo-ethylide (9.5 g, 36.5 mmol) and 5-tosyl-5H-pyrrolo[2,3-b]pyrazin-2-amine (8.77 g, 30.4 mmol, WO 2011/068881) in DMF (300 mL) for about 5 min and the resulting solution was heated at about 75° C. for about 16 h. The solvent was removed under reduced pressure and the residue partitioned between EtOAc and water (200 mL each). The organic phase was washed with brine (150 mL), dried over anhydrous magnesium sulfate, filtered and concentrated. Purification by silica gel column chromatography (0-50% EtOAc in DCM gradient) yielded 1-((7S,8R)-8-methyl-1,4-dioxaspiro[4.4]nonan-7-yl)-2-(5-tosyl-5H-pyrrolo[2,3-b]pyrazin-2-ylamino)ethanone (6.06 g, 42%) as a yellow amorphous solid. LC/MS (Table 1, Method b) Rₜ=2.44 min; MS m/z: 471 (M+H)⁺.

### Step E: (3R,4S)-3-methyl-4-(2-(5-tosyl-5H-pyrrolo[2,3-b]pyrazin-2-ylamino)acetyl)cyclopentanone

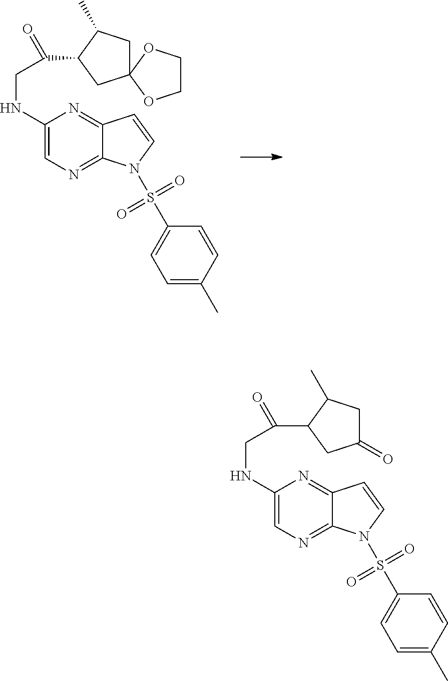 [SMILES: Cc1ccc(S(=O)(=O)n2ccc3nc(NCC(=O)[C@H]4CC5(C[C@H]4C)OCCO5)cnc32)cc1 ; Cc1ccc(S(=O)(=O)n2ccc3nc(NCC(=O)C4CC(=O)CC4C)cnc32)cc1]

To a solution of 1-((7S,8R)-8-methyl-1,4-dioxaspiro[4.4]nonan-7-yl)-2-(5-tosyl-5H-pyrrolo[2,3-b]pyrazin-2-ylamino)ethanone (6.06 g, 12.88 mmol) in THF (129 mL), 1N aqueous hydrochloric acid (38.6 mL, 38.6 mmol) was added at ambient temperature and the mixture was stirred for about 4 h. The reaction mixture was neutralized by a dropwise addition of saturated aqueous sodium bicarbonate; THF was removed under reduced pressure and aqueous phase was extracted with EtOAc (2×100 mL). The combined organic extracts were washed with brine (100 mL), dried over anhydrous magnesium sulfate, filtered and concentrated. The gummy residue was suspended in minimal amount of EtOAc and left overnight in the refrigerator. The precipitate was collected by filtration, washed with Et₂O and dried to yield a scalemic mixture enriched in (3R,4S)-3-methyl-4-(2-(5-tosyl-5H-pyrrolo[2,3-b]pyrazin-2-ylamino)acetyl)cyclopentanone (3.7 g, 67%) as a light brown solid, LC/MS (Table 1, Method b) Rₜ=2.22 min; MS m/z: 427 (M+H)⁺.

### Step F: (3R,4S)-3-triethyl-4-(3-tosyl-3H-imidazo[1,2-a]pyrrolo[2,3-e]pyrazin-8-yl)cyclopentanone

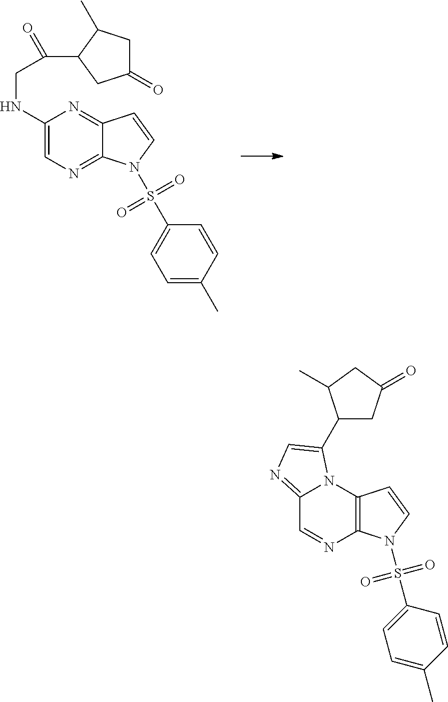 [SMILES: Cc1ccc(S(=O)(=O)n2ccc3nc(NCC(=O)C4CC(=O)CC4C)cnc32)cc1 ; Cc1ccc(S(=O)(=O)n2ccc3c2ncc2ncc(C4CC(=O)CC4C)n23)cc1]

To a solution of scalemic mixture enriched in (3R,4S)-3-methyl-4-(2-(5-tosyl-5H-pyrrolo[2,3-b]pyrazin-2-ylamino)acetyl)cyclopentanone (7.42 g, 17.4 mmol) in MeCN (102 mL), 2,2,3,3,3-pentafluoropropanoic anhydride (32.4 g, 104 mmol) was added and the reaction mixture was heated at about 80° C. for about 16 h. 1N aqueous HCl (18 mL) was added and the reaction mixture was stirred at ambient temperature for about 1 h. It was neutralized by a slow addition of saturated aqueous sodium bicarbonate and the organic solvent was removed under reduced pressure. The aqueous phase was extracted with EtOAc (2×100 mL); the combined extracts were washed with brine (120 mL), dried over anhydrous magnesium sulfate, filtered and concentrated. Silica gel column chromatography (20-100% EtOAc in DCM gradient) yielded a scalemic mixture enriched in (3R,4S)-3-methyl-4-(3-tosyl-3H-imidazo[1,2-a]pyrrolo[2,3-e]pyrazin-8-yl)cyclopentanone (3.75 g, 53%) as a brown solid, LC/MS (Table 1, Method b) Rₜ=2.17 min; MS m/z: 409 (M+H)⁺.

### Step G: (3S,4R)-3-(3H-imidazo[1,2-a]pyrrolo[2,3-e]pyrazin-8-yl)-4-methylcyclopentanone

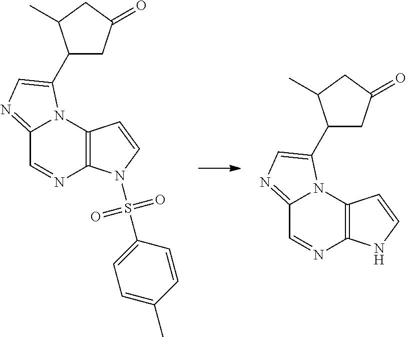 [SMILES: Cc1ccc(S(=O)(=O)n2ccc3c2ncc2ncc(C4CC(=O)CC4C)n23)cc1 ; CC1CC(=O)CC1c1cnc2cnc3[nH]ccc3n12]

To a solution of a scalemic mixture enriched in (3R,4S)-3-methyl-4-(3-tosyl-3H-imidazo[1,2-a]pyrrolo[2,3-e]pyrazin-8-yl)cyclopentanone (2.16 g, 529 mmol) in 1,4-dioxane (40 mL) was added 2N aqueous NaOH (13.22 mL, 26.4 mmol). The mixture was heated at about 50° C. for about 2 h. The reaction was cooled to ambient temperature and partitioned between EtOAc and saturated aqueous NaHCO₃ (50 mL each). Aqueous layer was further extracted with EtOAc (2×50 mL). Organic layers were combined, dried over anhydrous Na₂SO₄, filtered and concentrated to dryness to give a scalemic mixture enriched in (3S,4R)-3-(3H-imidazo[1,2-a]pyrrolo[2,3-e]pyrazin-8-yl)-4-methylcyclopentanone (1.26 g, 93%) as a brown solid. LC/MS (Table 1, Method b) Rₜ=1.56 min; MS m/z 255 (M+H)⁺.

### Step H: 8-((1S,2R)-4-(methoxymethylene)-2-methylcyclopentyl)-3H-imidazo[1,2-a]pyrrolo[2,3-e]pyrazine

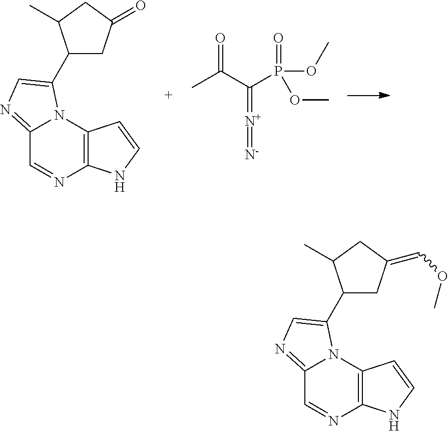 [SMILES: CC1CC(=O)CC1c1cnc2cnc3[nH]ccc3n12 ; COP(=O)(OC)C(=[N+]=[N-])C(C)=O ; COC=C1CC(C)C(c2cnc3cnc4[nH]ccc4n23)C1]

To a solution of dimethyl 1-diazo-2-oxopropylphosphonate (1.428 g, 7.43 mmol, Tet. Lett., 2006, 47 (11), 1729-1731) and a scalemic mixture enriched in (3S,4R)-3-(3H-imidazo[1,2-a]pyrrolo[2,3-e]pyrazin-8-yl)-4-methylcyclopentanone (1.26 g, 4.96 mmol) in MeOH (75 mL) was added K₂CO₃ (1.37 g, 9.91 mmol) at about 0° C. under N₂. The mixture was stirred at about 0° C. for about 30 min and then warmed to rt and stirred for about 21 h. The mixture was cooled to about 0° C., K₂CO₃ (1.37 g, 9.91 mmol) was added followed by dimethyl 1-diazo-2-oxopropylphosphonate (1.428 g, 7.43 mmol). The mixture was stirred at about 0° C. for about 30 min and then warmed to rt and stirred for about 20 h. The crude reaction mixture was filtered and the filtrate was concentrated. The residue was partitioned between EtOAc and saturated aqueous NaHCO₃. The aqueous layer was further extracted with EtOAc (2×50 mL). Organic layers were combined and concentrated to dryness. The material was purified by flash chromatography (0-5% MeOH in DCM gradient) to yield a scalemic mixture enriched in 8-((1S,2R)-4-(methoxymethylene)-2-methylcyclopentyl)-3H-imidazo[1,2-a]pyrrolo[2,3-e]pyrazine (0.901 g, 64%) as a mixture of E and Z isomers as a light yellow solid. LC/MS (Table 1, Method b) Rₜ=1.96 min; MS m/z: 283 (M+H)⁺.

### Step I: (3S,4R)-3-(3H-imidazo-[1,2-a]pyrrolo[2,3-e]pyrazin-8-yl)-4-methylcyclopentanecarbaldehyde

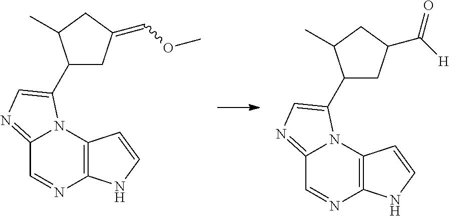 [SMILES: COC=C1CC(C)C(c2cnc3cnc4[nH]ccc4n23)C1 ; CC1CC(C=O)CC1c1cnc2cnc3[nH]ccc3n12]

To a solution of a scalemic mixture enriched in 8-((1S,2R)-4-(methoxymethylene)-2-methylcyclopentyl)-3H-imidazo-[1,2-a]pyrrolo[2,3-e]pyrazine (0.901 g, 3.23 mmol) as a mixture of E and Z isomers in THF (16 mL) at ambient temperature was added 1N aqueous HCl (9.7 mL, 9.7 mmol). The reaction mixture was stirred for about 20 h. Volatile solvent was removed under reduced pressure. Saturated aqueous NaHCO₃ was added dropwise until gas evolution stopped. The solvent was decanted; the gum was suspended in water and sonicated. The solid was collected by filtration, washed with water and dried in vacuum oven at about 60° C. for about 4 h to yield a scalemic mixture enriched in (1S,3S,4R)-3-(3H-imidazo-[1,2-a]pyrrolo[2,3-e]pyrazin-8-yl)-4-methylcyclopentanecarbaldehyde and (1R,3S,4R)-3-(3H-imidazo-[1,2-a]pyrrolo[2,3-e]pyrazin-8-yl)-4-methylcyclopentanecarbaldehyde (0.706 g, 81%) as tan solid. LC/MS (Table 1, Method b) Rₜ=1.71 min; MS m/z: 269 (M+H)⁺.

### Step J: 1-(((1R,3S,4R)-3-(3H-imidazo[1,2-a]pyrrolo[2,3-e]pyrazin-8-yl)-4-methylcyclopentyl)methyl)azetidine-3-carbonitrile and 1-(((1S,3S,4R)-3-(3H-imidazo[1,2-a]pyrrolo[2,3-e]pyrazin-8-yl)-4-methylcyclopentyl)methyl)azetidine-3-carbonitrile

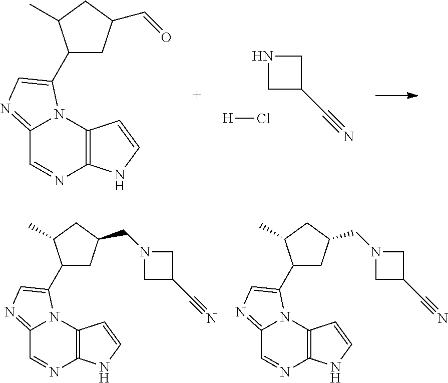

To a vial charged with a scalemic mixture enriched, in (1S,3S,4R)-3-(3H-imidazo[1,2-a]pyrrolo[2,3-e]pyrazin-8-yl)-4-methylcyclopentanecarbaldehyde and (1R,3S,4R)-3-(3H-imidazo[1,2-a]pyrrolo[2,3-e]pyrazin-8-yl)-4-methylcyclopentanecarbaldehyde (0.350 g, 1.304 mmol) and azetidine-3-carbonitrile hydrochloride (0.309 g, 2.61 mmol) was added MeOH (9 mL). The mixture was stirred at rt for about 1 h. Sodium cyanoborohydride (0.246 g, 3.91 mmol) was added in one portion. The mixture was stirred at rt for about 1 h. Solvent was removed under reduced pressure. The residue was purified by mass triggered HPLC (Table 1, method f). The desired fractions were concentrated under reduced pressure and the residue was purified by chiral chromatography (Table 2, Method 5) Detection methods were evaporative light scattering (ELSD) detection and optical rotation. The desired fractions were combined and concentrated under reduced pressure to yield 1-(((1R,3S,4R)-3-(3H-imidazo[1,2-a]pyrrolo[2,3-e]pyrazin-8-yl)-4-methylcyclopentyl)methyl)azetidine-3-carbonitrile (0.0675 g, 15%). LC/MS (Table 1, Method b) Rₜ=1.40 min; MS m/z: 335 (M+H)⁺) and 1-(((1S,3S,4R)-3-(3H-imidazo[1,2-a]pyrrolo[2,3-e]pyrazin-8-yl)-4-methylcyclopentyl)methyl)azetidine-3-carbonitrile (0.0764 g, 17%) both as white solids. LC/MS (Table 1, Method b) Rₜ=1.38 min; MS m/z: 335 (M+H)⁺. Jak3 IC₅₀=C

### Example #8

### 1-((1R,3S,4R)-3-(3H-imidazo[1,2-a]pyrrolo[2,3-e]pyrazin-8-yl)-4-methylcyclopentyl)piperidine-4-carbonitrile

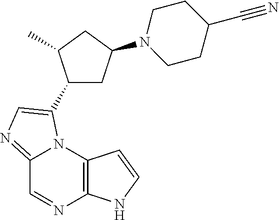 [SMILES: C[C@@H]1C[C@@H](N2CCC(C#N)CC2)C[C@@H]1c1cnc2cnc3[nH]ccc3n12]

### Step A: 3-methyl-4-(3-tosyl-3H-imidazo[1,2-a]pyrrolo[2,3e]pyrazin-8-yl)cyclopentanol

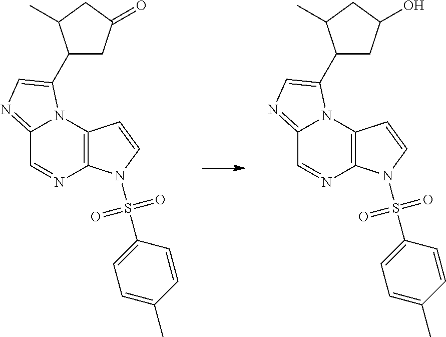 [SMILES: Cc1ccc(S(=O)(=O)n2ccc3c2ncc2ncc(C4CC(=O)CC4C)n23)cc1 ; Cc1ccc(S(=O)(=O)n2ccc3c2ncc2ncc(C4CC(O)CC4C)n23)cc1]

To a suspension of a scalemic mixture, predominately (3R,4S)-3-methyl-4-(3-tosyl-3H-imidazo[1,2-a]pyrrolo[2,3-e]pyrazin-8-yl)cyclopentanone (4.00 g, 9.79 mmol, Example #7, Step F) in MeOH (49.0 mL) at about 0° C. was added sodium borohydride (0.185 g, 4.90 mmol). The reaction mixture was stirred at about 0° C. for about 1 h. The reaction was quenched with saturated aqueous NH₄Cl (10 mL), warmed to ambient temperature and the organics were removed under reduced pressure. The residue was taken up in DCM and water (50 mL each) and the layers were separated. The aqueous phase was extracted with DCM (3×50 mL) and the combined organics were dried over anhydrous MgSO₄, filtered, and concentrated under reduced pressure to give a scalemic mixture, predominately (1S,3R,4S)-3-methyl-4-(3-tosyl-3H-imidazo[1,2-a]pyrrolo[2,3-e]pyrazin-8-yl)cyclopentanol (3.61 g, 90% crude) as a brown foam; LC/MS (Table 1, Method a) Rₜ=1.92 min; MS m/z: 411 (M+H)⁺.

### Step B: 3-methyl-4-(3-tosyl-3H-imidazo[1,2-a]pyrrolo[2,3-e]pyrazin-8-yl)cyclopentyl methanesulfonate

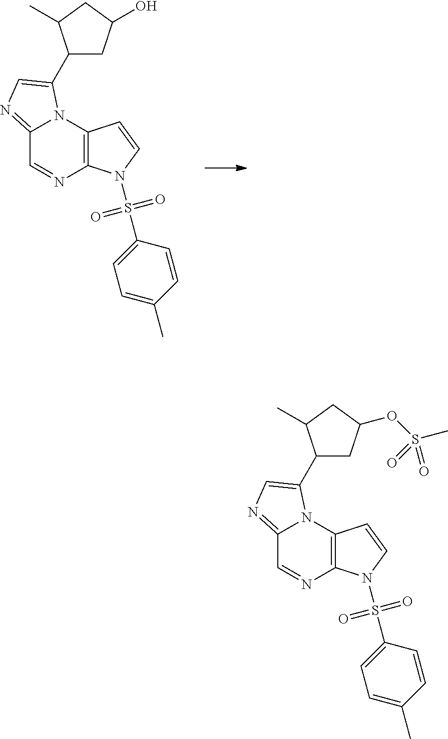 [SMILES: Cc1ccc(S(=O)(=O)n2ccc3c2ncc2ncc(C4CC(O)CC4C)n23)cc1 ; Cc1ccc(S(=O)(=O)n2ccc3c2ncc2ncc(C4CC(OS(C)(=O)=O)CC4C)n23)cc1]

To a solution of a scalemic mixture, predominate (1S,3R,4S)-3-methyl-4-(3-tosyl-3H-imidazo[1,2-a]pyrrolo[2,3-e]pyrazin-8-yl)cyclopentanol (3.6 g, 8.77 mmol) in DCM (35 mL) at about 0° C. was added TEA (3.7 mL, 26.3 mmol) followed by methanesulfonyl chloride (1.03 mL, 13.2 mmol). The reaction was left stirring at about 0° C. for about 2 h. Water (30 mL) was added and the reaction was warmed to ambient temperature. The layers were separated and the aqueous phase was extracted with DCM (2×25 mL). The combined organics were washed with brine (20 mL), dried over anhydrous MgSO₄, filtered, and concentrated under reduced pressure. The crude material was purified by silica gel chromatography eluting with a gradient of 0-10% MeOH in DCM to give a scalemic mixture, predominately (1S,3R,4S)-3-methyl-4-(3-tosyl-3H-imidazo[1,2-a]pyrrolo[2,3-e]pyrazin-8-yl)cyclopentyl methanesulfonate (3.83 g, 89%) as a tan foam; LC/MS (Table 1, Method a) Rₜ=2.16 min; MS m/z: 489 (M+H)⁺.

### Step C: 1-(3-methyl-4-(3-tosyl-3H-imidazo[1,2-a]pyrrolo[2,3-e]pyrazin-8-yl)cyclopentyl)piperidine-4-carbonitrile

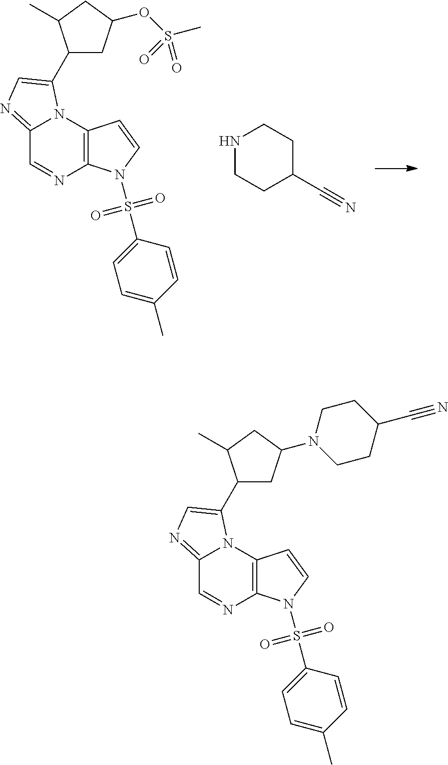 [SMILES: Cc1ccc(S(=O)(=O)n2ccc3c2ncc2ncc(C4CC(OS(C)(=O)=O)CC4C)n23)cc1 ; N#CC1CCNCC1 ; Cc1ccc(S(=O)(=O)n2ccc3c2ncc2ncc(C4CC(N5CCC(C#N)CC5)CC4C)n23)cc1]

To a solution of a scalemic mixture, predominately (1S,3R,4S)-3-methyl-4-(3-tosyl-3H-imidazo[1,2-a]pyrrolo[2,3-e]pyrazin-8-yl)cyclopentyl methanesulfonate (0.300 g, 0.614 mmol) and piperidine-4-carbonitrile (0203 g, 1.84 mmol, Oakwood) in DMF (1.3 mL) was added Hunig's Base (0.540 mL, 3.10 mmol) and the reaction was heated to about 80° C. for about 16 h. The reaction was cooled to ambient temperature, poured into ice water (about 10 mL) and stirred at ambient temperature for about 10 min. The precipitate was filtered off to give a scalemic mixture, predominately 1-((1R,3R,4S)-3-methyl-4-(3-tosyl-3H-imidazo[1,2-a]pyrrolo[2,3-e]pyrazin-8-yl)cyclopentyl)piperidine-4-carbonitrile (0.250 g, 81% crude) as a tan solid; LC/MS (Table 1, Method a) Rₜ=1.65 min; MS m/z: 503 (M+H)⁺.

### Step D: 1-((1R,3S,4R)-3-(3H-imidazo[1,2-a]pyrrolo[2,3-e]pyrazin-8-yl)-4-methylcyclopentyl)piperidine-4-carbonitrile

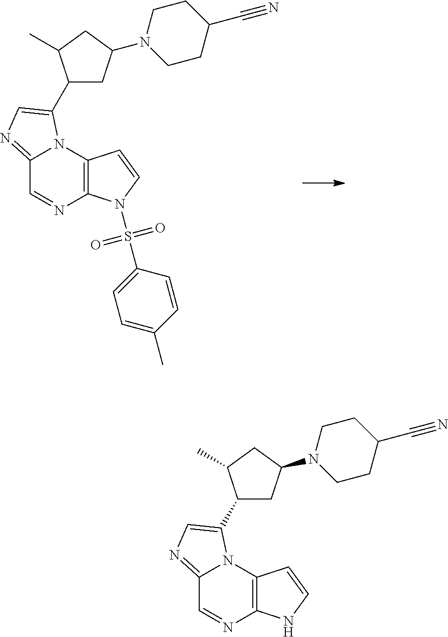 [SMILES: Cc1ccc(S(=O)(=O)n2ccc3c2ncc2ncc(C4CC(N5CCC(C#N)CC5)CC4C)n23)cc1 ; C[C@@H]1C[C@@H](N2CCC(C#N)CC2)C[C@@H]1c1cnc2cnc3[nH]ccc3n12]

To a solution of 1-((1R,3R,4S)-3-methyl-4-(3-tosyl-3H-imidazo[1,2-a]pyrrolo[2,3-e]pyrazin-8-yl)cyclopentyl)piperidine-4-carbonitrile (0.248 g, 0.419 mmol) in MeOH (1.5 mL) was added potassium cyanide (0.082 g, 1.258 mmol) and the resulting mixture was left stirring at ambient temperature for about 16 h. The solvent was removed under reduced pressure and the residue was partitioned between EtOAc and water (5 mL each). The aqueous phase was extracted with EtOAc (2×5 mL) and the combined organics were washed with brine (5 mL), dried over anhydrous MgSO₄, filtered, and concentrated under reduced pressure. The crude material was purified by RP-HPLC (Table 1, method d) and the fractions were concentrated under reduced pressure. The material was further purified by chiral HPLC (Table 2, method 7) to give 1-((1R,3S,4R)-3-(3H-imidazo[1,2-a]pyrrolo[2,3-e]pyrazin-8-yl)-4-methylcyclopentyl)piperidine-4-carbonitrile (0.025 g, 17%) as an off-white solid; LC/MS (Table 1, Method a) Rₜ=0.99 min; MS m/z: 349 (M+H)⁺. Jak3 IC₅₀=C

### Example #9

### 3-((1S,3R,4S)-3-methyl-4-(6H-pyrrolo[2,3-e][1,2,4]triazolo[4,3-a]pyrazin-1-yl)cyclopentyl)propanenitrile

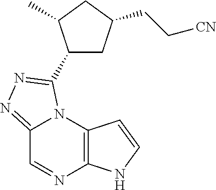 [SMILES: C[C@@H]1C[C@H](CCC#N)C[C@@H]1c1nnc2cnc3[nH]ccc3n12]

### Step A: 8-methyl-N′-(5-tosyl-5H-pyrrolo[2,3-b]pyrazin-2-yl)-1,4-dioxaspiro[4.4]nonane-7-carbohydrazide

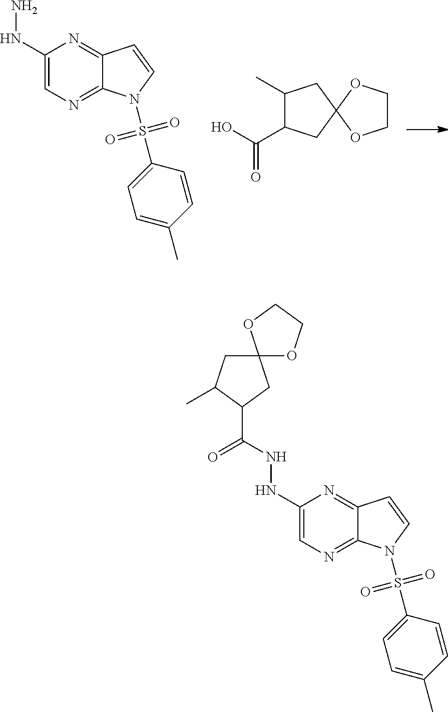 [SMILES: Cc1ccc(S(=O)(=O)n2ccc3nc(NN)cnc32)cc1 ; CC1CC2(CC1C(=O)O)OCCO2 ; Cc1ccc(S(=O)(=O)n2ccc3nc(NNC(=O)C4CC5(CC4C)OCCO5)cnc32)cc1]

A round bottom flask was charged with 2-hydrazinyl-5-tosyl-5H-pyrrolo[2,3-b]pyrazine (26.1 g, 86.0 mmol, WO2011/068881), a scalemic mixture, predominately (7S,8R)-8-methyl-1,4-dioxaspiro[4.4]nonane-7-carboxylic acid (16.0 g, 86.0 mmol, Example #7 step B), and DCM (430 mL). To the flask were added HATU (35.9 g, 95.0 mmol) and TEA (47.6 mL, 344 mmol) and the reaction mixture was stirred at rt for about 3 h, The reaction was diluted with water (250 mL) and the layers were separated. The aqueous layer was extracted with DCM (2×200 mL) and the combined organic layers were washed with brine (3×250 mL), dried over MgSO₄, and filtered Silica gel (about 120 g) was added to the filtrate and the solvent was removed under reduced pressure. The remaining silica mixture was purified on by silica gel chromatography eluting with a gradient of 0-5% MeOH in DCM to give a scalemic mixture, predominately (7S,8R)-8-methyl-N′-(5-tosyl-5H-pyrrolo[2,3-b]pyrazin-2-yl)-1,4-dioxaspiro[4.4]nonane-7-carbohydrazide (46.83 g, 116%, 85% purity) as a brown foam; LC/MS (Table 1, Method a) Rₜ=2.10 min; MS m/z: 472 (M+H)⁺.

### Step B: 1-(8-methyl-1,4-dioxaspiro[4.4]nonan-7-yl)-6-tosyl-6H-pyrrolo[2,3-e][1,2,4]triazolo[4,3-a]pyrazine

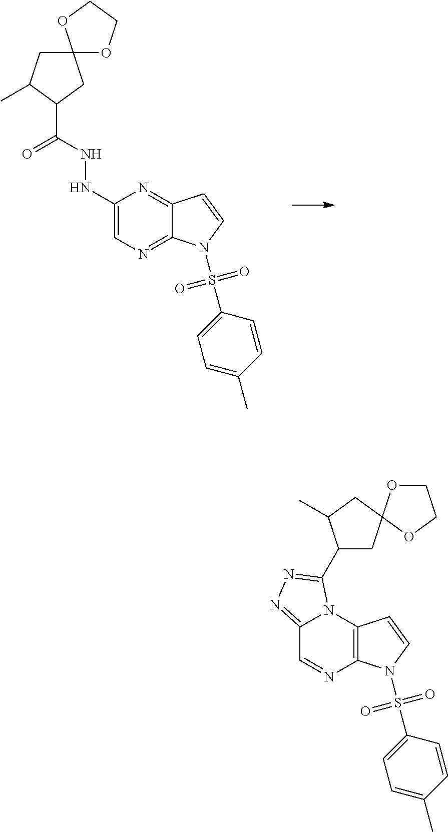 [SMILES: Cc1ccc(S(=O)(=O)n2ccc3nc(NNC(=O)C4CC5(CC4C)OCCO5)cnc32)cc1 ; Cc1ccc(S(=O)(=O)n2ccc3c2ncc2nnc(C4CC5(CC4C)OCCO5)n23)cc1]

To a solution of a scalemic mixture, predominately (7S,8R)-8-methyl-N′-(5-tosyl-5H-pyrrolo[2,3-b]pyrazin-2-yl)-1,4-dioxaspiro[4.4]nonane-7-carbohydrazide (46.8 g, 84 mmol) in 1,4-dioxane (500 mL) was added TEA (47 mL, 337 mmol), followed by the dropwise addition of thionyl chloride (7.4 mL, 101 mmol). The reaction was heated at about 75° C. for about 2 h. The reaction mixture was cooled to ambient temperature and the solvent was removed under reduced pressure. EtOAc (500 mL) was added and the organics washed with saturated aqueous NaHCO₃ (2×300 mL), followed by brine (2×300 mL). The layers were separated and the organic portion was dried over anhydrous MgSO₄, filtered, and concentrated under reduced pressure. The crude material was purified by silica gel chromatography eluting with a gradient of 0-60% acetone in heptane to give a scalemic mixture, predominately 1-((7S,8R)-8-methyl-1,4-dioxaspiro[4.4]nonan-7-yl)-6-tosyl-6H-pyrrolo[2,3-e][1,2,4]triazolo[4,3-a]pyrazine (34.95 g, 91%, 85% purity) as a brown foam; LC/MS (Table 1, Method a) Rₜ=2.21 min; MS m/z: 454 (M+H)⁺.

### Step C: 1-(8-methyl-1,4-dioxaspiro[4.4]nonan-7-yl)-6H-pyrrolo[2,3-e][1,2,4]triazolo[4,3-a]pyrazine

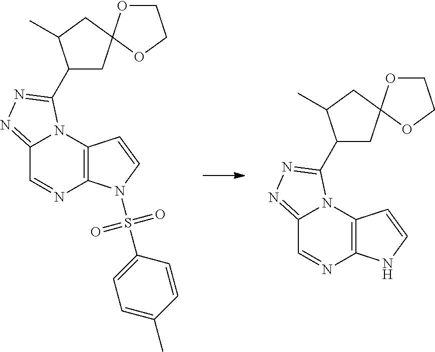 [SMILES: Cc1ccc(S(=O)(=O)n2ccc3c2ncc2nnc(C4CC5(CC4C)OCCO5)n23)cc1 ; CC1CC2(CC1c1nnc3cnc4[nH]ccc4n13)OCCO2]

To a scalemic mixture of predominately 1-((7S,8R)-8-methyl-1,4-dioxaspiro[4.4]nonan-7-yl)-6-tosyl-6H-pyrrolo[2,3-e][1,2,4]triazolo[4,3-a]pyrazine (34.9 g, 65.4 mmol) in 1,4-dioxane (262 mL) was added 2N aqueous NaOH (66.0 mL, 132 mmol). The reaction mixture was stirred at about 70° C. for about 2 h. The reaction was cooled to ambient temperature and water (150 mL) was added. The mixture was extracted with EtOAc (4×200 mL) and DCM (3×200 mL). The combined organic layers were dried over anhydrous MgSO₄ and filtered. Silica gel (about 65 g) was added and the solvent was removed under reduced pressure. The remaining mixture of silica gel and crude product was purified by silica gel chromatography eluting with a gradient of 0-10% MeOH in DCM to give a scalemic mixture, predominately 1-((7S,8R)-8-methyl-1,4-dioxaspiro[4.4]nonan-7-yl)-6H-pyrrolo[2,3-e][1,2,4]triazolo[4,3-a]pyrazine (16.1 g 82%) as a tan foam; LC/MS (Table 1, Method a) Rₜ=1.31 min; MS m/z: 300 (M+H)⁺.

### Step D: 1-(8-methyl-1,4-dioxaspiro[4.4]nonan-7-yl)-6-((2-(trimethylsilyl)ethoxy)methyl)-6H-pyrrolo[2,3-e][1,2,4]triazolo[4,3-a]pyrazine

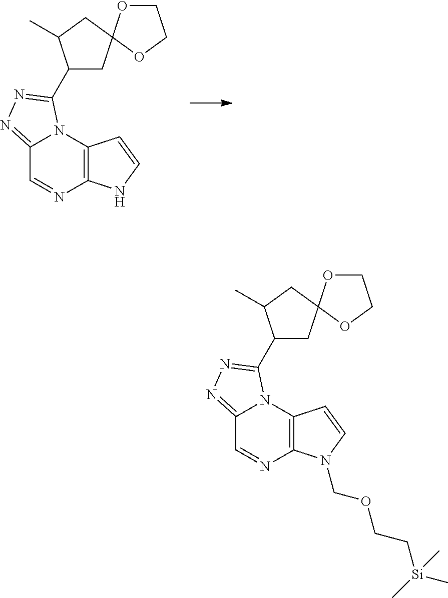 [SMILES: CC1CC2(CC1c1nnc3cnc4[nH]ccc4n13)OCCO2 ; CC1CC2(CC1c1nnc3cnc4c(ccn4COCC[Si](C)(C)C)n13)OCCO2]

To a solution of scalemic mixture predominately 1-((7S,8R)-8-methyl-1,4-dioxaspiro[4.4]nonan-7-yl)-6H-pyrrolo[2,3-e][1,2,4]triazolo[4,3-a]pyrazine (16.1 g, 53.8 mmol) in DMF (215 mL) at about 0° C. was added sodium hydride (2.37 g, 59.2 mmol, 60% dispersion in mineral oil) portionwise. The reaction stirred for about 30 min at about 0° C. and SEM-Cl (10.5 mL, 59.2 mmol) was added dropwise via syringe. The reaction mixture was warmed to ambient temperature and stirred for about 2 h. Water and EtOAc (150 mL each) were added and the layers were separated. The aqueous phase was extracted with EtOAc (3×200 mL) and the combined organics were washed with brine (6×200 mL), dried over anhydrous MgSO₄, filtered and concentrated under reduced pressure. The remaining dark oil was purified silica chromatography eluting with a gradient of 0.60% acetone in heptane to give a scalemic mixture, predominately 1-((7S,8R)-8-methyl-1,4-dioxaspiro[4.4]nonan-7-yl)-6-((2-(trimethylsilyl)ethoxy)methyl)-6H-pyrrolo[2,3-e][1,2,4]triazolo[4,3-a]pyrazine (15.1 g, 65%) as a tan solid; LC/MS (Table 1, Method a) Rₜ=2.48 min; MS m/z: 430 (M+H)⁺.

### Step E: 3-methyl-4-(6-((2-(trimethylsilyl)ethoxy)methyl)-6H-pyrrolo[2,3-e][1,2,4]triazolo[4,3-a]pyrazin-1-yl)cyclopentanone

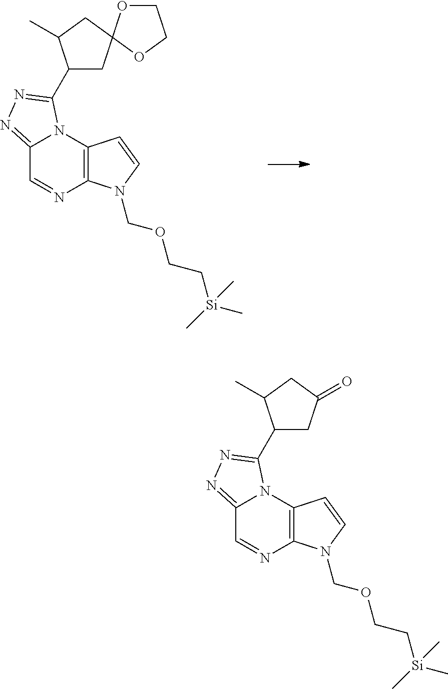 [SMILES: CC1CC2(CC1c1nnc3cnc4c(ccn4COCC[Si](C)(C)C)n13)OCCO2 ; CC1CC(=O)CC1c1nnc2cnc3c(ccn3COCC[Si](C)(C)C)n12]

To a scalemic mixture, predominately 1-((7S,8R)-8-methyl-1,4-dioxaspiro[4.4]nonan-7-yl)-6-((2-(trimethylsilyl)ethoxy)methyl)-6H-pyrrolo[2,3-e][1,2,4]triazolo[4,3-a]pyrazine (15.1 g, 35.1 mmol) in THF (146 mL) at about 0° C. was added 1N aqueous HCl (105 mL, 105 mmol). The reaction mixture was warmed to ambient temperature and stirred for about 16 h. The organics were removed under reduced pressure and the aqueous layer was neutralized with saturated aqueous NaHCO₃ to about pH 8. EtOAc (300 mL) was added and the layers were separated. The aqueous layer was extracted with EtOAc (2×100 mL) and the combined organic layers were washed with brine (3×100 mL), dried over anhydrous Na₂SO₄, filtered, and concentrated to about 50 mL. Heptane (about 300 mL) was added and the precipitate was filtered off washing with heptane (about 100 mL) and dried in vacuum oven at about 50° C. to give a scalemic mixture, predominately (3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentanone (12.26 g, 90%) as an off white solid; LC/MS (Table 1, Method a) Rₜ=2.20 min; MS m/z: 386 (M+H)⁺.

### Step F: diethyl 1-diaza-2-oxopropylphosphonate

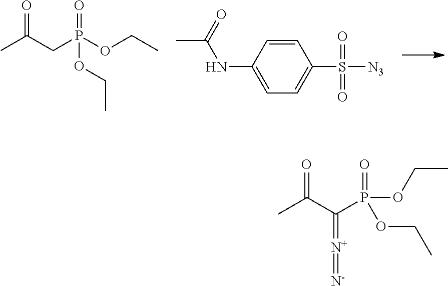

A round bottom flask was charged with diethyl 2-oxopropylphosphonate (50.0 g, 258 mmol, Alfa Aesar), toluene (850 mL) and THF (180 mL). The reaction was cooled to about 0° C. in an ice bath and sodium hydride (11.3 g, 283 mmol, 60% dispersion in mineral oil) was added portionwise over 10 min. The mixture was stirred for about 1 h at about 0° C. and 4-acetamidobenzenesulfonyl azide (64.4 g, 260 mmol) was added portionwise. The reaction was warmed to ambient temperature and left stirring for about 3 h. The solids were filtered off washing with DCM (600 mL) and the filtrate was concentrated under reduced pressure. The remaining yellow oil was taken up in DCM (about 200 mL) and filtered through a pad of silica gel washing with DCM (about 500 mL). The filtrate was concentrated under reduced pressure to give diethyl 1-diazo-2-oxopropylphosphonate (40.15 g, 71%) as a yellow oil; ¹H NMR (400 MHz, DMSO-d6) δ 4.19-4.07 (m, 4H), 2.22 (s, 3H), 1.27 (t, 6H).

### Step G: 1-(4-(methoxymethylene)-2-methylcyclopentyl)-6-((2-(trimethylsilyl)ethoxy)methyl)-6H-pyrrolo[2,3-e][1,2,4]triazolo[4,3-a]pyrazine

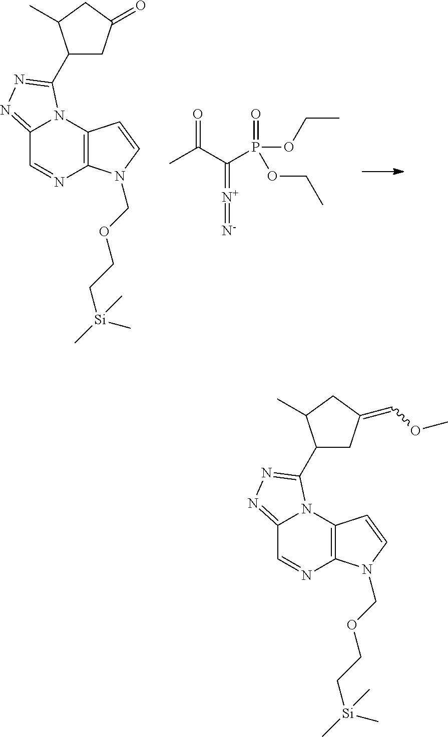

To a suspension of diethyl 1-diazo-2-oxopropylphosphonate (1.71 g, 7.78 mmol) and a scalemic mixture, predominately (3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentanone (2.00 g, 5.19 mmol) in MeOH (10.4 mL) at about 0° C. was added K₂CO₃ (1.43 g, 10.4 mmol). The mixture was stirred at about 0° C. for about 30 min and then warmed to rt and left stirring for about 72 h. The reaction mixture was cooled to about 0° C. and K₂CO₃ (1.43 g, 10.4 mmol) was added followed by diethyl 1-diazo-2-oxopropylphosphonate (1.71 g, 7.78 mmol). The mixture was stirred at about 0° C. for about 30 min, warmed to rt and left stirring for about 16 h. The reaction mixture was cooled to about 0° C. and K₂CO₃ (1.43 g, 10.4 mmol) was added followed by diethyl 1-diazo-2-oxopropylphosphonate (1.71 g, 7.78 mmol). The mixture was stirred at about 0° C. for about 30 min, warmed to rt and left stirring for about 16 h. The organics were removed under reduced pressure and the residue was partitioned between saturated aqueous NH₄Cl and EtOAc (100 mL each). The aqueous layer was extracted with EtOAc (2×100 mL) and the combined organic layers were dried over anhydrous MgSO₄, and concentrated under reduced pressure. The crude material was purified by silica gel chromatography eluting with a gradient of 10-75% EtOAc in heptane to give a scalemic mixture, predominately 1-((1S,2R)-4-(methoxymethylene)-2-methylcyclopentyl)-6-((2-(trimethylsilyl)ethoxy)methyl)-6H-pyrrolo[2,3-e][1,2,4]triazolo[4,3-a]prazine (1.70 g, 79%) as a yellow oil as a mixture of E and Z isomers; LC/MS (Table 1, Method a) Rₜ=2.58 min; MS m/z: 414 (M+H)⁺.

### Step H: 3-methyl-4-(6-(2-(trimethylsilyl)ethoxy)methyl)-6H-pyrrolo[2,3-e][1,2,4]triazolo[4,3-a]pyrazin-1-yl)cyclopentanecarbaldehyde

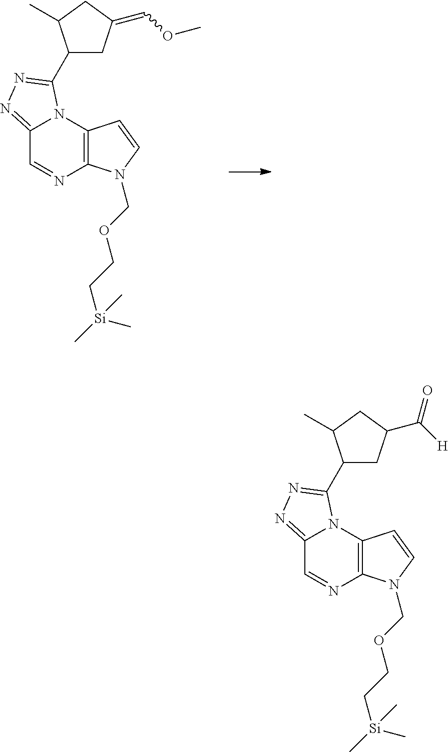 [SMILES: COC=C1CC(C)C(c2nnc3cnc4c(ccn4COCC[Si](C)(C)C)n23)C1 ; CC1CC(C=O)CC1c1nnc2cnc3c(ccn3COCC[Si](C)(C)C)n12]

To a solution of a scalemic mixture, predominately 1-((1S,2R)-4-(methoxymethylene)-2-methylcyclopentyl)-6-((2-(trimethylsilyl)ethoxy)methyl)-6H-pyrrolo[2,3-e][1,2,4]triazolo[4,3-a]pyrazine as a mixture of E and Z isomers (1.70 g, 4.11 mmol) in THF (20 mL) at about 0° C. was added 1N aqueous HCl (12.3 mL, 12.3 mmol). The reaction mixture was warmed to ambient temperature and left stirring for about 30 h. The organics were removed under reduced pressure and saturated aqueous NaHCO₃ was added dropwise until gas evolution stopped. The mixture was extracted with DCM (3×50 mL) and the combined organics were dried over anhydrous MgSO₄, filtered and concentrated under reduced pressure. The crude material was purified by silica gel chromatography eluting with a gradient of 30-100% EtOAc in heptane to give a scalemic mixture, predominately a mixture of (1S,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentanecarbaldehyde and (1R,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentanecarbaldehyde (1.07 g, 65%) as a yellow oil; LC/MS (Table 1, Method a) Rₜ=2.35 min; MS m/z: 400 (M+H)⁺.

### Step I: 3-(3-methyl-4-(6-((2-(trimethylsilyl)ethoxy)methyl)-6H-pyrrolo[2,3-e][1,2,4]triazolo[4,3-a]pyrazine-1-yl)cyclopentyl)acrylonitrile

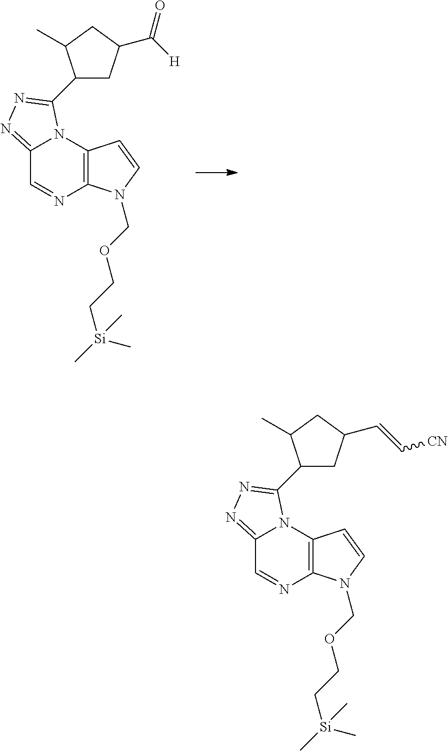 [SMILES: CC1CC(C=O)CC1c1nnc2cnc3c(ccn3COCC[Si](C)(C)C)n12 ; CC1CC(C=CC#N)CC1c1nnc2cnc3c(ccn3COCC[Si](C)(C)C)n12]

To a solution of diethyl cyanomethylphosphonate (0.527 mL, 3.25 mmol) in THF (8 mL) at about 0° C. was added sodium hydride (0.130 g, 3.25 mmol, 60% dispersion in mineral oil). The resulting mixture was stirred at about 0° C. for about 20 min. A solution of a scalemic mixture, predominately a mixture of (1S,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentanecarbaldehyde and (1R,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentanecarbaldehyde (1.00 g, 2.503 mmol) in THF (8 mL) was added dropwise and the reaction mixture was warmed to ambient temperature and stirred for about 1 h. Saturated aqueous NH₄Cl (5 mL) and water (10 mL) were added followed by EtOAc (25 mL). The layers were separated and the aqueous phase was extracted with EtOAc (2×20 mL). The combined organics were washed with brine (15 mL), dried over anhydrous Na₂SO₄, filtered, and concentrated under reduced pressure. The crude material was purified by silica gel chromatography eluting with a gradient of 30-100% EtOAc in heptane to give a scalemic mixture, predominately a mixture of 3-((1S,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentyl)acrylonitrile and 3-((1R,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentyl)acrylonitrile (1.20 g, 96%, 85% crude) as a orange oil as a mixture of E and Z isomers; LC/MS (Table 1, Method a) Rₜ=2.51 min; MS m/z: 423 (M+H)⁺ and Rₜ=2.53 min; MS m/z: 423 (M+H)⁺.

### Step J: 3-((1S,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentyl)propanenitrile

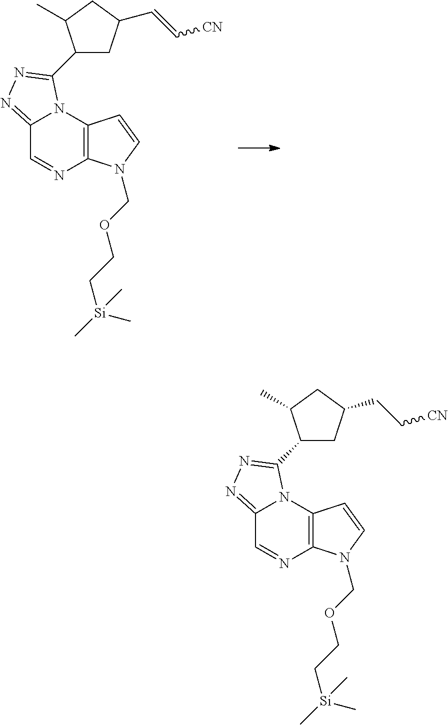 [SMILES: CC1CC(C=CC#N)CC1c1nnc2cnc3c(ccn3COCC[Si](C)(C)C)n12 ; C[C@@H]1C[C@H](CCC#N)C[C@@H]1c1nnc2cnc3c(ccn3COCC[Si](C)(C)C)n12]

A scalemic mixture, predominately a mixture of 3-((1S,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentyl)acrylonitrile and 3-(1R,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentyl)acrylonitrile as a mixture of E and Z isomers (1.2 g, 2.272 mmol) and Pearlman's catalyst (0.160 g, 0.227 mmol) were purged and evacuated with nitrogen (3×). THF (22 mL) was added and the mixture was purged and evacuated with nitrogen (3×) and placed under a hydrogen atmosphere via balloon. After about 2 h the reaction was filtered through a pad of Celite®, washing with ether (about 50 mL), and the filtrate was concentrated under reduced pressure. The crude oil was purified, silica gel chromatography eluting with a gradient of 0-50% acetone in heptane. The product containing fractions were combined and concentrated under reduced pressure and the material was further purified by chiral HPLC (Table 2, Method 8); Rₜ=14.1 min, or =negative] to give 3-((1S,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentyl)propanenitrile (0.650 g, 67%) as a brown foam; LC/MS (Table 1, Method a) Rₜ=2.61 min; MS m/z: 425 (M+H)⁺.

### Step K: 3-((1S,3R,4S)-3-methyl-4-(6H-pyrrolo[2,3-e][1,2,4]triazolo[4,3-a]pyrazin-1-yl)cyclopentyl)propanenitrile

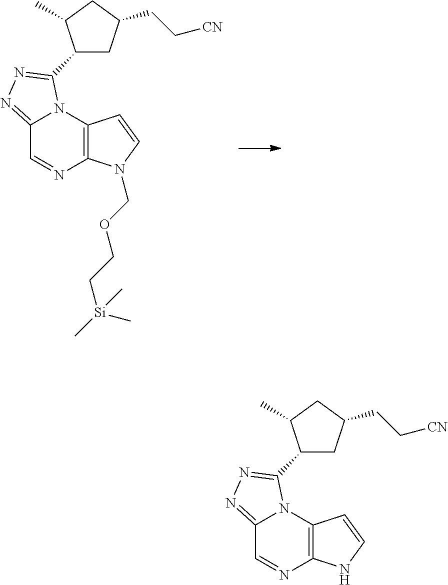 [SMILES: C[C@@H]1C[C@H](CCC#N)C[C@@H]1c1nnc2cnc3c(ccn3COCC[Si](C)(C)C)n12 ; C[C@@H]1C[C@H](CCC#N)C[C@@H]1c1nnc2cnc3[nH]ccc3n12]

To a solution of 3-((1S,3R,4S)-3-methyl-4-(6-((2-(trimethylsilyl)ethoxy)methyl)-6H-pyrrolo[2,3-e][1,2,4]triazolo[4,3-a]pyrazin-1-yl)cyclopentyl)propanenitrile (0.629 g, 1.48 mmol) in DCM (2 mL) was added TFA (1.14 mL, 14.8 mmol). The reaction was stirred at ambient temperature for about 6 h. The reaction was concentrated under reduced pressure and the remaining oil was taken up in 1,4-dioxane (3 mL). Ammonium hydroxide (1.15 mL, 29.6 mmol) was added and the reaction was left stirring overnight at ambient temperature. Water and DCM (10 mL each) were added and the layers were separated. The aqueous phase was extracted with DCM (2×10 mL) and the combined organics were dried over anhydrous MgSO₄, filtered, and concentrated under reduced pressure. The crude material was purified by silica gel chromatography eluting with a gradient of 0-10% MeOH in DCM to give (0.319 g, 73%) as a white solid; LC/MS (Table 1, Method a) Rₜ=1.51 min; MS m/z: 295 (M+H)⁺. Jak3 IC₅₀=C

### Example #10

### 3-((1R,3R,4S)-3-methyl-4-(6H-pyrrolo[2,3-e][1,2,4]triazolo[4,3-a]pyrazin-1-yl)cyclopentyl)propanenitrile

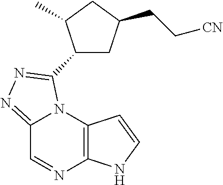 [SMILES: C[C@@H]1C[C@@H](CCC#N)C[C@@H]1c1nnc2cnc3[nH]ccc3n12]

### Step A: ethyl 4-hydroxy-2-methylcyclopentanecarboxylate

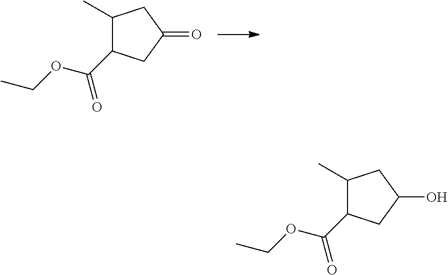

To a solution of a scalemic mixture, predominately (1S,2R)-ethyl 2-methyl-4-oxocyclopentanecarboxylate (5 g, 29.4 mmol, WO2011/06881) in ethanol (42 mL) at about 0° C. was added NaBH₄ (0.560 g, 14.7 mmol) portionwise. The reaction was left stirring at about 0° C. for about 1 h. The reaction was slowly quenched by the dropwise addition of saturated aqueous NH₄Cl (about 30 mL) followed by water (20 mL) to dissolve any solids. Ethanol was removed under reduced pressure and the remaining aqueous layer was extracted with MTBE (2×100 mL). The combined organics were washed with brine (50 mL), dried over anhydrous Na₂SO₄, filtered, and concentrated under reduced pressure to give a scalemic mixture, predominately (1S,2R,4S)-ethyl 4-hydroxy-2-methylcyclopentane carboxylate (4.99 g, 98%) as a colorless oil; ¹H NMR (400 MHz, CDCl₃) δ 4.29 (m, 1H), 4.23-4.11 (m, 2H), 3.37 (s, 1H), 2.83-2.79 (m, 1H), 2.40-2.29 (m, 1H), 2.29-2.18 (m, 1H), 2.06 (m, 1H), 2.02-1.93 (m, 1H), 1.45-1.34 (m, 1H), 1.33-1.24 (m, 3H), 1.04 (d, 3H).

### Step B: ethyl 2-methyl-4-(methylsulfonyloxy)cyclopentanecarboxylate

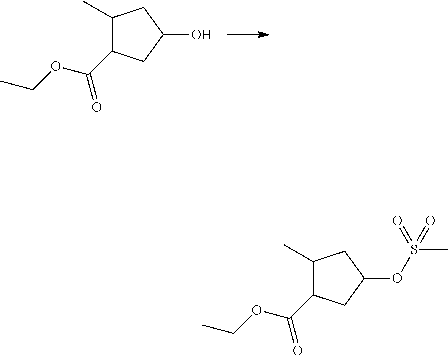 [SMILES: CCOC(=O)C1CC(O)CC1C ; CCOC(=O)C1CC(OS(C)(=O)=O)CC1C]

A solution of a scalemic mixture, predominately (1S,2R,4S)-ethyl 4-hydroxy-2-methylcyclopentanecarboxylate (4.99 g, 29 mmol) in DCM (31 mL) was cooled to about 0° C. and TEA (8.08 mL, 57.9 mmol) was added. Methanesulfonyl chloride (3.38 mL, 43.5 mmol) was added and the reaction was left stirring at about 0° C. for about 30 min and then warmed to ambient temperature and stirred for about 1 h. The reaction was partitioned between water (30 mL) and cyclohexane (150 mL) and the layers were separated. The organic phase was washed with brine (40 mL), dried over anhydrous Na₂SO₄, filtered, and concentrated under reduced pressure to give a scalemic mixture, predominately (1S,2R,4S)-ethyl 2-methyl-4-(methylsulfonyloxy)cyclopentanecarboxylate (6.78 g, 93% crude) as en orange oil; ¹H NMR (400 MHz, CDCl₃) δ 5.14-5.03 (m, 1H), 4.23-4.09 (m, 2H), 3.02 (s, 3H), 2.83 (q, 1H), 2.43-2.31 (m, 3H), 2.26-2.24 (m, 1H), 1.85-1.80 (m, 1H), 1.31-1.24 (m, 3H), 1.02 (d, 3H).

### Step C: di-tert-butyl 2-(3-(ethoxycarbonyl)-4-methylcyclopentyl)malonate

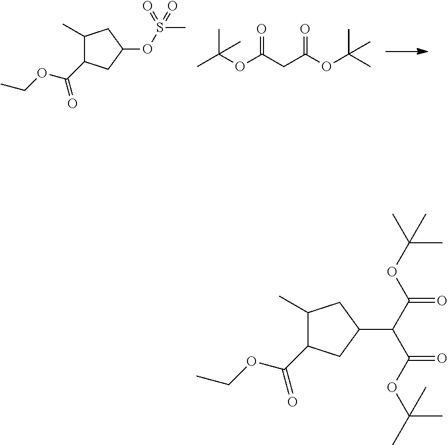

A solution of sodium tert-butoxide (2.31 g, 24 mmol) in THF (22 mL) was cooled to about 0° C. in an ice bath. Di-tert-butyl malonate (5.80 mL, 26.0 mmol) was added and the mixture stirred at about 0° C. for about 30 min. A solution of a scalemic mixture, predominately of (1S,2R,4S)-ethyl 2-methyl-4-(methylsulfonyloxy)cyclopentanecarboxylate (4.29 g, 17.14 mmol) in THF (22 mL) was added and the reaction was warmed to about 50° C. and left stirring overnight. The reaction was quenched with saturated aqueous NH₄Cl (50 mL) and was extracted with cyclohexane (2×100 mL). The combined organic layers were washed with brine (50 mL), dried over anhydrous Na₂SO₄, filtered and concentrated under reduced pressure. The crude material was purified by silica gel chromatography eluting with a gradient of 0-50% EtOAc in heptane to give a scalemic mixture, predominately di-tert-butyl 2-((1R,3S,4R)-3-(ethoxycarbonyl)-4-methylcyclopentyl)malonate (6.46 g, 102%) as a clear colorless oil with 0.75 mol % di-tert-butyl malonate; ¹H NMR (400 MHz, CDCl₃) δ 4.17-4.07 (m, 2H), 2.87 (m, 2H), 2.42 (m, 1H), 2.29 (ddd, 1H), 1.72 (ddd, 1H), 1.64-1.54 (m, 3H), 1.46 (s, 18H), 1.26 (t, 3H), 0.91 (d, 3H).

### Step D: 2-(3-(ethoxycarbonyl)-4-methylcyclopentyl)acetic acid

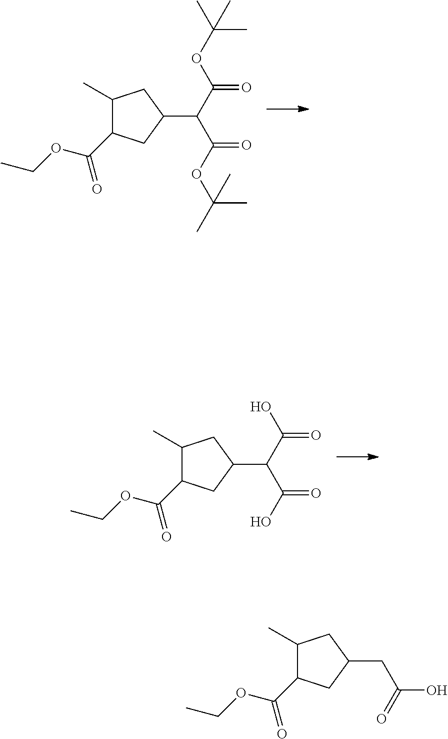 [SMILES: CCOC(=O)C1CC(C(C(=O)OC(C)(C)C)C(=O)OC(C)(C)C)CC1C ; CCOC(=O)C1CC(C(C(=O)O)C(=O)O)CC1C ; CCOC(=O)C1CC(CC(=O)O)CC1C]

A solution of a scalemic mixture, predominately di-tert-butyl 2-((1R,3S,4R)-3-(ethoxycarbonyl)-4-methylcyclopentyl)malonate (6.35 g, 17.14 mmol) in TFA (35 mL) was stirred at about 60° C. for about 2 h. The reaction was cooled to ambient temperature and the reaction was concentrated under reduced pressure. Toluene (35 mL) was added and the mixture heated to reflux for about 16 h. TFA (1 mL, 12.98 mmol) was added and the reaction continued stirring reflux for about 24 h. The reaction was cooled to ambient temperature and extracted with 10% aqueous K₂CO₃ (3×50 mL) and the combined aqueous layers washed with toluene (2×50 mL). The combined aqueous layers was adjusted to about pH 4 with 20% aqueous citric acid and extracted with MTBE (3×100 mL). The combined MTBE fractions were washed with brine (50 mL), dried over anhydrous Na₂SO₄, filtered, and concentrated under reduced pressure to give a scalemic mixture, predominately 2-((1R,3S,4R)-3-(ethoxycarbonyl)-4-methylcyclopentyl)acetic acid (2.55 g, 69%) as a light yellow oil; ¹H NMR (400 MHz, DMSO-d6) δ 11.97 (s, 1H), 4.18-3.90 (m, 2H), 2.88 (dd, 1H), 2.46 (d, 1H), 2.37 (m, 1H), 2.22 (d, 2H), 2.10 (ddd, 1H), 1.56 (ddd, 1H), 1.49-1.31 (m, 2H), 1.18 (t, 3H), 0.82 (d, 3H).

### Step E: ethyl 4-(2-hydroxyethyl)-2-methylcyclopentanecarboxylate

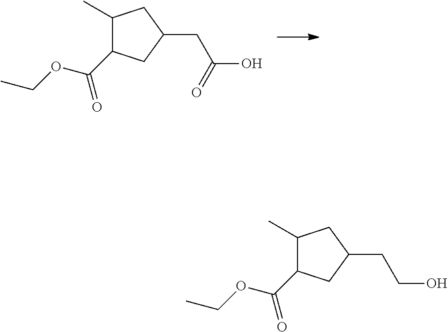

A solution of a scalemic mixture, predominately 2-((1R,3S,4R)-3-(ethoxycarbonyl)-4-methylcyclopentyl)acetic acid (2.53 g, 11.8 mmol) in THF (20 mL) was cooled to about 0° C. in an ice bath. 1M borane-tetrahydrofuran complex in THF (14.2 mL, 14.2 mmol) was added and the reaction was left stirring at about 0° C. for about 1 h. The reaction was warmed to ambient temperature and left stirring for about 4 h. The reaction was quenched with MeOH (50 mL), and the organics were removed under reduced pressure. The crude material was taken up and concentrated from MeOH (2×50 mL) to give a scalemic mixture, predominately (1S,2R,4R)-ethyl 4-(2-hydroxyethyl)-2-methylcyclopentanecarboxylate (2.18 g, 92% crude) as a colorless oil; ¹H NMR (400 MHz, CDCl₃) δ 4.17-4.08 (m, 2H), 3.68-3.61 (m, 2H), 2.92-2.83 (m, 1H), 2.48-2.37 (m, 1H), 2.37-2.28 (m, 1H), 2.28-2.18 (m, 1.64-1.61 (m, 4H), 1.49-1.37 (m, 2H), 1.26 (m, 1.6 Hz, 3H), 0.90 (d, 3H).

### Step F: ethyl 2-methyl-4-(2-(methylsulfonyloxy)ethyl)cyclopentanecarboxylate

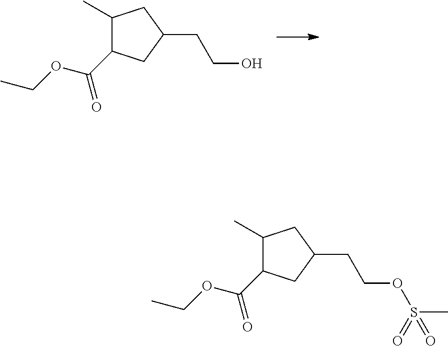 [SMILES: CCOC(=O)C1CC(CCO)CC1C ; CCOC(=O)C1CC(CCOS(C)(=O)=O)CC1C]

A solution of a scalemic mixture, predominately (1S,2R,4R)-ethyl 4-(2-hydroxyethyl)-2-methylcyclopentanecarboxylate (2.15 g, 10.74 mmol) in DCM (17 mL) was cooled to about 0° C. in an ice bath. TEA (3 mL, 21.5 mmol) was added followed by methanesulfonyl chloride (1.3 mL, 16.1 mmol) and the reaction was warmed to ambient temperature for about 2 h. The reaction was partitioned between water (20 mL) and cyclohexane (100 mL) and the layers were separated. The organic phase was washed with brine (20 dried over anhydrous Na₂SO₄, filtered and concentrated under reduced pressure to give a scalemic mixture, predominately (1S,2R,4R)-ethyl 2-methyl-4-(2-(methylsulfonyloxy)ethyl)cyclopentanecarboxylate (2.73 g, 91% crude) as a yellow oil; ¹H NMR (400 MHz, CDCl₃) δ 4.22 (t, 2H), 4.13 (m, 2H), 3.01 (s, 3H), 2.93-2.84 (m, 1H), 2.43 (m, 1H), 2.35 (dd, 1H), 2.26 (ddd, 1H), 1.81-1.73 (m, 2H), 1.72-1.63 (m, 1H), 1.51-1.44 (m, 1H), 1.44-1.37 (m, 1H), 1.26 (t, 3H), 0.91 (d, 3H).

### Step G: ethyl 4-(2-cyanoethyl)-2-methylcyclopentanecarboxylate

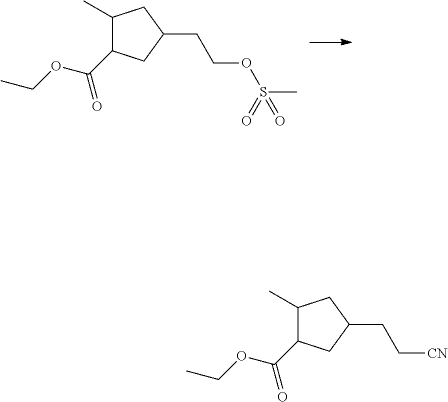

To a solution of a scalemic mixture, predominately (1S,2R,4R)-ethyl 2-methyl-4-(2-(methylsulfonyloxy)ethyl)cyclopentanecarboxylate (2.69 g, 9.66 mmol) in DMF (26 mL) was added sodium cyanide (0.95 g, 19.3 mmol). The reaction was heated to about 85° C. and left stirring for about 3 h. The reaction was cooled to ambient temperature and cyclohexane (60 mL) and water (30 mL) were added. The layers were separated and the aqueous phase was extracted with cyclohexane (2×60 mL). The combined organics were washed with saturated aqueous NaHCO₃ (2×20 mL) and brine (3×15 mL), dried over anhydrous Na₂SO₄, filtered and concentrated under reduced pressure to give a scalemic mixture, predominately (1S,2R,4R)-ethyl 4-(2-cyanoethyl)-2-methylcyclopentanecarboxylate (1.81 g, 89% crude) as a clear colorless oil; ¹H NMR (400 MHz, CDCl₃) δ 4.16-4.10 (m, 2H), 2.91-2.85 (m, 1H), 2.43-2.37 (m, 1H), 2.37-2.30 (m, 3H), 2.28-2.26 (m, 1H), 1.75-1.62 (m, 3H), 1.49-1.34 (m, 2H), 1.26 (t, 3H), 0.91 (d, 3H).

### Step H: allyl-4-(2-cyanoethyl)-2-methylcyclopentanecarboxylate

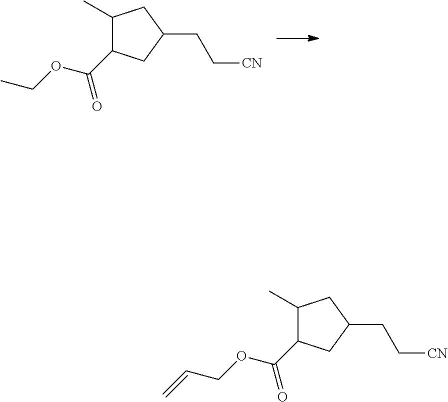 [SMILES: CCOC(=O)C1CC(CCC#N)CC1C ; C=CCOC(=O)C1CC(CCC#N)CC1C]

A mixture of allyl acetate (23.5 mL, 218 mmol) and titanium (IV) isopropoxide (5.6 mL, 18.8 mmol) was heated at reflux for about 7 h. The reaction was cooled, to ambient temperature and concentrated under reduced pressure. Allyl acetate (23.5 mL, 218 mmol) was added and the mixture was heated to reflux and left stirring for about 16 h. The reaction was cooled to ambient temperature and concentrated under reduced pressure. The residue was dissolved in allyl alcohol (38 mL, 557 mmol) and the mixture was added to a scalemic mixture, predominately (1S,2R,4R)-ethyl 4-(2-cyanoethyl)-2-methylcyclopentanecarboxylate (4.56 g, 21.8 mmol) and the reaction mixture was heated to reflux for about 7 h. The reaction was cooled to ambient temperature and concentrated under reduced pressure. Allyl alcohol (38.0 mL, 557 mmol) was added and the reaction mixture was heated to reflux and left stirring for about 16 h. The reaction was cooled to ambient temperature and concentrated under reduced pressure leaving an orange oil. The oil was partitioned between cyclohexane (50 mL) and 10% aqueous HCl (50 mL) and the layers were separated. The organic phase was washed with 10% aq HCl (2×25 mL). The combined aqueous layers were extracted with cyclohexane (50 mL), and the combined organic layers were washed with brine (25 mL), dried over anhydrous Na₂SO₄, filtered, and concentrated under reduced pressure to give a scalemic mixture, predominately (1S,2R,4R)-allyl 4-(2-cyanoethyl)-2-methylcyclopentanecarboxylate (4.76 g, 99% crude) as a yellow oil; ¹H NMR (400 MHz, CDCl₃) δ 5.92 (ddt, 1H), 5.33 (dq, 1H), 5.24 (ddd, 1H), 4.58 (m, 2H), 2.93 (ddd, 1H), 2.45 (ddd, 1H), 2.40-2.24 (m, 4H), 1.75-1.63 (m, 3H), 1.50-1.36 (m, 2H), 0.92 (d, 3H).

### Step I: 4-(2-cyanoethyl)-2-methylcyclopentanecarboxylic acid

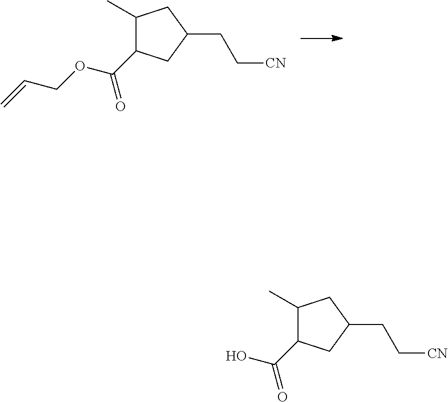 [SMILES: C=CCOC(=O)C1CC(CCC#N)CC1C ; CC1CC(CCC#N)CC1C(=O)O]

To a solution of a scalemic mixture, predominately (1S,2R,4R)-allyl 4-(2-cyanoethyl)-2-methylcyclopentanecarboxylate (4.76 g, 19.8 mmol) in toluene (27 mL) was added Pd(Ph₃P)₄ (1.14 g, 0.989 mmol) followed by pyrrolidine (4.93 mL, 59.4 mmol). The reaction mixture was stirred at ambient temperature for about 2 h. The reaction mixture was washed with 10% aqueous K₂CO₃ (3×50 mL), and the combined aqueous layers were extracted with toluene (20 mL). The pH of the aqueous layer was adjusted to about 4 with 5% aqueous citric acid and it was extracted with MTBE (3×50 mL). The combined organics were washed with brine (20 mL), dried over anhydrous Na₂SO₄, filtered and concentrated under reduced pressure. To a solution of the carboxylic acid in acetonitrile (37 mL) was added dicyclohexylamine (3.94 mL, 19.8 mmol) and the mixture was left stirring at ambient temperature for about 30 min. The solid was filtered washing with acetonitrile (about 15 mL) and left to dry in a vacuum oven at about 60° C. To a suspension of the dicyclohexylamine salt in MTBE (80 mL) was added 0.5 N aqueous HCl (40 mL, 20 mmol) and the reaction mixture was stirred at rt. After about 1 h MTBE (40 mL) and 0.5 N aqueous HCl (20 mL, 10 mmol) were added. The reaction mixture continued stirring at rt for about 3 h. The solids were filtered off, and the filtrate layers were separated. The aqueous layer was extracted with MTBE (2×50 mL) and the combined organic layers were dried over anhydrous Na₂SO₄, filtered and concentrated under reduced pressure to give a scalemic mixture, predominately (1S,2R,4R)-4-(2-cyanoethyl)-2-methylcyclopentanecarboxylic acid (2.66 g, 74% crude) as a yellow oil with solvent impurities of DCM (˜7 mol %) and MTBE (˜2 mol %); LC/MS (Table 1, Method a) Rₜ=1.82 min; MS m/z: 180 (M−H)⁻.

### Step J: 4-(2-cyanoethyl)-2-methyl-N′-(5-tosyl-5H-pyrrolo[2,3-b]pyrazin-2-yl)cyclopentanecarbohydrazide

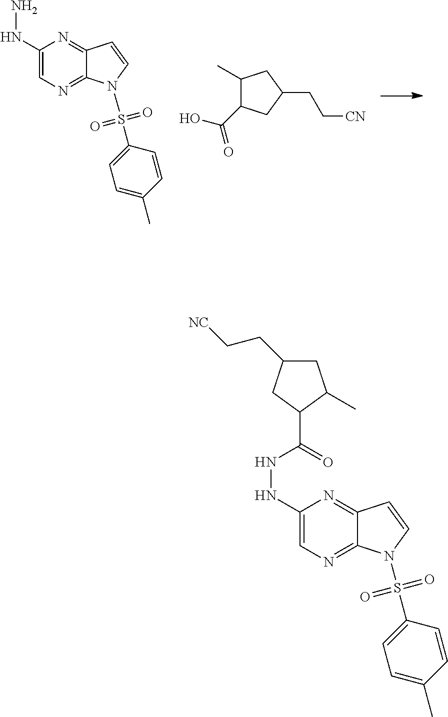 [SMILES: Cc1ccc(S(=O)(=O)n2ccc3nc(NN)cnc32)cc1 ; CC1CC(CCC#N)CC1C(=O)O ; Cc1ccc(S(=O)(=O)n2ccc3nc(NNC(=O)C4CC(CCC#N)CC4C)cnc32)cc1]

To a suspension of 2-hydrazinyl-5-tosyl-5H-pyrrolo[2,3-b]pyrazine (1.091 g, 3.60 mmol, WO2011068881) and a scalemic mixture, predominately (1S,2R,4R)-4-(2-cyanoethyl)-2-methylcyclopentanecarboxylic acid (0.652 g, 3.6 mmol) in DCM (10 mL) was added HATU (1.51 g, 3.96 mmol) followed by TEA (1.51 mL, 10.8 mmol). The reaction was left stirring at ambient temperature for about 1 h. 10% aqueous KH₂PO₄ and DCM (about 20 mL each) were added and the layers were separated. The aqueous phase was extracted with DCM (2×30 mL) and the combined organics were washed with brine (20 mL), dried over anhydrous MgSO₄, filtered, and concentrated under reduced pressure. The crude material was purified by silica gel chromatography eluting with a gradient of 0-70% acetone in heptane to give a scalemic mixture, predominately (1S,2R,4R)-4-(2-cyanoethyl)-2-methyl-N′-(5-tosyl-5H-pyrrolo[2,3-b]pyrazin-2-yl)cyclopentanecarbohydrazide (1.45 g, 86%) as a yellow foam; LC/MS (Table 1, Method a) Rₜ=2.08 min; MS m/z: 467 (M+H)⁺.

### Step K: 3-(3-methyl-4-(6-tosyl-6H-pyrrolo[2,3-e][1,2,4]triazolo[4,3-a]pyrazin-1-yl)cyclopentyl)propanenitrile

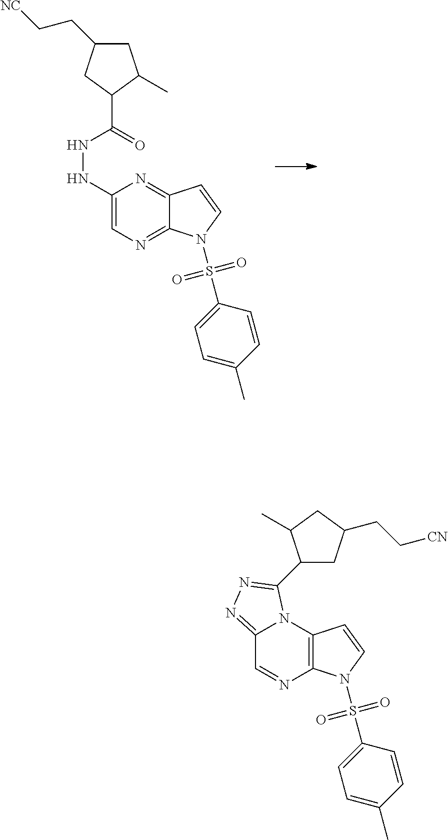 [SMILES: Cc1ccc(S(=O)(=O)n2ccc3nc(NNC(=O)C4CC(CCC#N)CC4C)cnc32)cc1 ; Cc1ccc(S(=O)(=O)n2ccc3c2ncc2nnc(C4CC(CCC#N)CC4C)n23)cc1]

To a solution of a scalemic mixture, predominately (1S,2R,4R)-4-(2-cyanoethyl)-2-methyl-N′-(5-tosyl-5H-pyrrolo[2,3-b]pyrazin-2-yl)cyclopentanecarbohydrazide (1.43 g, 3.07 mmol) in 1,4-dioxane (16 mL) was added TEA (1.71 mL, 12.3 mmol) and thionyl chloride (0.27 mL, 3.68 mmol) and the reaction was heated to about 75° C. for about 90 min. The reaction was cooled to ambient temperature and diluted with water (20 mL) and extracted with DCM (3×20 mL). The combined organics were washed with brine (20 mL), dried over anhydrous MgSO₄, filtered, and concentrated under reduced pressure. The crude material was purified by silica gel chromatography eluting with a gradient of 10-70% acetone in heptane to give a scalemic mixture, predominately 3-((1R,3R,4S)-3-methyl-4-(6-tosyl-6H-pyrrolo[2,3-e][1,2,4]triazolo[4,3-a]pyrazin-1-yl)cyclopentyl)propanenitrile (1.23 g, 89%) as a tan foam; LC/MS (Table 1, Method a) Rₜ=2.19 min; MS m/z: 449 (M+H)⁺.

### Step L: 3-((1R,3R,4S)-3-methyl-4-(6H-pyrrolo[2,3-e][1,2,4]triazolo[4,3-a]pyrazin-1-yl)cyclopentyl)propanenitrile

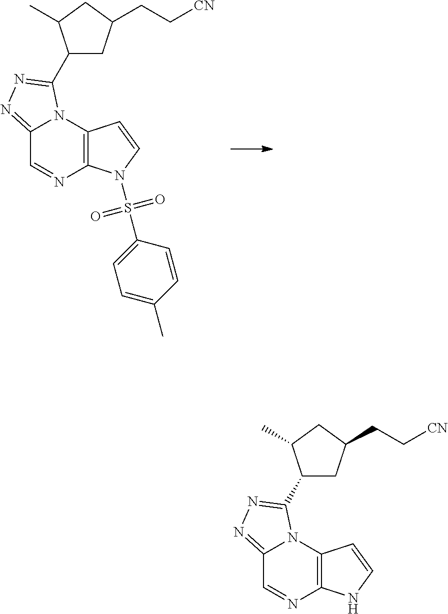 [SMILES: Cc1ccc(S(=O)(=O)n2ccc3c2ncc2nnc(C4CC(CCC#N)CC4C)n23)cc1 ; C[C@@H]1C[C@@H](CCC#N)C[C@@H]1c1nnc2cnc3[nH]ccc3n12]

To a solution of a scalemic mixture, predominately 3-((1R,3R,4S)-3-methyl-4-(6-tosyl-6H-pyrrolo[2,3-e][1,2,4]triazolo[4,3-a]pyrazin-1-yl)cyclopentyl)propanenitrile (1.22 g, 2.73 mmol) in MeOH (11 mL) was added potassium cyanide (0.533 g, 8.19 mmol) and the reaction was left stirring at ambient temperature for about 16 h. The solvent was removed under reduced pressure and EtOAc and water (15 mL each) were added. The layers were separated and the aqueous phase was extracted with EtOAc (2×10 mL) and DCM (2×10 mL). The combined organics were dried over anhydrous Na₂SO₄, filtered, and concentrated under reduced pressure. The crude material was purified by silica gel column chromatography eluting with a gradient of 0-100% ([900/90/10] DCM/MeOH/NH₄OH) in DCM to give a tan foam. The material was further purified by chiral HPLC (Table 2, Method 6) Rₜ=20.4 min, or =negative] to give 3-((1R,3R,4S)-3-methyl-4-(6H-pyrrolo[2,3-e][1,2,4]triazolo[4,3-a]pyrazin-1-yl)cyclopentyl)propanenitrile (0.62 g, 59%) as an off white solid; LC/MS (Table 1, Method a) Rₜ=1.51 min; MS m/z: 295 (M+H)⁺. Jak3 IC₅₀=B

### Example #11

### 3-((1R,3R,4S)-3-methyl-4-(pyrrolo[2,3-b][1,2,3]triazolo[4,5-d]pyridin-1(6H)-yl)cyclopentyl)propanenitrile

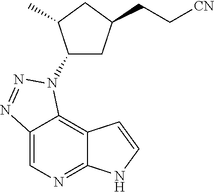 [SMILES: C[C@@H]1C[C@@H](CCC#N)C[C@@H]1n1nnc2cnc3[nH]ccc3c21]

### Step A: 3-(-3-amino-4-methylcyclopentyl)propanenitrile

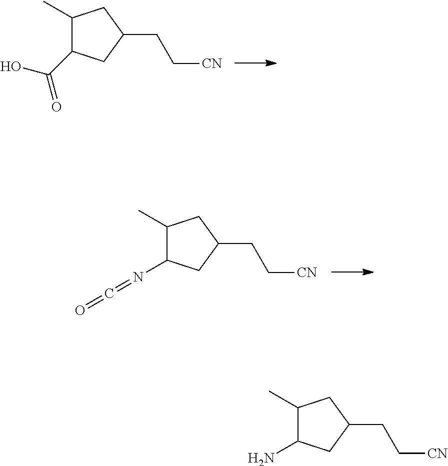

To a solution of a scalemic mixture enriched in (1S,2R,4R)-4-(2-cyanoethyl)-2-methylcyclopentanecarboxylic acid (1.66 g, 9.16 mmol, Example #10, Step I) in toluene (35 mL) was added DPPA (2.18 mL, 10.1 mmol) and TEA (2.81 mL, 20.2 mmol). The reaction was heated at about 100° C. After about 2 h, the reaction was cooled to rt and concentrated under reduced pressure. The resulting oil was dissolved in THF (70 mL) and then water (70 mL) and lithium hydroxide monohydrate (7.69 g, 183 mmol) were added at rt while stirring. After about 16 h, water (150 mL) was added and the mixture was extracted with EtOAc (3×200 mL). Brine (˜50 mL) was added during the second extraction to break an emulsion. The combined organic layers were washed with brine (100 mL), dried over anhydrous Na₂SO₄, filtered, and concentrated under reduced pressure. The crude material was purified by silica gel chromatography eluting with a gradient of 0-100% DCM/MeOH/NH₄OH (900:90:10) in DCM to give a scalemic mixture enriched in 3-((1R,3S,4R)-3-amino-4-methylcyclopentyl)propanenitrile (0.98 g, ˜70%, ˜88% purity by ¹H NMR) as an oil that was used without further purification; LC/MS (Table 1, Method a) Rₜ=1.22 min; MS m/z: 153 (M+H)⁺.

### Step B: 3-(3-methyl-4-(5-nitro-1H-pyrrolo[2,3-b]pyridin-4-ylamino)cyclopentyl)propanenitrile

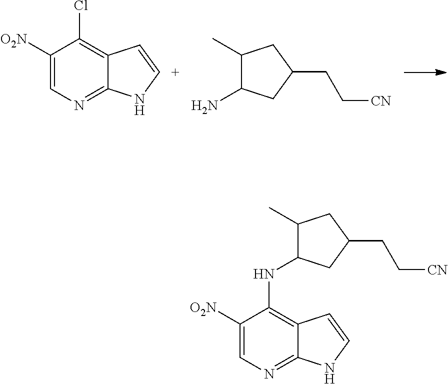 [SMILES: O=[N+]([O-])c1cnc2c(c1Cl)C=[C+]N2 ; CC1CC(CCC#N)CC1N ; CC1CC(CCC#N)CC1Nc1c([N+](=O)[O-])cnc2[nH]ccc12]

A mixture of 4-chloro-5-nitro-1H-pyrrolo[2,3-b]pyridine (1.20 g, 6.07 mmol, WO2011/068881), a scalemic mixture enriched in 3-((1R,3S,4R)-3-amino-4-methylcyclopentyl)propanenitrile (0.97 g, 6.37 mmol), and DIEA (2.12 mL, 12.2 mmol) in N-methylpyrrolidone (30 mL) was stirred at about 60° C. Mier about 21 h, the reaction was cooled to rt and poured slowly into a stirring ice-water mixture (˜150 mL). The resulting yellow solid was collected via vacuum filtration, while washing with additional water (100 mL), and dried in a vacuum oven at about 50° C. to give a scalemic mixture enriched, in 3-((1R,3R,4S)-3-methyl-4-(5-nitro-1H-pyrrolo[2,3-b]pyridin-4-ylamino)cyclopentyl)-propanenitrile (1.79 g, ˜94%, ˜87% purity by UV) as a yellow solid that was used without further purification; LC/MS (Table 1, Method a) Rₜ=2.01 min; MS m/z: 314 (M+H)⁺.

### Step C: 3-(3-(5-amino-1H-pyrrolo[2,3-b]pyridin-4-ylamino)-4-methylcyclopentyl)propanenitrile

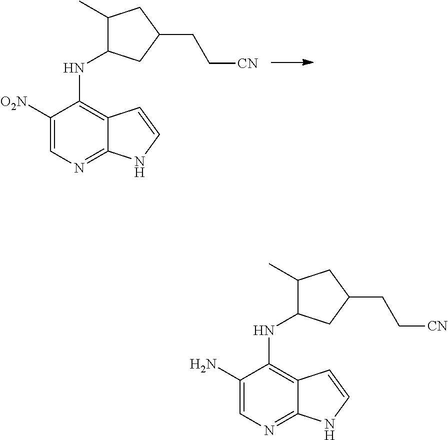 [SMILES: CC1CC(CCC#N)CC1Nc1c([N+](=O)[O-])cnc2[nH]ccc12 ; CC1CC(CCC#N)CC1Nc1c(N)cnc2[nH]ccc12]

To a scalemic mixture enriched in 3-((1R,3R,4S)-3-methyl-4-(5-nitro-1H-pyrrolo[2,3-b]pyridin-4-ylamino)cyclo-pentyl)propanenitrile (1.57 g, 5.01 mmol) in EtOH (50 mL) was added tin(II) chloride dihydrate (5.65 g, 25.1 mmol). The reaction was heated to about 70° C. After about 1.5 h, the reaction was cooled to rt., poured over ice water (˜150 mL), and then the pH was adjusted to ˜8 with saturated aqueous NaHCO₃. EtOAc (150 mL) was added and the resulting mixture was stirred at rt for about 1 h and then filtered to remove tin salts. The layers of the filtrate were separated. The filter cake was stirred with EtOAc (2×100 mL) and then filtered. Each organic filtrate was then used to extract the initial aqueous layer. The combined organic layers were washed with brine (100 mL), dried over Na₂SO₄, decanted, and concentrated. The crude material was dissolved in DCM and purified by silica gel chromatography eluting with a gradient of 0-75% DCM/MeOH/NH₄OH (900:90:10) in DCM to give a scalemic mixture enriched in 3-((1R,3S,4R)-3-(5-amino-1H-pyrrolo[2,3-b]pyridin-4-ylamino)-4-methylcyclopentyl)propane-nitrile with about 3 mol % DCM as an excipient (1.01 g, 70%) as a brown tacky foam; LC/MS (Table 1, Method a) Rₜ=1.62 min; MS m/z: 284 (M+H)⁺.

### Step D: 3-((1R,3R,4S)-3-methyl-4-(pyrrolo[2,3-b][1,2,3]triazolo[4,5-d]pyridin-1(6H)-yl)cyclopentyl)propanenitrile

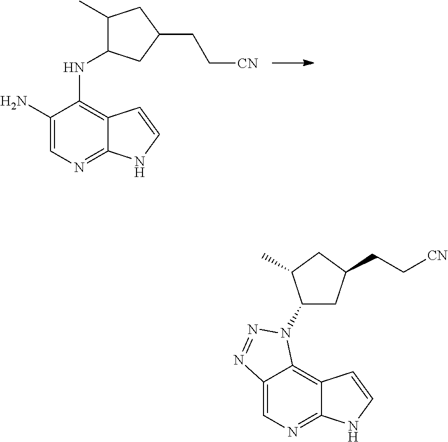 [SMILES: CC1CC(CCC#N)CC1Nc1c(N)cnc2[nH]ccc12 ; C[C@@H]1C[C@@H](CCC#N)C[C@@H]1n1nnc2cnc3[nH]ccc3c21]

A scalemic mixture enriched in 3-((1R,3S,4R)-3-(5-amino-1H-pyrrolo[2,3-b]pyridin-4-ylamino)-4-methylcyclo-pentyl)propanenitrile (1.01 g, 3.56 mmol) in THF (15.0 mL) and 6 N HCl (15 mL, 90 mmol) was cooled to about 0° C. A solution of NaNO₂ (0.369 g, 5.35 mmol) in water (3 mL) was added and the reaction continued stirring at about 0° C. After about 40 min, the reaction was quenched with 2 N aqueous NaOH (˜45 mL) while cooled in an ice bath. The mixture was extracted with EtOAc (3×100 mL). The combined organic layers were washed with brine (100 mL), dried, over anhydrous Na₂SO₄, filtered, and concentrated under reduced pressure. The crude material was purified by silica gel chromatography eluting with a gradient of 0-75% DCM/MeOH/NH₄OH (900:90:10) in DCM to give an off-white solid (0.83 g) which was further purified along with additional material (0.033 g) from a separate reaction via chiral preparatory HPLC (Table 2, Method 9); Rₜ=18.1 min, or =negative to give 3-((1R,3R,4S)-3-methyl-4-(pyrrolo[2,3-b][1,2,3]triazolo[4,5-d]pyridin-1(6H)-yl)cyclopentyl)-propanenitrile with ˜1 mol % DCM as an excipient (0.78 g, 71%) as a white solid; LC/MS (Table 1, Method a) Rₜ=1.76 min; MS m/z: 295 (M+H)⁺. Further drying at about 60° C. gave material with less than 0.5 mol % DCM. Jak3 IC₅₀=B

### Example #12

### 5-((3S,4R)-3-ethyl-4-3H-imidazo[1,2-a]pyrrolo[2,3-e]pyrazin-8-yl)pyrrolidin-1-yl)-5-oxopentanenitrile

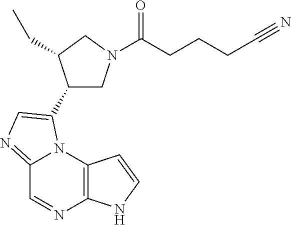 [SMILES: CC[C@@H]1CN(C(=O)CCCC#N)C[C@@H]1c1cnc2cnc3[nH]ccc3n12]

### Step A: (Z)-ethyl pent-2-enoate

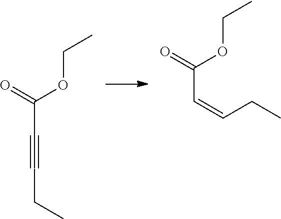

To a slurry of Lindlar catalyst (0.844 g, 0.396 mmol) THF (100 mL) and pyridine (10 mL) was added ethyl pent-2-ynoate (5.22 mL, 39.6 mmol). The reaction mixture was sparged with hydrogen for about 10 min and an atmosphere of hydrogen was maintained via balloon. After about 15 h the reaction mixture was filtered through a pad of Celite®, diluted with Et₂O (30 mL) and washed with saturated aqueous CuSO₄ (40 mL), followed by water (40 mL). The organic layer was separated, dried over anhydrous MgSO₄, filtered, and concentrated in vacuo to provide crude (Z)-ethyl pent-2-enoate (5 g, 98%). ¹H NMR (DMSO-d₆) δ 6.21 (m, 1H), 5.72 (m, 1H), 4.18 (q, 2H), 2.65 (m, 2H), 1.28 (t, 3H), 1.05 (t, 3H).

### Step B: (cis)-ethyl 1-benzyl-4-ethylpyrrolidine-3-carboxylate

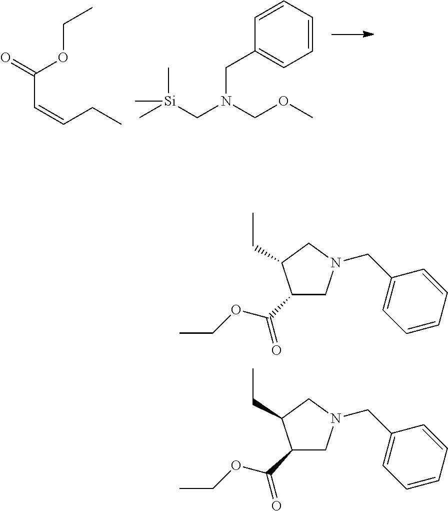

To a solution of N-benzyl-1-methoxy-N-((trimethylsilyl)methyl)methanamine (9.98 mL, 39.0 mmol) and (Z)-ethyl pent-2-enoate (5.0 g, 39.0 mmol) in DCM (50 mL) was added TFA (0.030 mL, 0.390 mmol) at rt. After about 2 days, the reaction mixture was concentrated in vacuo to provide crude cis-ethyl 1-benzyl-4-ethylpyrrolidine-3-carboxylate (9.8 g, 96%) as an oil. LC/MS (Table 1, Method a) Rₜ=1.62 min; MS m/z: 262 (M+H)⁺.

### Step C: (cis)-1-(benzyloxycarbonyl)-4-ethylpyrrolidine-3-carboxylic acid

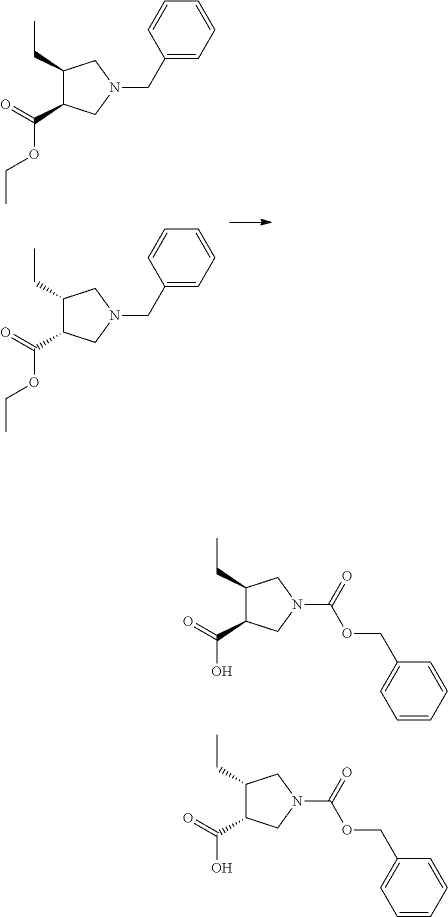 [SMILES: CCOC(=O)[C@@H]1CN(Cc2ccccc2)C[C@@H]1CC ; CCOC(=O)[C@H]1CN(Cc2ccccc2)C[C@H]1CC ; CC[C@H]1CN(C(=O)OCc2ccccc2)C[C@H]1C(=O)O ; CC[C@@H]1CN(C(=O)OCc2ccccc2)C[C@@H]1C(=O)O]

To a slurry of 20% by weight palladium hydroxide on carbon (5.37 g, 7.65 mmol) in EtOH (300 mL) was added cis-ethyl 1-benzyl-4-ethylpyrrolidine-3-carboxylate (40 g, 153 mmol). The reaction mixture was placed on a Parr shaker and hydrogenated at about 60 psi. After about 2 days the reaction mixture was filtered through Celite® and concentrated in vacuo to provide crude cis-ethyl 4-ethylpyrrolidine-3-carboxylate as an oil. To a flask charged with the crude cis-ethyl 4-ethylpyrrolidine-3-carboxylate (18 g, 105 mmol) was added a 6N aqueous HCl (200 mL). The reaction mixture was heated to about 100° C. After about 24 h the reaction mixture was cooled to rt and concentrated in vacuo. The residue was dissolved in 1,4-dioxane (200 mL) and water (200 mL). The pH of the reaction mixture was adjusted to about 10 with Na₂CO₃ and then benzyl 2,5-dioxopyrrolidin-1-yl carbonate (28.8 g, 116 mmol) was added. After about 15 h, Et₂O (200 mL) was added and the layers were separated. The pH of the aqueous layer was adjusted to 1 with concentrated aqueous HCl and Et₂O/EtOAc (1:1, 500 mL) was added. The organic layer was separated, dried over Na₂SO₄, filtered and concentrated in vacuo to provide cis-1-(benzyloxycarbonyl)-4-ethylpyrrolidine-3-carboxylic acid (21.8 g, 75%) as a brown oil. LC/MS (Table 1, Method a) Rₜ=1.80 min; MS m/z: 278 (M+H)⁺.

### Step D: (R)-1-(naphthalen-1-yl)ethanammonium (3R,4S)-1-(benzyloxycarbonyl)-4-ethylpyrrolidine-3-carboxylate

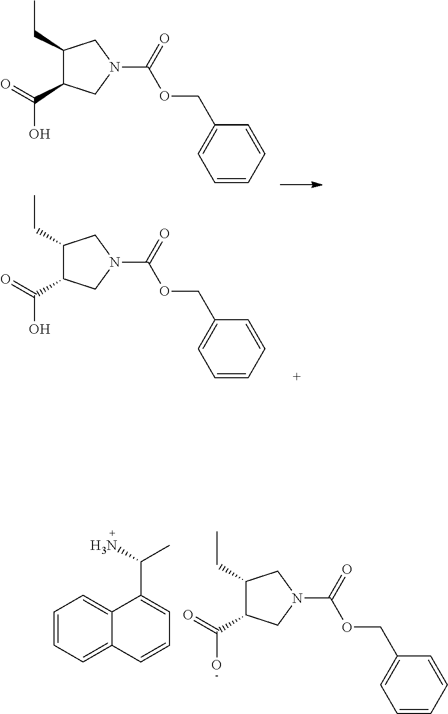

To a solution of cis-1-(benzyloxycarbonyl)-4-ethylpyrrolidine-3-carboxylic acid (375 g, 1149 mmol) in acetonitrile (2 L) was added (S)-1-(naphthalen-1-yl)ethanamine (68.9 g, 402 mmol) with stirring. After 15 h, the solids were collected and dried in vacuo to provide (S)-1-(naphthalen-1-yl)ethanammonium (3S,4R)-1-(benzyloxycarbonyl)-4-ethylpyrrolidine-3-carboxylate (64.85 g, 145 mmol, 13%) (er≧98:2, Rₜ=5.31 min, Table 1, Method g). The filtrate was concentrated in vacuo, dissolved in Et₂O (2 L) and washed with 1N aqueous HCl (2 L) and brine (1 L). The organic layer was dried (MgSO₄), filtered and concentrated in vacuo. The oil was dissolved in acetonitrile (2 L) and (R)-1-(naphthalen-1-yl)ethanamine (98 g, 575 mmol) was added with stirring. After about 15 h the solids were collected by filtration to provide a mixture of (R)-1-(naphthalen-1-yl)ethanamine (3R,4S)-1-(benzyloxycarbonyl)-4-ethylpyrrolidine-3-carboxylate and (R)-1-(naphthalen-1-yl)ethanammonium (3S,4R)-1-(benzyloxycarbonyl)-4-ethylpyrrolidine-3-carboxylate. Rₜ=4.77 min and 5.31 min, Table 1, Method g (er=85:15). The wet solids (350 g) were slurried in acetonitrile (3.5 L) and heated to about 80° C. with stirring. After about 4 h the mixture was cooled to about 40° C., filtered and dried in vacuo to provide (R)-1-(naphthalen-1-yl)ethanaminium (3R,4S)-1-(benzyloxycarbonyl)-4-ethylpyrrolidine-3-carboxylate (167 g, 32%). (er≧93:2, Rₜ=4.77 min, Table 1, Method g). LC/MS (Table 1, Method a) Rₜ=1.80 min; MS m/z: 278 (M+H)⁺.

### Step E: (3R,4S)-benzyl 3-(2-bromoacetyl)-4-ethylpyrrolidine-1-carboxylate

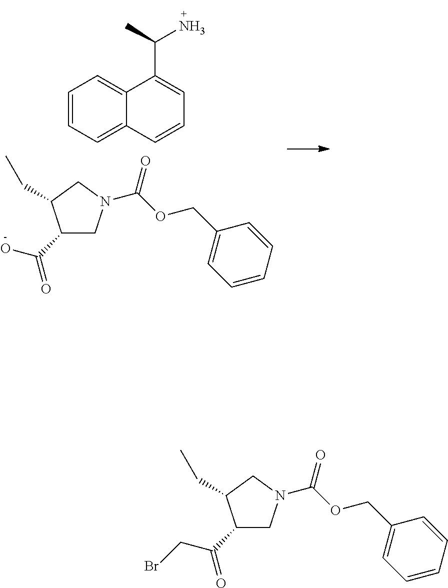

(R)-1-(naphthalen-1-yl)ethanammonium (3R,4S)-1-(benzyloxycarbonyl)-4-ethylpyrrolidine-3-carboxylate (25 g, 55.7 mmol) was added to a separatory funnel containing Et₂O (500 mL) and 1N aqueous HCl (350 mL). The mixture was shaken until all of the solids dissolved. The layers were separated and the organic layer was washed with brine (200 mL), dried (MgSO₄) filtered and concentrated in vacuo. The resulting (3R,4S)-1-(benzyloxycarbonyl)-4-ethylpyrrolidine-3-carboxylic acid was dissolved in DCM (200 mL) and DMF (0.043 mL, 0.557 mmol) and oxalyl chloride (2M in DCM, 55.7 mL, 111 mmol) was added. After bubbling ceased (about 4 h), the reaction mixture was allowed to stir for an additional about 2 h prior to being concentrated in vacuo. The crude acid chloride was dissolved THF (100 mL) and acetonitrile (100 mL), cooled to about 0 to −10° C. and TMS-diazomethane (1M in Et₂O, 195 mL, 195 mmol) was added while maintaining the temperature below about 10° C. After about 2 h, 48% aqueous HBr (63.1 mL, 557 mmol) was added such that the internal temperature was no more than about 10° C. After about 4 h, the reaction mixture was diluted with Et₂O (300 mL) and water (300 mL). The organic layer was separated, washed with saturated aqueous NaHCO₃ (300 mL) and brine (200 mL), dried over MgSO₄, filtered and concentrated in vacuo to provide (3R,4S)-benzyl 3-(2-bromoacetyl)-4-ethylpyrrolidine-1-carboxylate (19.5 g, 99%). The crude bromide was used without further purification. LC/MS (Table 1, Method a) Rₜ=2.37 min; MS m/z: 354, 356 (M+H)⁺.

### Step F: (3R,4S)-benzyl 3-(2-(tert-butoxycarbonyl(5-tosyl-5H-pyrrolo[2,3-b]pyrazin-2-yl)amino)acetyl)-4-ethylpyrrolidine-1-carboxylate

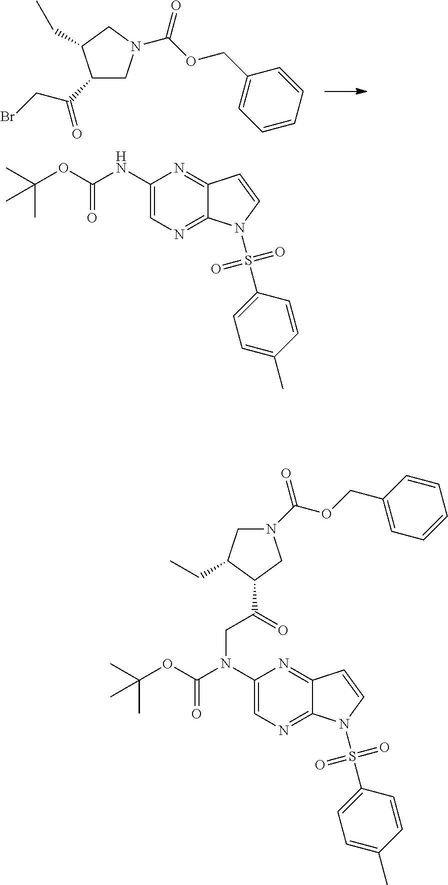

To a solution of tert-butyl 5-tosyl-5H-pyrrolo[2,3-b]pyrazin-2-ylcarbamate (28.1 g, 72.3 mmol, WO2011/068881) in DMF (100 mL) was added NaH (2.67 g, 66.7 mmol, 60% suspension in mineral oil). After about 4 h, the solution of anion was added slowly to a solution of (3R,4S)-benzyl 3-(2-bromoacetyl)-4-ethylpyrrolidine-1-carboxylate (19.7 g, 55.6 mmol) in THF (100 mL) at about 0° C. Additional DMF (30 mL) was added to aid in the addition. After about 1 h following the addition saturated aqueous NH₄Cl (200 mL) and EtOAc (500 mL) were added. The organic layer was separated, washed with brine (200 mL), concentrated in vacuo and purified by chromatography on silica gel (330 g) eluting with EtOAc/heptanes (20-80%) to provide (3R,4S)-benzyl 3-(2-(tert-butoxycarbonyl(5-tosyl-5H-pyrrolo[2,3-b]pyrazin-2-yl)amino)acetyl)-4-ethylpyrrolidine-1-carboxylate (22 g, 60%) as a solid. LC/MS (Table 1, Method a) Rₜ=2.98 min; MS m/z: 562 (M+H)⁺.

### Step G: 8-((3R,4S)-4-ethylpyrrolidin-3-yl)-3-tosyl-3H-imidazo[1,2-a]pyrrolo[2,3-e]pyrazine bis hydrobromide

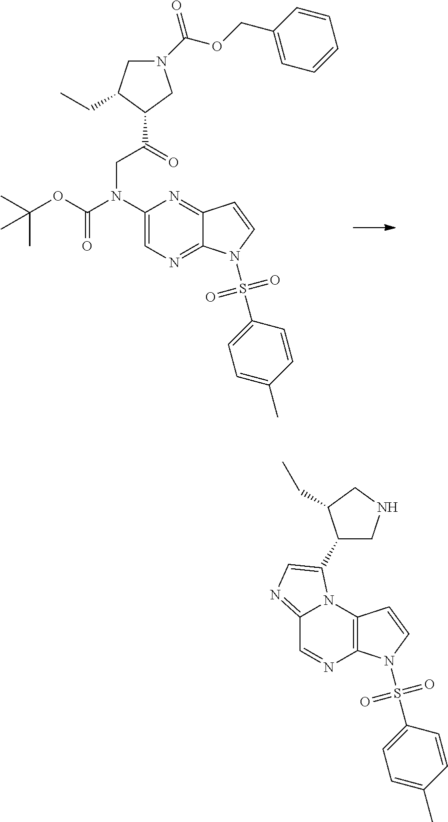

To a solution of (3R,4S)-benzyl 3-(2-(tert-butoxycarbonyl(5-tosyl-5H-pyrrolo[2,3-b]pyrazin-2-yl)amino)acetyl)-4-ethylpyrrolidine-1-carboxylate (40 g, 60.4 mmol) in DCM (500 mL) at rt was added TFA (46.6 mL, 604 mmol). After about 8 h the reaction mixture was concentrated in vacuo. The residue was dissolved in 1,4-dioxane (1 L) and the solution was degassed with nitrogen. After about 30 min, Lawesson's reagent (18.34 g, 45.3 mmol) was added and the reaction mixture was heated to about 65° C. After about 4 h, the reaction mixture was cooled to rt and water (200 mL) followed by sodium perborate monohydrate (30.2 g, 302 mmol) was added. After about 15 h, saturated aqueous NaHCO₃ (200 mL) was added. After about 2 h the reaction mixture was extracted with EtOAc (3×500 mL). The organic extracts were combined, washed with brine (300 mL), dried (Na₂SO₄), filtered and concentrated in vacuo. The residue was dissolved in 1,4-dioxane (1 L) and a 33% solution of HBr in acetic acid (99 mL, 604 mmol) was added slowly. After about 4 h, Et₂O (500 mL) was added and the solids were collected by filtration and dried in vacuo to provide 8-((3R,4S)-4-ethylpyrrolidin-3-yl)-3-tosyl-3H-imidazo[1,2-a]pyrrolo[2,3-e]pyrazine bis hydrobromide (32 g, 93%) as an orange solid. LC/MS (Table 1, Method a) Rₜ=1.51 min; MS m/z: 410 (M+H)⁺.

### Step H: 5-((3S,4R)-3-ethyl-4-(3-tosyl-3H-imidazo[1,2-a]pyrrolo[2,3-e]pyrazin-8-yl)pyrrolidin-1-yl)-5-oxopentanenitrile

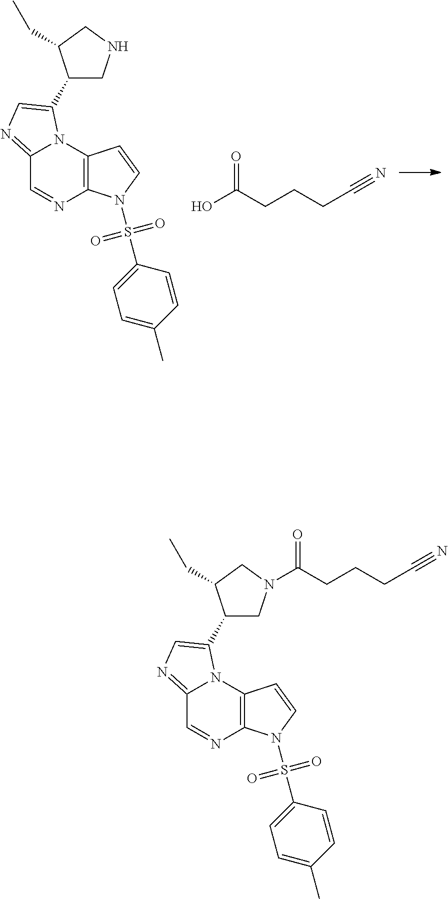 [SMILES: CC[C@@H]1CNC[C@@H]1c1cnc2cnc3c(ccn3S(=O)(=O)c3ccc(C)cc3)n12 ; N#CCCCC(=O)O ; CC[C@@H]1CN(C(=O)CCCC#N)C[C@@H]1c1cnc2cnc3c(ccn3S(=O)(=O)c3ccc(C)cc3)n12]

To a solution of 8-((3R,4S)-4-ethylpyrrolidin-3-yl)-3-tosyl-3H-imidazo[1,2-a]pyrrolo[2,3-e]pyrazine (5 g, 12.2 mmol) in DCM (20 mL) was added DIEA (2.1 mL, 12.2 mmol) and stirred at ambient temperature for about 3 min followed by the addition of 4-cyanobutanoic acid (1.45 g, 12.8 mmol, Best PharmaTech, Inc.) and HATU (4.64 g, 12.2 mmol). The mixture was stirred for about 18 h and to it was added saturated aqueous NaHCO₃ (10 mL) and the layers were separated. The aqueous layer was back extracted with DCM (15 mL). The combined organic layers were washed with brine (10 mL), dried over anhydrous MgSO₄, filtered and concentrated under reduced pressure. The crude material was purified by silica gel chromatography elating with a gradient of 0-5% MeOH/DCM to afford 5-((3S,4R)-3-ethyl-4-(3-tosyl-3H-imidazo[1,2-a]pyrrolo[2,3-e]pyrazin-8-yl)pyrrolidin-1-yl)-5-oxopentanenitrile as a yellow foam (5.59 g, 91): LC/MS (Table 1, Method b) Rₜ=2.00 min; MS m/z: 505 (M+H)⁺.

### Step I: 5-((3S,4R)-3-ethyl-4-(3H-imidazo[1,2-a]pyrrolo[2,3-e]pyrazin-8-yl)pyrrolidin-1-yl)-5-oxopentanenitrile

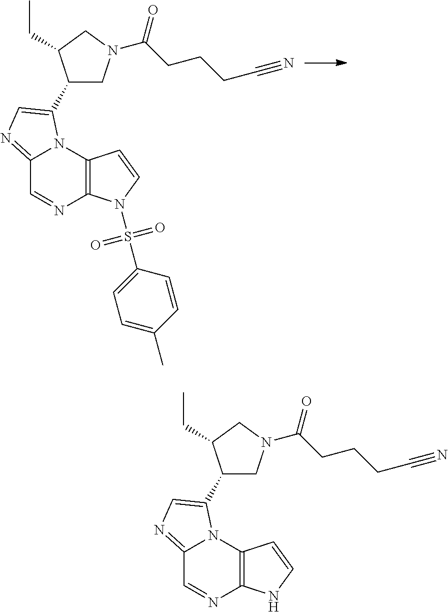

To a solution of 5-((3S,4R)-3-ethyl-4-(3-tosyl-3H-imidazo[1,2-a]pyrrolo[2,3-e]pyrazin-8-yl)pyrrolidin-1-yl)-5-oxopentanenitrile (5.59 g, 11.1 mmol) in 1,4-dioxane (22 mL) was added 2N aqueous NaOH (11.1 mL, 22.2 mmol). The resulting mixture was stirred and heated to about 40° C. for about 90 min. The reaction was cooled to ambient temperature and to it was added DCM (20 mL) and H₂O (20 mL). The organic layer was separated and washed with brine (20 mL), dried over anhydrous MgSO₄, filtered and concentrated under reduced pressure. The material was purified by silica gel chromatography eluting with a gradient of 0-10% MeOH/DCM to afford a yellow foam. The compound was further purified by chiral chromatography (Table 1, Method 10). Rₜ=9.1 min, or =negative The product was dried in a high vacuum oven at about 60° C. for about 2 days to afford 5-((3S,4R)-3-ethyl-4-(3H-imidazo[1,2-a]pyrrolo[2,3-e]pyrazin-8-yl)pyrrolidin-1-yl)-5-oxopentanenitrile (2.65 g, 68%): LC/MS (Table 1, Method b) Rₜ=1.41 min; MS m/z: 351 (M+H)⁺. Jak3 IC₅₀=B

### Example #13

### (3S,4R)-3-ethyl-N-(2-fluoroethyl)-4-(3H-imidazo[1,2-a]pyrrolo[2,3-e]pyrazin-8-yl)pyrrolidine-1-carboxamide

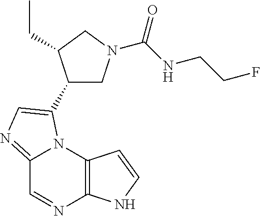 [SMILES: CC[C@@H]1CN(C(=O)NCCF)C[C@@H]1c1cnc2cnc3[nH]ccc3n12]

### Step A: (3S,4R)-3-ethyl-N-(2-fluoroethyl)-4-(3-tosyl-3H-imidazo[1,2-a]pyrrolo[2,3-e]pyrazin-8-yl)pyrrolidine-1-carboxamide

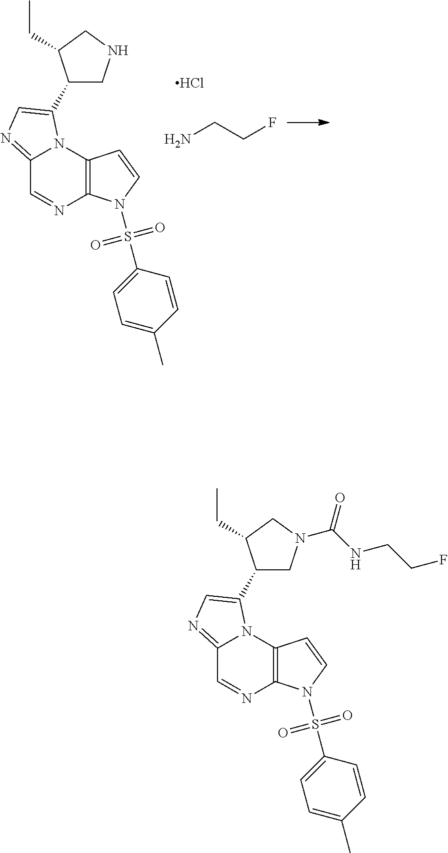

To a solution of 2-fluoroethanamine hydrochloric acid (2.11 g, 21.3 mmol) in acetonitrile (31.0 mL) at ambient temperature was added CDI (3.24 g, 19.9 mmol) and stirred for about 40 min followed, by the addition of TEA (1.86 mL, 13.3 mmol). The resulting mixture stirred for about 20 mm and to it was added a solution of 8-((3R,4S)-4-ethylpyrrolidin-3-yl)-3-tosyl-3H-imidazo[1,2-a]pyrrolo[2,3-e]pyrazine (5.45 g, 13.3 mmol, Example #12, Step G) and TEA (3.71 mL, 26.6 mmol) in acetonitrile (62.0 mL) and stirring was continued for about 1 h. An additional solution of 2-fluoroethanamine hydrochloric acid (0.93 g, 9.31 mmol) in acetonitrile (10.0 mL) that had stirred for about 1 h was added to the reaction mixture and stirring was continued for an additional 1 h. The reaction mixture was partially concentrated under reduced pressure and to it was added DCM (100 mL) and saturated aqueous NaHCO₃ (80 mL). The layers were separated and the aqueous layer was back extracted with DCM (2×80 mL). The combined organic layers were dried over anhydrous MgSO₄, filtered and concentrated under reduced pressure. The crude material was purified by silica gel chromatography eluting with a gradient of 0-7% MeOH/DCM to afford (3S,4R)-3-ethyl-N-(2-fluoroethyl)-4-(3-tosyl-3H-imidazo[1,2-a]pyrrolo[2,3-e]pyrazin-8-yl)pyrrolidine-1-carboxamide (5.44 g, 82%) as an off white solid: LC/MS (Table 1, Method b) Rₜ=1.96 min; MS m/z: 499 (M+H)⁺.

### Step B: (3S,4R)-3-ethyl-N-(2-fluoroethyl)-4-(3H-imidazo[1,2-a]pyrrolo[2,3-e]pyrazin-8-yl)pyrrolidine-1-carboxamide

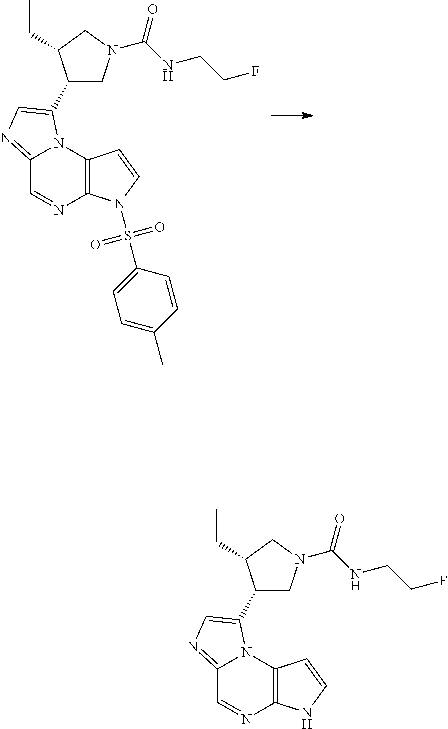 [SMILES: CC[C@@H]1CN(C(=O)NCCF)C[C@@H]1c1cnc2cnc3c(ccn3S(=O)(=O)c3ccc(C)cc3)n12 ; CC[C@@H]1CN(C(=O)NCCF)C[C@@H]1c1cnc2cnc3[nH]ccc3n12]

To a solution of (3S,4R)-3-ethyl-N-(2-fluoroethyl)-4-(3-tosyl-3H-imidazo[1,2-a]pyrrolo[2,3-e]pyrazin-8-yl)pyrrolidine-1-carboxamide (5.44 g, 10.9 mmol) in 1,4-dioxane (60.7 mL) was added 1N aqueous NaOH (21.9 mL, 21.9 mmol) and the reaction mixture was stirred at about 65° C. for about 1 h. The reaction mixture was cooled to ambient temperature and partially concentrated under reduced pressure. The aqueous solution was extracted with DCM (3×30 mL) and DCM/IPA (9:1, 2×70 mL). The combined organic layers were dried over anhydrous MgSO₄, filtered and concentrated under reduced pressure. The crude material was purified by silica gel chromatography eluting with a gradient of 0-10% MeOH/DCM to afford a solid that was further purified, by chiral chromatography (Table 1, Method, 2), Rₜ=11.3 min, or =negative The product was lyophilized to afford (3S,4R)-3-ethyl-N-(2-fluoroethyl)-4-(3H-imidazo[1,2-a]pyrrolo[2,3-e]yrazin-8-yl)pyrrolidine-1-carboxamide (2.2 g, 58.4%). LC/MS (Table 1, Method b) Rₜ=1.34 min; MS m/z 345 (M+H)⁺. Jak3 IC₅₀=B

### Example #14

### (3S,4R)—N-(2,2-difluoroethyl)-3-ethyl-4-(3H-imidazo[1,2-a]pyrrolo[2,3-e]pyrazin-8-yl)pyrrolidine-1-carboxamide

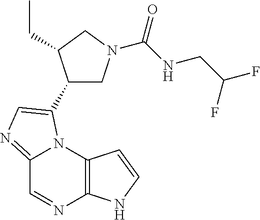 [SMILES: CC[C@@H]1CN(C(=O)NCC(F)F)C[C@@H]1c1cnc2cnc3[nH]ccc3n12]

### Step A: (3S,4R)—N-(2,2-difluoroethyl)-3-ethyl-4-(3-tosyl-3H-imidazo[1,2-a]pyrrolo[2,3-e]pyrazin-8-yl)pyrrolidine-1-carboxamide

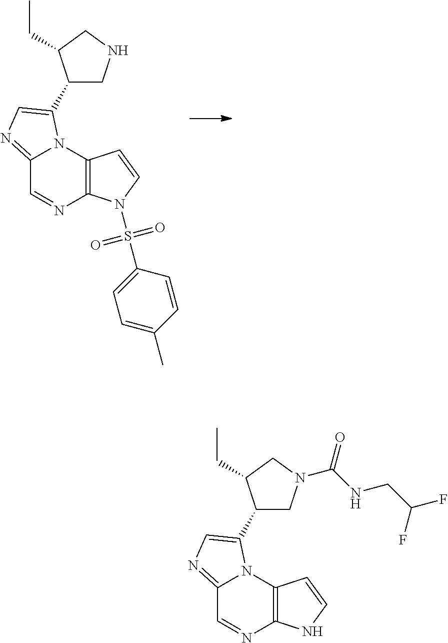 [SMILES: CC[C@@H]1CNC[C@@H]1c1cnc2cnc3c(ccn3S(=O)(=O)c3ccc(C)cc3)n12 ; CC[C@@H]1CN(C(=O)NCC(F)F)C[C@@H]1c1cnc2cnc3[nH]ccc3n12]

To a solution of 2,2-difluoroethylamine (0.11 g, 1.31 mmol) in DMF (0.6 mL) was added. CDI (0.21 g, 1.23 mmol) and the reaction mixture was heated to about 65° C. for about 16 h. The solution was then added to a solution of 8-((3R,4S)-4-ethylpyrrolidin-3-yl)-3-tosyl-3H-imidazo[1,2-a]pyrrolo[2,3-e]pyrazine (0.10 g, 0.24 mmol, Example #12, Step G) in DMF (0.9 mL) and heated to about 65° C. for about an additional 90 min. The reaction mixture was cooled to ambient temperature and concentrated under reduced pressure. To the crude residue was added EtOAc (75 mL) and H₂O (25 mL). The organic layer was separated and washed with H₂O (25 mL), brine (2×2.5 mL), dried over MgSO₄, filtered and concentrated under reduced pressure to afford crude (3S,4R)—N-(2,2-difluoroethyl)-3-ethyl-4-(3-tosyl-3H-imidazo[1,2-a]pyrrolo[2,3-e]pyrazin-8-yl)pyrrolidine-1-carboxamide.

To a solution of crude (3S,4R)—N-(2,2-difluoroethyl)-3-ethyl-4-(3-tosyl-3H-imidazo[1,2-a]pyrrolo[2,3-e]pyrazin-8-yl)pyrrolidine-1-carboxamide (0.13 g, 0.24 mmol) in 1,4-dioxane (1.8 mL) was added 1N aqueous NaOH (0.5 mL, 0.5 mmol) and the mixture was stirred at about 65° C. for about 2 h. The reaction was cooled to ambient temperature and to it was added DCM (20 mL) and saturated aqueous NaHCO₃ (20 mL). The layers were separated and the aqueous layer was back extracted with DCM (10 mL). The combined organic layers were dried over anhydrous MgSO₄, filtered and concentrated under reduced pressure. The crude material was purified by silica gel chromatography eluting with a gradient of 0-5% MeOH/DCM and dried in a high vacuum oven at about 60° C. for about 16 h to afford (3S,4R)—N-(2,2-difluoroethyl)-3-ethyl-4-(3-tosyl-3H-imidazo[1,2-a]pyrrolo[2,3-e]pyrazin-8-yl)pyrrolidine-1-carboxamide (0.07 g, 76%) as an off white solid: LC/MS (Table 1, Method b) Rₜ=1.39 min; MS m/z: 363 (M+H)⁺. Jak3 IC₅₀=B

### Example #15

### 1-((3S,4R)-3-ethyl-4-(3H-imidazo[1,2-a]pyrrolo[2,3-e]pyrazin-8-yl)pyrrolidin-1-yl)-4,4,4-trifluorobutan-1-one

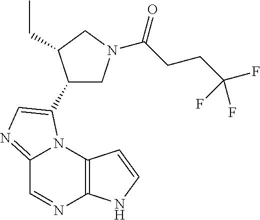 [SMILES: CC[C@@H]1CN(C(=O)CCC(F)(F)F)C[C@@H]1c1cnc2cnc3[nH]ccc3n12]

### Step A: 1-((3S,4R)-3-ethyl-4-(3-tosyl-3H-imidazo[1,2-a]pyrrolo[2,3-e]pyrazin-8-yl)pyrrolidin-1-yl)-4,4,4-trifluorobutan-1-one

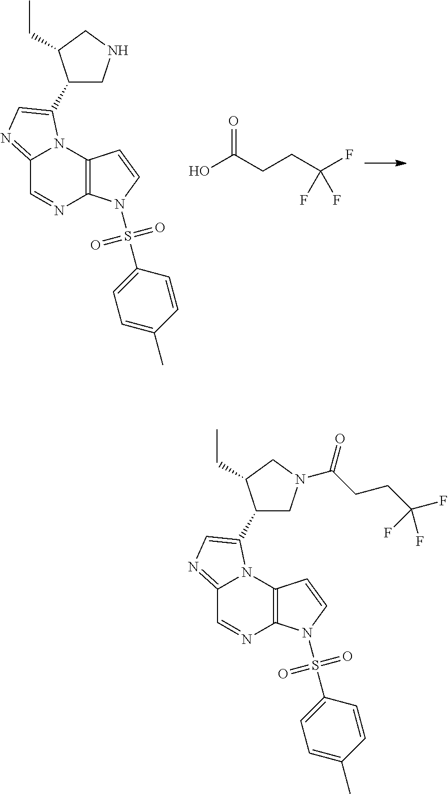 [SMILES: CC[C@@H]1CNC[C@@H]1c1cnc2cnc3c(ccn3S(=O)(=O)c3ccc(C)cc3)n12 ; O=C(O)CCC(F)(F)F ; CC[C@@H]1CN(C(=O)CCC(F)(F)F)C[C@@H]1c1cnc2cnc3c(ccn3S(=O)(=O)c3ccc(C)cc3)n12]

To a solution of 8-((3R,4S)-4-ethylpyrrolidin-3-yl)-3-tosyl-3H-imidazo[1,2-a]pyrrolo[2,3-e]pyrazine (0.10 g, 0.24 mmol, Example #12, Step G) in DCM (2 mL) was added DIEA (0.09 mL, 0.51 mmol), 4,4,4-trifluorobutanoic acid (0.04 g, 0.24 mmol, Matrix) and HATU (0.12 g, 0.32 mmol). The mixture was stirred, for about 1 h and to it was added DCM (10 mL) and saturated aqueous NaHCO₃ (5 mL) and the layers were separated. The aqueous layer was back extracted with DCM (10 mL). The combined organic layers were dried over anhydrous MgSO₄, filtered and concentrated under reduced pressure to afford crude 1-((3S,4R)-3-ethyl-4-(3-toxyl-3H-imidazo[1,2-a]pyrrolo[2,3-e]pyrazin-8-yl)pyrrolidin-1-yl)-4,4,4-trifluorobutan-1-one (0.13 g, 100% crude): LC/MS (Table 1, Method b) Rₜ=0.73 min; MS m/z: 534 (M+H)⁺.

### Step B: 1-((3S,4R)-3-ethyl-4-(3H-imidazo[1,2-a]pyrrolo[2,3-e]pyrazin-8-yl)pyrrolidin-1-yl)-4,4,4-trifluorobutan-1-one

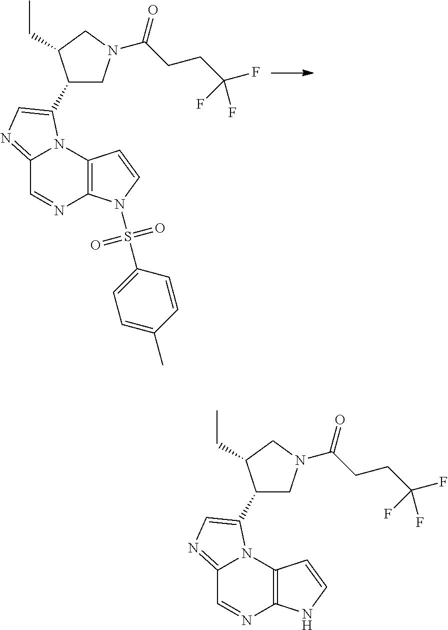 [SMILES: CC[C@@H]1CN(C(=O)CCC(F)(F)F)C[C@@H]1c1cnc2cnc3c(ccn3S(=O)(=O)c3ccc(C)cc3)n12 ; CC[C@@H]1CN(C(=O)CCC(F)(F)F)C[C@@H]1c1cnc2cnc3[nH]ccc3n12]

To a solution of crude 1-((3S,4R)-3-ethyl-4-(3-tosyl-3H-imidazo[1,2-a]pyrrolo[2,3-e]pyrazin-8-yl)pyrrolidin-1-yl)-4,4,4-trifluorobutan-1-one (0.13 g, 0.24 mmol) in 1,4-dioxane (1.8 mL) was added 1N aqueous NaOH (0.5 mL, 0.5 mmol) and the reaction mixture was stirred at about 70° C. for about 30 min. The reaction was cooled to ambient temperature and to it was added DCM (20 mL) and saturated NaHCO₃ (10 mL). The layers were separated and the aqueous layer was back extracted with DCM (10 mL). The combined organic layers were dried over anhydrous MgSO₄, filtered and concentrated under reduced pressure. The crude material was purified by silica gel chromatography eluting with a gradient of 0-5% MeOH/DCM and dried in a vacuum oven at about 60° C. for about 16 h to afford 1-((3S,4R)-3-ethyl-4-(3H-imidazo[1,2-a]pyrrolo[2,3-e]pyrazin-8-yl)pyrrolidin-1-yl)-4,4,4-trifluorobutan-1-one (0.06 g, 62%) as an off white solid: LC/MS (Table 1, Method b) Rₜ=1.66 min; MS m/z: 380 (M+H)⁺. Jak3 IC₅₀=B

### Example #16

### (3S,4R)—N-(2,2-difluoropropyl)-3-ethyl-4-(3H-imidazo[1,2-a]pyrrolo[2,3-e]pyrazin-8-yl)pyrrolidine-1-carboxamide

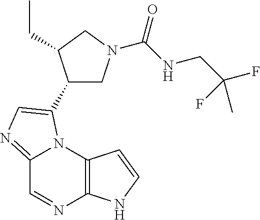 [SMILES: CC[C@@H]1CN(C(=O)NCC(C)(F)F)C[C@@H]1c1cnc2cnc3[nH]ccc3n12]

### Step A: (3S,4R)—N-(2,2-difluoropropyl)-3-ethyl-4-(3-tosyl-3H-imidazo[1,2-a]pyrrolo[2,3-e]pyrazin-8-yl)pyrrolidine-1-carboxamide

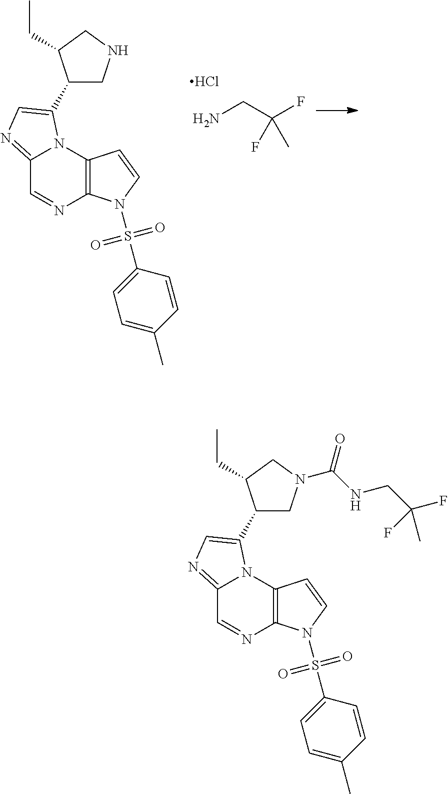

To a solution of 2,2-difluoropropan-1-amine hydrochloric acid (0.72 g, 5.47 mmol, SynQuest) in acetonitrile (4.0 mL) at ambient temperature was added TEA (0.50 mL, 3.42 mmol) and CDI (0.83 g, 5.13 mmol) and stirred for about 1 h. To the resulting mixture was added a solution of 8-((3R,4S)-4-ethylpyrrolidin-3-yl)-3-tosyl-3H-imidazo[1,2-a]pyrrolo[2,3-e]pyrazine (0.70 g, 1.71 mmol, Example #12, Step G) and TEA (0.50 mL, 3.42 mmol) in acetonitrile (8.0 mL) and stirring was continued for about 18 h. To the reaction mixture was added EtOAc (10 mL) and saturated aqueous NaHCO₃ (5 mL). The layers were separated and the aqueous layer was back extracted with EtOAc (10 mL). The combined organic layers were washed with brine (10 mL), dried over anhydrous MgSO₄, filtered and concentrated under reduced pressure. The crude material was purified by silica gel chromatography eluting with a gradient of 0-6% MeOH/DCM to afford (3S,4R)—N-(2,2-difluoropropyl)-3-ethyl-4-(3-tosyl-3H-imidazo[1,2-a]pyrrolo[2,3-e]pyrazin-8-yl)pyrrolidine-1-carboxamide (0.70 g, 77%) as a light yellow foam: LC/MS (Table 1, Method c) Rₜ=0.65 min; MS m/z: 531 (M+H)⁺.

### Step B: (3S,4R)—N-(2,2-difluoropropyl)-3-ethyl-4-(3H-imidazo[1,2-a]pyrrolo[2,3-e]pyrazin-8-yl)pyrrolidine-1-carboxamide

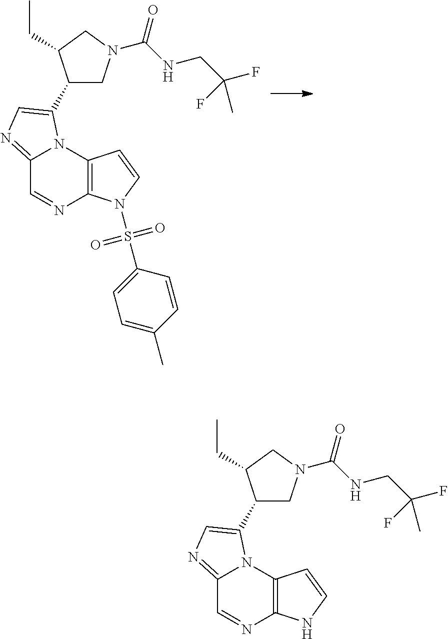 [SMILES: CC[C@@H]1CN(C(=O)NCC(C)(F)F)C[C@@H]1c1cnc2cnc3c(ccn3S(=O)(=O)c3ccc(C)cc3)n12 ; CC[C@@H]1CN(C(=O)NCC(C)(F)F)C[C@@H]1c1cnc2cnc3[nH]ccc3n12]

To a solution of (3S,4R)—N-(2,2-difluoropropyl)-3-ethyl-4-(3-tosyl-3H-imidazo[1,2-a]pyrrolo[2,3-e]pyrazin-8-yl)pyrrolidine-1-carboxamide (0.70 g, 1.31 mmol) in 1,4-dioxane (9 mL) was added 1N aqueous NaOH (2.6 mL, 2.6 mmol) and the reaction mixture was stirred at about 65° C. for about 1 h. The reaction was cooled to ambient temperature and to it was added DCM (20 mL) and saturated NaHCO₃ (10 mL). The layers were separated and the aqueous layer was back extracted with DCM (10 mL). The combined organic layers were dried over anhydrous MgSO₄, filtered and concentrated under reduced pressure. The crude material was purified by silica gel chromatography eluting with a gradient of 0-10% MeOH/DCM to afford a solid that was further purified by chiral chromatography (Table 2, Method 3). The detection methods used were UV (λ=340 nm) as well as optical rotation, Rₜ=12.8 min, or =negative The product was lyophilized to afford (3S,4R)—N-(2,2-difluoropropyl)-3-ethyl-4-(3H-imidazo[1,2-a]pyrrolo[2,3-e]pyrazin-8-yl)pyrrolidine-1-carboxamide (0.24 g, 47%): LC/MS (Table 1, Method b) Rₜ=1.51 min; MS m/z 377 (M+H)⁺. Jak3 IC₅₀=C

### Example #17

### (3S,4R)—N-cyclobutyl-3-ethyl-4-(3H-imidazo[1,2-a]pyrrolo[2,3-e]pyrazin-8-yl)pyrrolidine-1-carboxamide

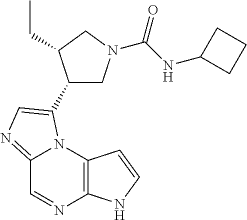 [SMILES: CC[C@@H]1CN(C(=O)NC2CCC2)C[C@@H]1c1cnc2cnc3[nH]ccc3n12]

### Step A: (3S,4R)—N-cyclobutyl-3-ethyl-4-(3-tosyl-3H-imidazo[1,2-a]pyrrolo[2,3-e]pyrazin-8-yl)pyrrolidine-1-carboxamide

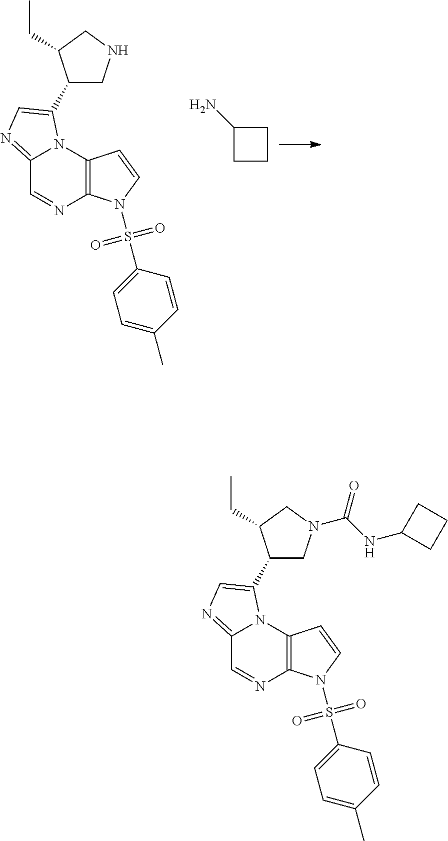 [SMILES: CC[C@@H]1CNC[C@@H]1c1cnc2cnc3c(ccn3S(=O)(=O)c3ccc(C)cc3)n12 ; NC1CCC1 ; CC[C@@H]1CN(C(=O)NC2CCC2)C[C@@H]1c1cnc2cnc3c(ccn3S(=O)(=O)c3ccc(C)cc3)n12]

To a solution of cyclobutylamine (0.199 mL, 2.344 mmol) in MeCN (3.42 mL) was added CDI (0.356 g, 2.198 mmol). The mixture was stirred for about 1 h upon which a white precipitate had formed. To this reaction mixture was added 8-((3R,4S)-4-ethylpyrrolidin-3-yl)-3-tosyl-3H-imidazo[1,2-a]pyrrolo[2,3-e]pyrazine (0.600 g, 1.465 mmol, Example #12, Step G) and TEA (0.408 mL, 2.93 mmol) in MeCN (6.83 mL). The combined reaction mixture was stirred at ambient temperature for about 3 h. In a separate flask, cyclobutylamine (0.199 mL, 2.344 mmol) and CDI (0.356 g, 2.198 mmol) MeCN (3.42 mL) stirred for about 1 hand then added to the above reaction mixture. Combined mixture was stirred for about 1 h and quenched with saturated aqueous NaHCO₃ (40 mL) and extracted into DCM (2×50 mL). The combined organics were dried over MgSO₄, filtered, and concentrated under reduced pressure. The product was purified on silica gel (40 g) using 0-6% MeOH in DCM. The product containing fractions were concentrated under reduced pressure and dried on high vacuum to afford (3S,4R)—N-cyclobutyl-3-ethyl-4-(3-tosyl-3H-imidazo[1,2-a]pyrrolo[2,3-e]pyrazin-8-yl)pyrrolidine-1-carboxamide (0.686 g, 92%, 92% purity). LC/MS (Table 1, Method a) Rₜ=2.12 min; MS m/z: 507 (M+H)⁺.

### Step B: (3S,4R)—N-cyclobutyl-3-ethyl-4-(3H-imidazo[1,2-a]pyrrolo[2,3-e]pyrazin-8-yl)pyrrolidine-1-carboxamide

To a solution of (3S,4R)—N-cyclobutyl-3-ethyl-4-(3-tosyl-3H-imidazo[1,2-a]pyrrolo[2,3-e]pyrazin-8-yl)pyrrolidine-1-carboxamide (0.686 g, 1.25 mmol) in 1,4-dioxane (6.92 mL) was added 1N aqueous NaOH (2.49 mL, 2.49 mmol). The reaction mixture was stirred at about 65° C. for about 1 h, cooled to ambient temperature and DCM (30 mL) and (10 mL) was added. The organic layer was separated; the aqueous layer was extracted with DCM (20 mL) and 10% IPA in DCM (2×20 mL). The combined organics were dried over MgSO₄, filtered, and concentrated under reduced pressure. The product was purified on silica gel (25 g) using 0-10% MeOH in DCM. The product containing fractions were combined and concentrated under reduced pressure to afford the product as a solid, which was purified by chiral chromatography (Table 2, Method 4) Rₜ=19.7 min, or =negative. The fractions were concentrated under reduced pressure, dissolved in DCM, and concentrated under reduced pressure to obtain a solid. It was dissolved in MeCN/water mixture and volatiles were removed in vacuo. The remaining solution was freeze dried and then dried under vacuum for about 1 h to afford (3S,4R)—N-cyclobutyl-3-ethyl-4-(3H-imidazo[1,2-a]pyrrolo[2,3-e]pyrazin-8-yl)pyrrolidine-1-carboxamide (0.275 g, 60%), LC/MS (Table 1, Method a) Rₜ=1.51 min; MS m/z: 353 (M+H)⁺. Jak3 IC₅₀=C

### Example #18

### (3S,4R)-3-methyl-4-(3H-imidazo[1,2-a]pyrrolo[2,3-e]pyrazin-8-yl)-N-(2,2,2-trifluoroethyl)pyrrolidine-1-carboxamide

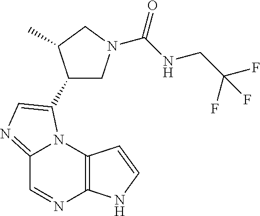 [SMILES: C[C@@H]1CN(C(=O)NCC(F)(F)F)C[C@@H]1c1cnc2cnc3[nH]ccc3n12]

### Step A: (Z)-methyl pent-2-enoate

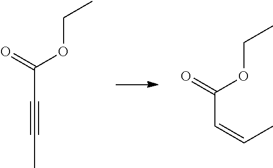

To a mixture of ethyl but-2-ynoate (50 g, 446 mmol) and Lindlar catalyst (9.49 g, 4.46 mmol) in 1 L round-bottom flask THF (1013 mL) and pyridine (101 mL) were added. The reaction mixture was degassed and purged with nitrogen (5×). The reaction was then blanked with a hydrogen balloon. After about 48 h the suspension was filtered through a pad of Celite® washing with THF (3×100 mL). The THF was removed in vacuo at about 40° C. and 200 mbar. The residue was diluted with Et₂O (1 L) and washed with water (3×200 mL) followed by 20% CuSO₄ aqueous solution (5×200 mL) followed by water (3×150 mL) and dried over MgSO₄, filtered and the solvent was removed in vacuo at about 40° C. and 200 mbar to give (Z)-methyl pent-2-enoate (45.3 g, 89%) as an oil. ¹H NMR (400 MHz, CDCl₃) δ 6.34-6.30 (m, 1H), 5.82-5.75 (m, 1H), 4.20-4.14 (m, 2H), 2.14 (dd, 3H), 1.31-1.27 (m, 3H).

### Step B: cis-methyl 1-benzyl-4-ethylpyrrolidine-3-carboxylate

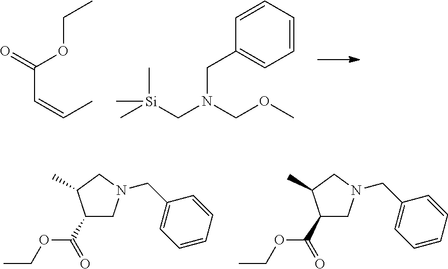

In a 2 L 3-neck round bottom flask fitted with a nitrogen inlet adapter, (Z)-ethyl but-2-enoate (68 g, 596 mmol) in DCM (784 mL) was added to give a yellow solution. About 10% of the N-benzyl-1-methoxy-N-((trimethylsilyl)methyl)methanamine (15 mL) was added to the solution followed by dropwise addition of TFA (0.496 mL, 6.43 mmol). After about 30 min the remainder of the N-benzyl-1-methoxy-N-((trimethylsilyl)-methyl)methanamine (137 mL) was added over about 2 h. After about 48 h the solvent was removed in vacuo to provide cis-methyl 1-benzyl-4-ethylpyrrolidine-3-carboxylate (137 g, 86% yield) as an orange oil. ¹H NMR (400 MHz, CDCl₃) δ 7.36-7.20 (m, 5H), 4.18-4.11 (m, 2H), 3.65 (d, 2H), 3.13-2.99 (m, 3H), 2.75 (dd, 1H), 2.60 (dd, 1H), 2.07 (t, 1H), 1.29-1.24 (m, 3H), 0.96 (dd, 3H).

### Step C: (cis)-1-(benzyloxycarbonyl)-4-methylpyrrolidine-3-carboxylic acid

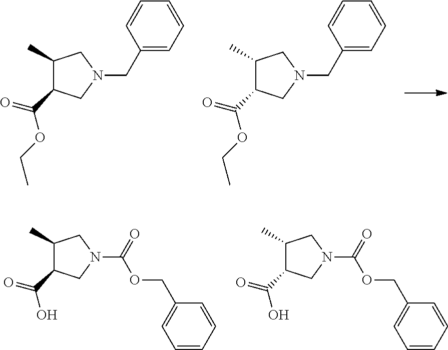 [SMILES: CCOC(=O)[C@@H]1CN(Cc2ccccc2)C[C@@H]1C ; CCOC(=O)[C@H]1CN(Cc2ccccc2)C[C@H]1C ; C[C@H]1CN(C(=O)OCc2ccccc2)C[C@H]1C(=O)O ; C[C@@H]1CN(C(=O)OCc2ccccc2)C[C@@H]1C(=O)O]

To a solution of (cis)-ethyl 1-benzyl-4-methylpyrrolidine-3-carboxylate (541 g, 2187 mmol) in water (1.5 L) was added concentrated HCl (1.64 L, 19.7 mol). The reaction mixture was heated to reflux. After about 15 h the reaction mixture was cooled to rt and partially concentrated in vacuo at about 50° C. The reaction mixture was diluted with water (500 mL) and washed with Et₂O (500 mL). The pH of the aqueous solution was adjusted to about 2.5 with aqueous NaOH (10 N) resulting in approx. (1.5 L) of solution. To the reaction mixture was added a slurry of 20% Pd(OH)₂—C, wet (20 g, 14.53 mmol) in water (15 mL). The mixture was shaken under 30 psi of hydrogen at about 50° C. for about 16 h. The reaction mixture was filtered and the organic layer was separated. The aqueous layer was partially coned in cacao at about 50° C. (removed approximately 1 L). The pH of the reaction mixture was adjusted to about 11 with aqueous NaOH (10 N). Dioxane (754 mL) was added followed by benzyl 2,5-dioxopyrrolidin-1-yl carbonate (572 g, 2297 mmol). The reaction pH was maintained at pH of about 10-12 by addition of 10N NaOH. After about 6 h Et₂O (1 L) was added and the aqueous layer was separated. Et₂O (2 L) was added and the pH of the aqueous layer was adjusted to about 2-3 with concentrated aqueous HCl. The aqueous phase was separated and the organic layer was dried over MgSO₄, filtered and solvent removed in vacuo to provide (cis)-1-(benzyloxycarbonyl)-4-methylpyrrolidine-3-carboxylic acid (570 g, 99%) as an orange oil. LC/MS (Table 1, Method a) Rₜ=1.64 mm; MS m/z: 264 (M+H)⁺.

### Step D: (R)-1-phenylethanammonium (3R,4S)-1-(benzyloxycarbonyl)-4-methylpyrrolidine-3-carboxylate

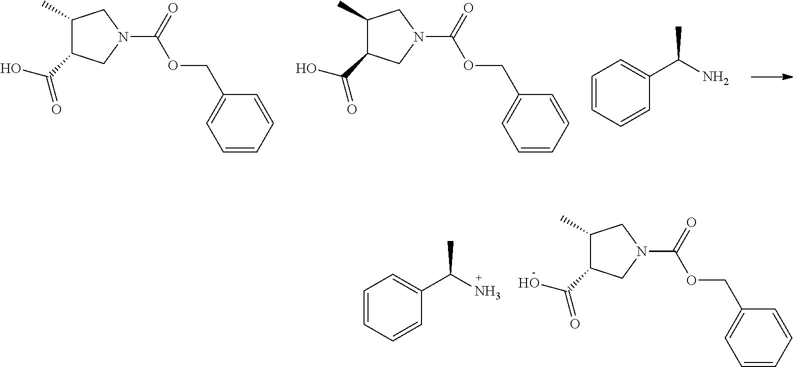

Cis-1-(benzyloxycarbonyl)-4-methylpyrrolidine-3-carboxylic acid (250 g, 950 mmol) was dissolved in acetonitrile (2 L) and (R)-1-phenylethanamine (0.06 L, 475 mmol) was added with stirring. After about 15 h the mixture was diluted with acetonitrile (1 L). After about 1 h the slurry was filtered, to provide a mixture of (R)-1-phenylethanammonium (3R,4S)-1-(benzyloxycarbonyl)-4-methylpyrrolidine-3-carboxylate and (R)-1-phenylethanammonium (3S,4R)-1-(benzyloxycarbonyl)-4-methylpyrrolidine-3-carboxylate (approx. 1:1) (Rₜ=3.94 and 4.22 min, Table 1, Method g). The solids were dissolved in hot acetonitrile (2 L) with stirring at about 80° C. After about 30 min the mixture was allowed to cool to about 40° C. and the solid was collected by filtration to provide a mixture of (R)-1-phenylethanammonium (3R,4S)-1-(benzylooxycarbonyl)-4-methylpyrrolidine-3-carboxylate and (R)-1-phenylethanammonium (3S,4R)-1-(benzyloxycarbonyl)-4-methylpyrrolidine-3-carboxylate (approx. 60:40), Rₜ=3.94 and 4.22 min, Table 1, Method g). The solids were dissolved in hot acetonitrile (2 L) at about 80° C. with stirring. After about 30 min the mixture was allowed to cool to about 50° C. and filtered to provide a mixture of (R)-1-phenylethanammonium (3R,4S)-1-(benzyloxycarbonyl)-4-methylpyrrolidine-3-carboxylate and (R)-1-phenylethanammonium (3S,4R)-1-(benzyloxycarbonyl)-4-methylpyrrolidine-3-carboxylate (approx. 90:10) Rₜ=3.94 and 4.22 min, Table 1, Method g). The solids were dissolved in hot acetonitrile (2 L) at about 80° C. with stirring. After about 30 min the mixture was allowed to cool to about 50° C. and filtered to provide (R)-1-phenylethanammonium (3R,4S)-1-(benzyloxycarbonyl)-4-methylpyrroline-3-carboxylate (71 g, 19%) (er≧98:2), Rₜ=3.94 min, Table 1, Method g). LC/MS (Table 1, Method a) Rₜ=1.64 min; MS m/z: 264 (M+H)⁺.

### Step E: (3R,4S)-benzyl 3-(chlorocarbonyl)-4-methylpyrrolidine-1-carboxylate

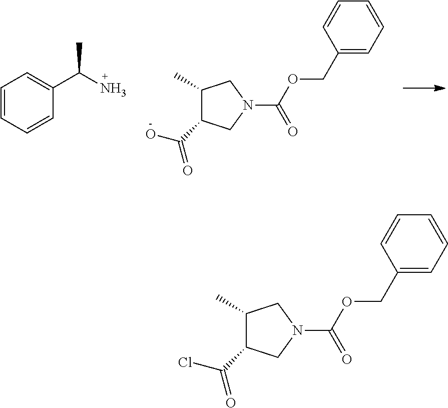

In a 250 mL separatory funnel (R)-1-phenylethanammonium (3R,4S)-1-(benzyloxycarbonyl)-4-methylpyrrolidine-3-carboxylate (3.843 g, 10 mmol) was partitioned between diethyl ether (100 mL) and 1 N aqueous HCl (0.309 mL, 10 mmol). The layers were separated and the aqueous layer was extracted with diethyl ether (50 mL). The combined organics were dried over magnesium sulfate and the solvent was removed in vacuo to provide crude (3R,4S)-1-(benzyloxycarbonyl)-4-methylpyrrolidine-3-carboxylic acid as a yellow oil. To a solution of crude (3R,4S)-1-(benzyloxycarbonyl)-4-methylpyrrolidine-3-carboxylic acid (2.716 g) in DCM (25 mL) was added DMF (0.040 mL, 0.516 mmol) followed by oxalyl chloride (2M in DCM, 10.32 mL, 20.63 mmol). After about 3 h, the solvent was removed in vacuo to provide crude (3R,4S)-benzyl 3-(chlorocarbonyl)-4-methylpyrrolidine-1-carboxylate as an oil. (2.8 g, 100%). ¹H NMR (400 MHz, CDCl₃) δ 7.40-7.28 (m, 5H), 5.20-5.07 (m, 2H), 3.80 (ddd, 1H), 3.60 (m, 3H), 3.31 (ddd, 1H), 2.82-2.70 (m, 1H), 1.15 (d, 3H).

### Step F: (3R,4S)-benzyl 3-(2-bromoacetyl)-4-methylpyrrolidine-1-carboxylate

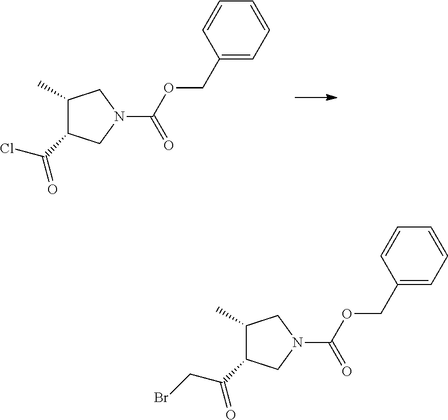 [SMILES: C[C@@H]1CN(C(=O)OCc2ccccc2)C[C@@H]1C(=O)Cl ; C[C@@H]1CN(C(=O)OCc2ccccc2)C[C@@H]1C(=O)CBr]

In a 250 mL round-bottom flask, (3R,4S)-benzyl 3-(chlorocarbonyl)-4-methylpyrrolidine-1-carboxylate (2.91 g, 10.33 mmol) in THF (25 mL) and acetonitrile (25 mL) was added to give an orange solution. The mixture was cooled to about 0° C. and (trimethylsilyl)diazomethane (18.08 mL, 36.2 mmol) was added dropwise via an addition funnel at a rate which kept the temperature below about 10° C. After about 2 h, 48% aqueous HBr (11.76 mL, 103 mmol) was added dropwise via an addition funnel at a rate which kept the temperature at less than about 10° C. After addition was complete the reaction mixture was stirred for about 90 min. Ether (50 mL) and water (50 mL) were added and the layers were separated and the organic layer was washed with saturated aqueous sodium bicarbonate (50 mL) and brine (50 mL). The initial aqueous layer was extracted with 1:1 ether/ethyl acetate (75 mL). The organic layer was separated and washed with saturated aqueous sodium bicarbonate (50 mL) and brine (50 mL). The combined organic layers were dried over magnesium sulfate, filtered and the solvent was removed in vacuo to provide (3R,4S)-benzyl 3-(2-bromoacetyl)-4-methylpyrrolidine-1-carboxylate (3.201 g, 91%) as an oil. LC/MS (Table 1, Method a) Rₜ=2.42 min; MS m/z: 340, 342 (M+H)⁺.

### Step G: (3R,4S)-benzyl 3-(2-(tert-butoxycarbonyl(5-tosyl-5H-pyrrolo[2,3-b]pyrazin-2-yl)amino)acetyl)-4-methylpyrrolidine-1-carboxylate

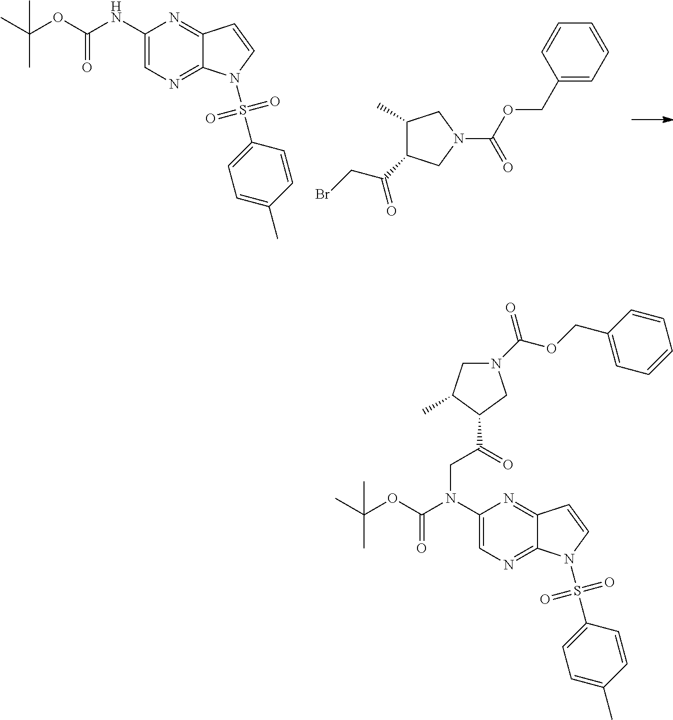

To a slurry of NaH (0.377 mg, 9.41 mmol, 60% in mineral oil) in DMF (12 mL) was added tert-butyl 5-tosyl-5H-pyrrolo[2,3-b]pyrazin-2-ylcarbamate (3.65 g, 9.41 mmol, WO 2011/068881) in DMF (12 mL). After about 2 h the mixture was cooled to about 0° C. and a solution of (3R,4S)-benzyl 3-(2-bromoacetyl)-4-methylpyrrolidine-1-carboxylate (3.201 g, 9.41 mmol) in DMF (12 mL) was added dropwise via an addition funnel, keeping the temperature below about 10° C. After the addition was complete, the mixture was stirred at ambient temperature for about 2 h. The reaction mixture was quenched with saturated aqueous ammonium chloride (30 mL) and diethyl ether (50 mL) was added. The organic layer was separated and the aqueous layer was extracted with diethyl ether (50 mL). The combined organic layers were washed with brine (50 mL) and dried over magnesium sulfate. The solvent was removed in vacuo and the crude sample was purified by chromatographed on silica gel eluting with EtOAc/heptanes to provide (3R,4S)-benzyl 3-(2-(tert-butoxycarbonyl(5-tosyl-5H-pyrrolo[2,3-b]pyrazin-2-yl)amino)acetyl)-4-methylpyrrolidine-1-carboxylate (2.64 g, 43%). LC/MS (Table 1, Method a) Rₜ=3.06 min; MS m/z: 648 (M+H)⁺.

### Step H: (3S,4R)-benzyl 3-methyl-4-(2-(5-tosyl-5H-pyrrolo[2,3-b]pyrazin-2-ylamino)acetyl)pyrrolidine-1-carboxylate

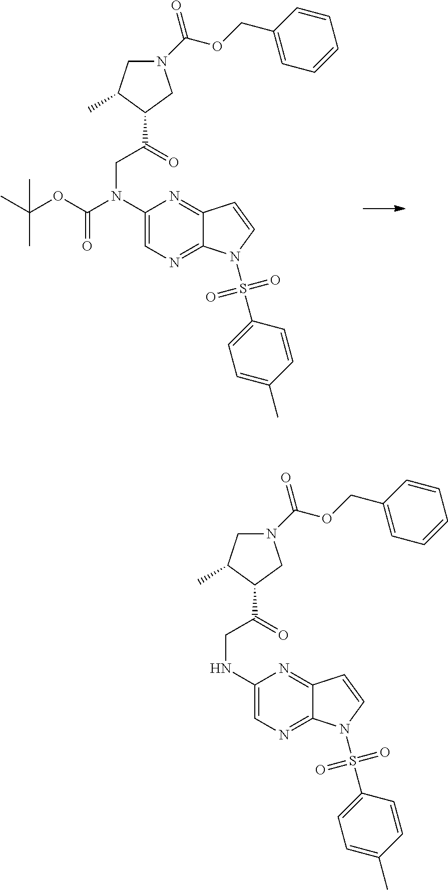 [SMILES: Cc1ccc(S(=O)(=O)n2ccc3nc(N(CC(=O)[C@H]4CN(C(=O)OCc5ccccc5)C[C@H]4C)C(=O)OC(C)(C)C)cnc32)cc1 ; Cc1ccc(S(=O)(=O)n2ccc3nc(NCC(=O)[C@H]4CN(C(=O)OCc5ccccc5)C[C@H]4C)cnc32)cc1]

In a 100 ml round-bottom flask, (3R,4S)-benzyl 3-(2-(tert-butoxycarbonyl(5-tosyl-5H-pyrrolo[2,3-b]pyrazin-2-yl)amino)acetyl)-4-methylpyrrolidine-1-carboxylate (2.64 g, 4.07 mmol) in dichloromethane (27 mL) was added to give a colorless solution. TFA (3.14 mL, 40.7 mmol) was added and the mixture was stirred at ambient temperature under nitrogen. After about 48 h the solvents were removed in vacuo. The residue was partitioned between DOA (50 mL) and saturated aqueous sodium bicarbonate (50 mL). The organic layer was separated, dried over magnesium sulfate, filtered and concd in vacuo to provide (3S,4R)-benzyl 3-methyl-4-(2-(5-tosyl-5H-pyrrolo[2,3-b]pyrazin-2-ylamino)acetyl)pyrrolidine-1-carboxylate (2.24 g, 96%) LC/MS (Table 1, Method a) Rₜ=2.57 min; MS m/z: 548 (M+H)⁺.

### Step I: (3S,4R)-benzyl 3-methyl-4-(3-tosyl-3H-imidazo[1,2-a]pyrrolo[2,3-e]pyrazin-8-yl)pyrrolidine-1-carboxylate

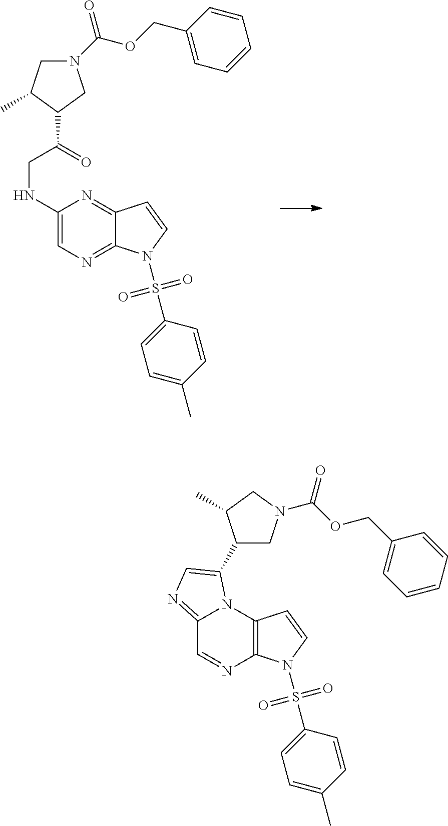

To a 100 mL round-bottom flask, (3S,4R)-benzyl 3-methyl-4-(2-(5-tosyl-5H-pyrrolo[2,3-b]pyrazin-2-ylamino)acetyl)pyrrolidine-1-carboxylate (2.24 g, 4.09 mmol) 1,4-dioxane (20 mL) was added to give an orange solution. Nitrogen was bubbled into the solution for about 10 min, then Lawesson's reagent (0.993 g, 2.454 mmol) was added and the mixture was heated at about 70° C. After about 15, the mixture was cooled to ambient temperature and water (5 mL) was added, followed by sodium perborate monohydrate (2.450 g, 24.54 mmol). After about 15 h saturated aqueous sodium bicarbonate (10 mL) was added. After about 1 h, ethyl acetate/ether (1:1, 50 mL) was added. The layers were separated and the organic layer was dried over magnesium sulfate, filtered and the solvent was removed in vacuo. The crude product was purified by chromatography on silica gel eluting with EtOAc/heptanes to provide (3S,4R)-benzyl 3-methyl-4-(3-tosyl-3H-imidazo[1,2-a]pyrrolo[2,3-e]pyrazin-8-yl)pyrrolidine-1-carboxylate (1.122 g, 51%), LC/MS (Table 1, Method a) Rₜ=2.60 min; MS m/z: 530 (M+H)⁺.

### Step J: 8-((3R,4S)-4-methylpyrrolidin-3-yl)-3-tosyl-3H-imidazo[1,2-a]pyrrolo[2,3-e]pyrazine

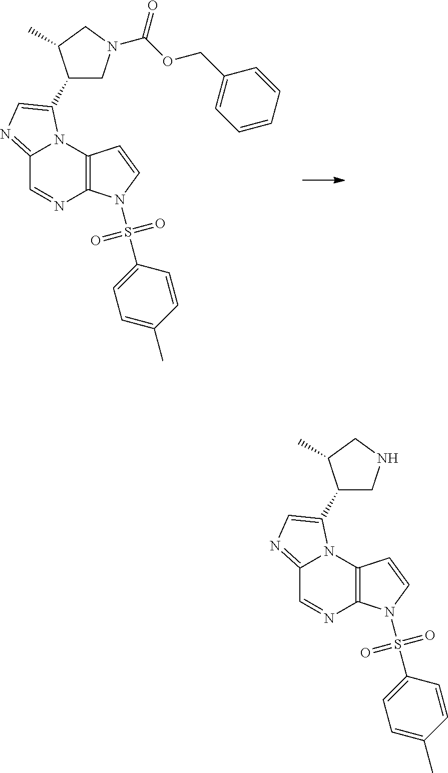 [SMILES: Cc1ccc(S(=O)(=O)n2ccc3c2ncc2ncc([C@H]4CN(C(=O)OCc5ccccc5)C[C@H]4C)n23)cc1 ; Cc1ccc(S(=O)(=O)n2ccc3c2ncc2ncc([C@H]4CNC[C@H]4C)n23)cc1]

(3S,4R)-benzyl 3-methyl-4-(3-tosyl-3H-imidazo[1,2-a]pyrrolo[2,3-e]pyrazin-8-yl)pyrolidine-1-carboxylate (0.257 g, 0.485 mmol) and 33% HBr in acetic acid (5 mL, 88 mmol) were added to a 25 mL round bottom flask and the orange mixture was stirred at ambient temperature. After about 2 h, EtOAc was added until precipitate stopped forming (approx. 15 mL). The solids were collected by filtration, washing with EtOAc. The solids were dried in vacuo. The solids were partitioned between EtOAc (20 mL) and saturated aqueous NaHCO₃ (10 mL). The organic layer was separated, dried (MgSO₄) filtered and concentrated in vacuo to provide 8-((3R,4S)-4-methylpyrrolidin-3-yl)-3-tosyl-3H-imidazo[1,2-a]pyrrolo[2,3-e]pyrazine (0.082 g, 39%). LC/MS (Table 1, Method a) Rₜ=1.62 min; MS m/z: 396 (M+H)⁺.

### Step K: (3S,4R)-3-methyl-4-(3-tosyl-3H-imidazo[1,2-a]pyrrolo[2,3-e]pyrazin-8-yl)-N-(2,2,2-trifluoroethyl)pyrrolidine-1-carboxamide

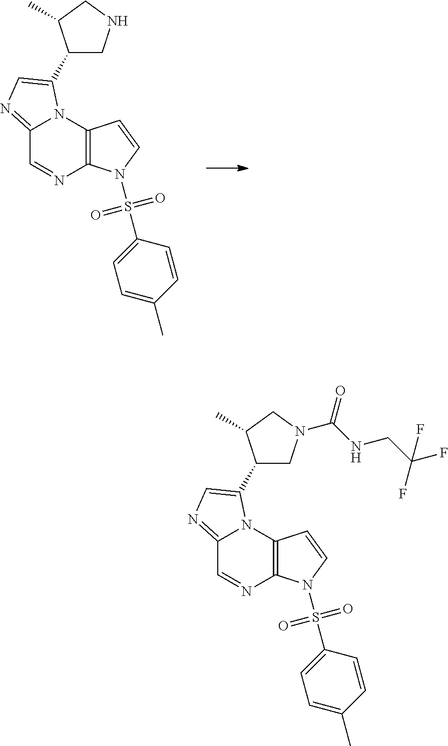 [SMILES: Cc1ccc(S(=O)(=O)n2ccc3c2ncc2ncc([C@H]4CNC[C@H]4C)n23)cc1 ; Cc1ccc(S(=O)(=O)n2ccc3c2ncc2ncc([C@H]4CN(C(=O)NCC(F)(F)F)C[C@H]4C)n23)cc1]

To a solution of 2,2,2-trifluoroethanamine (10.06 g, 102 mmol) DMF (18 mL) was added N,N′-carbonyldiimidazole (16.16 g, 100 mmol). The solution was heated under nitrogen at about 65° C. for about 24 h. In a separate flask, 8-((3R,4S)-4-methylpyrrolidin-3-yl)-3-tosyl-3H-imidazo[1,2-a]pyrrolo[2,3-e]pyrazine (0.400 g, 1.011 mmol) was dissolved in DMF (20 mL) to give a yellow solution. A portion of the original trifluoroenthanamine solution (0.318 mL) was added and the mixture was heated at 65° for about 1 h. The solvent was removed in vacuo and the residue was partitioned between EtOAc (25 mL) and water (15 mL). The aqueous layer was separated and washed with water (2×15 mL), dried over magnesium sulfate, filtered and the solvent was removed in vacuo to give (3S,4R)-3-methyl-4-(3-tosyl-3H-imidazo[1,2-a]pyrrolo[2,3-e]pyrazin-8-yl)-N-(2,2,2-trifluoroethyl)pyrrolidine-1-carboxamide (0.484 g, 87%) as an amorphous solid: LC/MS (Table 1, Method a) Rₜ=2.16 min; MS m/z: 521 (M+H)⁺.

### Step L: (3R,4S)-3-(3H-imidazo[1,2-a]pyrrolo[2,3-e]pyrazin-8-yl)-4-methyl-N-(2,2,2-trifluoroethyl)pyrrolidine-1-carboxamide

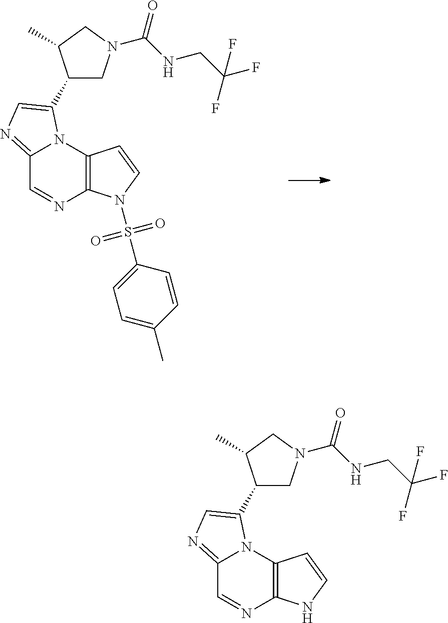 [SMILES: Cc1ccc(S(=O)(=O)n2ccc3c2ncc2ncc([C@H]4CN(C(=O)NCC(F)(F)F)C[C@H]4C)n23)cc1 ; C[C@@H]1CN(C(=O)NCC(F)(F)F)C[C@@H]1c1cnc2cnc3[nH]ccc3n12]

In a 25 mL round-bottom flask, (3S,4R)-3-methyl-4-(3-tosyl-3H-imidazo[1,2-a]pyrrolo[2,3-e]pyrazin-8-yl)-N-(2,2,2-trifluoroethyl)pyrrolidine-1-carboxamide (0.484 g, 0.930 mmol) and a 1 N aqueous NaOH (4.65 mL, 4.65 mmol) in 1,4-dioxane (25 mL) were added to give a yellow solution. The mixture was heated at about 55° C. for about 16 h. The reaction mixture was cooled to ambient temperature and the mixture was partitioned between water (40 mL) and EtOAc (240 mL). The layers were separated and the aqueous extracted with EtOAc (4×160 mL). The combined organics layers were dried over anhydrous magnesium sulfate and the solvent was removed in vacuo. The resulting solid was purified by normal phase chromatography on silica gel eluting with a gradient of 0-10% methanol in dichloromethane to give (3R,4S)-3-(3H-imidazo[1,2-a]pyrrolo[2,3-e]pyrazin-8-yl)-4-methyl-N-(2,2,2-trifluoroethyl)pyrrolidine-1-carboxamide (0.163 g, 48%) as an amorphous off-white solid: LC/MS (Table 1, Method a) Rₜ=1.55 min; MS m/z: 367 (M+H)⁺. Jak3 IC₅₀=C

### Example 09

### (3R,4S)—N-(2,2-difluoroethyl)-3-(3H-imidazo[1,2-a]pyrrolo[2,3-e]pyrazin-8-yl)-4-methylpyrrolidine-1-carboxamide

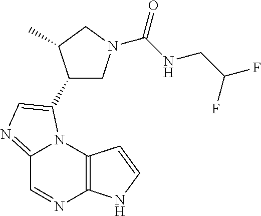 [SMILES: C[C@@H]1CN(C(=O)NCC(F)F)C[C@@H]1c1cnc2cnc3[nH]ccc3n12]

### Step A: (3S,4R)-3-methyl-4-(3-tosyl-3H-imidazo[1,2-a]pyrrolo[2,3-e]pyrazin-8-yl)pyrrolidine-1-carbonyl chloride

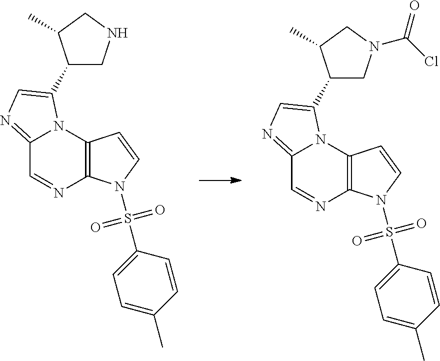 [SMILES: Cc1ccc(S(=O)(=O)n2ccc3c2ncc2ncc([C@H]4CNC[C@H]4C)n23)cc1 ; Cc1ccc(S(=O)(=O)n2ccc3c2ncc2ncc([C@H]4CN(C(=O)Cl)C[C@H]4C)n23)cc1]

To a solution of 8-((3R,4S)-4-methylpyrrolidin-3-yl)-3-tosyl-3H-imidazo[1,2-a]pyrrolo[2,3-e]pyrazine (1.125 g, 2.84 mmol, Example #18, Step J) in THF (100 mL) was added TEA (0.396 mL, 2.84 mmol) followed by 20% phosgene in toluene (1.4 mL, 2.84 mmol) and the mixture was stirred at ambient temperature for 3 h. The resulting precipitate was removed by filtration, and the solvent was removed from the filtrate in vacuo to give (3S,4R)-3-methyl-4-(3-tosyl-3H-imidazo[1,2-a]pyrrolo[2,3-e]pyrazin-8-yl)pyrrolidine-1-carbonyl chloride (1.117 g, 86%): ¹H NMR (400 MHz, CDCl₃) δ 9.10 (d, 1H), 8.13 (dd, 2H), 7.93 (d, 1H), 7.72 (d, 1H), 7.35 (d, 2H), 7.26 (d, 1H), 7.20-7.13 (m, 1H), 6.91 (s, 1H), 4.26-4.14 (m, 1H), 4.13-3.85 (m, 2H), 3.51 (ddd, 1H), 2.92-2.81 (m, 1H), 2.41 (s, 3H), 2.35 (s, 1H), 1.47-1.33 (m, 3H), 0.76 (t, 3H).

### Step B: (3S,4R)—N-(2,2-difluoroethyl)-3-methyl-4-(3-tosyl-3H-imidazo[1,2-a]pyrrolo[2,3-e]pyrazin-8-yl)pyrrolidine-1-carboxamide

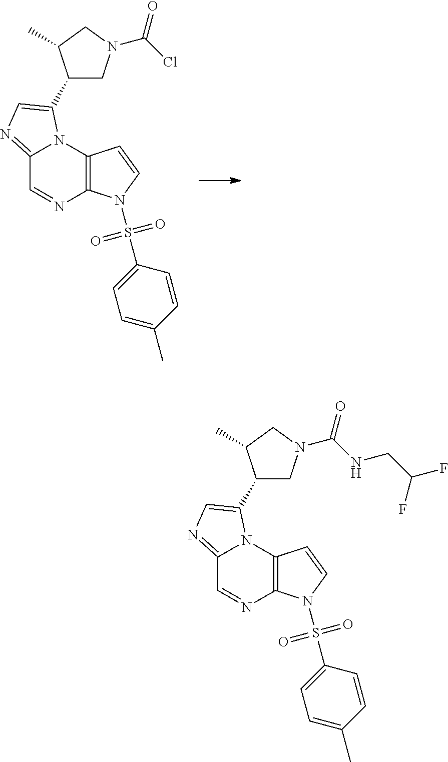 [SMILES: Cc1ccc(S(=O)(=O)n2ccc3c2ncc2ncc([C@H]4CN(C(=O)Cl)C[C@H]4C)n23)cc1 ; Cc1ccc(S(=O)(=O)n2ccc3c2ncc2ncc([C@H]4CN(C(=O)NCC(F)F)C[C@H]4C)n23)cc1]

To a solution of 2,2-difluoroethylamine (0.035 g, 0.437 mmol) in THF (5 mL) was added (3S,4R)-3-methyl-4-(3-tosyl-3H-imidazo[1,2-a]pyrrolo[2,3-e]pyrazin-8-yl)pyrrolidine-1-carbonyl chloride (0.200 g, 0.437 mmol) and the mixture was stirred at ambient temperature for about 16 h. The solvent was removed in vacuo and the residue was purified by normal phase chromatography on silica gel eluting with a gradient of 0-10% methanol in dichloromethane to give ((3S,4R)-3-methyl-4-(3-tosyl-3H-imidazo[1,2-a]pyrrolo[2,3-e]pyrazin-8-yl)pyrrolidin-1-yl)(morpholino)methanone (0.175 g, 77%) as an amorphous solid: LC/MS (Table 1, Method a) Rₜ=2.06 min; MS m/z: 503 (M+H)⁺.

### Step C: (3R,4S)—N-(2,2-difluoroethyl)-3-(3H-imidazo[1,2-a]pyrrolo[2,3-e]pyrazin-8-yl)-4-methylpyrrolidine-1-carboxamide

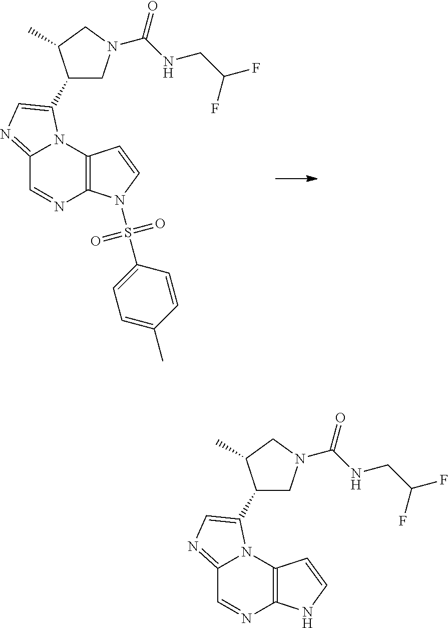 [SMILES: Cc1ccc(S(=O)(=O)n2ccc3c2ncc2ncc([C@H]4CN(C(=O)NCC(F)F)C[C@H]4C)n23)cc1 ; C[C@@H]1CN(C(=O)NCC(F)F)C[C@@H]1c1cnc2cnc3[nH]ccc3n12]

To a solution of (3S,4R)—N-(2,2-difluoroethyl)-3-methyl-4-(3-tosyl-3H-imidazo[1,2-a]pyrrolo[2,3-e]pyrazin-8-yl)pyrrolidine-1-carboxamide (0.175 g, 0.348 mmol) in 1,4-dioxane (10 mL) was added 1 M aqueous NaOH (1.74 mL, 1.74 mmol) to give a yellow solution. The mixture was heated at about 55° C. for about 16 h. The reaction mixture was cooled to ambient temperature and the mixture was partitioned between water (40 mL) and EtOAc (240 mL). The phases were separated and the aqueous phase was extracted with EtOAc (4×160 mL). The combined organic phases were dried over anhydrous magnesium sulfate and the solvent was removed in vacuo to give (3R,4S)—N-(2,2-difluoroethyl)-3-(3H-imidazo[1,2-a]pyrrolo[2,3-e]pyrazin-8-yl)-4-methylpyrrolidine-1-carboxamide (68.4 mg, 54%) as an amorphous solid: LC/MS (Table 1, Method a) Rₜ=1.43 min; MS m/z: 349 (M+H)⁺. Jak3 IC₅₀=B

### Example #20

### ((3R,4S)-3-(3H-imidazo[1,2-a]pyrrolo[2,3-e]pyrazin-8-yl)-4-methylpyrrolidin-1-yl)(morpholino)methanone

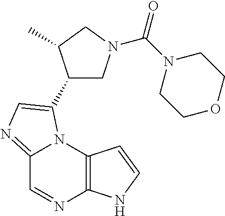 [SMILES: C[C@@H]1CN(C(=O)N2CCOCC2)C[C@@H]1c1cnc2cnc3[nH]ccc3n12]

### Step A: ((3S,4R)-3-methyl-4-(3-tosyl-3H-imidazo[1,2-a]pyrrolo[2,3-e]pyrazin-8-yl)pyrrolidin-1-yl)(morpholino)methanone

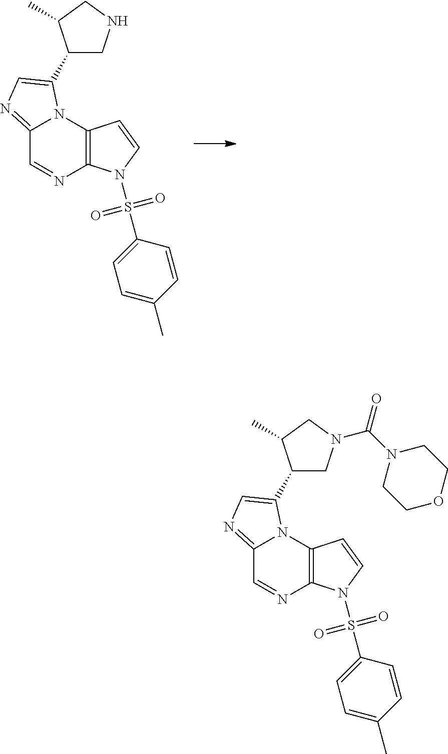 [SMILES: Cc1ccc(S(=O)(=O)n2ccc3c2ncc2ncc([C@H]4CNC[C@H]4C)n23)cc1 ; Cc1ccc(S(=O)(=O)n2ccc3c2ncc2ncc([C@H]4CN(C(=O)N5CCOCC5)C[C@H]4C)n23)cc1]

To a solution of 8-((3R,4S)-4-methylpyrrolidin-3-yl)-3-tosyl-3H-imidazo[1,2-a]pyrrolo[2,3-e]pyrazine (0.200 g, 0.506 mmol, Example #18, Step J) in THF (20 mL) was added TEA (0.070 mL, 0.506 mmol) followed by 20% phosgene solution in toluene (0.25 mL, 0.506 mmol) and the mixture was stirred at ambient temperature for about 30 min. Morpholine (0.088 mL, 1.011 mmol) was added and the mixture was stirred at ambient temperature for about 16 h. The solvent was removed in vacuo and the resulting solid was purified by normal phase chromatography on silica gel eluting with a gradient of 0-10% methanol in dichloromethane to give ((3S,4R)-3-methyl-4-(3-tosyl-3H-imidazo[1,2-a]pyrrolo[2,3-e]pyrazin-8-yl)pyrrolidin-1-yl)(morpholino)methanone (0.180 g, 70%) as an amorphous solid: LC/MS (Table 1, Method a) Rₜ=2.04 min; MS m/z: 509 (M+H)⁺.

### Step B: ((3R,4S)-3-(3H-imidazo[1,2-a]pyrrolo[2,3-e]pyrazin-8-yl)-4-methylpyrrolidin-1-yl)(morpholino)methanone

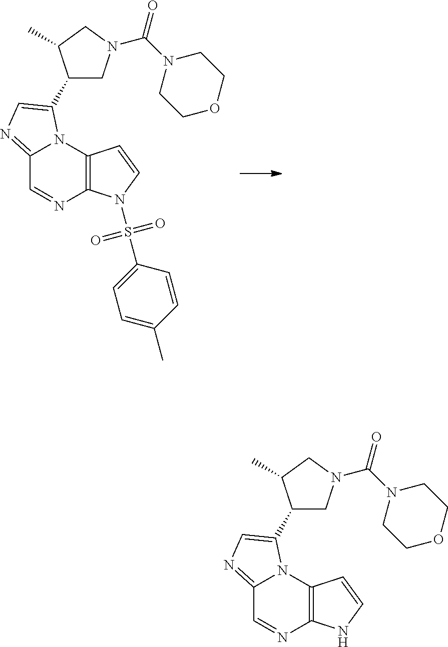 [SMILES: Cc1ccc(S(=O)(=O)n2ccc3c2ncc2ncc([C@H]4CN(C(=O)N5CCOCC5)C[C@H]4C)n23)cc1 ; C[C@@H]1CN(C(=O)N2CCOCC2)C[C@@H]1c1cnc2cnc3[nH]ccc3n12]

To a solution of ((3S,4R)-3-methyl-4-(3-tosyl-3H-imidazo[1,2-a]pyrrolo[2,3-e]pyrazin-8-yl)pyrrolidin-1-yl)(morpholino)methanone (0.180 g, 0.354 mmol) in 1,4-dioxane (5 mL) was added 1 N aqueous NaOH (1.77 mL, 1.77 mmol) to give a yellow solution. The mixture was heated at about 55° C. for about 3 h and then cooled to ambient temperature. The reaction mixture was partitioned between water (20 mL) and EtOAc (120 mL). The phases were separated and aqueous phase was extracted with EtOAc (4×80 mL). The combined organic phases were dried over anhydrous magnesium sulfate and solvent was removed in vacuo to give ((3R,4S)-3-(3H-imidazo[1,2-a]pyrrolo[2,3-e]pyrazin-8-yl)-4-methylpyrrolin-1-yl)(morpholino)methanone (0.0822 g, 65%) as an amorphous solid: LC/MS (Table 1, Method a) Rₜ=1.42 mm; MS m/z: 355 (M+H)⁺, Jak3 IC₅₀=C

### Example #21

### 1-((3R,4S)-3-(3H-imidazo[1,2-a]pyrrolo[2,3-e]pyrazin-8-yl)-4-methylpyrrolidin-1-yl)-2-cyclopropylethanone

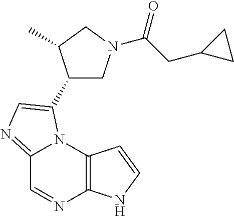 [SMILES: C[C@@H]1CN(C(=O)CC2CC2)C[C@@H]1c1cnc2cnc3[nH]ccc3n12]

### Step A: 2-cyclopropyl-1-((3S,4R)-3-methyl-4-(3-tosyl-3H-imidazo[1,2-a]pyrrolo[2,3-e]pyrazin-8-yl)pyrrolidin-1-yl)ethanone

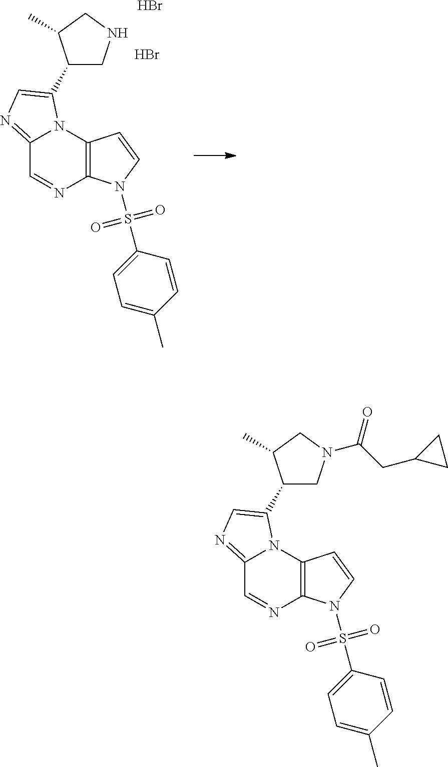 [SMILES: Br ; Cc1ccc(S(=O)(=O)n2ccc3c2ncc2ncc([C@H]4CNC[C@H]4C)n23)cc1 ; Br ; Cc1ccc(S(=O)(=O)n2ccc3c2ncc2ncc([C@H]4CN(C(=O)CC5CC5)C[C@H]4C)n23)cc1]

To a mixture of 8-((3R,4S)-4-methylpyrrolidin-3-yl)-3-tosyl-3H-imidazo[1,2-a]pyrrolo[2,3-e]pyrazine dihydrobromide (0.800 g, 1.44 mmol, Example #18, Step J), 2-cyclopropylacetic acid (0.158 g, 1.58 mmol, Matrix Scientific) and HATU (0.710 g, 1.87 mmol) in DCM (8 mL) was added TEA (1.20 mL, 8.61 mmol). The reaction was stirred at ambient temperature for about 16 h. The reaction was partitioned between DCM (25 mL) and brine (25 mL). The layers were separated and the aqueous layer was extracted with DCM (25 mL). The combined organic layers were dried with Na₂SO₄, filtered, and concentrated under reduced pressure. The residue was purified on silica gel using 0-3% MeOH in DCM to give 2-cyclopropyl-1-((3S,4R)-3-methyl-4-(3-tosyl-3H-imidazo[1,2-a]pyrrolo[2,3-e]pyrazin-8-yl)pyrrolidin-1-yl)ethanone (0.618 g, 89%) as an off-white foam: LC/MS (Table 1, Method b) Rₜ=2.02 min; MS m/z: 478 (M+H)⁺.

### Step B: 1-((3R,4S)-3-(3H-imidazo[1,2-a]pyrrolo[2,3-e]pyrazin-8-yl)-4-methylpyrrolidin-1-yl)-2-cyclopropylethanone

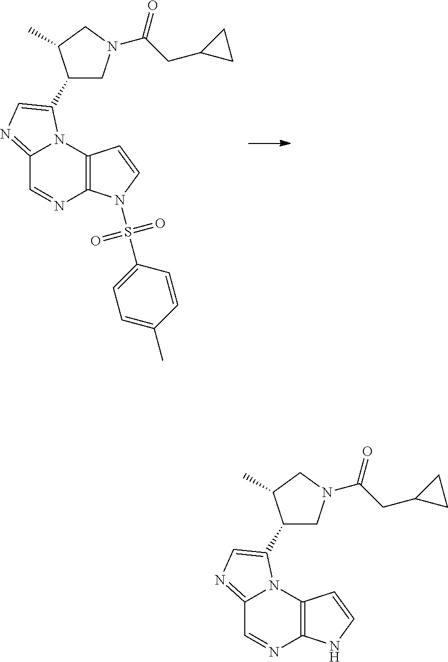 [SMILES: Cc1ccc(S(=O)(=O)n2ccc3c2ncc2ncc([C@H]4CN(C(=O)CC5CC5)C[C@H]4C)n23)cc1 ; C[C@@H]1CN(C(=O)CC2CC2)C[C@@H]1c1cnc2cnc3[nH]ccc3n12]

To a solution of 2-cyclopropyl-1-((3S,4R)-3-methyl-4-(3-tosyl-3H-imidazo[1,2-a]pyrrolo[2,3-e]pyrazin-8-yl)pyrrolidin-1-yl)ethanone (0.618 g, 1.28 mmol) in 1,4-dioxane (5 mL) was added 1 M aqueous NaOH (5 mL, 5 mmol). The reaction was stirred at ambient temperature for about 16 h. The reaction was partitioned between DCM (50 mL) and 5% aqueous acetic acid (50 mL). The organic layer was washed with brine (30 mL), dried with Na₂SO₄, filtered and concentrated under reduced pressure. The residue was purified on silica gel using 0-4% MeOH in DCM to give 1-((3R,4S)-3-(3H-imidazo[1,2-a]pyrrolo[2,3-e]pyrazin-8-yl)-4-methylpyrrolidin-1-yl)-2-cyclopropylethanone (0.295 g, 63%) as an off-white powder: LC/MS (Table 1, Method b) Rₜ=1.65 min; MS m/z: 324 (M+H)⁺. Jak3 IC₅₀=B

### Example #22

### 3-((1S,3R,4S)-3-ethyl-4-(6H-pyrrolo[2,3-e][1,2,4]triazolo[4,3-a]pyrazin-1-yl)cyclopentyl)propanenitrile

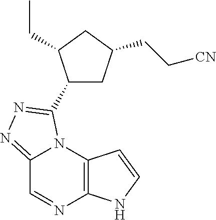 [SMILES: CC[C@@H]1C[C@H](CCC#N)C[C@@H]1c1nnc2cnc3[nH]ccc3n12]

### Step A: Ethyl-4-(2-tert-butoxy-2-oxoethylidene)-2-ethylcyclopentanecarboxylate

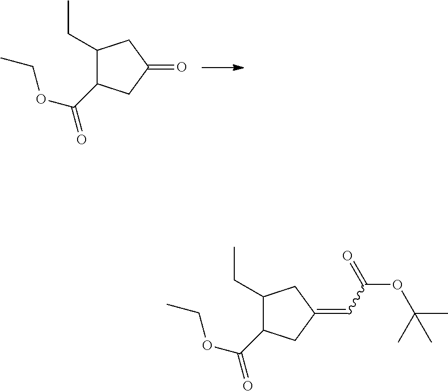 [SMILES: CCOC(=O)C1CC(=O)CC1CC ; CCOC(=O)C1CC(=CC(=O)OC(C)(C)C)CC1CC]

To a solution of sodium tert-butoxide (42.6 g, 443 mmol) in THF (400 mL) pre-cooled to about −2° C. was added tert-butyl 2-(diethoxyphosphoryl)acetate (126 g, 501 mmol) at about 10° C. over about 35 min. The resulting solution was stirred at 0° C. for 10 min then cooled to −5° C. To the solution was added a scalemic mixture enriched in (1S,2R)-ethyl 2-ethyl-4-oxocyclopentanecarboxylate (71 g, 385 mmol, WO2011/068881). The mixture was stirred at about −6° C. for about 14 h. The reaction mixture was diluted with heptane (500 mL). To the mixture was added aqueous phosphoric acid (113 g, 116 mmol) and water (100 mL). The organic layer was separated then washed with water (200 mL), brine (200 mL), 8% aqueous NaHCO₃ (200 mL), and brine (2×200 mL) sequentially. The organic layer was dried over sodium sulfate, filtered then concentrated under vacuum to light yellow oil. The crude oil was diluted with heptane (140 mL), then filtered through 220 g silica gel (230-400 mesh). The silica gel pad was rinsed with 1 L of heptane then with IL 10% EtOAc-90% heptane. The combined filtrate was concentrated under vacuum to give of a scalemic mixture enriched in (1S,2R)-ethyl 4-(2-tert-butoxy-2-oxoethylidene)-2-ethylcyclopentanecarboxylate as a mixture of cis and trans diastereoisomers with cis as a major (100 g, 100%) as an oil. ¹H NMR (major cis diastereomer, 400 MHz, CDCl₃) δ 5.72-5.70 (m, 1H), 4.18-4.07 (m, 2H), 3.18-2.46 (m, 6H), 2.33-2.05 (m, 1H), 1.51-1.39 (m, 10H), 1.30-1.21 (m, 3H), 0.99-0.81 (m, 3H).

### Step B: Ethyl 4-(2-butoxy-2-oxoethyl)-2-ethylcyclopentanecarboxylate

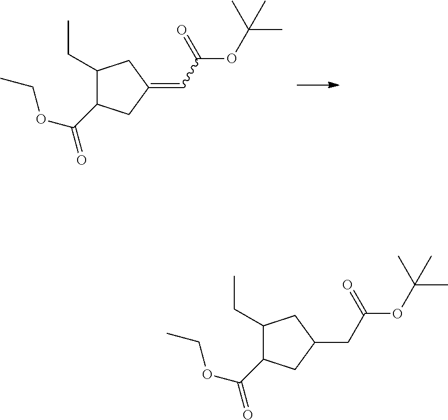

To a solution of scalemic mixture enriched in (1S,2R)-ethyl 4-(2-tert-butoxy-2-oxoethylidene)-2-ethylcyclopentanecarboxylate as a mixture of cis and trans diastereoisomers (92 g, 326 mmol) in heptane (280 mL) was added under nitrogen 5% Pd/Al₂O₃ (2.8 g, 330 mmol). The mixture was purged with nitrogen, hydrogen and hydrogen pressure set to about 40 psi. The mixture was agitated for about 16 h at rt. The reaction mixture was filtered through a filter and rinsed with heptane (300 mL) resulting in a colorless solution. The solution was concentrated under vacuum to give a scalemic mixture enriched in (1S,2R,4S)-ethyl 4-(2-butoxy-2-oxoethyl)-2-ethylcyclopentanecarboxylate (90.6 g, 97.4%) as a colorless oil, ¹H NMR (400 MHz, CDCl₃) δ 4.16-4.05 (m, 2H), 2.87 (m, 1H), 2.37-2.18 (m, 4H), 2.16-1.94 (m, 2H), 1.68-1.47 (m, 1H), 1.45-1.38 (m, 10H), 1.28-1.24 (m, 3H), 1.22-1.05 (m, 2H), 0.91-0.86 (m, 3H).

### Step C: 2-(3-(Ethoxycarbonyl)-4-ethylcyclopentyl)acetic acid

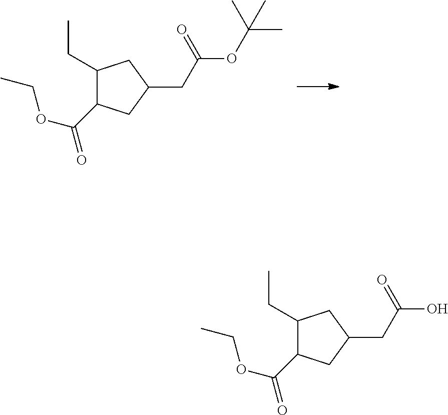

A solution of a scalemic mixture enriched in (1S,2R,4S)-ethyl 4-(2-butoxy-2-oxoethyl)-2-ethylcyclopentanecarboxylate (86.3 g, 303 mmol) in heptane (180 mL) was cooled to about 15° C. TFA (98 mL, 1275 mmol) was added. The solution was stirred at rt for about 1 h. Additional TFA (23.38 mL, 303 mmol) was added and the reaction mixture stirred at rt overnight. The reaction solution was concentrated under vacuum then diluted with heptane (200 mL). 20% aqueous Na₂CO₃ (395 mL) was slowly added with mixing. The organic layer was mixed with heptane (350 mL) and 85% aqueous phosphoric acid (107 g). The organic layer was washed with water:brine (22:3, 3×75 mL), then dried over sodium sulfate, filtered and concentrated under vacuum to give a scalemic mixture enriched in 2-((1S,3S,4R)-3-(ethoxycarbonyl)-4-ethylcyclopentyl)acetic acid (67 g, 97%) as an oil. ¹H NMR (400 MHz, CDCl₃) δ 4.16-4.05 (m, 2H), 2.94-2.86 (m, 1H), 2.55-2.01 (m, 6H), 1.68-1.35 (m, 2H), 1.30-1.11 (m, 5H), 0.99-0.81 (m, 3H).

### Step D: Ethyl 2-ethyl-4-(2-hydroxyethyl)cyclopentanearboxylate

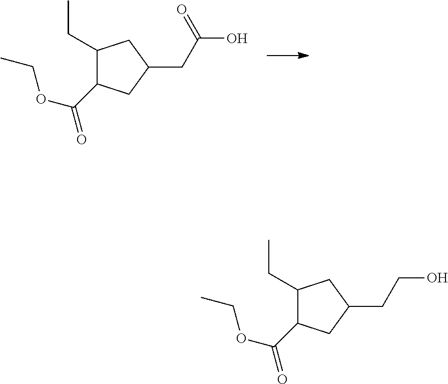 [SMILES: CCOC(=O)C1CC(CC(=O)O)CC1CC ; CCOC(=O)C1CC(CCO)CC1CC]

A solution of scalemic mixture enriched in 2-((1S,3S,4R)-3-(ethoxycarbonyl)-4-ethylcyclopentyl)acetic acid (38 g, 166 mmol) in THF (220 mL) was cooled to about −7° C. BH₃.THF complex (1M, 200 mL, 200 mmol) was added over about 32 min at less than about 5° C. The solution was stirred at about 5° C. overnight then cooled to about −1.5° C. followed by the addition of MeOH (120 mL). The solution was concentrated under vacuum. The residue was chased twice with MeOH (50 mL), then with heptane (60 mL) to give a scalemic mixture enriched in (1S,2R,4S)-ethyl 2-ethyl-4-(2-hydroxyethyl)cyclopentanecarboxylate (32.1 g, 90%) as slightly yellow oil. ¹H NMR (400 MHz, CDCl₃) δ 4.17-4.05 (m, 2H), 3.67 (t, 2H), 2.95-2.82 (m, 1H), 2.14-1.92 (m, 4H), 1.71 (q, 2H), 1.66-1.40 (m, 3H), 1.26 (t, 3H), 1.21-1.03 (m, 2H), 0.92-0.86 (m, 3H).

### Step E: Ethyl 2-ethyl-4-(2-(methylsulfonyloxy)ethyl)cyclopentanecarboxylate

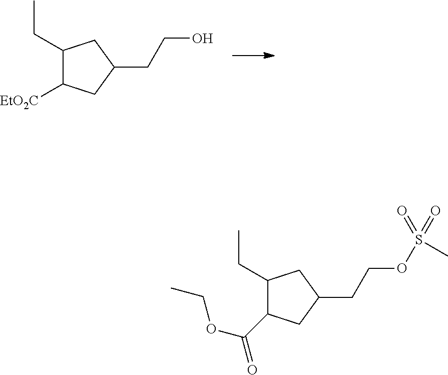 [SMILES: CCOC(=O)C1CC(CCO)CC1CC ; CCOC(=O)C1CC(CCOS(C)(=O)=O)CC1CC]

A solution of a scalemic mixture enriched in (1S,2R,4S)-ethyl 2-ethyl-4-(2-hydroxyethyl)cyclopentanecarboxylate (10.6 g, 49.5 mmol) DCM (60 mL) was cooled to about −6° C. Methanesulfonyl chloride (8.50 g, 74.2 mmol) in DCM (12 mL) was added drop wise at about 2° C. over about 25 min. The slurry was mixed at about −2° C. for about 3 h. To the yellow slurry was added heptane (60 mL) and phosphoric acid (11.41 g, 99 mmol) in water (30 mL) at about 10° C. The organic layer was washed with 15% brine (4×30 mL). The organic layer was dried over sodium sulfate then filtered and concentrated under vacuum. The residue was chased with heptanes (40 mL) to give a scalemic mixture enriched in (1S,2R,4S)-ethyl 2-ethyl-4-(2-(methylsulfonyloxy)ethyl)cyclopentanecarboxylate (14 g, 97%) as an oil. ¹H NMR (400 MHz, CDCl₃) δ 4.24 (t, 2H), 4.17-4.05 (m, 2H), 3.00 (s, 3H), 2.91-2.82 (m, 1H), 2.15-1.95 (m, 4H), 1.9 (q, 2H), 1.67-1.58 (m, 1H), 1.51-1.4 (m, 1H), 1.27 (t, 3H), 1.22-1.03 (m, 2H), 0.9 (t, 3H).

### Step F: ethyl 4-(2-cyanoethyl)-2-ethylcyclopentanecarboxylate

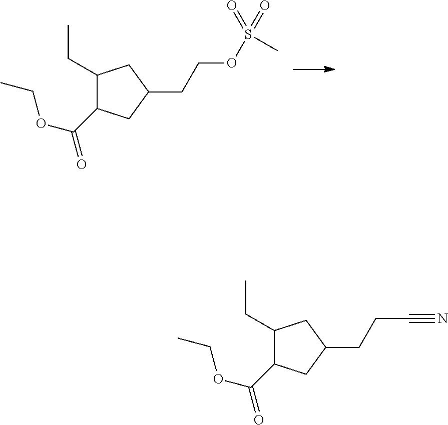

A suspension of scalemic mixture enriched in (1S,2R,4S)-ethyl 2-ethyl-4-(2-(methylsulfonyloxy)ethyl)cyclopentanecarboxylate (9.2 g, 31.5 mmol) and sodium cyanide (7.71 g, 157 mmol) in 1,3-dimethyl-2-imidazolinone (80 mL) was stirred at about 35° C. for about 20 h then cooled to about 5° C. and water (60 mL) was added. The solution was extracted with heptane (150 mL) then extracted with heptane (50 mL). The combined organic layers were washed with water (2×25 mL), dried over sodium sulfate, filtered and concentrated under vacuum to give an oil that was purified on silica gel eluting with 18% EtOAc-82% heptane to give a scalemic mixture enriched in (1S,2R,4S)-ethyl 4-(2-cyanoethyl)-2-ethylcyclopentanecarboxylate (6.3 g, 70%) oil. ¹H NMR (400 MHz, CDCl₃) δ 4.15-4.05 (m, 2H), 2.93-2.85 (m, 1H), 2.36 (t, 2H), 2.15-1.95 (m, 4H), 1.8 (q, 2H), 1.65-1.57 (m, 1H), 1.51-1.4 (m, 1H), 1.26 (t, 3H), 1.29-1.03 (m, 2H), 0.91 (t, 3H).

### Step G: (1S,2R,4S)-4-(2-cyanoethyl)-2-ethylcyclopentanecarboxylic acid

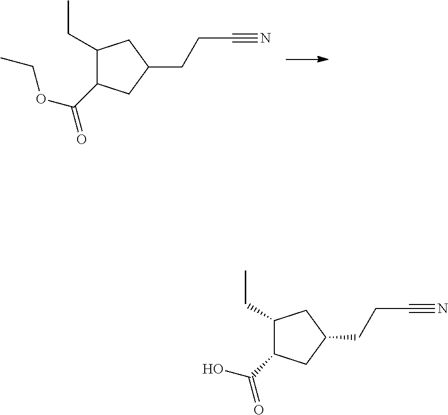 [SMILES: CCOC(=O)C1CC(CCC#N)CC1CC ; CC[C@@H]1C[C@H](CCC#N)C[C@@H]1C(=O)O]

A solution of a scalemic mixture enriched in (1S,2R,4S)-ethyl 4-(2-cyanoethyl)-2-ethylcyclopentanecarboxylate (5.00 g, 22.4 mmol) in MeOH (40 mL) was cooled to about −2° C. To the solution was slowly added aqueous 50% NaOH (10.75 g, 134 mmol). The mixture was stirred at about 5° C. for about 4 h, at about 8° C. for about 14 h, then at about 11° C. for about 25 h. The mixture was washed with 30 mL MTBE. The aqueous layer was cooled to about 0° C. followed by the addition of phosphoric acid (68.0 g, 139 mmol). The mixture was extracted with MTBE. The organic layer was washed with water (20 mL) then with brine (20 mL). The organic layer was dried over sodium sulfate then filtered and concentrated under vacuum to give 3.7 g of a scalemic mixture enriched in (1S,2R,4S)-4-(2-cyanoethyl)-2-ethylcyclopentanecarboxylic acid as a colorless oil.

This oil (2.1 g, 10.75 mmol) was dissolved in acetonitrile (22 mL) at about 65° C. and to this solution, a solution of (S)-1-phenylethanamine (1.303 g, 10.75 mmol) in acetonitrile (8 mL) was added. The solution was then cooled to rt over about 2 h. The solids formed were filtered and rinsed with acetonitrile (15 mL). The wet solid, was mixed with aqueous 10% H₃PO₄ (10 mL) and isopropyl acetate (20 mL). The organic layer was washed with brine (3×10 mL), dried over sodium sulfate, filtered and then concentrated under vacuum to give (1S,2R,4S)-4-(2-cyanoethyl)-2-ethylcyclopentanecarboxylic acid (1.1 g, 52%) as a colorless oil. ¹H NMR (400 MHz, CDCl₃) δ 2.98-2.88 (m, 1H), 2.36 (t, 2H), 2.16-1.98 (m, 4H), 1.82-1.76 (m, 2H), 1.65-1.50 (m, 2H), 1.33-1.24 (m, 1H), 1.15-1.07 (m, 1H), 0.95-0.92 (t, 3H).

### Step H: (1S,2R,4S)-4-(2-cyanoethyl)-2-ethyl-N′-(5-tosyl-5H-pyrrolo[2,3-b]pyrazin-2-yl)cyclopentanecarbohydrazide

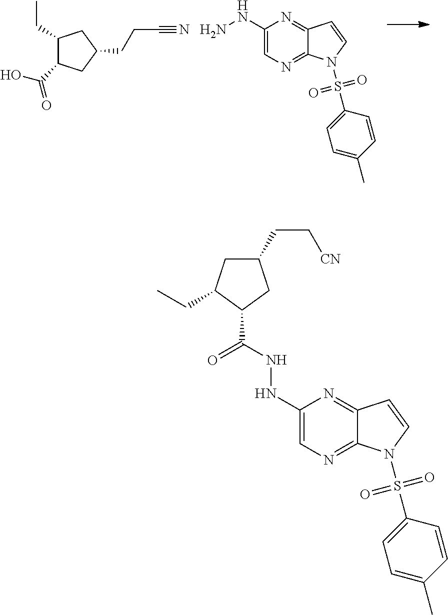 [SMILES: CC[C@@H]1C[C@H](CCC#N)C[C@@H]1C(=O)O ; Cc1ccc(S(=O)(=O)n2ccc3nc(NN)cnc32)cc1 ; CC[C@@H]1C[C@H](CCC#N)C[C@@H]1C(=O)NNc1cnc2c(ccn2S(=O)(=O)c2ccc(C)cc2)n1]

To a solution of (1S,2R,4S)-4-(2-cyanoethyl)-2-ethylcyclopentanecarboxylic acid (1 g, 5.12 mmol) in THF (5 mL) at about 0° C. was added 2-hydrazinyl-5-tosyl-5H-pyrrolo[2,3-b]pyrazine (1.55 g, 5.12 mmol, WO2011/068881), HATU (2.34 g, 6.15 mmol) and TEA (1.428 mL, 10.24 mmol). The mixture was stirred at about 5° C.-10° for about 1 h. The mixture was extracted with isopropyl acetate (25 mL). The organic layer was washed with brine (3×15 mL) and then concentrated under vacuum. The residue was chased with 20 mL×2 of isopropyl acetate and then dissolved in isopropyl acetate (30 mL). The solution was filtered through 3 g of silica gel pad and rinsed with isopropyl acetate (30 mL). The filtrate/rinse was concentrated under vacuum to yellow foam. The crude product was dissolved in isopropyl acetate (7 mL) at reflux then cooled to rt and stirred overnight. The product solids were filtered and rinsed with isopropyl acetate, then with heptanes. The solid was dried under vacuum at about 50° C. to afford (1S,2R,4S)-4-(2-cyanoethyl)-2-ethyl-N′-(5-tosyl-5H-pyrrolo[2,3-b]pyrazin-2-yl)cyclopentanecarbohydrazide (2.0 g, 81%) as a yellow solid. ¹H NMR (400 MHz, DMSO) δ 9.78 (d, 1H), 8.76 (d, 1H), 7.97 (d, 1H), 7.94-7.88 (m, 2H), 7.78 (s, 1H), 7.42-7.36 (m, 2H), 6.63 (d, 1H), 2.84-2.75 (m, 1H), 2.33 (s, 3H), 2.04-1.78 (m, 4H), 1.64-1.36 (m, 4H), 1.28-0.95 (m, 2H), 0.89 (t, 3H).

### Step I: 3-((1S,3R,4S)-3-ethyl-4-(6-tosyl-6H-pyrrolo[2,3-e][1,2,4]triazolo[4,3-a]pyrazin-1-yl)cyclopentyl)propanenitrile

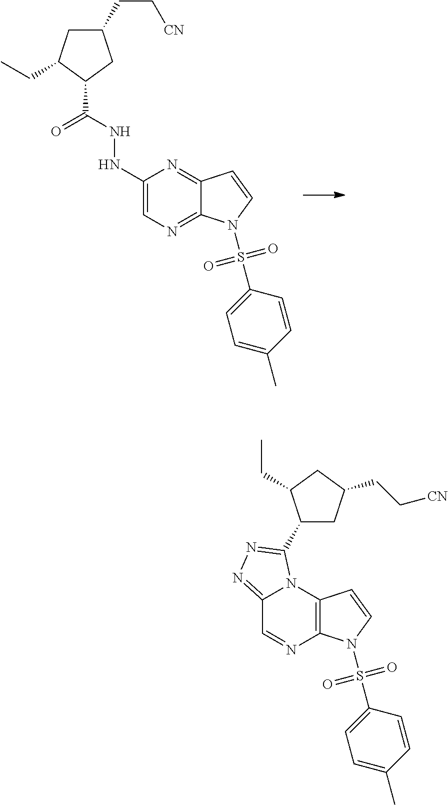 [SMILES: CC[C@@H]1C[C@H](CCC#N)C[C@@H]1C(=O)NNc1cnc2c(ccn2S(=O)(=O)c2ccc(C)cc2)n1 ; CC[C@@H]1C[C@H](CCC#N)C[C@@H]1c1nnc2cnc3c(ccn3S(=O)(=O)c3ccc(C)cc3)n12]

To a solution of (1S,2R,4S)-4-(2-cyanoethyl)-2-ethyl-N′-(5-tosyl-5H-pyrrolo[2,3-b]pyrazin-2-yl)cyclopentanecarbohydrazide (2.00 g, 4.16 mmol) in THF (35 mL) cooled in ice-bath were added TEA (2.32 mL, 16.6 mmol) and thionyl chloride (0.456 mL, 6.24 mmol). The mixture was stirred at rt for about 30 min then at reflux for about 3 h. The reaction mixture was cooled to about 5° C. followed by the addition of water (20 mL). The mixture was extracted with isopropyl acetate (25 mL). The organic layer was washed with brine (15 mL). 25% aqueous NaH₂PO₄ (15 mL) and brine (2×15 mL) sequentially. The organic layer was decolorized with charcoal (0.2 g), dried over sodium sulfate, filtered then concentrated under vacuum. The crude solid was chased with THF-heptane (8:2, 15 mL) and was dried under vacuum at about 50° C., to give 3-((1S,3R,4S)-3-ethyl-4-(6-tosyl-6H-pyrrolo[2,3-e][1,2,4]triazolo[4,3-a]pyrazin-1-yl)cyclopentyl)propanenitrile (1.8 g, 93%) as a yellow solid. ¹H NMR (400 MHz, DMSO) δ 8.06-8.00 (m, 2H), 7.46 (s, 1H), 7.46-7.42 (m, 2H), 7.43 (d, 2H), 4.10 (t, 1H), 365-3.50 (m, 2H), 245-2.22 (m, 4H), 2.20-2.04 (m, 2H), 1.97-1.71 (m, 4H), 1.26-0.99 (m, 2H), 0.96-0.75 (m, 1H), 0.77-0.50 (m, 4H).

### Step J: 3-((1S,3R,4S)-3-ethyl-4-(6H-pyrrolo[2,3-e][1,2,4]triazolo[4,3-a]pyrazin-1-yl)cyclopentyl)propanenitrile

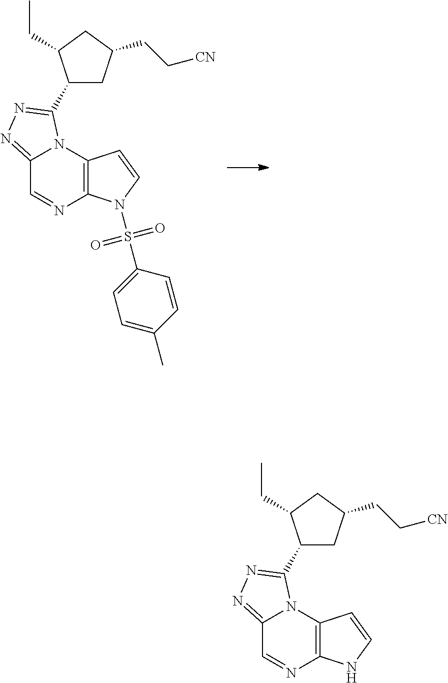 [SMILES: CC[C@@H]1C[C@H](CCC#N)C[C@@H]1c1nnc2cnc3c(ccn3S(=O)(=O)c3ccc(C)cc3)n12 ; CC[C@@H]1C[C@H](CCC#N)C[C@@H]1c1nnc2cnc3[nH]ccc3n12]

To a solution of 3-((1S,3R,4S)-3-ethyl-4-(6-tosyl-6H-pyrrolo[2,3-e][1,2,4]triazolo[4,3-a]pyrazin-1-yl)cyclopentyl)propanenitrile (1.80 g, 3.89 mmol) in THF (15 mL) was added 50% aqueous NaOH (1.245 g, 15.57 mmol) and water (8 mL). The mixture was stirred at rt for about 2 h, at about 30° C. for about 1 h, then at about 35° C. for about 3 h. The reaction mixture was cooled to rt followed by the addition of 85% aqueous phosphoric acid (11.4 g, 11.67 mmol). The mixture was extracted with isopropyl acetate (20 mL). The organic layer was washed with brine (15 mL), 8% aqueous NaHCO₃ (15 mL) and brine (2×15 mL), sequentially. The organic layer was concentrated under vacuum to slurry then cooled to rt. The solids were filtered and rinsed with isopropyl acetate then with water and heptane, sequentially. The solid was dried under vacuum at about 55° C. to give 3-((1S,3R,4S)-3-ethyl-4-(6H-pyrrolo[2,3-e][1,2,4]triazolo[4,3-a]pyrazin-1-yl)cyclopentyl)propanenitrile (0.8 g, 66%) as an off-white solid. ¹H NMR (400 MHz, DMSO) δ 12.5 (br, 1H), 8.82 (s, 1H), 7.48 (d, 1H), 7.00 (d, 1H), 4.12 (t, 1H), 2.58 (t, 2H), 2.43-2.28 (m, 2H), 2.20-2.09 (m, 3H), 1.79 (q, 2H), 1.21-1.10 (m, 1H), 0.94-0.73 (m, 1H), 0.62-0.52 (m, 4H). Jak3 IC₅₀=B

### Example #23

### 3-((1R,3R,4S)-3-ethyl-4-(6H-pyrrolo[2,3-e][1,2,4]triazolo[4,3-a]pyrazin-1-yl)cyclopentyl)propanenitrile

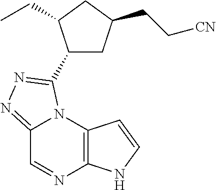 [SMILES: CC[C@@H]1C[C@@H](CCC#N)C[C@@H]1c1nnc2cnc3[nH]ccc3n12]

### Step A: Ethyl 2-ethyl-4-oxocyclopentanecarboxylate

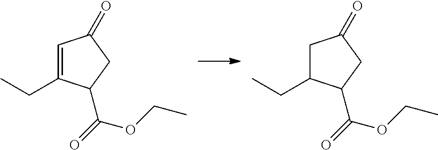

To a suspension of Hantzsch ester (500 g, 1980 mol), (2R,5R)-5-benzyl-3-methyl-2-(5-methylfuran-2-yl)imidazolidin-4-one (22.5 g, 82 mmol) and trichloroacetic acid (13.5 g, 82 mmol) in toluene (600 mL) was added ethyl 2-ethyl-4-oxocyclopent-2-enecarboxylate (300 g, 1650 mmol, WO2011/068881) and the resulting mixture was stirred at rt for about 7 days. Upon reaction completion the mixture was concentrated by rotary evaporation, the residue dissolved in heptane (2 L) and filtered. The organic layer was washed with 10% aqueous HCl (3×1 L) and the combined aqueous layers back extracted with heptanes (1 L). The organic layers were combined, washed with brine (500 mL), dried over Na₂SO₄, filtered and concentrated by rotary evaporation. The resulting residue was purified by fractional distillation (80-85° C., 0.3 torr) to provide a scalemic mixture enriched in (1S,2R)-ethyl 2-ethyl-4-oxocyclopentanecarboxylate (260 g, 86%) as a colorless oil. ¹H NMR (400 MHz, CDCl₃) δ 4.16 (m, 2H), 3.21 (ddd, 1H), 2.60-2.51 (m, 1H), 2.44-2.29 (m, 3H), 2.27-2.14 (m, 1H), 1.57-1.45 (m, 1H), 1.41-1.32 (m, 1H), 1.27 (t, 3H), 0.98 (t, 3H).

### Step B: Ethyl 2-ethyl-4-hydroxycyclopentanecarboxylate

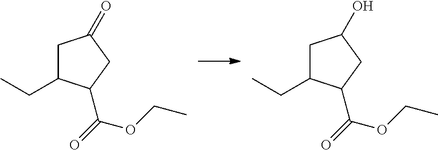

To a solution of a scalemic mixture enriched in (1S,2R)-ethyl 2-ethyl-4-oxocyclopentanecarboxylate (50 g, 271 mmol) in ethanol (250 mL) at about 5 was added NaBH₄ (5.1 g, 136 mmol) portionwise over about 30 min. The reaction was allowed to stir at about 5° C. for about 1 h. Upon completion, the reaction mixture was poured into saturated aqueous NH₄Cl (250 mL) and ethanol was removed by rotary evaporation. The product was extracted into DCM (2×250 mL). The combined organic layers were washed with brine (200 mL), dried over Na₂SO₄, filtered and concentrated by rotary evaporation to provide a scalemic mixture enriched in (1S,2R,4S)-ethyl 2-ethyl-4-hydroxycclopentanecarboxylate (50.5 g, 100%) as an oil. ¹H NMR (400 MHz, CDCl₃) δ 4.33-4.25 (m, 1H), 4.22-4.08 (m, 2H, 3.61 (d, 1H), 2.87 (m, 1H), 2.44-2.34 (m, 1H), 2.09-1.89 (m, 3H), 1.52-1.31 (m, 3H), 1.29 (t, 3H), 0.94 (t, 3H).

### Step C: 2-(3-(Ethoxycarbonyl)-4-ethylcyclopentyl)acetic acid

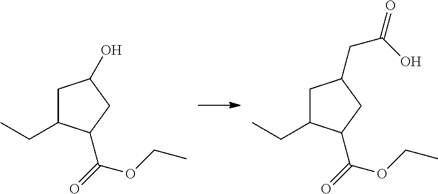 [SMILES: CCOC(=O)C1CC(O)CC1CC ; CCOC(=O)C1CC(CC(=O)O)CC1CC]

To a solution of a scalemic mixture enriched in (1S,2R,4S)-ethyl 2-ethyl-4-hydroxycyclopentanecarboxylate (50.5 g, 271 mmol) and TEA (76 mL, 542 mmol) in DCM (250 mL) at about 5° C. was added methanesulfonyl chloride (31.7 mL, 407 mmol). The resulting mixture was stirred at about 5° C. for about 1 h. Upon completion, cyclohexane (1 L) and water (200 mL) were added and the layers separated. The organic layer was washed with water (200 mL) and brine (200 mL). The organic layer was dried over Na₂SO₄, filtered and concentrated to an oil by rotary evaporation. To a suspension of NaOt-Bu (39.1 g, 407 mmol) in THF (200 mL) at about 5° C. was added di-tert-butyl malonate (91 mL, 407 mmol). After 30 min, the oil from above in THF (50 mL) was added, the mixture warmed to about 50° C. and the reaction was stirred overnight. Upon completion, the mixture was cooled to rt and partitioned between saturated aqueous NH₄Cl (200 mL) and cyclohexane (1 L). The organic layer was washed with saturated aqueous NH₄Cl (200 mL). The combined aqueous layers were back extracted with cyclohexane (500 mL) and the combined organic layers washed with water (200 mL) and brine (200 mL). The organic layer was dried over Na₂SO₄, filtered and concentrated to an oil. The oil from above was treated with TFA (300 mL) at about 50° C. for about 18 h. Upon completion, the TFA was removed by rotary evaporation and the residue taken up in xylenes (500 mL). After concentrating to approximately half of the original volume, the reaction was heated to about 130° C. and stirred for about 18 h. Upon completion, the reaction was cooled and the product was extracted into aqueous 10% K₂CO₃ (3×300 mL). The combined aqueous extracts were washed with xylenes (200 mL) and then the pH was adjusted to about 3.5 by the addition of solid citric acid. The product was extracted into MTBE (2×300 mL). The combined MTBE extracts were washed with brine, dried over Na₂SO₄, filtered and concentrated by rotary evaporation to provide a scalemic mixture enriched in 2-((1R,4R)-3-(ethoxycarbonyl)-4-ethylcyclopentyl)acetic acid (55 g, 89%) as an oil. ¹H NMR (major diastereomer, 400 MHz, CDCl₃) δ 7.79 (hr, 1H), 4.17-4.05 (m, 2H), 2.92 (m, 1H), 2.78-2.64 (m, 1H), 2.36 (d, 2H), 2.24 (m, 1H), 2.19-2.06 (m, 2H), 1.80 (m, 1H), 1.56-1.44 (m, 2H), 1.42-1.30 (m, 1H), 1.25 (t, 3H), 0.90 (t, 3H).

### Step D: Ethyl 2-ethyl-4-(2-hydroxyethyl)cyclopentanecarboxylate

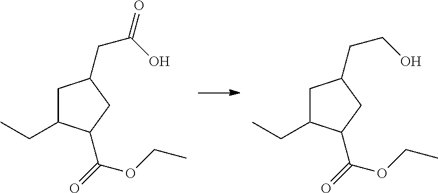 [SMILES: CCOC(=O)C1CC(CC(=O)O)CC1CC ; CCOC(=O)C1CC(CCO)CC1CC]

To a solution of a scalemic mixture enriched in ((1R,3S,4R)-3-(ethoxycarbonyl)-4-ethylcyclopentyl)acetic acid (54 g, 237 mmol) in THF (300 mL) at about 5° C. was added BH₃.THF complex (1M, 284 mL, 284 mmol). The resulting mixture was stirred for about 18 h while allowing it to warm to rt. An additional portion of BH₃.THF complex (1M, 50 mL, 50 mmol) was added and stirred at rt for about additional 20 h. Upon completion, the reaction was quenched with MeOH (500 mL) and concentrated by rotary evaporation. Methanol (500 mL) was added and then removed by rotary evaporation twice to afford the a scalemic mixture enriched in (1S,2R,4R)-ethyl 2-ethyl-4-(2-hydroxyethyl)cyclopentanecarboxylate (48.1 g, 95%). ¹H NMR (400 MHz, CDCl₃) δ 4.14-4.04 (m, 2H), 3.68-3.59 (m, 3H), 2.97-2.83 (m, 1H), 2.39-2.28 (m, 1H), 2.21-2.05 (m, 3H), 1.78-1.31 (m, 6H), 1.24 (d, 3H), 0.88 (m, 3H).

### Step E: ethyl 4-(2-cyanoethyl)-2-ethylcyclopentanecarboxylate

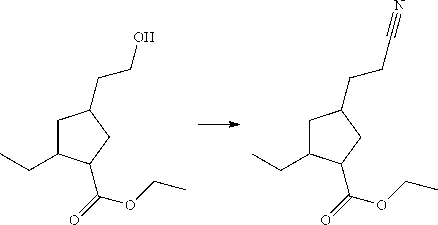 [SMILES: CCOC(=O)C1CC(CCO)CC1CC ; CCOC(=O)C1CC(CCC#N)CC1CC]

To a solution of a scalemic mixture enriched in (1S,2R,4R)-ethyl 2-ethyl-4-(2-hydroxyethyl)cyclopentanecarboxylate (48.1 g, 224 mmol) and TEA (62.6 mL, 449 mmol) in DCM (250 mL) at about 5° C. was added methanesulfonyl chloride (26.2 mL, 337 mmol). The resulting mixture was stirred for about 1 h. Upon completion, cyclohexane (1 L) and water (200 mL) were added and the layers separated. The organic layer was washed with water (200 mL) and brine (200 mL). The organic layer was dried over Na₂SO₄, filtered and concentrated to an oil by rotary evaporation. The oil was taken up in acetonitrile (300 mL) and a solution of NaCN (32.9 g, 672 mmol) in water (200 mL) was added. The resulting mixture was heated to reflux and stirred for about 18 h. Upon completion, cyclohexane (1 L) and water (700 mL) were added and the layers separated. The organic layer was washed with saturated aqueous NaHCO₃ (2×200 mL), water (200 mL) and brine (200 mL). The organic layer was dried over Na₂SO₄, filtered and concentrated to an oil by rotary evaporation. The product was purified by flash chromatography on silica gel (250 g) eluting with 20% EtOAc in heptane to afford a scalemic mixture enriched in (1S,2R,4R)-ethyl 4-(2-cyanoethyl)-2-ethylcyclopentanecarboxylate (38.3 g, 77%) as an oil. ¹H NMR (400 MHz, CDCl₃) δ 4.17-4.05 (m, 2H), 2.90 (m, 1H), 2.44-2.28 (m, 3H), 2.18 (ddd, 1H), 2.15-2.03 (m, 1H), 1.83-1.63 (m, 3H), 1.48-1.31 (m, 2H), 1.25 (t, 5H), 0.91 (t, 3H).

### Step F: (1S,2R,4R)-4-(2-cyanomethyl)-2-ethylcyclopentanecarboxylate, S-(−)-1-phenylethylammonium salt

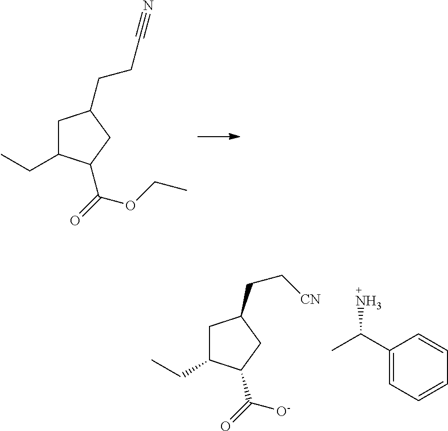

A solution of Ti(Oi-Pr)₄ (51 mL, 172 mmol) in allyl acetate (185 mL) was refluxed for about 18 h. After cooling to rt, the solvent was removed on the rotary evaporator. Additional allyl acetate (185 mL) was added and the resulting mixture refluxed for about 6 h, cooled to rt and the solvent removed on the rotary evaporator. To the residue of resulting Ti(Oallyl)₄ was added a solution of a scalemic mixture enriched in (1S,2R,4R)-ethyl 4-(2-cyanoethyl)-2-ethylcyclopentanecarboxylate (38.3 g, 172 mmol) in allyl alcohol (400 mL) and the resulting mixture heated to reflux for about 18 h. After cooling to rt, the solvent was removed on the rotary evaporator and the residue treated by refluxing in allyl alcohol for about 18 h. Upon completion, the mixture was cooled to rt and the solvent removed on the rotary evaporator. The residue was taken up in toluene (500 mL) and washed with 10% aqueous HCl (3×400 mL). The combined aqueous layers were back extracted with toluene (200 mL). The combined organic layers were washed with brine (200 mL), dried over Na₂SO₄, filtered and concentrated on the rotary evaporator. The residue was dissolved in toluene (400 mL) and solution of Pd₂(dba)₃ (3.15 g, 3.4 mmol) and triphenylphosphine (7.22 g, 27.5 mmol) in THF (50 mL) was added. Pyrrolidine (43 mL, 520 mmol) was added and the reaction mixture was stirred at rt for about 2 h. Upon completion, the product was extracted into 10% aqueous K₂CO₃ (3×400 mL). The combined aqueous layers were washed with toluene (200 mL) and the pH of the aqueous layer adjusted to about 3 with solid citric acid. The product was extracted into MTBE (2×500 mL), the combined organic layers were washed with brine (200 mL), dried over Na₂SO₄, filtered and concentrated on the rotary evaporator. The residue was taken up in acetonitrile (150 mL), S-(−)-1-phenylethylamine (18.5 mL, 143 mmol) was added, the mixture warmed to about 60° C. and allowed to cool to rt. After stirring for about 18 h, the solids were collected by filtration, washed with acetonitrile (20 mL) and dried in a vacuum oven to afford (1S,2R,4R)-4-(2-cyanoethyl)-2-ethylcyclopentanecarboxylate S-(−)-1-phenylethylammonium salt (35.8 g, 66%) as a white crystalline solid. ¹H NMR (400 MHz, CD₃OD) δ 7.47-7.30 (m, 5H), 4.37 (q, 1H), 2.80 (dd, 1H), 2.39 (dd, 2H), 2.32-2.22 (m, 1H), 2.19-2.00 (m, 3H), 1.85-1.73 (m, 1H), 1.66-1.56 (m, 5H), 1.55-1.45 (m, 1H), 1.41-1.29 (m, 2H), 1.26-1.14 (m, 1H), 0.90 (t, 3H).

### Step G: 3-((1R,3R,4S)-3-ethyl-4-(6-tosyl-6H-pyrrolo[2,3-e][1,2,4]triazolo[4,3-a]pyrazin-1-yl)cyclopentyl)propanenitrile

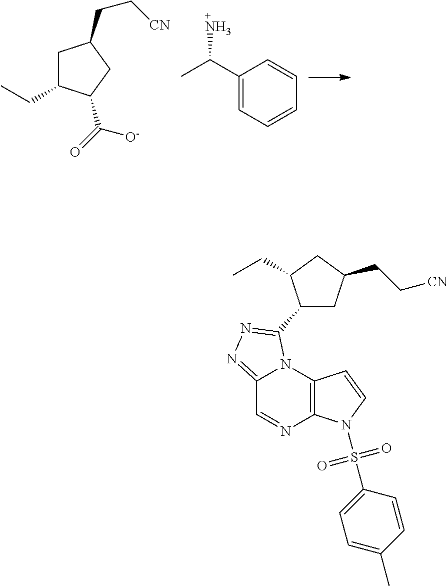

To the a suspension of (1S,2R,4R)-4-(2-cyanoethyl)-2-ethylcyclopentanecarboxylate S-(−)-1-phenylethylammonium salt (10.0 g, 31.6 mmol) in MTBE (100 mL) was added 30% aqueous citric acid (100 mL) and the mixture stirred until the solids dissolved. The layers were separated and the organic layer washed with 30% aqueous citric acid (100 mL). The aqueous layers were extracted with MTBE (100 mL). The combined organic layers were washed with brine (50 mL), dried over Na₂SO₄, filtered and concentrated on the rotary evaporator. The residue was dissolved in THF (100 mL); HATU (13.8 g, 36.3 mmol) and TEA (8.8 mL) were added. After stirring for about 30 min, 2-hydrazinyl-5-tosyl-5H-pyrrolo[2,3-b]pyrazine (9.6 g, 32 mmol, WO2011/068881) was added and the resulting mixture stirred at it for about 2 h. Upon completion, water (100 mL) and EtOAc (200 mL) were added and the mixture stirred for about 5 min. The layers were separated and the organic layer washed with water (100 mL). The combined aqueous layers were extracted with EtOAc (100 mL). The combined organic layers were washed with brine (50 mL), filtered through a short plug of silica gel and concentrated on the rotary evaporator. The residue was dissolved in THF (150 mL), TEA (17.6 mL, 126 mmol) and thionyl chloride (4.6 mL, 63 mmol) were added and the mixture warmed to about 65° C. Upon completion after about 3 h, the mixture was cooled to rt, quenched with 10% aqueous KH₂PO₄ (100 mL). The product was extracted with 2-MeTHF (150 mL). The organic layer was washed with brine and concentrated by rotary evaporation. The residue was crystallized from EtOAc (75 mL), the solids were collected by filtration and washed with EtOAc (20 mL) to afford 3-((1R,3R,4S)-3-ethyl-4-(6-tosyl-6H-pyrrolo[2,3-e][1,2,4]triazolo[4,3-a]pyrazin-1-yl)cyclopentyl)propanenitrile (6.5 g, 45%) as a white crystalline solid. ¹H NMR (400 MHz, DMSO) δ 9.04 (s, 1H), 8.10-7.96 (m, 3H), 7.45 (dd, 4H), 4.11 (dd, 1H), 2.58-2.49 (m, 2H), 2.44-2.31 (m, 6H), 1.81 (ddd, 2H), 1.69 (m, 3H), 0.94-0.70 (m, 2H), 0.57 (t, 3H).

### Step H: 3-((1R,3R,4S)-3-ethyl-4-(6H-pyrrolo[2,3-e][1,2,4]triazolo[4,3-a]pyrazin-1-yl)cyclopentyl)propanenitrile

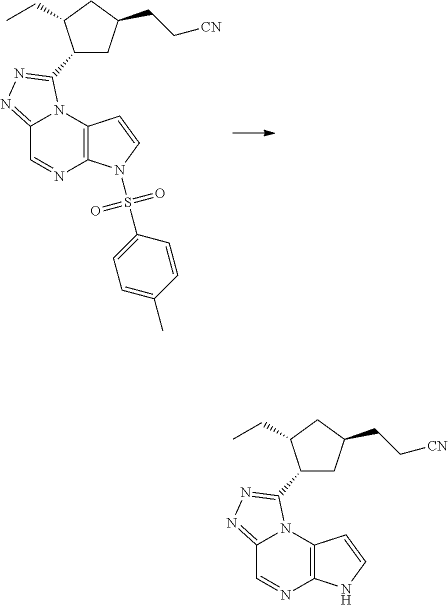 [SMILES: CC[C@@H]1C[C@@H](CCC#N)C[C@@H]1c1nnc2cnc3c(ccn3S(=O)(=O)c3ccc(C)cc3)n12 ; CC[C@@H]1C[C@@H](CCC#N)C[C@@H]1c1nnc2cnc3[nH]ccc3n12]

To a solution of 3-((1R,3R,4S)-3-ethyl-4-(6-tosyl-6H-pyrrolo[2,3-e][1,2,4]triazolo[4,3-a]pyrazin-1-yl)cyclopentyl)propanenitrile (1.0 g, 22 mmol) in THF (5 mL) was added 10% aqueous NaOH (5 g, 12.5 mmol) and the resulting mixture was warmed to about 45° C. After about 18 h, the reaction mixture was cooled to rt and the layers separated. The organic layer was washed with 10% aqueous NaOH (5 mL) and brine (5 mL). The product was crystallized by the addition of water (15 mL). The solids were collected, washed with 20% THF in water (1 mL) and water (1 mL). After drying in a vacuum oven at about 50° C. 3-((1R,3R,4S)-3-ethyl-4-(6H-pyrrolo[2,3-e][1,2,4]triazolo[4,3-a]pyrazin-1-yl)cyclopentyl)propanenitrile was obtained (0.55 g, 84%) as a white crystalline solid. ¹H NMR (400 MHz, DMSO) δ 12.66-12.45 (m, 1H), 8.85 (d, 1H), 7.51 (t, 1H), 7.01 (1, 1H), 4.26-4.05 (m, 1H), 2.59 (m, 3H), 2.50-2.38 (m, 2H), 1.94-1.82 (m, 2H), 1.78-1.65 (m, 3H), 1.02-0.89 (m, 1H), 0.88-0.72 (m, 1H), 0.58 (t, 3H). Jak3 IC₅₀=B

### General Procedure A

In a 4 ml, vial was added a carboxylic acid (10 mg, 0.08 mmol), followed by 2-(1H-7-azabenzotriazol-1-yl)-1,1,3,3-tetramethyl uronium hexafluorophosphate methanaminium (30 mg, 0.08 mmol), TEA (27 μL, 0.20 mmol) and (1S,3R,4S)-3-ethyl-4-(6-tosyl-6H-pyrrolo[2,3-e][1,2,4]triazolo[4,3-a]pyrazin-1-yl)cyclopentylamine (27 mg, 0.063 mmol) in dimethyl acetamide (2 mL). This mixture was stirred at about 60° C. overnight. The crude mixture was concentrated to dryness MeOH (0.5 mL) and 1M aqueous NaOH (0.5 mL) was added and allowed to stir at about 60° C. for about 4 h. The reaction was filtered, checked by LC/MS and concentrated to dryness. The residue was then dissolved in DMSO:MeOH (1:1, 1.4 mL) and purified through reverse phase HPLC to afford the target compound.

### Exemplification of General Procedure A

### Example #24

### N-((1S,3R,4S)-3-ethyl-4-(6H-pyrrolo[2,3-e][1,2,4]triazolo[4,3-a]pyrazin-1-yl)cyclopentyl)-2-methyloxazole-4-carboxamide

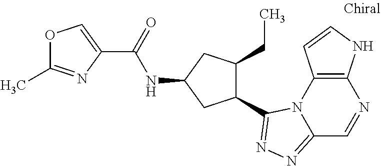 [SMILES: CC[C@@H]1C[C@H](NC(=O)c2coc(C)n2)C[C@@H]1c1nnc2cnc3[nH]ccc3n12]

In a 4 mL vial was added 2-methyloxazole-4-carboxylic acid (10 mg, 0.08 mmol), followed by 2-(1H-7-azabenzotriazol 1-yl)-1,1,3,3-tetramethyl uronium hexafluorophosphate methanaminium (30 mg, 0.08 mmol), TEA (27 μL, 0.20 mmol) and (1S,3R,4S)-3-ethyl)-4-(6-tosyl)-6H-pyrrolo[2,3-e][1,2,4]triazolo[4,3-a]pyrazin-1-yl)cyclopentylamine (27 mg, 0.063 mmol) in dimethyl acetamide (2 mL). This mixture was stirred at 60° C. overnight. The crude mixture was concentrated to dryness, and MeOH (0.5 mL) and 1M aqueous NaOH (0.5 mL) was added and allowed to stir at about 60° C. for about 4 h. The reaction was filtered, checked by LC/MS and concentrated to dryness. The residue was then dissolved in DMSO:MeOH (1:1, 1.4 mL) and purified through reverse phase HPLC (Table 2, Method 10) to afford N-(1S,3R,4S)-3-ethyl-4-(6H-pyrrol[2,3-e][1,2,4]triazolo[4,3-a]pyrazin-1-yl)cyclopentyl)-2-methyloxazole-4-carboxamide. Jak3 IC₅₀=B.

The following examples were prepared using General Procedure A:

TABLE B
The following products were prepared from (1S,3R,4S)-3-ethyl-4-(6-tosyl-6H-
pyrrolo[2,3-e][1,2,4]triazolo[4,3-a]pyrazin-1-yl)cyclopentylamine as detailed in General
Procedure A

| | | | Rₜ | MS | Jak3 |
|---|---|---|---|---|---|
| Ex # | Product | Name | (min) | (M + 1) | IC₅₀ |
| 25 | 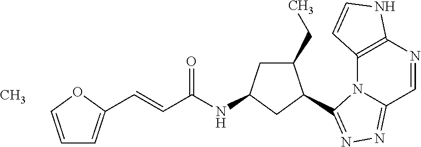 | (E)-N-((1S,3R,4S)-3- ethyl-4-(6H- pyrrolo[2,3- e][1,2,4]triazolo[4,3- a]pyrazin-1- yl)cyclopentyl)-3-(5- methylfuran-2- yl)acrylamide | 0.69 | 405 | B |
| 26 | 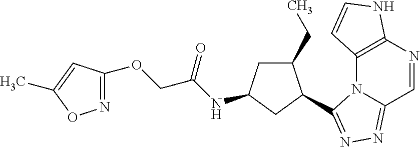 [SMILES: CC[C@@H]1C[C@H](NC(=O)COc2cc(C)on2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-2-(5- methylisoxazol-3- yloxy)acetamide | 0.61 | 410 | C |
| 27 | 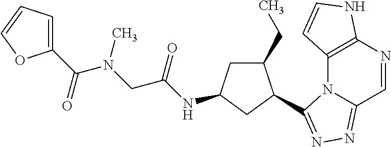 [SMILES: CC[C@@H]1C[C@H](NC(=O)CN(C)C(=O)c2ccco2)C[C@@H]1c1nnc2cnc3[nH]ccc3n12] | N-(2-((1S,3R,4S)-3- ethyl-4-(6H- pyrrolo[2,3- e][1,2,4]triazolo[4,3- a]pyrazin-1- yl)cyclopentylamino)- 2-oxoethyl)-N- methylfuran-2- carboxamide | 0.57 | 436 | C |
| 28 | 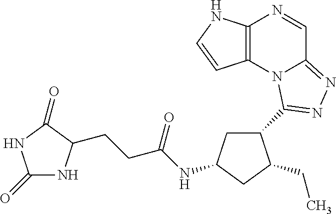 [SMILES: CC[C@@H]1C[C@H](NC(=O)CCC2NC(=O)NC2=O)C[C@@H]1c1nnc2cnc3[nH]ccc3n12] | 3-(2,5- dioxoimidazolidin-4- yl)-N-((1S,3R,4S)-3- ethyl-4-(6H- pyrrolo[2,3- e][1,2,4]triazolo[4,3- a]pyrazin-1- yl)cyclopentyl)pro- panamide | 0.51 | 425 | B |
| 29 | 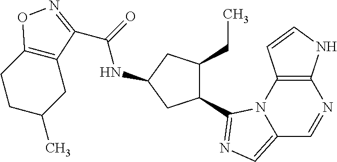 [SMILES: CC[C@@H]1C[C@H](NC(=O)c2noc3c2CC(C)CC3)C[C@@H]1c1ncc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-5- methyl-4,5,6,7- tetrahydrobenzo[d]isoxa- zole-3-carboxamide | 0.75 | 434 | B |
| 30 | 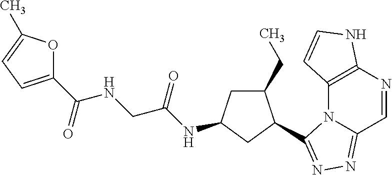 [SMILES: CC[C@@H]1C[C@H](NC(=O)CNC(=O)c2ccc(C)o2)C[C@@H]1c1nnc2cnc3[nH]ccc3n12] | N-(2-((1S,3R,4S)-3- ethyl-4-(6H- pyrrolo[2,3- e][1,2,4]triazolo[4,3- a]pyrazin-1- yl)cyclopentylamino)- 2-oxoethyl)-5- methylfuran-2- carboxamide | 0.59 | 436 | B |
| 31 | 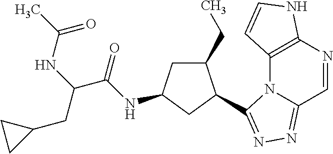 [SMILES: CC[C@@H]1C[C@H](NC(=O)C(CC2CC2)NC(C)=O)C[C@@H]1c1nnc2cnc3[nH]ccc3n12] | 2-acetamido-3- cyclopropyl-N- ((1S,3R,4S)-3-ethyl-4- (6H-pyrrolo[2,3- e][1,2,4]triazolo[4,3- a]pyrazin-1- yl)cyclopentyl)pro- panamide | 0.6 | 424 | C |
| 32 | 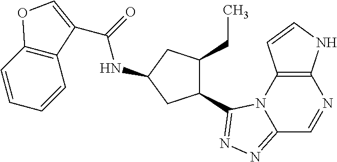 [SMILES: CC[C@@H]1C[C@H](NC(=O)c2coc3ccccc23)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)benzo- furan-3-carboxamide | 0.72 | 415 | B |
| 33 | 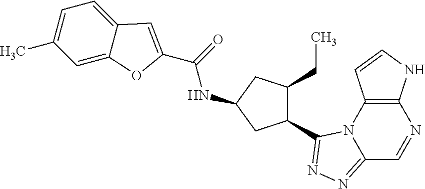 [SMILES: CC[C@@H]1C[C@H](NC(=O)c2cc3ccc(C)cc3o2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-6- methylbenzofuran-2- carboxamide | 0.75 | 429 | B |
| 34 | 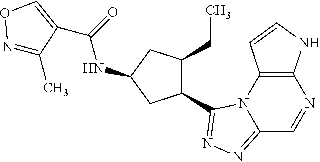 [SMILES: CC[C@@H]1C[C@H](NC(=O)c2conc2C)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3- methylisoxazole-4- carboxamide | 0.61 | 380 | B |
| 35 | 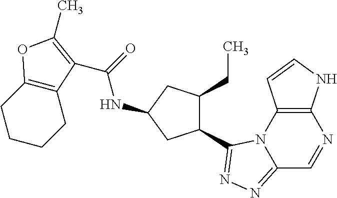 [SMILES: CC[C@@H]1C[C@H](NC(=O)c2c(C)oc3c2CCCC3)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-2- methyl-4,5,6,7- tetrahydrobenzofuran- 3-carboxamide | 0.77 | 433 | B |
| 36 | 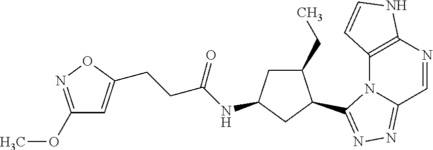 [SMILES: CC[C@@H]1C[C@H](NC(=O)CCc2cc(OC)no2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3-(3- methoxyisoxazol-5- yl)propanamide | 0.6 | 424 | B |
| 37 | 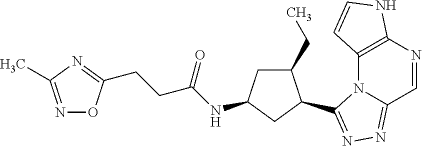 [SMILES: CC[C@@H]1C[C@H](NC(=O)CCc2nc(C)no2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3-(3- methyl-1,2,4- oxadiazol-5- yl)propanamide | 0.57 | 409 | B |
| 38 | 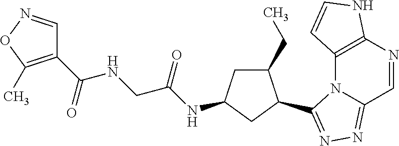 [SMILES: CC[C@@H]1C[C@H](NC(=O)CNC(=O)c2cnoc2C)C[C@@H]1c1nnc2cnc3[nH]ccc3n12] | N-(2-((1S,3R,4S)-3- ethyl-4-(6H- pyrrolo[2,3- e][1,2,4]triazolo[4,3- a]pyrazin-1- yl)cyclopentylamino)- 2-oxoethyl)-5- methylisoxazole-4- carboxamide | 0.61 | 437 | B |
| 39 | 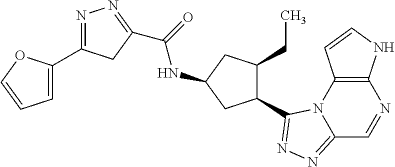 [SMILES: CC[C@@H]1C[C@H](NC(=O)C2=NN=C(c3ccco3)C2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-5- (furan-2-yl)-4H- pyrazole-3- carboxamide | 0.64 | 431 | A |
| 40 | 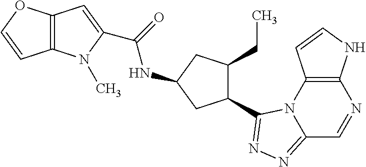 [SMILES: CC[C@@H]1C[C@H](NC(=O)c2cc3occc3n2C)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-4- methyl-4H-furo[3,2- b]pyrrole-5- carboxamide | 0.69 | 418 | Not tested |
| 41 | 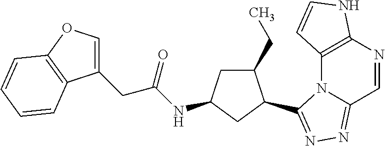 [SMILES: CC[C@@H]1C[C@H](NC(=O)Cc2coc3ccccc23)C[C@@H]1c1nnc2cnc3[nH]ccc3n12] | 2-(benzofuran-3-yl)-N- ((1S,3R,4S)-3-ethyl-4- (6H-pyrrolo[2,3- e][1,2,4]triazolo[4,3- a]pyrazin-1- yl)cyclopentyl)acetamide | 0.69 | 429 | B |
| 42 | 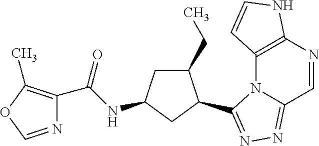 [SMILES: CC[C@@H]1C[C@H](NC(=O)c2ncoc2C)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-5- methyloxazole-4- carboxamide | 0.61 | 380 | B |
| 43 | 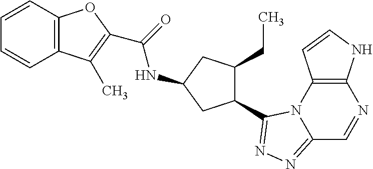 [SMILES: CC[C@@H]1C[C@H](NC(=O)c2oc3ccccc3c2C)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3- methylbenzofuran-2- carboxamide | 0.76 | 429 | B |
| 44 | 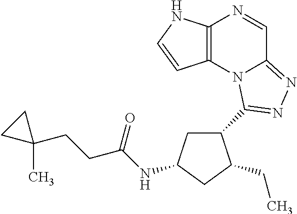 [SMILES: CC[C@@H]1C[C@H](NC(=O)CCC2(C)CC2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3-(1- methylcyclopropyl) propanamide | 0.69 | 381 | B |
| 45 | 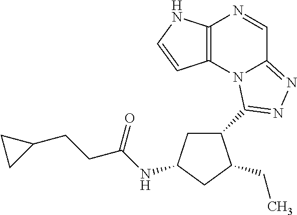 [SMILES: CC[C@@H]1C[C@H](NC(=O)CCC2CC2)C[C@@H]1c1nnc2cnc3[nH]ccc3n12] | 3-cyclopropyl-N- ((1S,3R,4S)-3-ethyl-4- (6H-pyrrolo[2,3- e][1,2,4]triazolo[4,3- a]pyrazin-1- yl)cyclopentyl)pro- panamide | 0.64 | 367 | B |
| 46 | 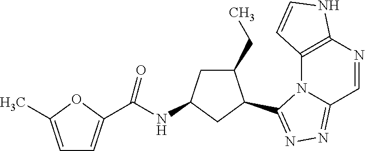 [SMILES: CC[C@@H]1C[C@H](NC(=O)c2ccc(C)o2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-5- methylfuran-2- carboxamide | 0.64 | 379 | C |
| 47 | 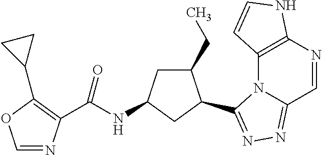 [SMILES: CC[C@@H]1C[C@H](NC(=O)c2ncoc2C2CC2)C[C@@H]1c1nnc2cnc3[nH]ccc3n12] | 5-cyclopropyl-N- ((1S,3R,4S)-3-ethyl-4- (6H-pyrrolo[2,3- e][1,2,4]triazolo[4,3- a]pyrazin-1- yl)cyclopentyl)oxazole- 4-carboxamide | 0.67 | 406 | C |
| 48 | 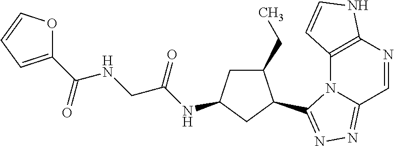 [SMILES: CC[C@@H]1C[C@H](NC(=O)CNC(=O)c2ccco2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-5- methylisoxazole-3- carboxamide | 0.56 | 422 | B |
| 49 | 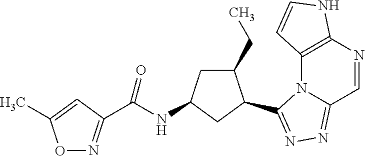 [SMILES: CC[C@@H]1C[C@H](NC(=O)c2cc(C)on2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-5- methylisoxazole-3- carboxamide | 0.63 | 380 | B |
| 50 | 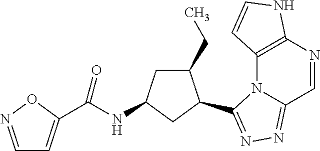 [SMILES: CC[C@@H]1C[C@H](NC(=O)c2ccno2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)isoxazole- 5-carboxamide | 0.59 | 366 | B |
| 51 | 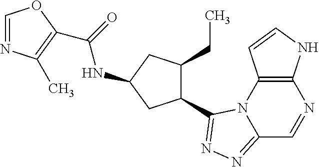 [SMILES: CC[C@@H]1C[C@H](NC(=O)c2ocnc2C)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-4- methyloxazole-5- carboxamide | 0.59 | 380 | B |
| 52 | 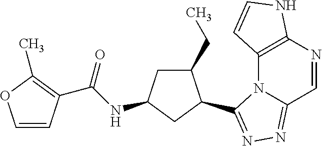 [SMILES: CC[C@@H]1C[C@H](NC(=O)c2ccoc2C)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-2- methylfuran-3- carboxamide | 0.66 | 379 | B |
| 53 | 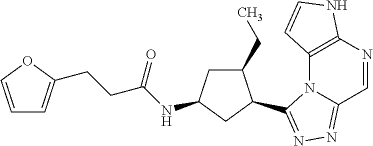 [SMILES: CC[C@@H]1C[C@H](NC(=O)CCc2ccco2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3- (furan-2- yl)propanamide | 0.64 | 393 | A |
| 54 | 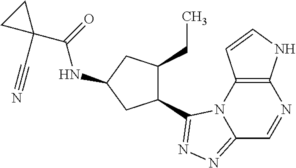 [SMILES: CC[C@@H]1C[C@H](NC(=O)C2(C#N)CC2)C[C@@H]1c1nnc2cnc3[nH]ccc3n12] | 1-cyano-N-((1S,3R,4S)- 3-ethyl-4-(6H- pyrrolo[2,3- e][1,2,4]triazolo[4,3- a]pyrazin-1- yl)cyclopentyl)cyclo- propanecarboxamide | 0.61 | 364 | B |
| 55 | 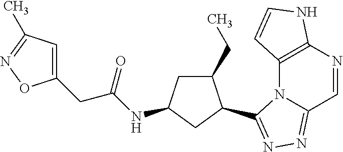 [SMILES: CC[C@@H]1C[C@H](NC(=O)Cc2cc(C)no2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-2-(3- methylisoxazol-5- yl)acetamide | 0.58 | 394 | B |
| 56 | 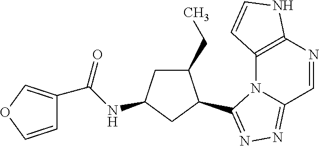 [SMILES: CC[C@@H]1C[C@H](NC(=O)c2ccoc2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)furan-3- carboxamide | 0.61 | 365 | B |
| 57 | 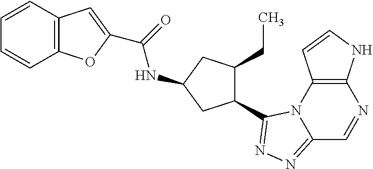 [SMILES: CC[C@@H]1C[C@H](NC(=O)c2cc3ccccc3o2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)benzo- furan-2-carboxamide | 0.71 | 415 | B |
| 58 | 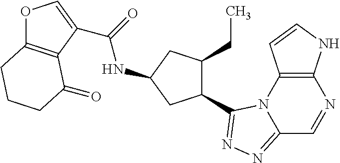 [SMILES: CC[C@@H]1C[C@H](NC(=O)c2coc3c2C(=O)CCC3)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-4-oxo- 4,5,6,7- tetrahydrobenzofuran- 3-carboxamide | 0.65 | 433 | B |
| 59 | 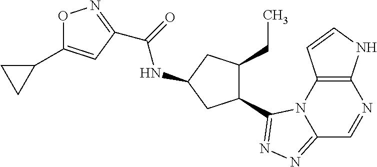 [SMILES: CC[C@@H]1C[C@H](NC(=O)c2cc(C3CC3)on2)C[C@@H]1c1nnc2cnc3[nH]ccc3n12] | 5-cyclopropyl-N- ((1S,3R,4S)-3-ethyl-4- (6H-pyrrolo[2,3- e][1,2,4]triazolo[4,3- a]pyrazin-1- yl)cyclopentyl)isoxazole- 3-carboxamide | 0.69 | 406 | B |
| 60 | 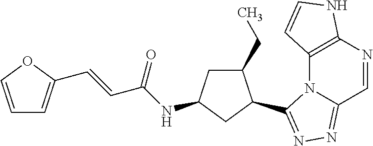 [SMILES: CC[C@@H]1C[C@H](NC(=O)/C=C/c2ccco2)C[C@@H]1c1nnc2cnc3[nH]ccc3n12] | (E)-N-((1S,3R,4S)-3- ethyl-4-(6H- pyrrolo[2,3- e][1,2,4]triazolo[4,3- a]pyrazin-1- yl)cyclopentyl)-3- (furan-2-yl)acrylamide | 0.65 | 391 | B |
| 61 | 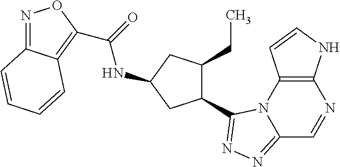 [SMILES: CC[C@@H]1C[C@H](NC(=O)c2onc3ccccc23)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)benzo[c] isoxazole-3- carboxamide | 0.7 | 416 | Not tested |
| 62 | 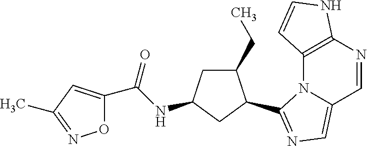 [SMILES: CC[C@@H]1C[C@H](NC(=O)c2cc(C)no2)C[C@@H]1c1ncc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3- methylisoxazole-5- carboxamide | 0.61 | 380 | B |
| 63 | 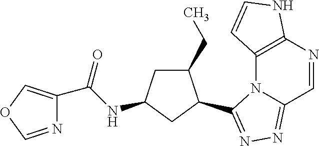 [SMILES: CC[C@@H]1C[C@H](NC(=O)c2cocn2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)oxazole- 4-carboxamide | 0.57 | 366 | B |
| 64 | 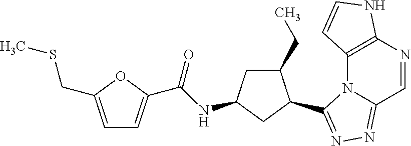 [SMILES: CC[C@@H]1C[C@H](NC(=O)c2ccc(CSC)o2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-5- (methylthiomethyl)furan- 2-carboxamide | 0.67 | 425 | B |
| 65 | 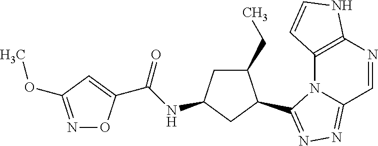 [SMILES: CC[C@@H]1C[C@H](NC(=O)c2cc(OC)no2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3- methoxyisoxazole-5- carboxamide | 0.63 | 396 | B |
| 66 | 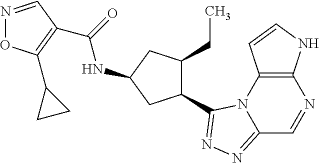 [SMILES: CC[C@@H]1C[C@H](NC(=O)c2cnoc2C2CC2)C[C@@H]1c1nnc2cnc3[nH]ccc3n12] | 5-cyclopropyl-N- ((1S,3R,4S)-3-ethyl-4- (6H-pyrrolo[2,3- e][1,2,4]triazolo[4,3- a]pyrazin-1- yl)cyclopentyl)isoxazole- 4-carboxamide | 0.79 | 406 | B |
| 67 | 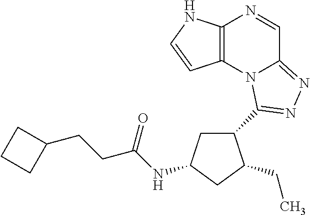 [SMILES: CC[C@@H]1C[C@H](NC(=O)CCC2CCC2)C[C@@H]1c1nnc2cnc3[nH]ccc3n12] | 3-cyclobutyl-N- ((1S,3R,4S)-3-ethyl-4- (6H-pyrrolo[2,3- e][1,2,4]triazolo[4,3- a]pyrazin-1- yl)cyclopentyl)pro- panamide | 0.7 | 381 | B |
| 68 | 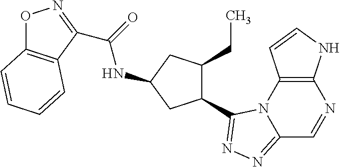 [SMILES: CC[C@@H]1C[C@H](NC(=O)c2noc3ccccc23)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)benzo[d] isoxazole-3- carboxamide | 0.73 | 416 | A |
| 69 | 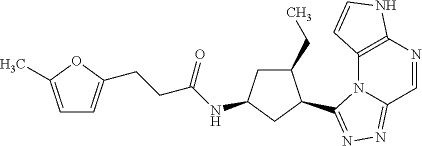 [SMILES: CC[C@@H]1C[C@H](NC(=O)CCc2ccc(C)o2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3-(5- methylfuran-2- yl)propanamide | 0.67 | 407 | B |
| 70 | 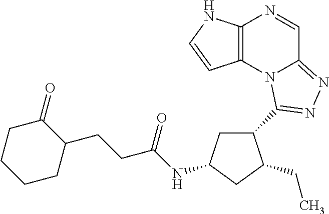 [SMILES: CC[C@@H]1C[C@H](NC(=O)CCC2CCCCC2=O)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3-(2- oxocyclohexyl)pro- panamide | 0.63 | 391 | C |
| 71 | 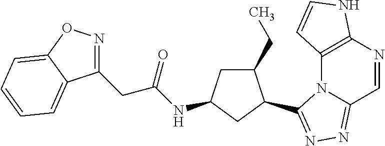 [SMILES: CC[C@@H]1C[C@H](NC(=O)Cc2noc3ccccc23)C[C@@H]1c1nnc2cnc3[nH]ccc3n12] | 2-(benzo[d]isoxazol-3- yl)-N-((1S,3R,4S)-3- ethyl-4-(6H- pyrrolo[2,3- e][1,2,4]triazolo[4,3- a]pyrazin-1- yl)cyclopentyl)acetamide | 0.66 | 416 | B |
| 72 | 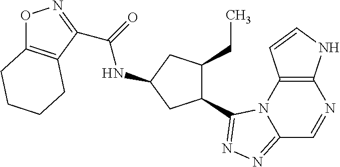 [SMILES: CC[C@@H]1C[C@H](NC(=O)c2noc3c2CCCC3)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-4,5,6,7- tetrahydrobenzo[d]isoxa- zole-3-carboxamide | 0.7 | 380 | B |
| 73 | 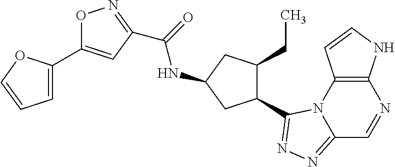 [SMILES: CC[C@@H]1C[C@H](NC(=O)c2cc(-c3ccco3)on2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-5- (furan-2-yl)isoxazole- 3-carboxamide | 0.71 | 366 | A |
| 74 | 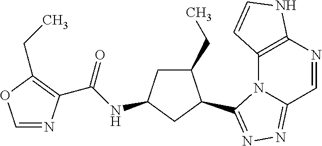 [SMILES: CCc1ocnc1C(=O)N[C@H]1C[C@@H](CC)[C@@H](c2nnc3cnc4[nH]ccc4n23)C1] | 5-ethyl-N-((1S,3R,4S)- 3-ethyl-4-(6H- pyrrolo[2,3- e][1,2,4]triazolo[4,3- a]pyrazin-1- yl)cyclopentyl)oxazole- 4-carboxamide | 0.66 | 425 | B |
| 75 | 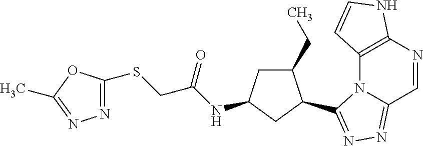 [SMILES: CC[C@@H]1C[C@H](NC(=O)CSc2nnc(C)o2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-2-(5- methyl-1,3,4- oxadiazol-2- ylthio)acetamide | 0.52 | 396 | B |
| 76 | 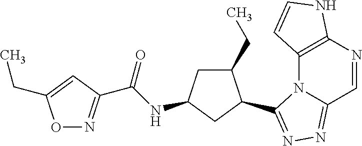 [SMILES: CCc1cc(C(=O)N[C@H]2C[C@@H](CC)[C@@H](c3nnc4cnc5[nH]ccc5n34)C2)no1] | 5-ethyl-N-((1S,3R,4S)- 3-ethyl-4-(6H- pyrrolo[2,3- e][1,2,4]triazolo[4,3- a]pyrazin-1- yl)cyclopentyl)isoxazole- 3-carboxamide | 0.68 | 406 | B |
| 77 | 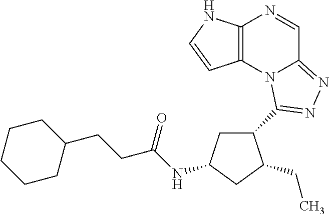 [SMILES: CC[C@@H]1C[C@H](NC(=O)CCC2CCCCC2)C[C@@H]1c1nnc2cnc3[nH]ccc3n12] | 3-cyclohexyl-N- ((1S,3R,4S)-3-ethyl-4- (6H-pyrrolo[2,3- e][1,2,4]triazolo[4,3- a]pyrazin-1- yl)cyclopentyl)pro- panamide | 0.77 | 381 | B |
| 78 | 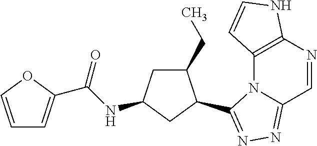 [SMILES: CC[C@@H]1C[C@H](NC(=O)c2ccco2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)furan-2- carboxamide | 0.69 | 365 | B |
| 79 | 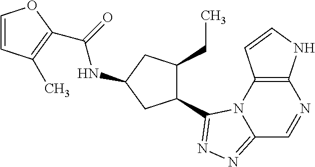 [SMILES: CC[C@@H]1C[C@H](NC(=O)c2occc2C)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3- methylfuran-2- carboxamide | 0.65 | 379 | B |
| 80 | 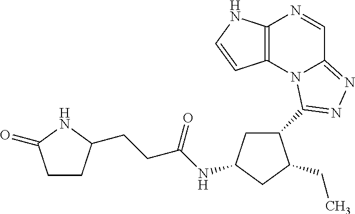 [SMILES: CC[C@@H]1C[C@H](NC(=O)CCC2CCC(=O)N2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-3-(5- oxopyrrolidin-2- yl)propanamide | 0.54 | 410 | B |
| 81 | 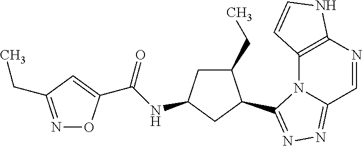 [SMILES: CCc1cc(C(=O)N[C@H]2C[C@@H](CC)[C@@H](c3nnc4cnc5[nH]ccc5n34)C2)on1] | 3-ethyl-N-((1S,3R,4S)- 3-ethyl-4-(6H- pyrrolo[2,3- e][1,2,4]triazolo[4,3- a]pyrazin-1- yl)cyclopentyl)isoxazole- 5-carboxamide | 0.65 | 394 | B |
| 82 | 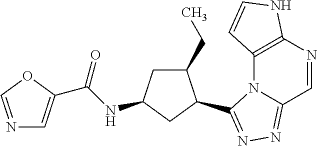 [SMILES: CC[C@@H]1C[C@H](NC(=O)c2cnco2)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)oxazole- 5-carboxamide | 0.56 | 366 | B |
| 83 | 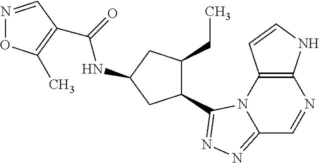 [SMILES: CC[C@@H]1C[C@H](NC(=O)c2cnoc2C)C[C@@H]1c1nnc2cnc3[nH]ccc3n12] | N-((1S,3R,4S)-3-ethyl- 4-(6H-pyrrolo[2,3- e][1,2,4]triazolo[4,3- a]pyrazin-1- yl)cyclopentyl)-5- methylisoxazole-4- carboxamide | 0.72 | 380 | Not tested |

What is claimed:

1. A compound of Formula (I) 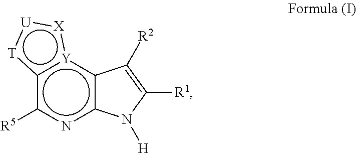
pharmaceutically acceptable salts, pro-drugs, biologically active metabolites, stereoisomers and isomers thereof wherein
T is N, U is N, X is CR³ and Y is N; or
T is N, U is CR⁴, X is CR³ and Y is N; or
T is N, U is N, X is NR³ and Y is C;
R¹, R² and R⁵ are each independently hydrogen or deuterium;
R³ is -A-D-E-G, wherein:
A is a bond or optionally substituted (C₃-C₁₂)cycloalkylene;
D is an optionally substituted (C₁-C₈)alkylene, an optionally substituted (C₃-C₁₀)cycloalkylene or an optionally substituted (C₂-C₁₀)heterocyclylene;
E is —R^{e}—, —R^{e}—C(O)—R^{e}—, —R^{e}—C(O)O—R^{e}, —R^{e}—N(R^{a})—R^{e}—, —R^{e}—N(R^{a})C(O)—R^{e}—, —R^{e}—C(O)N(R^{a})—R^{e}, —R^{e}—N(R^{a})S(O)₂—R^{e}—, or —R^{e}—S(O)₂N(R^{a})—R^{e}—, —R^{e}—O—CH₂—C(O)N(R^{a})—R^{e}—, —R^{e}—N(R^{a})C(O)—CH₂—O—R^{e}—, —R^{e}—C(O)N(R^{a})CH₂C(O)N(R^{a})—R^{e}—, —R^{e}—N(R^{a})C(O)CH₂N(R^{a})C(O)—R^{e}—, —R^{e}—C(O)N(R^{a})CH₂C(O)N(R^{a})—R^{e}—, R^{e}—N(R^{a})C(O)CH₂N(R^{a})C(O)—R^{e}, —R^{e}—S—CH₂C(O)N(R^{a})—R^{e}—, or —R^{e}—N(R^{a})C(O)CH₂—S—R^{e}—;
G is halogen, —CN, —CF₃, an optionally substituted —(C₁-C₆)alkyl, an -optionally substituted —(C₁-C₆)alkyl-heteroaryl, an optionally substituted —(C₃-C₁₀)cycloalkyl, an optionally substituted heterocyclyl or an optionally substituted heteroaryl;
R⁴ is hydrogen or deuterium;
R^{a} and R^{b} are each independently hydrogen, deuterium, an optionally substituted (C₁-C₁₀)alkyl or an optionally substituted (C₁-C₁₀)heterocyclylene; and
R^{e} for each occurrence is independently a bond, an optionally substituted (C₁-C₁₀)alkylene, an optionally substituted (C₂-C₆)alkenylene, or an optionally substituted (C₁-C₁₀)heterocyclylene.

2. The compound of claim 1 wherein A is a bond or an optionally substituted cyclopentyl.

3. The compound of claim 1 wherein D is an optionally substituted (C₁-C₆) alkylene, optionally substituted cyclopentylene or optionally substituted pyrrolidine.

4. The compound of claim 1 wherein E is a bond, an optionally substituted (C₁-C₆) alkylene, —C(O)—, —(C(O)—(C₁-C₃)alkylene-, —(C₁-C₃)alkylene-C(O)—, —N(H)—(C₁-C₃)alkylene-, —(C₁-C₃)alkylene-N(H)—, —N(H)—C(O)—, —C(O)N(H)—, —N(CH₃)—C(O)—, —C(O)N(CH₃)—, —(C₁-C₃)alkylene-N(H)—C(O)—, —C(O)N(H)—(C₁-C₃)alkylene-, —(C₁-C₃)alkylene-N(oxetanyl)-C(O)—, —C(O)N(oxetanyl)-(C₁-C₃)alkylene-, —N(R^{a})S(O)₂—(C₁-C₃)alkylene-, —(C₁-C₃)alkylene-S(O)₂N(R^{a})—, optionally substituted azetidine or optionally substituted piperidine.

5. The compound of claim 4 wherein G is F, —CN, —CF₃, an optionally substituted —(C₁-C₆)alkyl, an optionally substituted —(C₃-C₆)cycloalkyl, an optionally substituted morpholine, an optionally substituted oxazole, an optionally substituted 1,2,4-oxadiazole, an optionally substituted pyridazine, or an optionally substituted pyridine.

6. The compound of claim 1 wherein
T is N, U is N, X is CR³ and Y is N;
wherein R³ is -A-D-E-G and
A is a bond;
D is optionally substituted cyclopentylene;
E is an optionally substituted (C₁-C₆) alkylene, —C(O)—, —N(H)—C(O)—, —C(O)N(H)—, —C(O)N(H)—CH₂, —CH₂—N(H)C(O)—, —O—CH₂C(O)N(H)—, —N(H)C(O)CH₂—O—, —C(O)N(CH₃)CH₂C(O)N(H)—, —N(H)C(O)CH₂N(CH₃)C(O)—, —N(H)C(O)C(H)═C(H)—, —C(H)═C(H)C(O)N(H)—, -optionally substituted (C₁-C₃)alkylene-C(O)N(H)—, —N(H)C(O)-optionally substituted (C₁-C₃)alkylene, —C(O)N(H)CH₂C(O)N(H)—, —N(H)C(O)CH₂N(H)C(O)—, —S—CH₂C(O)N(H)—, or —N(H)C(O)CH₂—S—; and
G is CN, CF₃, an optionally substituted benzofuranyl, an optionally substituted cyclobutyl, an optionally substituted cyclopropyl, an optionally substituted cyclohexyl, an optionally substituted benzo[c]isoxazolyl, an optionally substituted benzo[d]isoxazolyl, an optionally substituted furanyl, an optionally substituted furo[3,2-b]pyridinyl, an optionally substituted furo[3,2-b]pyrrolyl, an optionally substituted isoxazolyl, optionally substituted 1,2,4-oxadiazolyl, an optionally substituted oxazolyl, an optionally substituted pyrrolidinyl, an optionally substituted tetrahydrobenzo[d]isoxazolyl, an optionally substituted tetrahydrobenzofuranyl, or a 2,5-dioxoimidazolidinyl.

7. The compound of claim 6 wherein the compound is
(1R,3R,4S)-3-ethyl-4-(6H-pyrrolo[2,3-e][1,2,4]triazolo[4,3-a]pyrazin-1-yl)-N-(2,2,2-trifluoroethyl)cyclopentanecarboxamide;
3-((1S,3R,4S)-3-methyl-4-(6H-pyrrolo[2,3-e][1,2,4]triazolo[4,3-a]pyrazin-1-yl)cyclopentyl)propanenitrile;
3-((1R,3R,4S)-3-methyl-4-(6H-pyrrolo[2,3-e][1,2,4]triazolo[4,3-a]pyrazin-1-yl)cyclopentyl)propanenitrile;
3-((1S,3R,4S)-3-ethyl-4-(6H-pyrrolo[2,3-e][1,2,4]triazolo[4,3-a]pyrazin-1-yl)cyclopentyl)propanenitrile;
3-((1R,3R,4S)-3-ethyl-4-(6H-pyrrolo[2,3-e][1,2,4]triazolo[4,3-a]pyrazin-1-yl)cyclopentyl)propanenitrile;
N-((1S,3R,4S)-3-ethyl-4-(6H-pyrrolo[2,3-e][1,2,4]triazolo[4,3-a]pyrazin-1-yl)cyclopentyl)-2-methyloxazole-4-carboxamide;
N-((1S,3R,4S)-3-ethyl-4-(6H-pyrrolo[2,3-e][1,2,4]triazolo[4,3-a]pyrazin-1-yl)cyclopentyl)benzofuran-3-carboxamide;
N-((1S,3R,4S)-3-ethyl-4-(6H-pyrrolo[2,3-e][1,2,4]triazolo[4,3-a]pyrazin-1-yl)cyclopentyl)-4-methyl-4H-furo[3,2-b]pyrrole-5-carboxamide;
N-((1S,3R,4S)-3-ethyl-4-(6H-pyrrolo[2,3-e][1,2,4]triazolo[4,3-a]pyrazin-1-yl)cyclopentyl)-3-(1-methylcyclopropyl)propanamide;
3-cyclopropyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)-5-methylfuran-2-carboxamide;
1-cyano-N-((1S,3R,4S)-3-ethyl-4-(6H-pyrrolo[2,3-e][1,2,4]triazolo[4,3-a]pyrazin-1-yl)cyclopentyl)cyclopropanecarboxamide;
N-((1S,3R,4S)-3-ethyl-4-(6H-pyrrolo[2,3-e][1,2,4]triazolo[4,3-a]pyrazin-1-yl)cyclopentyl)-5-methylisoxazole-4-carboxamide
(E)-N-((1S,3R,4S)-3-ethyl-4-(6H-pyrrolo[2,3-e][1,2,4]triazolo[4,3-a]pyrazin-1-yl)cyclopentyl)-3-(5-methylfuran-2-yl)-acrylamide;
N-((1S,3R,4S)-3-ethyl-4-(6H-pyrrolo[2,3-e][1,2,4]triazolo[4,3-a]pyrazin-1-yl)cyclopentyl)-2-(5-methylisoxazol-3-yloxy)acetamide;
N-(2-((1S,3R,4S)-3-ethyl-4-(6H-pyrrolo[2,3-e][1,2,4]triazolo[4,3-a]pyrazin-1-yl)cyclopentylamino)-2-oxoethyl)-N-methylfuran-2-carboxamide;
3-(2,5-dioxoimidazolidin-4-yl)-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)-5-methyl-4,5,6,7-tetrahydrobenzo[d]isoxazole-3-carboxamide;
N-(2-((1S,3R,4S)-3-ethyl-4-(6H-pyrrolo[2,3-e][1,2,4]triazolo[4,3-a]pyrazin-1-yl)cyclopentylamino)-2-oxoethyl)-5-methylfuran-2-carboxamide;
2-acetamido-3-cyclopropyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)benzofuran-3-carboxamide;
N-((1S,3R,4S)-3-ethyl-4-(6H-pyrrolo[2,3-e][1,2,4]triazolo[4,3-a]pyrazin-1-yl)cyclopentyl)-6-methylbenzofuran-2-carboxamide;
N-((1S,3R,4S)-3-ethyl-4-(6H-pyrrolo[2,3-e][1,2,4]triazolo[4,3-a]pyrazin-1-yl)cyclopentyl)-3-methylisoxazole-4-carboxamide;
N-((1S,3R,4S)-3-ethyl-4-(6H-pyrrolo[2,3-e][1,2,4]triazolo[4,3-a]pyrazin-1-yl)cyclopentyl)-2-methyl-4,5,6,7-tetrahydrobenzofuran-3-carboxamide;
N-((1S,3R,4S)-3-ethyl-4-(6H-pyrrolo[2,3-e][1,2,4]triazolo[4,3-a]pyrazin-1-yl)cyclopentyl)-3-(3-methoxyisoxazol-5-yl)propanamide;
N-((1S,3R,4S)-3-ethyl-4-(6H-pyrrolo[2,3-e][1,2,4]triazolo[4,3-a]pyrazin-1-yl)cyclopentyl)-3-(3-methyl-1,2,4-oxadiazol-5-yl)propanamide;
N-(2-((1S,3R,4S)-3-ethyl-4-(6H-pyrrolo[2,3-e][1,2,4]triazolo[4,3-a]pyrazin-1-yl)cyclopentylamino)-2-oxoethyl)-5-methylisoxazole-4-carboxamide;
N-((1S,3R,4S)-3-ethyl-4-(6H-pyrrolo[2,3-e][1,2,4]triazolo[4,3-a]pyrazin-1-yl)cyclopentyl)-5-(furan-2-yl)-4H-pyrazole-3-carboxamide;
N-((1S,3R,4S)-3-ethyl-4-(6H-pyrrolo[2,3-e][1,2,4]triazolo[4,3-a]pyrazin-1-yl)cyclopentyl)-4-methyl-4H-furo[3,2-b]pyrrole-5-carboxamide;
2-(benzofuran-3-yl)-N-((1S,3R,4S)-3-ethyl-4-(6H-pyrrolo[2,3-e][1,2,4]triazolo[4,3-a]pyrazin-1-yl)cyclopentyl)acetamide;
N-((1S,3R,4S)-3-ethyl-4-(6H-pyrrolo[2,3-e][1,2,4]triazolo[4,3-a]pyrazin-1-yl)cyclopentyl)-5-methyloxazole-4-carboxamide;
N-((1S,3R,4S)-3-ethyl-4-(6H-pyrrolo[2,3-e][1,2,4]triazolo[4,3-a]pyrazin-1-yl)cyclopentyl)-3-methylbenzofuran-2-carboxamide;
N-((1S,3R,4S)-3-ethyl-4-(6H-pyrrolo[2,3-e][1,2,4]triazolo[4,3-a]pyrazin-1-yl)cyclopentyl)-3-(1-methylcyclopropyl)propanamide;
3-cyclopropyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)-5-methylfuran-2-carboxamide;
5-cyclopropyl-N-((1S,3R,4S)-3-ethyl-4-(6H-pyrrolo[2,3-e][1,2,4]triazolo[4,3-a]pyrazin-1-yl)cyclopentyl)oxazole-4-carboxamide;
N-((1S,3R,4S)-3-ethyl-4-(6H-pyrrolo[2,3-e][1,2,4]triazolo[4,3-a]pyrazin-1-yl)cyclopentyl)-5-methylisoxazole-3-carboxamide;
N-((1S,3R,4S)-3-ethyl-4-(6H-pyrrolo[2,3-e][1,2,4]triazolo[4,3-a]pyrazin-1-yl)cyclopentyl)-5-methylisoxazole-3-carboxamide;
N-((1S,3R,4S)-3-ethyl-4-(6H-pyrrolo[2,3-e][1,2,4]triazolo[4,3-a]pyrazin-1-yl)cyclopentyl)isoxazole-5-carboxamide;
N-((1S,3R,4S)-3-ethyl-4-(6H-pyrrolo[2,3-e][1,2,4]triazolo[4,3-a]pyrazin-1-yl)cyclopentyl)-4-methyloxazole-5-carboxamide;
N-((1S,3R,4S)-3-ethyl-4-(6H-pyrrolo[2,3-e][1,2,4]triazolo[4,3-a]pyrazin-1-yl)cyclopentyl)-2-methylfuran-3-carboxamide;
N-((1S,3R,4S)-3-ethyl-4-(6H-pyrrolo[2,3-e][1,2,4]triazolo[4,3-a]pyrazin-1-yl)cyclopentyl)-3-(furan-2-yl)propanamide;
1-cyano-N-((1S,3R,4S)-3-ethyl-4-(6H-pyrrolo[2,3-e][1,2,4]triazolo[4,3-a]pyrazin-1-yl)cyclopentyl)cyclopropanecarboxamide;
N-((1S,3R,4S)-3-ethyl-4-(6H-pyrrolo[2,3-e][1,2,4]triazolo[4,3-a]pyrazin-1-yl)cyclopentyl)-2-(3-methylisoxazol-5-yl)acetamide;
N-((1S,3R,4S)-3-ethyl-4-(6H-pyrrolo[2,3-e][1,2,4]triazolo[4,3-a]pyrazin-1-yl)cyclopentyl)furan-3-carboxamide;
N-((1S,3R,4S)-3-ethyl-4-(6H-pyrrolo[2,3-e][1,2,4]triazolo[4,3-a]pyrazin-1-yl)cyclopentyl)-benzofuran-2-carboxamide;
N-((1S,3R,4S)-3-ethyl-4-(6H-pyrrolo[2,3-e][1,2,4]triazolo[4,3-a]pyrazin-1-yl)cyclopentyl)-4-oxo-4,5,6,7-tetrahydrobenzofuran-3-carboxamide;
5-cyclopropyl-N-((1S,3R,4S)-3-ethyl-4-(6H-pyrrolo[2,3-e][1,2,4]triazolo[4,3-a]pyrazin-1-yl)cyclopentyl)isoxazole-3-carboxamide;
(E)-N-((1S,3R,4S)-3-ethyl-4-(6H-pyrrolo[2,3-e][1,2,4]triazolo[4,3-a]pyrazin-1-yl)cyclopentyl)-3-(furan-2-yl)acrylamide;
N-((1S,3R,4S)-3-ethyl-4-(6H-pyrrolo[2,3-e][1,2,4]triazolo[4,3-a]pyrazin-1-yl)cyclopentyl)benzo[c]isoxazole-3-carboxamide
N-((1S,3R,4S)-3-ethyl-4-(6H-pyrrolo[2,3-e][1,2,4]triazolo[4,3-a]pyrazin-1-yl)cyclopentyl)-3-methylisoxazole-5-carboxamide;
N-((1S,3R,4S)-3-ethyl-4-(6H-pyrrolo[2,3-e][1,2,4]triazolo[4,3-a]pyrazin-1-yl)cyclopentyl)oxazole-4-carboxamide;
N-(1S,3R,4S)-3-ethyl-4-(6H-pyrrolo[2,3-e][1,2,4]triazolo[4,3-a]pyrazin-1-yl)cyclopentyl)-5-(methylthiomethyl)furan-2-carboxamide;
N-((1S,3R,4S)-3-ethyl-4-(6H-pyrrolo[2,3-e][1,2,4]triazole[4,3-a]pyrazin-1-yl)cyclopentyl)-3-methoxyisoxazole-5-carboxamide;
5-cyclopropyl-N-((1S,3R,4S)-3-ethyl-4-(6H-pyrrolo[2,3-e][1,2,4]triazolo[4,3-a]pyrazin-1-yl)cyclopentyl)isoxazole-4-carboxamide;
3-cyclobutyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)benzo[d]isoxazole-3-carboxamide;
N-((1S,3R,4S)-3-ethyl-4-(6H-pyrrolo[2,3-e][1,2,4]triazolo[4,3-a]pyrazin-1-yl)cyclopentyl)-3-(5-methylfuran-2-yl)propanamide;
N-((1S,3R,4S)-3-ethyl-4-(6H-pyrrolo[2,3-e][1,2,4]triazole[4,3-a]pyrazin-1-yl)cyclopentyl)-3-(2-oxocyclohexyl)propanamide;
2-(benzo[d]isoxazol-3-yl)-N-((1S,3R,4S)-3-ethyl-4-(6H-pyrrolo[2,3-e][1,2,4]triazolo[4,3-a]pyrazin-1-yl)cyclopentyl)acetamide;
N-((1S,3R,4S)-3-ethyl-4-(6H-pyrrolo[2,3-e][1,2,4]triazolo[4,3-a]pyrazin-1-yl)cyclopentyl)-4,5,6,7-tetrahydrobenzo[d]isoxazole-3-carboxamide;
N-((1S,3R,4S)-3-ethyl-4-(6H-pyrrolo[2,3-e][1,2,4]triazolo[4,3-a]pyrazin-1-yl)cyclopentyl)-5-(furan-2-yl)isoxazole-3-carboxamide;
5-ethyl-N-((1S,3R,4S)-3-ethyl-4-(6H-pyrrolo[2,3-e][1,2,4]triazolo[4,3-a]pyrazin-1-yl)cyclopentyl)oxazole-4-carboxamide;
N-((1S,3R,4S)-3-ethyl-4-(6H-pyrrolo[2,3-e][1,2,4]triazolo[4,3-a]pyrazin-1-yl)cyclopentyl)-2-(5-methyl-1,3,4-oxadiazol-2-ylthio)acetamide;
5-ethyl-N-((1S,3R,4S)-3-ethyl-4-(6H-pyrrolo[2,3-e][1,2,4]triazolo[4,3-a]pyrazin-1-yl)cyclopentyl)isoxazole-3-carboxamide;
3-cyclohexyl-N-((1S,3R,4S)-3-ethyl-4-(6H-pyrrolo[2,3-e][1,2,4]triazolo[4,3-a]pyrazin-1-yl)cyclopentyl)propanamide;
N-((1S,3R,4S)-3-ethyl-4-(6H-pyrrolo[2,3-e][1,2,4]triazolo[4,3-a]pyrazin-1-yl)cyclopentyl)furan-2-carboxamide;
N-((1S,3R,4S)-3-ethyl-4-(6H-pyrrolo[2,3-e][1,2,4]triazolo[4,3-a]pyrazin-1-yl)cyclopentyl)-3-methylfuran-2-carboxamide;
N-((1S,3R,4S)-3-ethyl-4-(6H-pyrrolo[2,3-e][1,2,4]triazolo[4,3-a]pyrazin-1-yl)cyclopentyl)-3-(5-oxopyrrolidin-2-yl)propanamide;
3-ethyl-N-((1S,3R,4S)-3-ethyl-4-(6H-pyrrolo[2,3-e][1,2,4]triazolo[4,3-a]pyrazin-1-yl)cyclopentyl)isoxazole-5-carboxamide;
N-((1S,3R,4S)-3-ethyl-4-(6H-pyrrolo[2,3-e][1,2,4]triazolo[4,3-a]pyrazin-1-yl)cyclopentyl)oxazole-5-carboxamide; or
N-((1S,3R,4S)-3-ethyl-4-(6H-pyrrolo[2,3-e][1,2,4]triazolo[4,3-a]pyrazin-1-yl)cyclopentyl)-5-methylisoxazole-4-carboxamide.

8. The compound of claim 1 wherein
T is N, U is CR⁴, X is CR³ and Y is N;
R³ is -A-D-E-G, wherein:
A is a bond or optionally substituted (C₃-C₆)cycloalkylene;
D is an optionally substituted (C₁-C₄)alkylene, optionally substituted (C₃-C₁₀)cycloalkylene or an optionally substituted (C₂-C₁₀)heterocyclylene;
E is —R^{e}—, —R^{e}—C(O)—R^{e}—, —R^{e}—C(O)O—R^{e}, —R^{e}—N(R^{a})—R^{e}—, —R^{e}—N(R^{a})C(O)—R^{e}—, —R^{e}—C(O)N(R^{a})—R^{e}—, —R^{e}—N(R^{a})S(O)₂—R^{e}—, or —R^{e}—S(O)₂N(R^{a})—R^{e}—;
G is halogen, —CN, —CF₃, an optionally substituted —(C₁-C₆)alkyl, —CH₂-heteroaryl, an optionally substituted (C₃-C₁₀)cycloalkyl, an optionally substituted heterocyclyl or an optionally substituted heteroaryl;
R⁴ is hydrogen;
R^{a} and R^{b} are each independently hydrogen, an optionally substituted (C₁-C₁₀)alkyl or an optionally substituted (C₁-C₁₀)heterocyclylene; and
R^{e} for each occurrence is independently a bond, an optionally substituted (C₁-C₁₀)alkylene or an optionally substituted (C₁-C₆)heterocyclylene.

9. The compound of claim 8 wherein D is —CH₂—, optionally substituted cyclopentyl, optionally substituted piperidinyl or optionally substituted pyrrolidinyl.

10. The compound of claim 9 wherein E is a bond, —C(O)—, —C(O)O(C₁-C₃)alkylene-, —N(R^{a})—C(O)—, —C(O)N(R^{a})—, —C(O)—N(H)-optionally substituted (C₁-C₃)alkylene-, -optionally substituted (C₁-C₃)alkylene —N(R^{a})C(O)—, (C₁-C₃)alkylene, —C(O)—(C₁-C₃)alkylene, —(C₁-C₃)alkylene-C(O)—, —(C₁-C₃)alkylene-OC(O)—, —CH₂—N(H)S(O)₂—, —N(H)—CH₂—, —CH₂—N(H)—, —S(O)₂N(H)—CH₂—, or optionally substituted azetidine.

11. The compound of claim 10 wherein G is F, —CN, —CF₃, an optionally substituted (C₁-C₄)alkyl, —CH₂-oxazolyl, optionally substituted cyclopropyl, optionally substituted cyclobutyl, optionally substituted azetidinyl, optionally substituted morpholinyl, optionally substituted oxazolyl, optionally substituted piperidinyl, optionally substituted pyrazinyl, optionally substituted pyridazinyl, optionally substituted pyridinyl or optionally substituted pyrimidinyl.

12. The compound of claim 11 wherein R^{a} and R^{b} are each independently hydrogen, CH₃ or oxetanyl.

13. The compound of claim 12 wherein the compound is
N-((1S,3S,4R)-3-(3H-imidazo[1,2-a]pyrrolo[2,3-e]pyrazin-8-yl)-4-methylcyclopentyl-5-methylpyrazine-2-carboxamide;
N-((1S,3S,4R)-3-(3H-imidazo[1,2-a]pyrrolo[2,3-e]pyrazin-8-yl)-4-methylcyclopentyl)pyridazine-3-carboxamide;
N-((1S,3S,4R)-3-(3H-imidazo[1,2-a]pyrrolo[2,3-e]pyrazin-8-yl)-4-methylcyclopentyl)-N-methyloxazole-4-carboxamide;
(1S,3S,4R)-3-(3H-imidazo[1,2-a]pyrrolo[2,3-e]pyrazin-8-yl)-4-methyl-N-((2-(trifluoromethyl)pyrimidin-5-yl)methyl)cyclopentanamine;
1-((1S,3S,4R)-3-(3H-imidazo[1,2-a]pyrrolo[2,3-e]pyrazin-8-yl)-4-methylcyclopentyl)piperidine-4-carbonitrile;
1-(((1R,3S,4R)-3-(3H-imidazo[1,2-a]pyrrolo[2,3-e]pyrazin-8-yl)-4-methylcyclopentyl)methyl)azetidine-3-carbonitrile;
1-(((1S,3S,4R)-3-(3H-imidazo[1,2-a]pyrrolo[2,3-e]pyrazin-8-yl)-4-methylcyclopentyl)methyl)azetidine-3-carbonitrile;
1-((1R,3S,4R)-3-(3H-imidazo[1,2-a]pyrrolo[2,3-e]pyrazin-8-yl)-4-methylcyclopentyl)piperidine-4-carbonitrile;
5-((3S,4R)-3-ethyl-4-(3H-imidazo[1,2-a]pyrrolo[2,3-e]pyrazin-8-yl)pyrrolidin-1-yl)-5-oxopentanenitrile;
(3S,4R)-3-ethyl-N-(2-fluoroethyl)-4-(3H-imidazo[1,2-a]pyrrolo[2,3-e]pyrazin-8-yl)pyrrolidine-1-carboxamide;
(3S,4R)—N-(2,2-difluoroethyl)-3-ethyl-4-(3H-imidazo[1,2-a]pyrrolo[2,3-e]pyrazin-8-yl)pyrrolidine-1-carboxamide;
1-((3S,4R)-3-ethyl-4-(3H-imidazo[1,2-a]pyrrolo[2,3-e]pyrazin-8-yl)pyrrolidin-1-yl)-4,4,4-trifluorobutan-1-one;
(3S,4R)—N-(2,2-difluoropropyl)-3-ethyl-4-(3H-imidazo[1,2-a]pyrrolo[2,3-e]pyrazin-8-yl)pyrrolidine-1-carboxamide;
(3S,4R)—N-cyclobutyl-3-ethyl-4-(3H-imidazo[1,2-a]pyrrolo[2,3-e]pyrazin-8-yl)pyrrolidine-1-carboxamide;
(3S,4R)-3-methyl-4-(3H-imidazo[1,2-a]pyrrolo[2,3-e]pyrazin-8-yl)-N-(2,2,2-trifluoroethyl)pyrrolidine-1-carboxamide;
(3R,4S)—N-(2,2-difluoroethyl)-3-(3H-imidazo[1,2-a]pyrrolo[2,3-e]pyrazin-8-yl)-4-methylpyrrolidine-1-carboxamide;
((3R,4S)-3-(3H-imidazo[1,2-a]pyrrolo[2,3-e]pyrazin-8-yl)-4-methylpyrrolidin-1-yl)(morpholino)methanone; or
1-((3R,4S)-3-(3H-imidazo[1,2-a]pyrrolo[2,3-e]pyrazin-8-yl)-4-methylpyrrolidin-1-yl)-2-cyclopropylethanone.

14. The compound of claim 1 wherein
T is N, U is N, X is NR³ and Y is C;
R³ is -A-D-E-G, wherein:
A is a bond;
D is an optionally substituted (C₃-C₁₀)cycloalkyl;
E is —R^{e}—;
G is —CN; and
R^{e} is an optionally substituted (C₁-C₁₀)alkylene.

15. The compound of claim 14 wherein the compound is
3-((1R,3R,4S)-3-methyl-4-(pyrrolo[2,3-b][1,2,3]thiazolo[4,5-d]pyridin-1(6H)-yl)cyclopentyl)propanenitrile.

16. A method of treating a disease comprising administering to a patient in need thereof an effective amount of a compound of Formula (I) 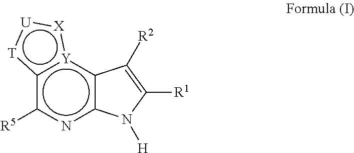
pharmaceutically acceptable salts, pro-drugs, biologically active metabolites, stereoisomers and isomers thereof wherein
T is N, U is N, X is CR³ and Y is N; or
T is N, U is CR⁴, X is CR³ and Y is N; or
T is N, U is N, X is NR³ and Y is C;
R¹, R² and R⁵ are each independently hydrogen or deuterium;
R³ is -A-D-E-G, wherein:
A is a bond or optionally substituted (C₃-C₁₂)cycloalkylene;
D is an optionally substituted (C₁-C₈)alkylene, optionally substituted (C₃-C₁₀)cycloalkylene or an optionally substituted (C₂-C₁₀)heterocyclylene;
E is —R^{e}—, —R^{e}—C(O)—R^{e}—, —R^{e}—C(O)O—R^{e}—, —R^{e}—N(R^{a})—R^{e}—, —R^{e}—N(R^{a})C(O)—R^{e}—, —R^{e}—C(O)N(R^{a})—R^{e}, —R^{e}—N(R^{a})S(O)₂—R^{e}—, —R^{e}—S(O)₂N(R^{a})—R^{e}—, —R^{e}—O—CH₂—C(O)N(R^{a})—R^{e}—, —R^{e}—N(R^{a})C(O)—CH₂—O—R^{e}—, —R^{e}—C(O)N(R^{a})CH₂C(O)N(R^{a})—R^{e}—, —R^{e}—N(R^{a})C(O)CH₂N(R^{a})C(O)—R^{e}—, —R^{e}—C(O)N(R^{a})CH₂C(O)N(R^{a})—R^{e}—, R^{e}—N(R^{a})C(O)CH₂N(R^{a})C(O)—R^{e}, —R^{e}—S—CH₂C(O)N(R^{a})—R^{e}—, or —R^{e}—N(R^{a})C(O)CH₂—S—R^{e}—; or
G is halogen, —CN, —CF₃, an optionally substituted —(C₁-C₆)alkyl, an optionally substituted —(C₁-C₆)alkyl-heteroaryl, an optionally substituted —(C₃-C₁₀)cycloalkyl, optionally substituted heterocyclyl or an optionally substituted heteroaryl;
R⁴ is hydrogen or deuterium;
R^{a} and R^{b} are each independently hydrogen, deuterium, an optionally substituted (C₁-C₁₀)alkyl or an optionally substituted (C₁-C₁₀)heterocyclylene; and
R^{e} for each occurrence is independently a bond, an optionally substituted (C₁-C₁₀)alkylene, or an optionally substituted (C₁-C₁₀)heterocyclylene.

17. The method of claim 16 wherein the disease is rheumatoid arthritis, Crohn's Disease, juvenile rheumatoid arthritis, juvenile idiopathic arthritis, psoriasis, psoriatic arthritis, ankylosing spondylitis or dry eye.

18. The method of claim 17 wherein the compound is a compound according to claim 7, 13 or 15.
